SEQUENCE LISTING (2 sequences)

SEQ ID NO: 1 - DNA, 134237 bp, Spodoptera frugiperda nuclear polyhedrosis virus
  ref <308>: DATABASE ACCESSION NUMBER: ATCC PAT-121214
  ref <309>: DATABASE ENTRY DATE: 2014-05-09
  ref <313>: RELEVANT RESIDUES IN SEQ ID NO: (1)..(134237)
atgtatactcgttacagctataacccatctttgggtcgcacctacgtgtacgacaacaagttctacaaaaatctaggttcggtcatcaaaaatgccaagcgcaaggagcacttagctctccatgaaatcgaggagaggactttggaccctctggaaaggtacgttgtcgccgaagatcccttcctgggacccggcaaaaaccaaaagctgactctgttcaaagagatccgtatcgtcaagcccgacaccatgaagctggtagtaaactggagcggcaaagagtttctcagggaaacttggacccgtttcatggaagacagcttccctatcgtgaacgaccaagaaattatggacgtgttcctagtgatcaacatgagacccactaggcccaacagatgtttcagattcctggcgcaacacgctctccgttgcgaccctgactacgttcctcacgaagtcattcgcattgtggagcccgtgtacgtaggaaacaacaacgaataccgcatcagcctggccaagaagggcggcggctgccccgtcatgaaccttcactctgagtacacgcactccttcgaagagttcatcaaccgtgtcatctgggagaacttctacaaacccatcgtctacgtaggaaccgactctggtgaggaagaggagatcctccttgaactgtcgctcgtgttcaagatcaaggagtttgctcctgacgcacccctgtacaacggacccgcgtactaataaacgataccacaaattcaaataaatcatacacacaattcttttaacaaaatttattaacattttacaaagtcataacatctaccattaatttatcaaattttagataataattgcgtaaggtatcagcgtcatcggcagtaatctgctttttagctagaattatattaatagcggtcatcagcgtagtgatagattcgggcggctccattggaagctgctctaatttttttttcaacaaaacatattgagttttcaaagattcaatctccgacggagaggccaaccaatcgttatcttcttccggtatctcgctaccgctgctcgtcgacgaaggttgtatcacgttacgtctatgtagaatactcatcgcggactcggagcgagccatgagtggtgcttccgcgggcagcgaagatttaggtttggtggcagcgggcggattttgtaaagctttaagtttatcttcagatatgggagttttacgtttgagcctttctctgagttcgctttggaaatctacaggtttcggcgaaggtttatcatctttcgtcgattgtactactactttagaagttggtggtgggggtaaaggaatttctgacgtaggaggtggtgacatcatcagtggaggaggtggagggggaggcggtatattcaccgaaacgggggcattcggcgtggaaattaaattaggcattggcggcggcggaggagcaaagatatccatctgtggcggcgaagttgatactttgatcggcgaaggaggaggttgagatagtattgcatcatcgttattttcatctaatgttatcggtgctgatggcggcggtggcggtggaggtggcgcataagattgttcggcacccatttttggcgttgactgtggaatggatgtcgagacgggtgctttattcggtaacgcagatgaaactgtttcggcggagggttttgtttgatctagagtaactatttctgtgaacagtttgttgatatcgttttcctcgatctggtataatttgtaaaggtctaaaaatgtttttaataaagcgctaagattacttatgttgttttcattgacaatgctttctaaaatattttccaacttagatttaaagcgactactgtcgttgacgtggcgcaccatgtttttgagttgcgtaatttttgcgctcatagctacagggatcgatgtacttgctggcgcagcatctacacggaccattactttattttcgtagatagcccaggctaattgtaagacctccattagagtgcgagaattcagagtcacttcgtcgtcatacgagctcatgttctcgtcgcgataacacaccgcatcgaacagttccgtagacacgggtttggttaatttttgaaaaagatccacgacattattctgaggattttgtagaaattcatacacggtggtctgcatagtagccatggccaagaacggacttactaataataataatatggacactactctgcaagagatcaccgacttttactcggacctcgaacttgattgtgaacataaaacactcaacggtagatacggtaaactatccgtgtggaaacacaagcccacacaaaaagtgtttgtaaaaaaacaaatcaagctgaagcactacaatgaagtcgagccgatgatacatacgctaatgaagaaaaatccatattacataaatttatattacagtatcacaacgttgaagagtcatgtactcatcatggactacataaagggaggtgatctttttgaactgctaaagatggaagagtatctcagcgtcgacgaaactaggctcattatcgggcaactgtgcgaagggctgcacgctctacacaaacatcattacgttcataacgatatcaagttggagaatgtgctgtacaatagatacaaacagatatacatcgcagactatggcttgtgcaaagttgcgggccagcaatcgtgtctcgacggtacgctagactattattctccggaaaagatactgggtcgccgatacgactatcactttgactggtgggctgtgggaatcatggcgtacgaactcatcaccggcgaacatccgttcaagggcgccgacgaagacgatagcttcgacattgaaaagttagaggaaagacaacaaagaaaactagtgtttaaaaatagaagcgttcctgcgaacgcacaatcatttattacgggaatgttaaagtacaatctaaattacagactaaataattacaacgacataataaaacatgtgtttcttaaaatctaataacattaatataatagtatcacaataaagctatatatcagaaacaattacaagactttaattttttggttttttcgaaggaggaggttgcatagagtcatcatcgtcatgatcctcgcttacaacatggctaatacgagtaatcgtaacgccaattttctttggtttaaataaatttacatcatcattgacaattattacatcattttcatcgtcgttgtcgctatcatcaagaggttcgtacggagtacatttataaattactggctctttttctaattttataacagttctgttgtcgttagggtcacttttgttaacgtactcatattgataataggttacaggttcaatttgcgcattactgtttaatagttcgcgtttaattttaggtttttgttcccgcggcggagtagacgatgaagatgatgtaacaacattatttgatgtaacaacattattagcggtcgcgacatcagtagtattgtcattgacattatcgatattagtattttcattaatattcatagtgtcttcgtccgcgttatcaatttcgttatcgtcgtcactgtcgtcactgtcgctgttattattttcgacatcagtgtccgcgttagtttcgactacgttttcatcatcttcgttatcactatgtacgctctcgacatcactatcattattgttatcgttattagtgttattagtgggagcgacaatattcgaagatgtactcattagtggaaaattagttagttcgttgtcggtgtcgttgtccatgttttctttgtcggacaactttttacacgcgccgcagcttttaattttgaatagactcttgaacgcgcatttagagcaaagtttgtggttacaatttttgtaatgataatagatgtgaccgcgtttacacttgtcgcactcggctataacgttgttaagtcggtacatcattagattatacgtatactttgccgtaagagttttgatatttttttgtaaagaaataaaagtactctgtaccgcgggaatgatcacgttacgaatagactcgatagattttctagtctgacattcaaaatctattgttttcatgtctggcgttaaagattgtacgcgttcgtgcaactgagtacaatcgatgagcacttcttccaaaggcgcggtcattaaacgcgaaacgaaattttttataatagaatagttttcatcaatagttttgttactgtaatgatctagatacctctgcatacagataatgacatgctgtaacactatagatttagtgagcaaagttaaaaggttacgaaagccgttgctctccttgctaacctccatgtttgcgatgacattgtaaaattcagtgtatctatcggtgaacaggtaatactgcttgtacgtaatcttaacaaacttccaagttttggtgaggatgtctacctccccgacgtgatgaaagggggccgtctgaaccatctgcaggtacacactaacacttttgcgcacggggttgtgcgaaaattgtatgtgtttatgaggcttgagcgcctagaacaatcgaaaaaaaaaaccacatgaataattttgaaaataaccccaaccacaaaaacgttaatgatatacacatacagatggacagaacaaagaacgataaacatgaacagggataaacatgaacaggacactcacctcgttattgttagaagtccagaaattcattatggcggtggtggcgcgtaacagggtgaatcacggtgctggcggtgctgatgaaaaaggcactcggcttgatgcacagcaaagatagcttcaaactgcaacgcttgaatgaaggcaaagatcgttgtatgggcatatataccctattatcgcggatgtatgatgatatcatctgcgataacgtcggtcacgtacaccgccgccagtcacgtacgccatagtcggtcacgtacgcacggtcgttgcactcggtcggtcgaggtcaccgctcacgccaccggcgcacgagcatccggtcacgtaggccattggtgccgtgtgcacgcagtcgttgaccgcattaggttcaaggaaggcctgtgcgtcatcaacttttttgcagtgcaaaaaagtaccttaaagtgtagtatatggagagcatattctacagtgtagactatgctagtataatagtctacgattcgaactattgttctgtatatagatatgccatatctaatcaacatttttgcactacaaaaaagtacactaccatttttatcttgaaaaggaggggatacgggcttatatataaagcgtcggcgacgatgagcatcagttcaagttggttgaacattctcaacgcactaactaacgtttgtgaataccttccacagtactacttcaccaccagcatcatgtacgcaaacgagcacatcaactcttcggtcgagcagcttcgtggcttcgtggacgacgagctgttggacgagctgggcttcaactttgagcaacagcagcagcagcagcgccaggcgaaggaggagcaagacggcggcgacgacatgcagacaactaacgatgacgctgattctatttgtgagttaactaacgagctagataatttgctctgtgaagatgaaaacgaggaggttactagtaactctaataattttgactctaaccctccgctcatcgattactctgccgacagtgacattaagaaaactatgcgcagcattcatactacgtttggcatactggccgagctagataagttgcgcagtattataaaggttaaacttgcaaactttaaagacaacaatgtaagcaatgataccaccaacaacgacaacaccaacgtaagcaatactcttgacgaaaagattgcaaactatgagcctagcgatgaaacttatgaaccatcttcatcagaatttttggattcggaatccaagtttcgtcctgaagaaatgaaagatatcatcataaactacattcatctgtacttgttgaaacatcacaaaactaactttgtctctgattccgttaccaaaaccctcagagaagacttgagaatttggatgcttctcggcattcagctattgtctaacgtcgcagatgttgaactgtataatgttgctactaaacaatgggccgatatctttaaagaacttacagatgccgtcgagacactgaccattgactctaaagagttttacactacaaagagactgcgcaacgttatcggagacatcttcttcttcgataacctttacaataacggtgcgtgtaagtttcacgaaaatgtttctgaaatctttgaatacattgataaatgttttgcaccagtagatgacgatatcgtgtctcgattggtaaatgatgatattctgtctcaattgcaaactatgaacaatctggcatccgaatgctgtgtatctgtcacgccgtttaaagctgaagacgtctctaacgatgataacatgccttcgttgtttagcgagtcgcgtgttaagagcacacgcaaactctcgcacaagtttaagccgcgtcgtaactctaaaaaggcccgtcgtgctctcaagtttgagcaaatgtacatataattgtattgtgtgtaaaaactatgtgttattgtgataactattattgtaaaactgtgttataataatgttgactgttatgttgtaataatttttatgtgtctttaaaatgtgtctatatattgtatctctatatattattgtaagtttgttattaaaatttaataaatacttttaactcaacaaacatggtttttcgtcctctccgtaatgttaacaaaacctatagcaatattacatcatttgtggcagacaacaatagtgtagtcaatgcgagtccatcaggattcagaaatgtactcaataaacccacatctgtacctgcgggaaataatcaaatagtacccggctataacttgccgaacaataactttatttctacatcagaactaaacagtattatgagaaacaatgacactaacggtatgcgcagagtctttggtaacacacccaccaacaatgactacaatagtttaactcaaattagacgagcagataacatacccgacgctaacatgcacagtaaacagttgaaacgagacgctgtgaaaaataacaatccttcgactcgcaccaacactcctgaaggtatacaaaacagccttaaccaaaatccctctctcaacgattacctacagggtatgaaaactgccggcaaagtggctctgctaggagtaggtgtatacttaacgtttgaaggtatatctttgatacaagacattagggaagcattggccagaacgggcggcagctaccacaccacaggcatagacggaggtaacgaagtttccgtatgtttgcttaggtacaggacttgtcgtttaccagatgttatcgataatgaaaaagtcactatatgtcgtaacgacccactgattgcagacattaatcaattacaaaacatatgcgcaaactttaactatgaagaagaaggtagtgtatgtcgcgccagcgaccccaacgccaatcccgacagtcctcaatacctggacatatctaacctaggcgtagatcaaatgatcaattgtatagaaccatacgactttggtgatcttatcgctgaccttggccttgacgggttgctaggtgaaaatgggctgttcaccaagtcgtccaatagcagtaaaagcattagtgatagtttgttgcccgcaattctgatgattggtatcattatctttatcgtgttcataggctattttatctttaaaagactaggcaacacgcaaacgcagactatacaactagaacctatacgtactagtgtgccgatgacctcggtacctattcaggtggtacgataagaatcaaccgaaaaggatataaaaggacgatcctgccgaagcactacacttcaacatcgcccaacaaactggcagcacgagcagcaaatactcggtaagttttttcttttaacaatctttaccattgattatgttgttatgattgttgtatagtgtgttgagctaaaggggttttcttctttttccatagaaaatgaagaagtttatgtacaacaagggcggcaagaacaccacggtgtctgccaaagtcaacacgaatctctcgccggacgagaagcccaaagatctgcgcggacgtttcctcagcaaccaaaattatcggctgatgaagtccgttctccgattctccagcgagtatgtgcaaggcgtgcggcgcgtcaatgatcttgtggagatgaagtgcgacgacaccaagtgtaaagagatcacgacaaacgccaagtgtcaaggagaatgcggtaaaatttttaacaatggtaaatttttgttctgtgtctacgacacttcggctgttgatgcagcagcttcatctagcggcgaagccaagtttaaactagtttgtaatctctgttctaacgattattcgtacaacaacaggtacgaagttttacaactgtttcctaccgtttctcagcaagtggtagaacgtctatgtgagattggttttttgactaaatacatctttcccattaaactaaactataccgagacacaaactaaaaccgaagtcattaactatcatgatttttacaagatggttaaatctatcatcaaagaaaagaaaagcaacgaacaaatcacagagattaaactcagcacctatggtcgagacttgttcgtcgagactgatgatttttgcgttatgagaaaagacagtaacgagtttaacgaaaccgtctacgagttggacttctttcctaaacaaagttccatgttagagtttttgaaaatttacaaagacaccaaacctctcacatacttttatacagttaaaaaacgagtctacttgagcatctttgattatgcagtgtattttcctttcaagtgcgtactattttgtaaagtttgtacaaagaacaaaatgtatctaaagacacatcccgttttgtattgtagcaagtgcggttttaccgatcccatgttctttaacaattccgcttttctagagtctaccatatttctggctaaatgtgtaaagtacaaaactatgccagtagcccgcatcttttactacgacatgactttgtacaaaacttttataaagaaaccttttgttaaaaaataactccatgtgataatcgatagttttgtaagatctctatatattaatctcttagcacgtaagcaagcaatgttaatcgatagttttaagcaagcaatgttaaccaaatattgatactctttagttttaagcaagcaatgttaaccactctttagttttaagatatacattgatactctttacattataatctctatattattcttttttatattaataaatttttatatcaaacatataatttttcattaactaaccaccatcaaacactattcaactaaccaccatctttgctttcggcgaaacacttcactgaaagcaaacatcaataggatgatgacgtaatatacttccaactttgctttccgcgaaacactttaccgaaagcaaacattgttaaaattatgcgcaatagaacatgatatcattccaataaaaacatgacatcattctacctaccaagaagatgacatcatgtatcaaactttgctttccgcgaaacactttaccgaaagcaaagatcgaggacatcgacctacagtagagttgttaatctttgctttccgcgaaacacttcaccgaaagcaaagttcatacaaaatgatgatatcatcacatcgcatgacatcatcctaaaacatgatatcatcctaataaaatcatgacatcatcctactgccacaaactttgctttcaacaaaatacttcgccgaaagcaaacatcaatgttaaaatgatctcatcatatgatgatgcaataaaaacaggatgttaaataaaactacacgatcgtcgacgttatgtttgttttaaagattatagaacttttagaaaattattctttaaagttttaaaactaaatagtttgttacagttaatctttgcaatcgcatacactttatcgattgttatagttaaactttgattgttatagttaaacttgtttttaattggtaatctttgaaacgataccattttcaagattgtgaaaatttaacatttgttaaaatttttcaaaatttccactttgttgaactttatgtagtttccaagatcgttaatgaaaaacattatttagtgcatgatcttgaactaacaactatttgcaagatcgtaatgtacaaacgcttactaaaaattctttgagatggatacaaagtcaatcccattacacaaaacttaaagacgatcttgaaaacaccatagatttcgagtaaacggatttttgtaaattttgttaaaaagtttgcaaatcgcgttacgacgaaaactctatcgttttcaagatcatattagttaaactcgataactaatattagttaaactcgattattagttaaactcgataactaatagttttcaatagacaatttcgaaaacaccataggtttcaagataataagatttgcaaacttttgcaataaaatttacaaaaattcatttactcgaaatctatggtgttttcgagattgtttattgaaaactaaaaccaagtgctccatgtttgctttcgtcgaagaatgtcatcgaaagcaaagatcgactttatctctgctgacgtcatttgacctcgatcgctaactgcgcagtattaaatgcgcaataaatctatttttagcgatgatttaataatgcggacattcaatgatgtttgctttcggcgaagcgtttcgctgaaatccaagatggttacacatatctattgacgtcatacaattgtttcacaatatttaatcatccatacaagatccgcgtagagacatatcgtttcgccgaaaaagcaagggtaacgattcgttaccctttgtgtttcgaggtcgatgatatgattatcggcgaaacgtgaccggtctacgtgacttttgtaacgttccgcgcattaacattaattacacgaaatgggttatcggtacaatgattgtgttctataagcgataatgtcgttcgacttggtataaaagaacgatcctgcaagtgatgctcacagtcttgaccacgaacactcaacatgttgtgcctcaaacaagttttcttctacatcacggcggcagcgttcctgctggttccgagcgccaccaccagcaccaccagcaccaccagcaacgctgaagatatcatcaaagttactcagctgccttcaacttctggtctgtatttccaacacattaaccgtatgcagtttgtccagaacatttggcactttgttatcgaaatggaccatggttctgtgttctaccgcctgcagaccatctataaccaagctcaaaagatacacaatgaattaacgaaaatctattataaatcgagcgataataaacgttacgatgattgtaatagcatgagatttataaaattggacttggaaaatattatccataatattatacccaatcttgcacagcaacataacctgctggatcaaaaagtacccgtcactccgtccactgccaacttgaagaaggttaatttagaggaaaaaacaaacacgcgttcgaaacgtggaattttgaactttgtcggccaagtggacaagtatttgtttggcgttatggacagcgatgacgcccatgaacttcacatgctcgctaacaccaccaacagtttgaattcccaagttaaacaattgacggacgagcttattgttttgagcaaatatgtggatcagttatcgagttatagcgacaaagaaactgtacaatgtagaataattgctgaaaattttaacattttgtgtagacagttagatgaagtagctacgttgtacaacaaactagatttggctgtggacaatgccaagttgaatcgtctcaactcgtttgtcatctctcccgaacgactcctcaacgagatgaggaacgtgagcggtcatcttgccggtctctcttggccggtgccgcttaccaacaaggcggtgcacaccctagtcgacaatgtgatcaacgtgcacgtgttcgttacggccgaacgtaagttgttgtttatcgtcgaagtgcctttagtgagcagtgaagcttttgacgtgttccatagtattcctttgccgtattgtgacagtgaccacaaatgtgccgttctattgcccgatagtaaatatttaggcgtatctctcgaccgtagaagttatgtgcgtctggacgatactaatacttgccgcattaccgataaaatcatgttgtgtttcaagccacaaataatctacgatgtgaatcaggccaagttgtgcgacattaaaattttcataaagaacgacaaagacattgattataatagagactgtgacattagagtgggcaagtttgagagtgaattgttttacgcgacctctgactacaataattggttgtacgttttacagaacgacatcgatctcaatattcaatgtataccctccgatactcgtcctgatggtatgtcaattgaacctgttgttttgcacgccggtgtgggtttgatccatgccacgggcaaagataattgtaaacttactacgaaaaagtcccgattgactgtccacgatctgtacaataatttgaatagtgtcatcgaaatacctatcggtttaacgttcaacttttctttggctctacaggacattgataaaattactgtcgataatatgaaaattaataacgatctggaacacaccaaccttcacgagttaactaaccgtttgtacgacttgcggcgccagatcgccaacaataccatcttttccggtgatcaaatttacgatgataacaatgacatttttgccggtgtgtcacaatggtttgccggaatcggtattaattttcattacataaaaatcattgtcatgtggattatactggctctgttaactttagccgtaattaaaatttatcgtacttgttgttctggcgcgtgtagcactttgtgtggtggctgctgtagaaaattaaagtttcgtcgtacggctagcgatcacactgtagtgcgacgcgacgaccgagaaatgtattatttgaacactttgcctagaaaacaaaagaacaacaagtttcagactaagttagatgctcctagaaagtatcatatggaagatgaagatatcgatattgatcttttacaaagaaacataacttttgatgataaaatataaatgtgtatgttataatgtaaatttatgttaatgtaaatgttaataatggatctgtgtataaaataaaaccaaaaaattttttatattaatatatttttataaaaaagtgtatacagttgttttatttattgtccagcaaactatcaatcttgccgttaataacatcaatctttcgactgttgattactatcaagtctatagaatgtttggtcatgttgtgtaacttttccaagttttgcactatagtttcatgatagtcagacatgagttttgtgacatcgtcaaactttgtcgtagtagtttcgtagattaacaatagaaattttttaacgaggttaatttcattggccatgctgtttgcataccacaagtatgctgcgagcaaaactagtgccgctccggaaaaattcatcattgtcgtagactccctcttactttttacgattcatcatgtctttaatggacttgaaaccgtccaacattgtgtcgttgctgaaggcaatcttattggtcaagtccgcttgcatatttttaatattttccaaagcgttattaatgtttgcgaatagagaagcgtgttctgctcgaatcatgtcacgcaatagttcatcattattaccactgttgttgttgttattttttaaattgtcgtagtttgattcgtacaacgtagaagattgattattggttatacattcttgtagtgtacaaagttgagttttaatatcgagtagaggatctacgatttgactctgtgcgcccaccagcaaatcgcctatcaactgtttcaaagttaataattcgggacgatgaacaccttgcggtggcggtttgtacattgacagatatttgccgagagaaaagacgtgaacgtaatttttattgttgcgtgttagtttctgagagttgtgagtgtttgcccacatgaccgcgggactgaatccccgaatgctggctatgggagctatgagctgtgcggctgcgttgagttcgacgtaaccgtcacgatcttcgtcgctgttttcaataatcaccacttctagcagtttatcattgtattgaaattgtaacgacgaattatcggaggcggaggcggtaccggcgctgttggtggcaaagttgttacttgcgtagctatcgaacatttttcaagcatcttattgtacacagatggataagataatttcattttatgaagtcaatctacccatcgtatcgcaatatgttttattcgtcttcgccctatatgcgcacttaccgcagttgcggtaacgacactatgatgaattacaacaacaaccacgaaacactacgtcacgatctgcaaaccttaaaatatcaagtttacgaaatgtgccaacagtctaccacggatcgtaacctatgcgaccgcatcaaatcttccatagacacgtcgagcagtagttacgttgttcgccctgtagtgccgaccaccaaaactattctcagcaacaacaacaagaaaaatggcggcggaacagttactgttgtggacaccgtcaaatattgaaactatcgacaaaaactcggactatagcgttctgctaagcgacgtgccattgttacaagtgaccgcgttgacgcctttcctcgacaatggactacgtgtcaagatcaacggtctacggttgttctacgtgattaaaaattccatgaacaacaacaacgataaccacaacatgggcaatgtaaaagttttcaaaggaaccaaaagtaaaaaaaacgtgtgtttcgccgaagcgtgtaccaagtccgaaatagtacaagtgctacagagaaaactgaacatgcccgattgtatgcaaaaatttttgatggactttcaagtttgtcctaggggcaatcgttatcgtaaacgctttatattcaattcgtacatagtgaacgttttaacttgcaccaaatgtaacaagctatgtcttgtcaaagccatggcacaattatacaaccacgacgataaatgtgtacaagagtttgatagattgttattcaaaaacgcaactttgtacaagcctccgaactgtgaaaacttgaaaaacaaagacaaactatgtttcaaaatgggcaactgtaaaggaactaatcctatttgtaactattgaaataataaacatgtgtgttataaaaatacaaattttttttattaattacaaattttcatttataaaagatttcacggtgtccacagaagtgttcacctttagcaaacgtcttgaacgttttgtgtaatcgatgtgtttcatgtccaactcttcgttgatacaatgcatggccaggagcgggttaggatgtacgctatcgtagatcatttccatattgttaaattttcgtttgagagcaccgtgtctccgctgctgagccgctagaaacgccacctctgtgctctcgccgtcctctgacggttttacaaatacagagagacgaggatgtttggtcgaatctcgcggaaaacgaaccatttcatagcgaggaccgttgccgttgttgttacacgcgttggcgttgttgttgttgttgaaaaatgatgcgctgtcgttgatgtctaggattcctgacgaatttgaatcggaaaatagttgctttttatttctatggtaatcctgcaaacgtttatacaggtcatcgacgttgttcaacatgttaactttattgtgtaactctgaaatttgtacgttgaaccattcgaaacgttcgatgacttgagttttgaactcatcgtttgtacgtacaacttcgctgttgtgctctttaatggtgtcgacagcggccaaaactttactcaatttctcatctatcggcgaaggtataaacttgttttcgagttcgagatacacatcttcgatgatccacgttataaactctgctttgttcttgaaatccaagttatccaacaattgtactacgccatgtttgttgatgcaaattaaactattgttgtcgtcatgatgattgttaaacactaactcgttaatggtgcgtttgtacttgtcatcgaccaatttatcgatggctctttgaggatctgggtatcccatggcttcggcaaactttgctcccgctatccacacttcgtcgttgaacaaatagttgatcgtaaacgaaaattgatcatcaaactttaaacgtttacttttaaaaaggtacgagaatcctgcaagtcccgttctttttttcttcggtaatgctgtttgttcttcttcttcttgttccttttcgttgttgtggtggtcattttcatcatcatcatcgttgtcgtagtatccaaataattttgcgagaaactgaagcaacattttttaagatttggtagtagttagtaaatacgaaaacttttttcctttatgattataactgtagggtacacgaatttgactgttattacaaaacacgtgcttatctacattgggccaccaatacaatagtggtttgtcgtcgatgatcttctcgtctttgtacttggcaattgcttttaaaaatgattcagcaaaacacccttcgggcaccgccgatgtaacgtccaacgcgtcgggcggcacaaacaccttatagtattgttcgcgcgtctctttcgaagcgtgcatgggaaatttatctattcgaagccaaacgtgaatacctctattgccgctgtgcatgatcctcgaaatgttgttcttgaaaaagtttctaaacgtttcggctgccacgttaatcttgatttcgagatctttggaattttccgcttccacgtcaacgtcgatgacccattctctgccgccgttgtcctccaaaggtttcacgtgtacgtcactgatgcgatagcgtttcaagtattgcgcaaattcatcgcaagtggcgaaaaatacgtttgggtggtaccatctgtgaccatcgaaaaaaccaaagcgacgacaatcgttgaaggatacgttgtgccacatttgttctatttgatcaggagaatacacaaacttttttctaaacatgattctcgcccagaggtccgagtgctgaatgcaggataagaatgatgccatttgcgttattaactagcgcgcttatcacatcatatattgaaaataatataactttattacgcagcaacagcgacgatgatagcagcaacggcggcggcgaagataaactaaaagatatcgaaaccgtgtttgcaattatcatggaggagatatcgaaaattgaaaaaacggaaaatagcgatgttagctacaccaaaataattatcggcatactagtgctgatggcgttgttcactttgaaaacgaaaatataccgcatgtcaagttgcttaaggaaaaaaagactctacaaagctcgtgaagaaatacccctcgaaaatattactattcaggaactcaactacaatgtaatagaatcgccggcgacgtcttcgcctccaccacctttaccccctaaattacttgagcgctagacgccagttcgttaatcataccgcacgaatttactccgcggcgaacgcggacgtacccatcttcgccgtaatcgctaccccaagaatttttgatgatccaatagggtacattgttttctacgccgtaacctactaggagcacggcatgattgagaccattgtttttgcagaaactgacaattcctccgtaataatcggtaagatctaccgcgtcgacggcaatggctatgggtccgacgtaacgtaacaaatcctcgagacgctcctcgttcatcagaacgtaacgatagcaatttcgtacaccggccgcaaacttgtgaggtttcaaagcgcacggctgacgctctgctttgtatggatagtcaaactcttgttcgactccgcccatcctcataatttgttcgtatgcggtgtgaatcaacccaccatcgcatcccatgtctacaaaatcgcaatctaccaactgttgttcggccaagtctattaacctatcgtattttatggcgtactggctttccagagcccctagtcctgcaaaggcccaacacgcaccgcacataccctgatctttgaccgaagtgactttgttcaaagttcgccaatcaaaattggccggtcgttgtcgttgagccggtccgtcgaccactaccgtttcgcaaaaatttgctcccaactctcccgaagccagacccgtatggcggatgactatttcattcttggtcatgtccgcaaaacgattgatcttgtaaactgcagaatcgttacgactattcttttggtttatcgattcaatgttgtgtctgaatatgttgtagcgatactttttttcatcttcgctcttgtattgtttattgtattgcgcaatgaatttttcaaagtaaagcggtgcgctattaatattgtaaagcataggtttgttgttgttgttgtgatttgcatgattatcttggcgcgttaacgccgcgtttagtaacagcaaaagtatgagtaatttgttcattataaattcaataaacaataggtacttaaatacaccgatgtttgctttcaacgacaattttagttgaaagcaaagatgaaataaaacaattgaaaaatttattagttatttatttaattaataatcatcaaaacttcccatatcgtctaatatatccatcaaaaaatcatcttcatccaaagaaccgtcaaaatcaacaaattcaaatggtaaatttaacaaacagtatgcacatttagaacaaaacaaaaccatatagaaatctttccacttgttttgagttacaaatttttttatgcttaaaaggcaaatcgagcgttcgtaacattgtgcacaatatttactcaaaatataagacgaatgttgaagacaatttgaataccccatcaaaacttttgctaaattcatcggcattaaacattcgggaaatgcgattttgtgaagtcgaaagaatgcgtaaaacggcacagttcttaatttgcgcatcaataataatttagaagttgaagaattgctagcttcgaccaatctcaaaattttatcaaaagcaattaaacgattataacactccacaatatttttcatatttattcccgttcccaaatatcgcattaaaagcgtatcttttgtgacatcgtatgaatcttcctgttttaatgtatactcgcaaaattttccatcaatcaacagaggcaaattcgtaatgtcttcgttgttgaaaaatttttttagcaacgtatcctcggccataacataattttcgggttgagtctttacgtatgtttggaaaagacaatccgccagcagcggcaatcgtgcgacgcagtcttcttttaaatattttgccaatcgaaagtcacgattcgggcaatagtcgaaaggacgagtatcaaaatactttttaaatagtccttgggacagtagaatattatctgcgcgttcatgatctccggttactgcgataaatgtatcgtaatccgcgtattctagaattttccactttatctctgtgggcagcaaacccaacaaagattgttcctcgtcgttgttggccacagaatttgtcatcatcgttccacacttgatctttttatagtgtcgcgtcaagcaaactactggctctgtcacgttcgccaaccgtttatatactacacaaacacatatctttatctttataaggtttaattatataatgccctatattattacgttgttcttggcttgcgtggtgttatctgccgcaaaagcagctattcccggcgtgccgagtattgattggtcggatcgaaactatgcactcgtaaagattgatcacgaagccacatcgtatgaaaaattgatcaaaagcacgtccaatgtcgaagtgccggtgtcgtggaacgtatggtcaggcgactctggtgacgtagcttacgtactcttcgacggagtgcaaatgtacaaaggtgatgccggcgtgaaaaaagcaactgtgaacgcgagcaaaggaggcaaatacaacatgaccgtgaaactttgcaatgttgacggctgttcaacaagcagcgcggttcaggtcaccatcgccgacaccgatggatctcatttaaaacctttgatttatgactacattgaaaataataaacgtttcgagaagcgtaccgataaaatcattgctgcatattttgtagaatggggcgtttatcctagacaatttcccgtcgacaaggtacccgttcccaatttgtctcatctgctatatggctttgtgccaatttgcggcggagatggtatcaacgacgcgttaaagttgtcccagccggatagttatttagctttgcaacgttcgtgcgcgggccgagataattttaaagttgccatacacgatccttgggccgctctacagaaaccacaaaaaggagtatcctcttggaacgaaccgtacaaaggaaactttggtcagttgatggcaacgaaactggcgtatcctcatcttaaagtgctaccttcaatcggcgggtggactttatccgatccgttttatcatatgcacgatccgatcgtacgccaagttttcatcgattctgtggaagagtttttgttgacgtggaaattctttgacggcgttgacatagactgggagtttccgggaggcaaaggtgcgaatccgaacgtgggtgatcccgaacgcgatcgagaaacgtatacgcttttacttcaagagcttcgtttgcgattggacgcactcggcgtcaaaactaacagatactacgccctaacttcagctgtaagtgcgggtaacgacaagatcgccgtagtaaattacaccgaagcgcaaaaatacttggatgtaatatttttaatgacgtacgactttaaaggctcatggagtaacacggaactcggacatcacaccgctttgtttgcgccaacttggcgtcccgacgaaccctattgtgccgaccgcgccgtagaactaatgttgaatcaaaacgtgcccgccggcaagatagcgctaggtgtggccatgtacggccgcggttggacaggtgtggtcagcgacaatgacgacaatccatttttgggcatcgctaccggccccgtgcccggcacgtgggaagcgggtgttgtagattatcgtcaaatattgcacaatatttcacaatatgattacgaatacgacgacgtggccaaaggcgcgtacgtgtacaatcgcgaaaccggcaatctaatcacgtacgacgatccaaagtccgttgccgataaaacaaagtacgtgcttgatcacgagctggccggcgtgtttgcgtgggagatcgacgccgataatggggatttactgaacgccatcaacaagggtttgggcggaaagcaagcagattatttgtatgacgaacaacaacaacaacaagttataaaagatgaattgtaaaaggtttaaataaagaataatacaaattacatgtaaaatacatttatttccatttcaattcgaaataacaaagttgacggcaaatgctacactttggtgtgatgtccttgtttttgatgaatttgcgcgattttatgtagaaaaaagtagcattttctcggcagtcgtggcaaatcgctcgtttgtcgatcgatgtacaggttatatgcattggtttaatctaatctacaacagtatatctaatcattttaacgcaatcggtcgtatataaatttgcgcagtgaatttactgcattgttatcaacataatcatcagcatcatgaagaacgttagcaacattgtctaccaatcgctgtgtttcaacgcgaacgcacccctcgtactgttcggccagcgtttgatacaggacaacgagcgccaggcaaagtacgttttgcgcgatcttgtgtccaagggttacagcttgtgcgttgtcgttgatcgtacagagataataactaaaatacaccccatcactaagaaaaaaattaaaattgacgtatctaataaatacattaataggatgaagaagacgctgataaagtggggctatcctgtgaaatttgcaaaatttgacgagtttaaaggatacaagtacgacattgacacggacgactgggaccgagtcgactacaatggaccccaagaagacatcctcgtgacgatcgacggaaagcaagtcacgaaacaagtgcccatgttagttcgtaacgattatattgtaaactattatacagacttgaaattgaaatacaataaaaattcataatgcaacacttgcctattattcaaatataatggtatcatcctatcttatatctttgcattgtactgttcaaatacatttgtattgttgtcattgtctcaaagacgttgtgtgcagataaaatattatcggcaatctacatctaatctttgttagtgttcgtgtataaaagccgtacgttatccttgtggttattaaacacgcaagtaaaatgttgaagaaaactacactgttgactttgataatcatattcctcgagttcagccacagcatgctgaacagcctgctcggcatacccagatacaacgtcttgaaaaatctgagcatgtccatgtacggggacaaatcgttgctgatgacgcgactggaaattattacgggagcgattgcgtttgtggtgagcctgttctttgtcctgagcaaattaaagaaggagaacgctttgcggtataaagttattctgctgctgctgccgattgtgggcgcgatagcctcggcggtgacggtgagcatactcatcgattttcaaggcattctgacggcgccatgggtgtacacggtggcgatcgtgccctcgatcacgggcgagtcgttccttttcgagacgtgcatgtccgacatcgttgcgctcatcgtcaaagacaagcagcagcgcgtctcgctgttcctgtgggtgaagggcggaaagttggcgggcatatgtttcacgcaaatcgtattgccgctgctcaaagtcggcgaagacgatatgctcgagatcgccacgcccgtcgtgtgtataatgttgagcgtcactggagtgctgttgacgtccgtagtggtgttcgtgcccgaagacgaaacgactgtcgtgcaaataaccgcaccgacgccgacgccatcgatagagctgcagcagcgcataaacgaccctgaatttcaacaaaatctcgaagaaataacctacgtcattgacgatgactatatcatcagagaaaaagaacctgaattgtcttccaatataaagttgctgtgggactttgtaaagagtttgaacagatatcatgtggtgttgtgttcgttgatcatgttccacagcgcccagaggggcgagtacaagttcacctacttgtttctggccaacaatctaaactttgagcctcgacatttgcgcatcataaacggcggccaatacctcctcttcaccgtctccgtgtacgtgatggggttcgtgattcggcacactagccacacgcacatcaccctcatcgcgttcatagtgtcgatgctgttcagcacggcggcgagggtgtgccagattgtttcgtgggacctcaacaactttattgtatggtgcgcgtccgcgttgctgtcgacgccgggaccgctggcctaccaggtgatgcagcaagtcatgtacaagaaactctacaacgaacagctcgtcggaatcatactgctctcggtggacaagtttgtgtccatacccatcatacagctgtaccagatcgcgtaccagcaactgcagatatcgccgttttacgtgacgctgacgctcatgctgctcaccaccgttggtggactgtcgacgaaaaccatgcgaacgtggttaaactcttaaggttattgattgtattcaaaaataaaaactatattttactgtacacaattttttatttattagtaccattataagtttaaactaaacgcataaaatgcacactttactcataatattatactttgtcgtcgccgctgtgaatggccacggctatttgtctcagccggtggcgcgacagtacaaatgttttcgagacaatcacttttggtggccggacaacggcgacgagattccagatcccgcttgcaggatcgcatacaaaagcgtttacgcaaagtacagaagcatgggcgagtcgcatggcgtcgctgccaacgcggcacagtacatgttccaacagtattacgagtatgcggcactagccggccccaactacaacgacttggactacataaaaaaacatgtcgttccgcataaattatgcgccggcggtgccgatgaccgcttacatccgtttggcgacaagtcgggtgtcgacgaaccgacacctttatggcacgccacctcattatttcacaattccaaagaaaaataccaaagcggccatcaaatggtcattcacttttgtcccaccgccgtgcacgaacctagctatttcgaagtgtacgtgtctcggcccgaatacaattacaccaccgaactgacttggaacgatctagaacttatcggaggagacggttcgcagttgataaagaacgacggcagcgacgattcatgtactgccgacgaactgtatactatacccgttcgagttccgtttaggtcacaaaaattcgtaatctttgttcgatggcagagaaacgacgttgccggcgaaggtttctacaactgcgccgacgtagagttcgacaagtacagcttacgtcaccaccatagtcattaaaattccatttgcgattcggccatgtgcagctgtagcagaaacgattggttgggtcgaatttcgcgtttgtccaagacgagctggtacgcttcggacaaactgatttcgcaatatttcataatgaaatacaccactatggtcgcgctgcgagatatacccgcgtgacaatgaacatatacgtttttgccttcgtccattttgcgctttataaaattgtacaataaatcaaagtattgcattatgtttgccgattgattgtcgtgaatgtacacgtatagataatcttctggcaaaaggccaaggtttgtaacgttaatcatcgaatcgtcccagatgctgacgatggcattgatatttttatcttcaacaaaaaccttgaaatcgtcccagttgtaaattattccgcccagatataaattatttaaaatttgtgtcacgttcaccaagtttccgttcgagagacgcaaaaaattattgttgctatccgaagagttgtccatggtgacgtaagtttcttatatactggtttccgtttggccaagtataaaataaatttttggggtcacctacttactgtgtttggtcatctgtcaccatgaacggttgctttggtgtcctcattttattttttgcgatatgtgggtcgtcggttgtgaaatcggccaggattttggccgtcttccctacaccctcttttagtcatcatagcgtgtacaaagtgtacattcaagcactggtcgaaaaaggacacgaagtcgtagtcgtcaaatccaccaacaacgtaaactacaaagatattaacgacaattacgccgacgactatgaagcgacacgctacaacgtcaccgaaattgatgcgacgctatcgcaagattatttcaaaaaactcatgaagagggcccaagtgtttcgaaagagaggtctcgtcgccgacagctactctgtcaccgcagataactacatgggaatcgtgcgcatgatgagcgatcaatttaaactgcctgctgtgcaaaaatttttaaacaacaaaaaacaaaaatttgatttactcatcaccgaagcgttcattgattacacgttggtgtactcgcatctattcaacgatatacccgtgattcaaatctcgtcgggctacgctgtagctgaaaactttgaaactatgggtgcggtcggcagacatcccgtctactaccccaacttgtggagagataaattttacaatctcaacgtgtgggatctgatcaacgaactgtacgtcgaactgaggttatacaatgaattttataaattggccgatcaacagaatcgtttgttgaaagaacagtttggtcaagacacgccgaccatacaagatctacgcaatcgagtcgaactacttttcgtcaatacgcatccggttttcgataacaatcgacctgtgccaccgagcgttcaatatctaggttctctacatttgacccacaaacatccgaaaccgatatacggcacaatcggcgaactcctcgataatgcaacaaatggagcgatttacgtcagcttcggctcgggcatcgacaccgaagagatggagtcagagtttatcgaaatgctactcaagacgtttgaagcgttgccgtatctggttttatggaaatacgatggctatttgaatagaatgcctgaaaatgtgtatattcaatcatggttcgagcagtacgacctgttgcaccataaaaacattcgagcattcgtcacccagggaggagttcagagcacagacgaagccgtcgaagcgctcgtgccggtggtcggcatgccaatgatgggagaccaagctttcaacactaacaaatatatggaattgggaataggtagagtggtaaatacggttagtgtaaattccaaagaattgattgacgcaatcacggatgttgtcgaaaatccaaattatcgcaaaaaaataaacgaattacgccacgttattcgtcatcaggcaattacgcctttacacaaggcaatttggtacacggaacacgttattaacagtaaaaatcggcgaggtgaaacgatgttaaagaccaaggcctcgaatgtcaattatagcgactacgtcatgtcatacatatttgtgccattggcttcgtacacaattatgaaccatttgcgtcaattgctgagacttaattttgtgtaacaggatatgtaattgtatgttatcaatgtgtaataaatattcattagaattatgagtttgtatttttgacgtgtgtgtctctgttacgccgccgcgcaaacacccactgcgcaaggtttatatataaaaggtcgttgccgaatcggtgttgttcagtattcaaatcgacatcgcgcacttgctaccatggctacaataaggaataaaagtctgttgcgaagcatagaacaggagaataaatttcgtcaagttcccgtgtccgacttgaaaaaggtatctcgcgcgatatcaatacttcaacagtcgaacgcaaatttagctaaacatgtaaataatttacaaagttattacgagagaaaatataaaaataaagtgttggaactccaggcggatctgcagagaaaaactcaggaaattgcgcaaatacaaaaagactttgttgtcgaatacctgtttatcgtacgtatggataattgtgtgtacatgtttgatgatttttcaaacgtaaacgcgatgctcggagaagataagcaatgtcgagtaatattgtgtcgaatatctaaatcggccgctgccgaacgagccctggtcgccgcgttggccgtgtccaagtgccaagaacgtgttctaatcaacaacaatgaattgatttttcaactgtcttcagatttggatgaatttgaaaacgaggtgcgaataatattttcgtaatttacaatgactactactactacgacgacaactggccactgttgttgcgcaattaaagtgtttgccaatgacgctgaattagttcctgtcgcaaaattttacaatgcagccgaaaagttgataactttagactataatatattttttagttgtagtgaactaaaagtgatactagaatcatcgcaacgagacaaatttttacaagccttgtttcaatgcggcaataaatacatgtgtattgtgaatcgctctcgagacaagccgatattatttgacggcttcgtgaatgagttcgaggacgagtgtaaaactaaaagtttcaacgttggaaatctcaaagagttgaacgaattgcacggactttgtgtacgagaaatggctcgtgcgatggaatctcctacgattttacatgtttatgtaaacgaggctataatctcagaccatcttgccaaacacgaccacggcgccgactatgagcaattgttgaataaaatacgggtaaacatagatggcggcggtattgccaatgtgatcgcacctcctcgatgttcaaaaaaattcaacaaaaccaagtgggcgccggtgtcgtgtaaaacgggtaaacatttacttacagcaatcttaacttttaggaaaattatacacaatcaatgatttatttatattttatttattagtcaaataacggcaatgtacattcatcgttaacataaaacttttgtttgttaaattgtagcgtagaagatattaaattattagttttattattgtaatgtttaataattgtaattgtaattactgtgttacaagatactcctatgaaaattaaaactacaatgactgtaattgatattaaagagacttttacaaattttacgtgtacgtttgatttaccatccttttcaatcatagtaaaatttccatgaccgagttctggctcctcaatctttggtgtcgtcgtcgtcgtggttgatgatggcactgtggttgttgttgttgttgtcgtcgtcacaggtgtaaaaatgtattgttgataggtggtagtgttgtttaaagaagaacgcggcatcactttggttaaacaatacctgtagggttcgtggcgaaattgacggatatacttttgagtattgtcgaatagccaaatgggtgtttcgttgaacgcgttgagtaggcctcgagtatgattattatacaatctgtagaagcgcaggaacaaatttttattgaacatggcagccagatgaataatcgtattgttgagagcgtcaacgggtttggtcaagtgtatggcggctatgtgttgttcttgtaaattgggtaaatgttcgacgtaactgcgaagactgcccgttacgttgaccgcggctggagggatgaacgtttggcatgctagcgcaattttttcatacttttgcaccgtttgtttgagttcgtaatttgtcgcggccaaagccaagattgtttctttacgaacgggcatgttcatcaacatttggctcatgacttcttcgtttcgacacgaaacgttcgacttgtacgaatagttattgtcgatgttaaagtatttgtcgacgatgcgcatgggatccgaacgatcgatcaacattagagaaacgataaactttatcaatcctttttgtaaaaagtccagatccagactcgtgacttcgtatcggttcaatttgattattctttgcggatacacgattgaatcgtttttaatgttgttttcaatgtacaatctttgcggtgtcaggacgaacatacaatcgtcaaatctaaccgtgattacgttgtgacacttttcaaaggccacattcgatatcattttcaagtagttattttgaaccatatccttgacgcgcaattgattctttctgtcgagatagatacattcttcgtatttgtttagtttgaaaattttaaactcatgctcgagcgcatcgtttgtcggaatcgaatagttgtcggtcaagattgcgttcctcaataattgtggatgtttttcgaaaagaaacgatacgagagcactgcccatgggatagagcatgtcgtcgttgtagtttgatcggacaatgtcgtcgatggttttgtgttgatagttacgcatattgaaatagtttcgccagtagcattgttcgtaaccgaagcgattagccgtaccttcgatgaaccatcgagattcttttatatttttcatgttgaccatacgattcgttgtgtacatcaaacaatgaaatagttcatgattgaaagcatgaggaacaatgtcttctttgacaaaataaacggcggccgtgggtttgttgtttctgaacagggtgatgcctccgtttttaatgctgggcgtgatcaatagtcccattcgattgtattcgctacgattcgcgtacacgtacatgtccacgttaaacatttcatgtataaacgacacgtttaaaaaattgtagaatcgtttaaagttatcgtacacgatcttgctctgttcttgcatcttgttgagcacgtttaaatcttgaatattgtggtgtagcgtaatttttacaatgccgctgtaaatggtggtactttgcgggaactcggttatttcgaccgtttcgtacaatgtatcgaatttttttcgttcataatcctgcagcttggacacgtaatgaacgtaagcagcttgtttcacgtgctgtttagacattttattcaacggatactgcaatgcgacattgatgacacgtatggtcgcgtcgtcgatctccgtcttgtagtattttgaaaattttctatattttctcatagcagcgtacatcttcaatattgcgtcgagtttcgtcggcgaccatataacaaaattgttgtagaaatgttgaaacgtcgcccagctatctttgaaacgtagaatagtttcgacgtaataatatccaatttcggtgccgatctcgtaaagtctatcgcctagtatttgcatgtcgtacgatatattttcgttgacaatcacaaaatttttcaaatgtttcaccacggtaaagatcatgggttccgaaggaattttacgacgaagataatcttgaatcagcgtaatccaatcaatgaaacggcgcaatccggtcactctggtaaagttaaacttttccgttttgaatttggccaatatttcgtttccattgcaagggtggcggttgatgacgatagaggtagtagtggttcgattatttttaccactacaactttcaaagtaatcttgaacattgcactttattgaatttattaaaatcacagcgacgaaaattgatacgacggcgaaacgcatggttggtgcgttatgaatacataaaaatcatcgacgatgcagtattttatagcaaaattttatataagcaccattgaatatttattgattatttgatgtgctaaattataatgtcggtgaataaattcgctttaacaacaattaatatgtacgacaagatcgagcacctgacgcgtgcgctgaacgacttgcaagactttgtcgatagtcacgtaaccgaacgtgattatctcggtctagggtatacgtcacgagaaaagtttgtgaacgatgtcgtcgacgctatactatacattcaggttcgtgagagttttatcaaattatgtacggacgacaatgaaaactttaacactttgacgtcgcgtcgactagtcaacattgaacagtacaaatgtaaaaattgcggtacgttaaacaacttttgttccacgatcaacgataacaacgacgaagacagcgacaacgacgaaaaacacaacaacgctattgttagcgacacgaacaacaatgcgtatcgtcgaaacgctatgcttttcatatcaaaatggacaattttcaaacttttgaccgaattatttttatgggcacgatgttggctttggggctattgtgaaaaaccatggtacattttgtggagcaaaaagaataaataatcattgtttgcgcgcattttcataagtacaccaccagtcttgtatcgtctttgatgatatacgctcgtgtcaacaatcatgcagctccaccaacagcgacagcaacaaaacgaaaggataacaatactggcggaaaaagaaaacaatctacgcaatcaatacgacaataaagtactggccgttttgaaaaaaggacaatttacggacgccgtaaagaacgaattgaaaataatggcagccgaacagtttggcatcgaagaacagctttacaatcttcgacacaacaacaccgacaatcgtcgaaaagattttattaataatttcaacgaactggtcgattttaacaacgagcacattgaacacatgtataacaacgatgaaaacgatgatttgctcattaaaaagtacaacattaccgaaaaaccaaaggctattcaggatgtgttcaagaaaaacaacaggaaatttatcaaaattttaaaacaatttatcgataaacgcaacgtttacagatcgaacgaaaacgttgccttgttggaagaattggttttgctcaagtgtaatctaattaaacatttgtgtgtcatggaaaagttgtgtaaataaaacaacatcataaaacaacatcattataatgctttgtattttatttaaaaccaattggtatgaatgccactatctacagaatccgagtcgctaaagttgtcatatccgtcggtatcgtcgatgtaatcttcaatgttcttcaacttgttgttatccataaaaaagcgactgattttgtcttttgtatgttcctttattatccttggataatcgacgactattctagtatcaaaattgggcaacgcggtcaatacaaaaattttattaaagttgtcattgtacatacattcaaaagtcttttctattaaaagcgttttatcggcatccacttgcaaaatttcgccttcttcaaatatttcaattttattaaaaacccttgtcacattttcaaaatgttcacacaccatcattgcgccttttctattgactatgcccaaatataaaaaaaattgaacttttcttaattttctaataatattaacattatcgtttatgtcaaaaaaataattggcttttaaaaacactctacaatacatccgctttgttgctggtgttgtcgtcatcgtggttaaaaatccaacttttaggcggcgacggtagcagtaaactttgatgatccaaacaaaaatttacagtagcagatgttgttggtattgcagtcgatgttactataggttgattatttttcggcgtatcgtaatatagttcttttttatcttcaaggtcgtaagataattttgatactgacgtggcgacagatttcgatttcgcattgtaataaacattgtgcaacttccacaaattgtaacaattcaacaccatcaacaacaagatcataaactgcaatcgaaatctatattcttttatgaacgtcaactcgtcatagtagtattcaatacggcaagaagcacaataaatgtacttgtcgacagtgaagcagtttttgccatagattgtgttgtagtccaatcgctctatgccgttccagcactttgaatctttatcgtagctgcgaacttcgacgtctagtatgggtacgtggccgtatctcacgatccagttgtacgtgcaaaagaacaccattgtggtcaacatggcgtgagaacacagcaacgtcagaatcatcagtagataatccgatttgcgtagttttttcgttagtaaaacgcccacaccgacgagtatgagattgataccgtagacgagcacaaagccgctacagttaaagacgggcgtgtcgttttcataatcgatgagcaatgcgtactgccgagcttgaattccggccaatccgagagatgtgaacacgataccgacggtgatggtcacgatcgagcgcgcaatacaactaagtgccaccgtcatgtttttactgttactgatcgtttgtgtgatactctttctctttttattatgcaaacccatgtacgacgcccacgctcaaataaaaaaatctcaaaaggcgtacaatgaaacggtcgatgaacgcatatattacatgcaaaacgttctacaacgtagaagattcgtgccgttgagcgcgctgccaaatattatttttaacaccaatctcggcacaattaacgagggcgaacaaaaatgtctttcggtgccgatatacgtaggtttcggcaacacgccaaatttcgactgcgccgctttgtgcgataatccgacagcttcctacttttacgttaatcaatatgataaattcgtgataaacggtcaaatgctaacgcaaggcggatactgtacgacgagcagcgttcctagaaactgtaatcgagaaactagcgtagtgttgcacagtctaaaccaatggacgtgtatcgccgaagatcctcgctacttcagcggacctcaaaacataaaccaagtggcgggacgccagcacattgatcgtatcgcaccgggccaagcgaaccgcaacgtacttttcgataggctgctcggcgtcgaagtggacgtgactcgaaacacgttccgcagccattgggacgaagagatgcccgacggcagcggtcgtagattcgaaatgcgttgtaacgctctcgacgatcataacaatagaatgttcgtgaatccctttaatcccatcgaatgtttacccaacatttgtaccaacgttaactttgtgtcgccggacgtcaggcccaactttgaaacgggcgaatgcgagtgcggcgactttgccgtgacacgcgtccgccacgtcgatccaaacgatcgtacatcgctgtgcgccgccgtcacggacacttacgacgagagaacgcgatctctgcagtatcgcgtagaatgtatgaacatggacatgcctgtggaaaaatatagacgaaacatgctctggtgtccgtccaacattttcaccaccaacacggacaacgcattccttttcactgtacccggatcgtttcccacgtcgggtaacggtttagacgaacccacatggcgtttcatgatggaggttcagagtagaatagatttcggccaaattcgagcttcgccgcccgtctaactctaagagtctataaaatgtataatatattgttgattgtcttgttgatctttatcattctaatagtctacaacaacattactcttttacaatacgttcaacaagactatattcctgttctgacgagattcgacaatacacatgtgccgttgatcgaaccacctaccgaaatcgtcatcgaaggaaacacgcatgaatgccacaaaacgctcacgccgtgctcgactcacatggactgcgatgtgtgtcgagaaggtttagccaattgccagtactttgaaaacaaaaccatcatcaccatcaccgacgaagacaacgtggaacgccagttcacaatcgaaccgggcgaatcgtattgtatggctcttgaccgcgaacgcgctagatcctgcaatcccaacacgggcgtttggattctcgcccaaagtcctgtaggtttctctttactctgcagctgcctgacgcccggtctagtgacacagctcagtctatatcacgattgcgatataccgataggatgtcaaccgcacggcaatatcatcagcatcaacgaaagacctatgcgttgctcgtgcgaagtcggctatgtcgctgactttaacaccgaaacacaaactccctactgtagaacaagacgcatacgcgatgtcatacaaaatcctgactttttcccattggcgccgtgctcttggccatacatacccatcgaacatcccggtctcgaccctgcctacttacaatcgacaaacgcaagaaacgcgtgcgtcatcgatccatgcaccgtagaccccatcaccggacaacaggtcgtgggttggctggtgacgcggtatctcaacgatgaagacaaagatactcagtttttctgtaattgttcagcgggacacaatctgtttggtgtgtacaatgaccaaccgaacatgattcggccgagcgccgagaaactcgttaacgcttgcattcaacctttcaacgtacacgttgctcaattgcccgccatcgagtacaaatggttttggggacagagaaatctatacacgtccgacgatgacgtggtcgccactgtgagacccgaccaaatcagttcgccgagatatcgtcgcatgcttttcacatatctcacacctcacccgtttttccccgaaagtgtcaactttatggtgatgaaattctctaccgcctacactcccatattcaacgaagcagatagaaactatcataatttgtttacgcattactatgaactaaaccatcggagcgctacgtgcttctatccgggtttcggtagatgtgtcgttcacgatcacacaaattgtattcgacggttcggcagtgttcaagtgggcacggcggaaaatttaaaaggcacccaatgttatttaagtcgagaccgatggtggattcgaatctggtacaagccgcaagtgtacaccaatagacgatatgctgtggcgttgtacgtgaacggtcttttcttcgttatcaacacaagagactttagaacggttcgttttgtgagcgccaccgatcttttgatcaatcaaaacgacctcaacaattctctaatcactctactcaacacgtaccaccacatatcggtggtttgaagaaaacatggacaatgtcaccgaagcattattactggccaaacaatatgaaaaacgtcaattgtacacgaaatccatggcttgttacaatttggccatacattttttagtgttgcttaaacgcaaaagattgcaagaaatagttttacgaatgattgacgataaaattctacattgtgtcagagaacggaagaaaatcaatgaattaaacaaacaaatcattttaaaaaagtatatattaatcgatgataatgatgtatattaaacgtacgacatacaatatcttagaatagtttgatggaaaaacataaaaggatgaatgtttatagtgatattcttcgcagccaccagctttgaaacgacagtttcgacatgttccgtgcgatcgtcgtccagagtgcgaatactgaattcttcgtcgtggtaataaaaattctttggtatggctccttcctttgttttattcaccttcgacgaatccaagtagacaacgtcaggaacttcgtttgtaatttcgtgtttcatacgatcgactattgtggcgttgacgacaacgctattattgagattcttcgttgacgacaattctgatgaattggtcagaatgattacatcagaattattcattatttcatcatacatttcgtctatgatcatcgacatgtctggtatttttttaaccgtagacgacgacgaaccttcttcaagtttttgtagttttttaacacgaggaggattcttgcacgtctttggaggcatgtacgtcaactttttactactgtacgatttacatcttttaaagtgcctcgtcgagtttttgagaaacatgtgcgccgactcaaaatcgtccaacagcttttccttcttcaacagcaagacgcggtgcgttttaccggcagcgtctatggccagatatgcggtgcggttgttttcaaattgtttaatcaaaaaacgattacgaaaactgtcgtagtattcgttccacaaacattcaaaagtaggcgttttcgaggaaaatacatatccgcactcgtttatacataaaaaactacagtgtgcagatgaccgtaataaaatcttgcgattgggttgagcgatgcgatgaaacacggtgtcgttggagttttcaagtttcgttccgtcaatgtcgcccacgagattcatacgcagcagataatgattaaaatataatacaatattattctgcgttccggtaagagtgtcgttgatcgacaacaatgttggcgatgatgtcttcgacgacgttgatgtagagttcttgttggaatcggtagtatttttcttcaccaccgccaccatagaatcattactctttgccgtcgtatcgatggctagtccacttgcgatggttgcgaccagaagcaaaattgaaattactttaacgaacatgataatgccgtgatgttcggttacgacgaactaacgatgaaactacaaatgcttgcctttttatactaaccatcaaggttagtcggtgcgcaattaaccgcaaacacttgacgtcagtgttatacttacttaataaatttaaaactttaatttaaaatcaataataggtcgtataaaggtcgattcaattgctatttgcatatcattgaacgacaagtatcggttcttcaaggtggtggacaatggagcgttttgaattaatttcggaccaactgagctggaacaaatgtttcgctgataaattcgccgcctgctactacatcgatcctcgaagagcgtccatgaaaacgatggccttgttcaggttggcgtcgtttttcatcgccgccgcactcatcgtcgccgccatttacgaagcgtcgaaccgaaacgatttcatgttatactacacccattggtcattactggcgctgatgataatgtgtttttccggatcgctaacctcaatattcatcatgcaacaaacgtacggcaacattatttatattccaaagcatgctgtagtgtttcgcacgttgtacaacattgcttgtacggcaaacattgtatcgactataggatactttgtgataacgttcacctatccgaacgcgaccaaaaaagtaatcaatcacgttatacattcggcaaacacgttgttggttttgatggaagtgatcgtaaacgccgtacccatgagattgtttcacgtgtaccagccgttgttgtataccttggcatatgcgacgtttgcatacctctaccaccattacaccggtcgagctatatacaaatgtttggaatgggaagaccaacgcgaactgacaaaactaacaataggtttcatcatattaatgtttgtagtgtacatgtttttgtatgtaattagttttattaaaaacaaaatgttaaaattgtaatctcaacaacaaacacaaaatttcaaattgtataatctcatttattttaacattaaataatatttacaataatctttgtgttttattggtgtggtgtcgtcgttgatttgttcgtcgcagcaaaaggttctcaaagcaccgtgatcggtacagtagacgccgtgatcggccagctttacgtcgattccgttgtttatcaacgattgttgtctcttattgtggctgatgtaatttttgtttgagattgccgacattttttcaatactactatcgccacattgcttgtttctatcgataatttccgatatcggcgcttcaatgtcaaacggtagatcgcaatagatacatttcaattcgccaaactcgtacaccaacccgctttcggccaattgttgtgcgtcggacaagctaaagtgatttgtcaaagttttcaatctattctgttcagacttgtagagtaatcgatgtctattcttttgtacgtatctttcgaacaatcgtttttcaatttgatattctttgatgtaaaacgtttcgtttctgagcacgggcaagataacgtacgaattgtaaaatttgaccacgtacacacacaacggcgcgttcatgacgtacatctgtctttgcatttgacggtaatgaggatcggtttgttcgacggcgaataaaaagtcaccatgcttgttcaccgacaacgccgtgtgtttgactcgataacgatcgttacgattgccgaaagttttacgcatttcctcgaaattggtctctttgtacgtgtgcggacatttaatttccaacggcacaaatacttctttggcgtcatcgcccgtcgtcgttttaaccacaaagtacgcatcgggcgaagcggaaaacaatccaaactcggacatgaacattccgcagttgagaacttggtcgacgaccttgccgcctatggtggtttcgatgacgttgatgacttgttgtatgagaaacttgtcgttttttaatagtttctcttgacgcaagccgtaagacatggccgccgattgaggcaccgacgtcgcatagctacaatttccagaagctgtttgtcgatccaaacgcagcatgttccatagcggattgagagattgttctctagtagcgtgttcgacaaacaggatgtcgtttcgagacagcctaaacttgggcgattgaagcgtcgacacataattattatacgaatatttatctataatttccatttgacttttcgtcaatttacgtgtttccatcatgatgtttaattatcaagtgtgattttattcgcacccttacaccatacgatctttgctttcaacgaaacacttcaccgaaagcaaagattgataggatgacgcaatattaaatcatcgctaaaaataacactattgcatcattcaatacgatctttgctttcaacgaaacactttgccgaaagcaaagatcacattgttaactgcgcaataaagtcttcaatctttgctttcggcaaagtgtttcgcggaaagcaaacatcatcgaaccatgatatcatcgactgacgcaatagttatttttagcaatgacatcagcacacaatgtttgctttcagcgaaacacttcaccgaaagcaaagattgtatacgaatgatgaaattgtggcgcaataaagtctttaatctttgctttcggcaaagtgtttcgctgaaagcaaagattgcgattattgcgccataatttcatcattctatggcgtcatcttatcgaagtttgctttcgtcaaagtgtttcgcgaaaagcaaagattgtgtgtttaatcaatatattttgtcatcaaataatctaaacattgagtattgatcatgcattgtaaactatcgccgtcgctgttgttggctagaaaacctaaaacaaaatttagcactcgttgtttgttgaaaatcatcctttccagttgtattatcctgtcgaacgcatacactatggttttaaacgctgcgttcttctgtacaacacttgtcctaacataagaaaaacacagggcatagaacatttgttgtgcattagacttggacgtatctatcaagttgttgatatttaagatcacgtcaagcaattgatttggatttttcaacttgataaaattcatcatggaacatttacagaatcgtttgttaaaacacagagcaatcccttacatttctaaaaagtttctcaacgatcggttgaccgatctagtgctgcgtaatgccgataaaacgtttcatcgaagaatcttgccggcaacggttcgagcgctcgacacgttgagtgtgatcaaaggaggtgctgccatagccgttcatttgaacaacaacacgcctacccatctatccgatctcgatctggaagtgtacgtcgacgacgagaatgttaccattgaaaatttatcatcgtacgtgtcgttggaacgagtcgaacaagaactcaagacgatctgtgaaaactattacgacaacatcgaccaagcgctggccacgatcaacattaaccgattgatgaacaacacgtatgcgcgtgataaaatgttcatctttaaatcgtacatcaacgaagccatcgaatttgttcccgatcacgttcgtttctgcctcaaccacgcgcagccgttcaagagcaccgtgtctatcgtgaacgatgattatttcctcgttcgatactcgttcaacgtgcacatgaaaagctttacaccgatcagattgcacaaggaaaacaaccacatcaccaatctacaattctttcctttggacgtgttctttttagacgtgagcgtcaaacgttctcccgcttcttttgttaacatgtactcgttgggtaatatctttggcgttgacgtgtatatagaaaacattaaatatctcatcgctgatcaactcgaatgtatattgtttaacattttcaacttttactggaacaaagttgaatcgaggatcgatagagttggcaatcttttaaacgcacaaaccgattactttatacccaccaatcatgaaatcgaacgttacgaaaagattgtcaaacaaacacatcaacgattcacagctcgtgatgttaaaccattgttgtacgctttaggaccgctcggtcctcaagccatcattgaattgtattttttaaatcgtttcgacaacaacatcaaatacattactcatcaggtcaattttccctatcatgtttgggaacaggattactattctaaatgttggaaacaatttttgtacattttaaacaatctttataatcttaaatacaatgttgaaatttaacaattgttgttttattattcacaataggcttcctttatactatcgggaacggtccatggtacagagtcgttttcaaagtacagcatgttgatgtgaacggctacgcttctaatgtctccggttggcgacaatacgtgcagaaccacatgtcccgtcggataatagtccaccgattctggcacgtagtatccgtcgttgatgcgcactttaaattgctcaggtataagcaggttcatcaaacgatcattttcgctgtcgaatatgtatttttcgtcgacgtcgaacaattcaatttttacatcgtgcattccattgtcgagtagagtttgagagaattgtatgtttgaagtgtctttgtcgaaagatgccaccgcgggataagccattgtggtcacgactgcttattttaacagccgaataattaataaaaattttttgaaaaaaaaactttattcaatatattcaacaacaaccttaggattatgtatgttatgctcgaatattagttttgaatttaacaacatgattatttcctcgtaataaaaagcaattggtctgttggttacttccaaaaacttcctaatcgtcataccataactcaaacagctattataattgtcgttccaaacgtcatacactttcttgaacgcaatgggatttaaaaattgtttcatttcttccagtcttttctttaacgcagtcaaagcttcgttcttgagcatcgattgaatgtttctcttcatttcattggaaactttaatgttattggtaacgttaaagtaatactgttgcagtaattttgtatctggacaaacttcaataaacgaattcttcctcttgagacacgtttgatactctttcaacgccgctacaaaattcaaatgatccacatacattatcggagaaattgtaaacatcatcctgttcaagcgtctctcttcgtcatcgtcttcgtttaaaaaaactctattaaactttttatccgttacttttgcatcgaaaccatacggatacacattgttctgcaaataaaactctttgtaaaacacatacaaaactttggaattttcctcccattcaatgtcgtcaacgtatcgcagcagatactcgacaatattttttttgatacggtccaaaaatcccgaataataaaatatattccaaatcttgtggtagtttaacatctccaagtttaacgtctttaaacgccatactccttcgttccaaacttcgggcagagttctatgatctacgtcacagatttttattccgtaatcaataacgcagttaatcttactctcaatgctagagtctccttcgagaattgcagccaccgttttgtttgcgttatgtaatctcaacatccaatcggtaaaaaaattattttgtatatttttatatcttctatgtcgttgaacagcatcgaacacaggatccagtttgcgtaaacgatccaaattgaaagaaaaattgtaatatttttcaacaaattcttcaaacttgaacagcgccaacagatcaaccacttcgtcggccatgtattcgatcgaaccgacccaatactcgctggtccaccatgcgtacttgtaacttggcgaaacgttcattgtagtctcttcttcggcgacacaatacattacaatcttgacttcagtactttatatacattatgcgctggcgtacgtgaccatttatcaatagattaattgattaaattgtgttatcaaggcgtacgtgacttgtttatctataaatgaagtgcatacgtgactgataaactttcaggaaagtatatataacgtcgccgacgctaaaacattaatcattcatcgtagctgtgcatcatggaaccctctacttccactgcaaccaaacgccgcaacgaagacgccgacaacaacggcagcaagtcaaagattctcatggtcgagaaccaacattgggtgaatattacatctaaaccgtttgaagctatcaacttgatttggtacaatatgaatatcaaagataaaataaaaatgttggccgtcatcaacaaggaatcagagtttgacgttttgctgcgactgactaaaccgactgcgtttgccgtgaccagcttgggtaacgacaccaactttgccgtttttaacgacgcaatagccatgactattacgccgttcaaagacacgagttatgatcaaatatatcgaacttattgcggtatcgtcgacattagccatgatgaaaaatattacttgtatccatttgaaaaattttacgacatcgacactactgactatgtcgaaggcaaagcggacacttcgaaagccatcgaagttaacaattcctactcggtctacgaatatacacgcatggtgatcgatggaaaaatgtacgacacttatgaagaaatcaagatgccgcccgtcgaaattacacactgtaactgtttggtaaaaaccaagatcgtttttgagagttcaaagagaagaaacatgaagccaaagataaatcatacgtttcccaacaacgatcgaccaatcatcaatccgtcgcccagcgaatatttactattgagcaattattacgctgatgatattatttatgttaaatacttggacaatcctttgattgaaaatgtaataaagttttaaaaatgtgtatgtgtttattttttatttaaaggaggttgcagaatacccaatattgtttcgagtaaatctagaatgttgttgacggtttgttgtagttcggcaataatgtttcctaaattcaaaccgttaatagcttgcagtaaattgtttagtgtggcgttaatgtttgtaacgctagatgaaagattgttcaatacggcattgattcccgtcaattcgttgcgcagttgttcggacaattggtttatggcattgagtaaattgttggacaaagcagtgaggcgcgtatctatttccgccagcaatctgttaaaatcgccggtgatgtcacccaaaccttcaacggcctctagtatttgtgctacttgagcaccgatggtgctttgttgtaaacgtatagcgttcaaaagattggacaattcgagatgttggttagcgttgttgacggtgagttggttgaggttggtggcgatgagatcgttttgacgcgctatgcgttccaatagctcagtatgatggttatgatgatggtggtgatgatgatgaggacagcatctggaagatcgacgtctagaacgcgatcgagaacgcgatctacgacgtcgagggctcggcgatcttcgactcggcgacctgcgacggggactgggcgagcgacgtcgcggtgacacggatttgcgtcgaggcgagcacgagcggtggcgacgacgttcttcgtacacttcggcgacgaacaacgtcatcaaataatcggaaacttgagaattgacgcgattgcacagattgcccagtccgtataaatctacaaacaccttgttaccgtcatgagaacaatggttaggacaacgaaaatcgacccaacattttttgtgtcgaggcgcaatactttgcaaagtggacgcgggcaatcttaaaaggtgaagtacgtcatcggcgctgacccacaaaacgcagaattggtcaaagtacaccgagacgtttatatcttgacatttttttgtaatcaacgacattatattgtaacaaaaagtaaatttttgttaaaagacttactaacagccaaaataaataaaatcacatatataaagttttaaaatcgtttattgttgacgaaagttatacatttcaacaacattgtaaattctatccaacaacaattccacgtcctcgtactggtccacgattttcatgtcgtgttcgtcgttgtcgtcgttgtgattgataaaatttaaaaaatccacaggttcattttcatgcgtcactgcgctcttgcaaaccgccagacaatcgccgttgatacacatgacggcgtcgacgacgtcaaagtgtcgcaaaattttataatacacgtccttctgatccaggtttaggtggttgtgaattgaaatgacaaagtttactaatttcattcttgtaaaacaaaaaacaataaacaggctcgggcttttttctatatatgatcagttcaaagtagtacaatacagaatcgttaacgtgtttcaagtcggttttaaactttaaccatttagctatcctgttggccgctattgaaacttgaacttttttcctaatgttaaatccaccggttaattcgcgtcgtaacaattgcctacacaacaaactatcgattcgttcgcgcgacaacattgttgaatgaccgcgaaacagtactgcttttcgtttaaatacacgcaataaattagattattaagaactgtagccaataataccgcaggcaatgcgagctcccgaattacctgtcgttttactgagaacgtgaccgcccacacccaaatcgtcacgatccgcatgaatcactagactgcgacctataatactgttttcgccgaaaagggtcattacgttgtcgatcttgtctatttcagttaaactgtttgactttttcgcttcaatgttacctaaatcgccgacgtgacgctctatcgaattgggtgcgccatgattgcgaccgaaaggattgaaatgttcgccggcggacgtacatccgttactggtgtcgccgaattcgtgcacgtgaaatccatgaagacctttggttaaatttacgacatagccttttatgtacaaaagatgattgggcgattgttgttcgaatattacttgaccatgaacgacgtcgtcgcttgatagtatacaaatggccttcattattatggtttgccttaacaatgatgcgcgttgtcattattcacacatactaagcaagatgaataataatgccttcatcgtcgatatacggaatcgtcgtggcactagtcgtattgttcatcgtatgtttttatataatgggaacgattcaaactttaaacaacagagaaacccatgaaatcgtcaaagcaaacgtcaatcctcaaaccgccatggacattgtgttcgatcgtaacggtgtcgtggattgtaacagcactcgactcccttgcatctccgatcaacagtgccaagataattgtttgattcaaaccgcaatcggcgggatagcgtgtaacaacgggttttgcggcaccagggacgcttctttcaccggcagacctgacgatttcgaatgcgatcctaaactgggtctgatcaaagcgtatgtggcgagcgagtttgtcgtcgatcaaatgtgcatcagcacgtaccgagacattgtggacgacttgggcgaaccgagaccgtatctctgcgacaatggcgttctggacattgatttaattagtaaacaattttccgccgccgattgtacttgttccagcggatacactaaaatgttatttaaccaaaccgcgttggctagatccattccagtgtgtattccgaaccgatcgagaaacgtatttagcaaaatttacgaagtgttgcaataaaatgttgtcgcacgatataatatgttatgcttgcgaaaagcacaccaacaaaaagtacaacaagatcctcgtcgagtttgttaataatcacgcaaacaccaacatttgtacactgatggatcgaatactgctggcgttcgaagtggacgtcgtgtacgagattgttgaaaatgtaaaatccatcatttgggccgtcgtgaagattgtgtatgcgcgcaatcgaaatttaccatcgtttcaacatcacctgccgttcataagttttgctgccaccctcatcccgcagattgcaaactcaaaggatctatacaatttttccaaatcgttatacaaacgtttctgcgtgtacgagatgaaagaaaactacggcactttcgacgacgtaatatcaattttaaaaaaattagattctttatgtacagtagtagaataaagtattattatagctgtatacttgtcttttatttgttgtgtgttatattgaattcaataagtaagctatacaatatgaatttgcaacaagataacgagtcgcttatatcacaatacacatctgttcaaaagcgaataatcaacactttaaaattgatcgttgattataaaaataacaatgacgacattgaaaccttgcacgattacaaagaatatattaaagaaaacaacaactatcttcaaactttacgcattgtaaagatgaacttggaaaacatcaacgatggcaactcgaaaagaataattgaaaatttagaagacgacgagaaaacattgataggcgtcaacgatctgttgcatagaaaaattcgaaatgcgtcatctgcgacgttgccttcttcgtttgtacccaacataatccaacagtataacgtcatcgacaaattgaacgatttggtcgacaaggtgggttttttcgagtttagaaatacaccttacgcggccgaattggaacgactgatgaacgttaacgtgtttcaaattcgactacacaacagggtaaatttgaatcgtttagattgcgcagcaatcatactacaagcgctcatcaaagagaataatagatcaattgatttttcaaagtttgtccacggcgacagtccgttgatgttggaaaagttaaaatgtctattgtattacgtgtttgaagtgtgcaagtttatggtggagaacgatgaagtggcgctcacgacgccaatctctatacatcattacattatagaccctaaaactatatctttgggcgacaactatacaaacatcaacattgacaacgtatttgtcgacaagtttgcgccgtacaataaatacgaagcggcagaggtggaggaatctactacgtacacgattctctatatcaacggcaacataaacgatcgcatgttcgacgaatacgctacccatcaagacgtttggtacatgaaatgtcccgaactgtatgttttacctcattttatcaaagataaacttggcgaaaacgaatcgtacatcatacacaacgtgaaacaatataacattctcactaaccaatcgtacaacacgaaacgcgtccacgacgctgaaatgtacaacaaaccgctgccgatgcaaaactttttaatttacgaatcgtgcgattacaaaacgtacacggacattgatcaatccgacatcaagcatctggatagggaaattgctaaactgatgtcgggcatccactacgaacaggccacttctagtaatacgtcattaatattccgttcgggcccgcacaactgtcacgacaataggacgtttcaatttttaatcgaagttctagtgtgttccaacgagggcagtaaattgtattattgcgcatcaaactttgaacaacaaaacgaactcaacgacacgttagaatgtatccaaaactacacagtatcacaactatacaacaagctggccaattataatttcaacacgacaggaccgatgaatttttatcgtagtagatagtagtaagtgatcggtcgttgaaatggagtcgatcaatattgatgattttgcccggcagctgatagccgacaaatgtagctcgctcatcgaatcttcagacatgttacccttcaacaatctggcaatgttaaaaacggtccgcgacgagtactttaaaaatccaacggcaaaaaactatgaaaacctaaaaaagatattcacgcacaccaaatactttgacgactccatcgactacaaagactttagtagacgtattctgttaatagccatcaagtttggtttaaacaaaagtaaagaacactttcaatcgtataaaggtatactcgaagtcgccatcaaacgtctcgacacgatcaatcccgatttacgcagttctaaaagggctctgttgcaacactaccacgaatgtttggaaaactttgacggtccacgcaaagatgaacaccatctcatcacgttcgccaaggaaatagccacgaaaatcttcatcgataccatcgacttgtacaactacaacaacaagagtcccatcgatttcaacgaaaccggcaccaaatctatagaggctccttcaaaaataactccgctactcgaaactgccattgcagagaatagaaaacgaaagctgccaaaaacgcccgactataaaatagctacgcctttgtttcaattgtaataaaagacattctggtatattatacacattgttttattattcattatttacaaagtaatcttcatcattgtaaaagttaatgttacaattattagcaaaataactatcgctaggtaaagtaccattcacgtcgtcgccgcagaagaaagaattttttcgatccagttcgatgccgtatttcgccaacaaataatcaatgctgccagtcatcggctttcgatacttgtcgttcttcgaagctatcatcacgatcatcggcaaacccagtatttcacaaatgcttctaattttatgctccacgtcttgtacggtgattttattgaaacttatgcccagttgattggtgacaataacaatagtgtacttgttattgatcaaattcaaaaaagtttcgtaaatgttgccgtacttccattgccagtcgaaacgatctcgcggaaaaacattgccacttttagttacgatcaaagtgttgtccagatcaaaaccggcaatcttaatcttgttattcaccttgggaaatttaggtaaacgacactcgaaaacatacatcgaatcgtcgacaacgtaccaattgtaaattattttcatcaggcattgctcaatttccgcttgctgctgtgcgagatcttttgaaaaatcaataacgtactcgtacgcgttgtagtaacgagtcatttgttcgaatcgttcattctgctttttcacatagtcttcggtgatgagatcgatgccgttgtttcgtttgcgcatcattttcactacatgagactcggtgccgggttttacgcgtaaaaacaccagtttccagtcatcgaagaaattcattttagcgctctgttcgaaaattttacacgaatcctcaagattgatgtctcttctgatcgcctcgtacaccaaagattccatcggatgacggtcgtagacgttcaacgttttattgtcgacatctcgatcgtccgtcatcaatctatgcgctatccaaaagagactgcccacacggtgatccattttgtactcatcgttaaactctttgtaatcgctcagatgtacagaaatgttgttggattttttgtcgagctttttcaacaaagtggttttagtggtgcaagccactccacccaatgctaaacggtatgacatgtttgcaaattgtgtgtcgcatcaagcgacaatattgattttatactctgttacaatttcattatctcgaaacaccaagggtaacgattcgttacccttgctttttcgtcgaaaactacaccagtttcgtcgacatcaacacagctaaacaatctttgctttcggtgaagtgtttcgctgaaagcaaacttcgataaaatgacgcaataaaatgataaaattattgtgcaatagttacaatgttggctttcggtgaagtgtttcgctaaaagcaaagattgtgattattgcacaatgatttaattttattgcgtcatacaaaattatttttagaaatgatgtcaacggtgctcgatgttggctttcgtcaaagtgtttcgctgaaagcaaagattgatgaaatcattgtgcaattgttcgatctttgctttccgcgaaacacttcaccgaaagcaaacattgataataaagataacaaaatggtatttaacggtctcttcgtttgcgatcaacacacatcgagattgaaatgacaatcaaaatgaacacattaaaattcacaaagtcgcccaatggctacacgcctcgtatggcgaccgcgggcgctgctggatacgatctttacacgcctgtcgatttcatcataaaacccggccgtcaaattatcatcgatacagaagtaagcatacaattgccggaaggaacgtatgcgcagattgcaagccgatccggcaacgccgtcaaatacgaagttgtagttttggcgggcgtcatcgacaacgactatcgcggctccatcaaagttttgctgcgcaatttgggaaagaagaatcgacagtttcaacgaggcgacagaattgcgcagttgatcataaacaattattacaagttttcgtggaaacaggtggacgaattgtgcgacacggatcgcggcgaacaaggattcggatcgaccggtcaatgatgtgtatataaatatatgtattgtttgtgttttatctttagtttaaatcagaatacacgataaaaatgacgttgtctaaagttttatttgaaccctgcaatcttcaaacgttggccttttccaaagtcgtatacaatgaaaaaattaaacaaaaatttgaacagtatcatttgcccgaatattataaaaagtttttgtggttcaactacatcaactcgtacacgttaacgtgtacaggtcttttagaatatgtggataaaacgcaagatttgaaaggggctttggattgtctactgaagcacaacaattattgtcatgaaaaggaaaagttcaactggaacatcaaagccgactgcgatttcttcaatattaacattttagaagagtttgacaatttgattagttgtaatgtaatgtacctgtcgaaaactaacatgttaaagtttgtaatgttgtatatgtatattttgttgaattttgaacattttctacctcaatctttttttgtgttctattacaatttaacggatttgtgtttcgagtgtgatgttgataaaatgttatttccaaatttgatgactttattcgtcgatcacgacgacatgatcttgtgtatcaacaatttaataatgaatcccatacatagatgtaaaaaatgtagcaatttcttgtttaacattgaatgaatatagataaattattagtataaatatacgttgttttattaaaagtgtccataaggtgaaaagaaaatgtttgcgtacgttacgttggtcatgctcggagacgagtacgttaaaggcgcaaaggttttagctaaaagtcttttagcgagcaacaccactcacgctttggtgtgtatggtaacgtgcgacgtcagcgctcaggctagaaacgaactcgcacaaatctacgatcgtgtcgtcgatgtagattacatcgtttacgagtgtccgtcgatgttgacgaaaagacagaatcaaatgtacggcaaatggatcgacaaatcgtttacgaaatggcaatgtctcaaattgaccgactatgaaaaaatcatatacctagacgccgatcatttggtagtgaaaaatatcgatcatttgtttaatctaaaagcgccggcgatatgttttaccgatgacaattacggatactacgatcgtttgcagtttggagacaccattccgccgaatagcgtagccacgttcatgcgatacaataaaattttatgtaaagccggcacagtgttgttcgagcccaacttgaccttgtatcacaccatcctcaactatctaaacgcgttcaacaaatacttgacaaagtctcattttcacaacggtttcgatgaacaagttttactacaggcgttgatacatctcgacatcccaattacgcagttgtcgattctgtacgcgtggaatgccggatcctattatagattatgcaagaaacaagaaccttacgtcatcaactattacggcgatgtaaaaccgtggcatttcaacgatcgttgttgcatcgactacatggacgtgtacatttggaaatactttgaaaacatgaacattgtaaaaaagattcaataaaaatgcttgtgtaactattaaagttttttattcactcacacatataagatatcaatacaatgtggtggttgtacgtgtttatgattgtacttttcattatattcgtagtcatcataatgtggcaaacgaacattgttattgtagatttagaaacacaaaattacttgaaggatcttgaatattacaattattacaattattataacaatgacaacgaagaagaagaagaacaaaactatgacgacgatcgaaatgatttagacgactttgaaaaacactacatcgacacgttgcctttgaaatttttacaaaaggcagagaaaatatcgaaacccagtcggcaatttagctacgatgacaacatctttatcggcctcgatccttggacacgtgtcgccgatttcggtacacttcttcatactctcatcggttacggtgttcgtttccgagacgtcgacgatccgctgtatctcaatttcgatcttgccgaacaattgtaccaagccatgtacaccatctacgaccatttacccattccggcgccgacgcaacaagcgccatggggcaatcctgccgattggtatcacttcagcataactatgcccgagtgtttccaacacgtttgtatagtgttgcgtggattctttgatttgacacagctcacagaatcgttgttgtactactatttgccttcgccgactcattccatgggctggcaacgtacggccggcaacgccatacgcatgtgtttaccgtacacgtacggacaactattgcgaggttattacttaaacgaaatagcaaaagaacaacaagtcaagtacgtgatagatttaatacatttttcactcgtcaaatccggcaacggcgtacactacgactacgtttatttcgatcacattgacgtcagagcatatggttatttgatcaacagctatttcacattcagctattataactatcttttcggcgaaaacacggtcaatttagacaacgtcaatagatcgctcatgctcatcagtaactatcgcggttttgctaatcccgctctcatatctcgcaccggttcgactcattccgaagtgttggctcactttatcgaatatcccaacggcgtcgtcgccgcagactttaataaaattttgaccgtcagaaacaaactctatttcggatcgttggtaggacaaacttttaacgttgcctattacgaagcggatacgacgaatagtttacacgctccattgtgggtgatgacgcgcaaaatctggtcgaaccaaagccgcgctcttcgctatagaaacttgggcttggaatcgggcattctgctcacgaccaacttgaacggtgtcgtcgacgtgccgtccactagcaccagcaccagtagtttctatcctaccattgcgtataccgctatcgctaccacgaacacggccggagtcatgatcatgcatgttcgtttcgaagagttgaatatcgagtttcatagttacacactgtatcatcgaaacggcatgttccatctttacgatagaataaaaagtcttcaaactatcgccaataacgcaagatgcgtagttctcacgcgagacaccacttttgaacctaaatggattacatcatcaaacattctgcatgcaaacggcgtcgtcgccaagcaccataatatcatcaacaatagtagtttgtccaatttcgacgtgaggacttttgacaatttagatttacaaacggccgaacagataatcagcgctgacgcaatcaacaacggcgttggcgtggcgtgtttcagtttattggctcaagaaacgttcaatgcagacaacacgataatagaacgcgtcttcgacaccgacatatttatcatcaccaccgataataataacattagctgcgtaattgattttccgattgtggtgctcaaagatgatgaaactcgtcaattgactatcaacgacgccacaaacgcttcgaaaactacgcatcatttaaatttcgataaaatcattcgacccctgtctttgttgtcgttgtcggtggacaatttaaaaatatcttcttcggatatggttcgcgatcatgatcgattcactcttttcgacgttcatggtaaccaatttaaatttacgtttacgtaggtgccggaggagcgagaaccgacgtcggtatctgcgccgttttttgaggtcggttaagatcgatcatggcgggtgcggttcgcgcgcgcagatccaaattggccaagaatgttcgttgtcgttccaatttttgcgcagtagaaatttccatactcttgttgatgctaaccacgagaattatgaacaacaatagtattacaaagaccacaatacaacatatgaccagtaaaaagtctctgagaaacgattgattgcgcgacaccaattgttctagctgatcctgattcagcaccgaggacgcggtgttggtttcgaaaaaacgagcgttgcgcctcatgtcgtcgtcctgtgctggctaggtgctcttaaaaaaagtaattgtatctggccgccgctacaattagattgtctatacgtgtcgcttcgtatagtttgtcgcgcatcgtgttcaaatgaaacggcgtattgatgtcggagtagtcgaaagctacgggattgtcttcgaccatgttgttgggcaccggcccgaaagaagtgattgcgtagctgtcgtttttgcaatagagacgaacttttcgttcgcggtccaaactgtttagtttcacgattatatcgggtacgatcaagtagggcaataccaaaacttgaaacatgttgataaagtttacaatagtctgtgacgaatccggcaccaatagtagttgttgagcgcgtttgataaactgcaaatttggaatgttaacgtacggacagtgtacgcttctgtcgcccttgatcaatctcctgaccaattcgtttttgagttcgacgtaaatgtcttgcacgtcggcgtcggacatctcaaacgcgaatcgagcaggcgttttacgatactcgaaaatagtcagtatacgcttcacgttaagttgtcgcacctttttaggagtttcgcgcaaaaagcatatgagaaagttgttttgtgtgtcatcggtcatgacgttgaactttgccaacggagtttcaccagcaatgggagtgtaatcgcgtatcgtttttgatagtatttgtataggacacatgggcgtgttcgagtcgactaccattgttttacagtagaacagactgtcggtgagcagtacgctgaaaacgcacgtttctccgacgcccacttcggtgatgggaaatatgggattccagtagacgatggtggcggaggcgttttttgtaggtgcgcgactctccaccttttccgaccattcgtgcctttgtaccattaccgagctaaactctgaggaaaaagttttgtctacaaattgaatatcttcctcggtgggcacgtacacgatcagattgcgtacataggcaccgccgaggtctttttgcggatggacgcgattgtaaggaaaagcaaagtaacgatcgctgatgtagacacaaatattaaacgggcacgtcatctttcaatcggtgcactttaactacgctcaataacgtttctggtatataattgtattggtagtacaacagctctttcaactgaaatttatttattcttagtgttaacaaaacaattatagccacgacgaatatcacgacgagcaacagcacatacacgatcatgctagaatatctgacgtggaaacgaccatcaacttaatgacattgtccacgggtccgacgtatacgcgattttgattgaatcgatactgaattctgttgtattctttttgcacgaccgtgtcgtacacgtgcaggacattgataattttttggttttgatacggcgtcgtcggcgacgacagcagttgatctagaaattgtcgatagaaccacaaaaagttgtatcttatcatgaacaagcctacacgatcaacgtcgttggtcaacgaagtcaaactgctgcgtagatagttgaaattgcgcgcactattgatgaaatagtttgcggatcgcgcaatgaaccacaatactttggtgatgagagcatcgctcgatccgtaatacgctagcggttcgagcaacttgtaaaagtacacattttcattgatcacggtcaaattcattttgttaatgttcgttatggtggagttgtaattttgagcgctcactttgacatcgctcaacccgttcgtaggacacgtgagcaacgaagaaggcacgtcgatcggcaccacggttacaatttgcataatcaactttggcacaatggtgttggattgatatctttgtttcacgttgtcgataaaagcctcatcggaaaccatattgcttgttgtcgacgaagacgaagacgaagacgaagacattgtacccggagtcgtttgatcgggcactcttttagatttagaaacatttggaagaggagcacgcatttttcgtttacgacgcatagtttgcgtcatttgctggtaatcttcttcgtcggtgctactgacagcgacggcagcgacattaccggacgaaggagcagtaaatttaatgggtttatgttcagagtaacgatttaataaattttttacaacattattggtgatttgattgaactcttgaggattgatgttgatttctaatgtgtaagcttgttttttggcgacaaggtccagtatgctcactacggccgctctcacagtaggcggcagagattccaacagttcaggattgttgcgtacacgttcggcgtaatagttgacatcggtaactacgcagttgaccggtccgtattttttgtaaatttttataaatctttgcaaatcatcgttgaggtcacaatctacagagtcgagttcgatgccgtctttgaaataatcttcgtatacgtcgttttgaatgagcttttcgaaatcaaacaacgattgtttcgtcaacgattgtagactgttggagtcgagcaattgttttgtcaaacttttaattttaaccatgttcatttctagatcatcgttgccgttggtgtcgttttcactgtcgtcttctgaattcgattcgctatcaatgtcattgtcgttttcgtcgtcgttacgttctatcaaaactgtcgtgtccgatgaacctatcgatctcgcagaaggaggaggaaaagacgaagtggacggttgaggcaacgtgtcttcgttggtcgattcgagaatctgacgcaagttgtcgatgacgttgtctaatgcgtacttggcgatgccgatcgatgaagattccgcgtttttaacgcgcaacatgaaatttttaatcgtttcgcgctgattaggatgaattttgagcaaatatttactcaacaatctagcatattgatattttatttcaaaagtgtttctttctatagtgttttcaatgttgccgcctcggttcatattgttgataatataagtagcaaagtaaaacgtgtaacacaatgaatcctgttgaatacacgttgaggttcaacaaatttgacgtgtttataaatgttaaatttaaagtaaacttgagtgtggatgaaattgattccttagcgttcttatactccaaatactttaatcaaagtgacaatgtgcgcgttaaaggtttaacttttttcaatgaattcaacaaatgtgttgaatttgtaaaagcaaactttgaaagcaagcaagaaaacaacgacgtcaaaaagatcttttccatattctttaaagatgaattcatgagtcaggtgcccaaattcagagacattttaaattatctacaaaaatactttaaaccgatacaaaaacctaacgtgcccgccatttcggcgctatgtaacaattgttccgtcaacaccatcgcctgcttacaatgtaaaatcaactatttgtcggagagcatcaccaccttcgatacgggcattcaagacggctgggatatattcttacgtcccatgttcggtctaccgctgctactcaccgtactactaaagaccgagtttaacgaagagtttcaagtcgacgatctaatcacaaattcgttttcgcaattcctatacaatctgctgtgcgataaagcgaccgccaattttgtcgatcacaaggcgtgcgcaccaatcatcaaacaatgccagcgcgtcacggtttcgttgcgtgatcacgatctagaacgtttgctgtgtatgttacgcaacaataattcgtgcgactcgaaactctttatgccgttcaaagtgttcattgtcgaactggcgagaaagacaaaaatcaagccggtcaagatcaacaaaatggcttcggtagtgtttaccggcttttatctacggcaatatttagaagcggctccgaacaaaactcgcaccgccgccgaactcgaactgcgcaacgtttgcaggttcattttgaacaagtacaccgatgatcagttggaatactttattaggaagctagaaaacataaaaatcgatctgtttaaggaatcgatggagcagtacattgtgtccgaaaactttataagacatttagtttacaaatacaaactagacgaagaactcaaagtgctgctagaagaaaatgtcgacacgtcgccgttacaatagatccgacgacacggacgacgacgaagatattaacgctaatcaactgttgcaaaacctcaacgaaacacacacggtcgccgatttgatcctcaacgacacggaccaccagaaacgcgtcgccatcggcgccatgagcagacactcggccatcgccaagaccatcctcgacaacatcgacgaagacgaatcattgcgtctcggcaccgtgaacactattaacgtgttaaagttaatgtctgatatttacgataataaaatacccattgttaacaatcaataagtgagtatcttaaccagtcaaaatgagttctgttatgctttttcttgagatcgaaaggatcaaaaataaaatcgatagtgaaatgaacatggccatatggccaaaattttttcctttgctcgccgatcccgacagccacattaacctatcgctcagcgaatttcaacagtttctagtgacggtcgcgcaaatggcgcgcaccgctcagatcgagaataacgctgcgttagcgagcgccgctcgagacaacacattgagcggtgtcgccgaacaaccgccgccgcctccttctacttcacagtcgagaaacattctcaacttgttcggcggcggcggaggcgtgtctcgacgaaaagacattaatgcgtccgacgccgtcaatcttgccacgttccgtaaaaactgtcaaaaactcatcaaacactatacgttgagcaacacttcatcgtcagagtttagagtggcggacattgtttcgtgtatggtttatcttgcaaaatcgcccaaattcagacccctgtactctcttttggaattgtccatgaccgacgagtacgattgtatgcccagctacaccgccgacgaaatgcatcatattgtcgacctgatcaaaagtcttctagatatgcccacgtctttgatcgatttcggcagcgtaaaactgctaaagagcacgttcaacaaggccatgaactatcccatcactagattcgctcgagtcatactcttaccgaccgcttcgttggccagcgacaaacgctgtacgatcgaggaattgttagtagaaagaggacaagaaatcaataaactcgaacctcaacagtatttgaattcgagcgaaggcaccaaaattccctattgcgacgacgaaaactttattaacgatctgctgaaattgattgacgatttcaatctgcaccgtatgttttacaacgcagcaaactcaatcttttacacgaccatggaaaactacgcggtggccaattgtaaattcagcgtagacgactacaacaacatattcaaagttatggataatttaagagagtatcaaactcaatgcggcagcagcacactgatcaattccaaagataagatagattctctcaacatatttttaggaagcggcgaatactcgtcgtccggcacttcatcaaaacgtaaaaaatattaattattataaggtagaaatataaaactcgagtgtgttgtattgcatagtcaaagtttaatatacaaaaagaatgtatcgtcgcgcatccaccggtcggcgtcgtagtagtagacgtagatcaagtcatcgtcgcagcagcagcggcggcggttatcgtagaagaagcagcagcggtcacagacggcgcagcagcaggcgcagatcgagccaccgcagacgctccagcggctatcaccgaagacccggcaggccgcgtcgtcgttccggaggatacaggcgtcgtcattattagttaaccgtgacgatgcggttacacttttgacaatatttaacaaacgaaacggcttcgtccccggcccgcatctgtttctcgacaacgacaaatttatgttcgcactctttcagcgaaaggccgttcaacgacggcaaaccgattgtgtattttgtatgttgtaaataaattatatcgtcagtgagaatgtttctttttgtgaacatacgtttcttcctcatacacttgctgttgtttgcttcagcaatctttttttttagcaaaatggtcttccaattgatcgagtacttgaataggatattgtcgatcggttctttttttagctttggtggtttactgtcaaattttttacttttaatgacgttgacgtagtttttgatttgatcgaggcgacgaaccaacaattcgcacggacacttttccggcaagctgtcgcgttgaatgtactcgaaaatctccgtgtataatttgaaatcgttgatagtgttgatgaacagtttatgaatacaatcctcggaaagacgtatttgtttgcgttcgcgttccacattgctgacggccggtatataagcatacaacgaatgaaacaaatgtcccgtgtggacaaaattaaacgttttattcttcacattggccgagtagttggttactagaaagtcgatcagttccttgtacgcttttcgacgacggaattctttaaagattaaaaataaactgtaacaatcatgctgacttggaccgttctgcagcgtcgagacgtcttcttgcgccgacacattcttgtgttggccgaaagcgccgacttggcatgcgttagctttagacatttgaaaatgtttgaattcgtaatctttgcttttgacgcgaaacaacgtatcgacacacgagactatatggtgcagattataaaatgtgccgacaacatgaccgacatacgatcaaacttgaaactggcgtataaaacgtccgcgttaggacacgtgtatgttattaatgaaaagattccaatgtacgggtttcttaaagaatggtacttgcaaaactatctcgaagtttatcaaattaaatacgacaagtacgtgtgggaaataccgcacgttatcgtctttgatctcgacaacacgctcatcagcgacgaagaacacgtgcagatccgcgacgagtttgtttatgaaagtttgacggagctaaagaacaagggctgtgttttggtgctatggtcctacggaaatagggagcacgtcacgcattccatgaacgaaaccgatctcaacggttatttcgacattgtaatatgcggcggtcaaagattaaccgatctgcgaagtcgcgtcatcgtcgacaacagttccgacatggtgtttgtagagaaacctttttatttagacgttgacctcaccaacgatcgcctgccgaaatctcctagaatcgttttatactacctccgcaagattggagtcaactacaacaaaactttgacgctcgtcgacgatctcaaagataacaattattcgtacgactattttttgaatgtcagccgatgtattgaacctcgtcgtgattgggaaaagtatcatcatcagttgattgcaaacattgacgattacgaaagtaatcatttattgtaagcaaatttacaatgattaaaacaatatttgttatattaatagtgattattgtgataacaatattaatggttttttatttacaaaatttaaaaaatgaaataaacgaagcacgatacttgccaaattacaattacaacgatatattttatgatcgtgaaactggcaaaacgtacaattgtcctcgatcgtgtcccgtttttagctttaagaaacaaaaatgtgttagaggcaatcttcaagcttacaacgtttgtccgtctaataaattcggtaacatccgacacaagtatagatgtgatgtgtttgtattttgtgcacaaaattcatcgaatgtggtatatttatgctcagaaacaacgtgttttgataatgagcaaggtatgtgtgtgccatgtccttcaaaaaactgtagttcaaacgatagcattgaatgtacagattgttgtgaaataaaagacgaaaattaaacacatttttttattgtcaacattttttgtatagcagtcttgaatctttccgcatcttcagaatcggtaaagctcagcgatcgtgattttttcttcaatgattcttccgaaaattcatttttcaaactttgcgtaacgtttgtccaatccaacatcggattaggccgtttcgattctacgataatttcgtaattgtaaaatttacgtttcaacttttgcgctctaacgtacttttgttggcctacgatcacttcaaaatcattgttgtttttagctatacaaatcatgggcatctttgtatcgtcggcaggatactgaaccactctttcggaaacatcgtccaacttgtcgagtagtttattgatttgcttgtctttttgtaacattatttcgttagttttggatacggtgtcctgcaacttggtgtacatgttgttgacgttgttcaataacttttcgatcatggcatttttatgctcggtcactttgctcaattgtacaatcaaattttgaacttgttcatccttgtttttcatttgtacttgaaaatgtttgacgatttctttttcttcggtcagcgccgcctgcggtgaatatttgccggtacacaaaacttgcggtatcacttcttcgaacatccattcttgcaactcgaccgcgtacgacaatttgcttttcataatcaactgaatgacgccgctcttgttgatcattatcgtttgcgggtgtatgtagagaacgttgggacatggtagcaaattgttaatttttagtaatctcgcgttgactatgaccgatggcatcaatgttttaatgtcgccaaacgcaatcttgtacttgtcgtccacgtgatcgatgatggcttttttagtgttttcatattttaacattgtagcgatatctttgccgacgaaaaacacctcgtcgttgtcgccgatcacatatcgcaactcaatttcttcgtcgccaaatttaaacaagtttattttaacagaggccatgtctgctgtacgaatactttcaaaatgaaatgaaacacaattttcacattgtaaaataattatatttttctgtatatctttaaatatcttacactttcataggatttacaattatcatagtcgaaatcgttgttaatgtcggcaaagtgttcgagaaaccccgctaaaaattgaaacatttcatacttgtaaaagggtacattttcgcttatgtcgattatcggattgatgcgcagattcttgtccaagtcgtcttcgttaaatatcacgtccctctgcacaaacttgtacggtatcgcgtccaacaaccgatagtttcttggattgagccgaatcgttttgtcgaaagcatcgcatacgacgaataaattatacccgttgtcgttgaagcaccgatagaacactcgatccggcaaactgcacgcaataatgttcttgtcttcccaacaatagtaattgtgcagtctcttgtacaatagcgcttccattgtaacgtttctcttagtcgtcgatgattgtacaacgtttaataaagaacaagtttatatactttattttatcagtacatatccgaaatatatcagataattgatgatatcgagtatactgtacgcaatctcgtcgaccgtgagcactatacgtgcgtcgctaatattgttttttaacgttaaaaagtgttcgagaatcactaaactgttaatgttcgtacagaccgtgttgtatatgcccgagtcgttgatggattcgatgtcgtttttcgtgaatcgaaccagagaaaactcgcgcggacaaaacacactcgacgattccaaataatagaagagcgttccgtcgagattgtacaacaatacattttcgggtttaatgacaaacaaacgttctattcgatcgggcggcaccgacaagtcatacacttctatatacgctccaaactgtaaagtgttcttgtggtcatttattaattttaatacttgagttcgataaggatttaaatagagaagcgcaatcaaaaatattagtagacacataatggcgactgcggagataagtgttgacattatattcgatgaaatttacaatgatatcgatcaacaaaaattgtaccaagtcgacacgctcaacaacgccgataacgtgattctagtcaatagcgtgtctcaaaagaccaagaccatcgatacgtacgataattttgtaaagcttctcaccttaatgaccaacacgcaaacggacacaaacgaccgagtcgtgtgttccaaagatcacgaaggcgtcgccataaaaccgcacgattggtgcgcacgtcagaacatctttcgcatgtttgtgcgcccgtttatcgacgccgaaaactatagtcgtgtcaaggatcagatatgtttcaaaaaattcatatcgagcaactttcccggatacgccaacaagtgcgtcaagtcgggagactattattattggcccaacatggccgtcatatactgcggctggaacatttacctaaagaaacagttcaacatcaacatggactgttccataccgttgatcaacaatcgtcgtttgggcaacgtgaacctgttcatcaacaatcccgaagatttccttaacgtcgaactgagcctcgtgactagcgacaaccgaacgctgtttgtcaacggtcgatcaatgtttaccgaagacgacgacgatttgttcattatcaccatggacgacgataccgaatcgaaatgtaaaatcaagccggaactcgtgtatagcaacaaaaatttttttgactacataaaagatgacatcagcctcaagaattgcgtcaccgccgacgattacaaagatatcttgtacattgacctcaaaagtttacgcgtgtttgaagataaaatcatgcctagtaacgctatcgttaccaaggaaaaattaaaagtatttcacaacatcacgccatccagcgaaaacataaagatgatgcaacaatgtatatcggattgcgtcaccctgatcaaggaaaaaatgatcgaggtgatgtcacacacggatcaggcggacgccagcgttctcagaaattattttatcaaaagcgactttgtcaactttgactacatcattatcgttttgtggagaacgatctcgaagaatgaagacatgaacttgaccaagaccgatttgaaattatactttgagttgctatgcgaaacgatctttggcgaaaagggccccgcttacgacatggccaaagagagatgcgaaccgtattgtttgctaacgccgaaaatatttttgagattgtgcaatcattggaccatgttcaccaacgaaaatccttgcgtaactttggccttttattacgccatacattttttgatttattgcaagcatggcaactgggaatacacgtacgagaacgccaaaacgtgcggcacgatccccgaagtgctatgtgctggttttttcaaaaagattgtgtcgtcgggcaacatgacgtttgtcttcaacggcaaacattacgtttcggtgcgcaaagatgacgacctgtacaaacttacggaaaattgcacgagcatcacgatgcccagtatcaagtttaacaattggaaatacatgtacttcaccgacgaaggcgtctacaatcttttcatcaacgattttcatagcggttgtccgtttttgatgggcaatactctggtcaaatctttaacgcacaaaaacgagaaaatctatctgcctcaaacggtcatccagtacatgctggacacgggcaaggttgagattgaaatttacaagatttaccacatggccaaagtgtgtcgtgaaatcaaaaatgtcaaaaacaacatgacttcaatattggcgtttaacaattgcgcgtcgtgcaagtttgaagaacaaaaaactctcaacgatttgtacagagaaatttgggcgtacaatcatgacgaactcataatcaccggcgtctatctcaacgacattaaaatgtccgacttgattatcaatcttaaatgctacgagtgtaaagaacatatgcacagtccgtacaaaagagactgttcctgtttgaacaaactcgagattgacgttaaagcgttcaaagttgttctcatcatggaactgtttacgaattgtaaagagatactggaattggtatggtcgttgctgtacacgtcatcgctgtacactaccattttgttgtacaaatattcgtgcgacaacaacatgaagctgttgcatgattacggagagtttttcaccgccaatcgtaccaaaatcatcaacttgctctacaattggttggacaacattgattttgtagattcgtttgttgcgtcgttgtcttgtcccgaggtgtttttgaatcgattgaaaaattccgtacttaacaactcgaacgacgttaatgaattcgaaactatcgacgccattgaaaagtttatcaatagcgatagcctgagcgttgtgtcttacagcggcaacaatgacgacgacgacaatgacaacaacgacattcataattatgaaaaatcttttttaaaagacttttactacaagtattacacaacgttgagcaatctgaaaacatggaacgtttggtgggacaaactgatcgtgatgcgtcacaacgacgatctcaacacgtggctaacgagattctttatgaggatattcatgacgaaaatggaacttgaaggttacccgtacgtttttatcaaacaagtggtcatcggctatttgtactttagacaattcaccaactttaattacaccaatagtttggtggtcatgcatttcgccgccggtaccggcataccgaccgattacgagaaatgttgtttgtacctcaacggcaagcctggctcgggcaaatctagtttttttgccgttttagaaccttcggtggtcgtgcataaacacgattcggcaaagtacacgctcaccaaaaaagacaccaacgaaatggaagcagacaagatgatttcacaactttacgtcatcaacgaaatgaaggtgtgcgatgactcattctttaaaagtacagcagattcgacgaaaagcaacagcgtttgcagaaagtacgaaggtagccaaaagtacgaagccaactttaagctgttgatcgtcaacaataaacctttgcacatttccgattatgacaaaggagtgagaaataggtttgccgtcatttacacggaccacttgttcgaagaaaacgtaaagtttaacggatccatttactggcacatcaagaacaaaatgtatcccatggagaagagctacatcgacgaaatggccaaacccgtgcgcatgtttctatcgcacattctaatgtacaaacgcaactctagcgacggatacgtgtcgtacaagtgttttctagaaaaggaccccatacacaatcacaatctaatgtgtctcgacgtcaacaacagcccgttgaacgcgctcatctatgtgctaaaggtgaacgtaaagccgggcgcgagaatggtggacgagtctaaaattgaaaaaatgatcgaactggccgtgccgtacgtcgaaacgatgctccacgagctgctggtcaccaaacgtaccaacgtggcccaacgtaccgccatgctgtttgacgcattcaaaagaaagtttaaaaagtactacttggaagagagaaaaatattctacaacattgacatggcatggaacaagggagatttcaacattaatcaacctgaatttatatgttagtcaaataaaaaaaatatctatgtacaaaatggtttattaataataaaaattcttaaaacaataatacaattgttttattattgtagcctaaaggatttttcgtttttgatgaaaagcgttccggcgtcgttgatctgtgtgtagtcccattctctgagtttgagttgaacgttggcgtttgtagtgtacacggtgatggggtatgaagcgtcgcgaagatcgggaaccgacgtggtcgtagaatcgatcaggcaatacaccattccgtcggcttcgaatcgggccatgttcaccgattcctttatcttgttcgagtccaagtttttgaatacgataaacatgttcttgaactgtttaatgtcaaagttggcagctacgcggttcgaagccgagctggattgagccgtctcttgcagcgtgcctaggaacacgcagttggtgcccactgatttattgccctcttccacgatatcggtgtaagtcaacggtctttcggcgacgtaaatcttgctaatcttgttttcgccgtgcaggacgcgaagtgattttattttggcgttgttgaatttcaacgtggtcgtgccgttttcgttgacggtaatcgaaccgctgctgtcgttgagactgggcgacgattcgttgatagagttcatgtttagtttattgttggtccacaaatagtacaataacgccagtacgattagtatcactactattgttccgatcattttgaagcgatttcaacaatcagattgtccgataaatgttttaatagcttacttaaattattttcttccaaattgatattattcggcaacacgattattatacgactttgtaatctgaacaatttaaatatactcgtcaagtaatctagaataatgtttaaaatgttcttgccgaacgcagtatcgtcgaccaattcaacatacgtcttatacattagaagcgcgtcgatcaacacgtgatttttgcagtagtttccgctgctgtgtactactatatcattcaaagttttcctcattagtaaggcctcttcttttttcacctcgtacaacacactgagtttatcgcaagactcgtcgtcgaggcattcgataacaccgccaaagtaaacgacgtcgtcatcgtttggtttatcgttgttgatcccttcaggcatatcgactagatacaaatagattattcgttcgttactattcattatgatacctcaatcgccgttgttcacacaatacaaagacagttttctgttgtacacttttcgtcacttggaccgaatacgcgcatcaaagtccaaacagttgagtaaaattttagccagcgaattaacttatctatacgagattgcgtgcctgataatttataaagatgttcaaaacgaagagattgaaaagctcaaggaatggtctctgcagctgtctccagaatttgatttggaacaaatgaaactgttgtttcgagaaaaggtccaagagttaaacttgcgcagccttcaaccaaaaaattactcgtacacgtttacgacaatatggaacactattcattttatcgcgctgctcatcgacgacatgatagcgaatcgggaaaagatgacatacgattttatcaccaatcacttgcgacaaatgaaaattttatattataatttatttttcaaactggattgtgccatgtgtcgtgatcattacatgacggtcaaaggctttctaatacaatcgatcgaacggatcgaactgtgtctctatcgagaacgctacggcgaaaagatcaccatggtcgacgagataaccgtgcaaaacgtcaacgacaacgttttaatgaaacacggcatgttatacacgacaatggtgtttcataatcacatcaacgactatcgatggattcaaagaaacattaaacctcccgtcaactatgaaaagatgaagtggtccacctacaaagccatgttagaattgaaataaaacacaattgtttcaaataaaaataatttaatacactacaatcttaattaggcaccaaacgatctggacgctgtttaataatctttatatcgtcgtccgtgataattgcttcgtatattgcgccgttttcaagtttatctatcgtcggcgaaatcaatgtgttctgctgcaacagcggaccttctaaatttttaaacagtttcatttcggcgtcgtattccacttcgacggttttaacgcgtttgtacttgacgtagttcatttccgtgtttaacactacaaacccatcggtgggcatggtcgagtatccattttgcacaaacggtggatcgaagaattgttgaaacttcaccgaaacacattgattcgtcatcgtacgcaaacaaatcaaggcgttgttaaatttttgcgctaaaaaatttatcgaaaccacggcgtcgtcgggcgctatctgatacggatcgagagatatttcgtattgagtgcgattgttataattgtacttgaaaacgtgcaacagatcggtgacgtataatactttctgatcgatgagttcgcattgaaacgccaccacgttgttcaactcgaaaagaccgggctctatctcgccggagaacatttgcatgtcgtccatgaacacgatcataaaatttttcgtgcaaaatcctttaccgcgtatgccgtccaatttcaaagcccacttgtaaatgctatcgttttcgagatcatagtctgcaatgtcatcgagaagcttttcgtcttcgaatttgcggtagattatgttattctgcagggtcgtgtacggtaacaacggactgatattttgatgcgcagttattttatcaatttgtctaattacgtcactcatatactctagaacgttgtaatcaatagcgttttcctcgtactcgtactcgagccgtatagctgccaaaatttcatcggaccccaaatggctttctttaatcaatttttcgtttttgttttgcagcaagttccagagcattatctgcttcgatgccattaaagaatcgaatttatcaattagacttcgattaaagtaaatgtgttcaaatttaatttcaaactccggacactcttcgttcttgtacacgatacattttacgatacgattcaatatcggagacttgtctttgctcggctcttctgtactttctcgatccacgaacggaatcaacacgttttcgttgacgtagacaaactttttcaagtttacacattttttgattacactcgaaaaggagccatcggaaaacagacgtgtcctcacgttgttctcgtcgacgaaatcatagtagtgttcaaaggctttaaactttttagaaatataagaatctaaaataatatacagcaagtcttggctgaaatttattgaataagaaatttcgttttcgacaaccatggcactgacgcctatttcatcgactgccacgaaacgtaataacttttgtattttcggagcggttcaatcgtttgacccgtgttacacgtacacgaaaacttgttctcccgatgccgctttcgacgacggatggtacatttgcgagtatcacgcttcgatacgatttaaaattgaaaaaatggttatgcctattcctgacggagagggcagaacctattacagaacggtcggcaagagtctggtcagcgacaaagccgagggcagcgagcgtattctcatacccacagcggacaattacgaaacggtgttgaatatcaacgcaatgtcgttaccggagcagctaattttccatatgatatacaaaaatcgagagaaacaggagcgcgtctgtcaaatgctacagttcaacgagcatttccaaacggaaatttacaagataatcaattccgtatacaacaacacgagaaacgtgctcgcaatgacggatcccactcaatattgctccagggtcagccagaacagcactcgggtctacggcgttaacgatgaaaacgatatcgccgaacagaccatcaatcgaatgcccggcttcctgcagaatctcatcaacaagtgcgtcgctcctcaaactatgaacattgaaaaccaaacgctagaatttcgcaattgcgctacctgccgcatcgacaccaccggcttggttgccgacgttaaactatataatcccatcgaacccaagtacaaatcgaaacgggacgaaaattatctaatcgtagataacgtatttaaatttcgaggaaacactaaagcgctccagacttctctcaaccgatacgaacaataccccgtcgtggttcccctcattctcggcagcgaaattgtggtgaccgccaacagtttgaagcaggttcctttgccgcgcatcctgcccctgcccgaagaagtcaatcctcctattccaccagtaaacgtggtgcccgtataagtggtgtgaccttcaaaatgacttttatttaacctctacatggacattgaccccattaaataataataataacgataatctattgataaagttatttttttggaaaattatcattgcatacgtgactgataataataatagcgtacgtgactgatatagcaaggatttcactatataaaccgtcgacgaaaatgtaattgcattcagtcgccaccatggatttagtgacaatacagtgcatagtgtgtctcgtcgagaccacaatagatcacaccaaagtcgaagaagtttttgtggtgccttttttaaagttgaacaactgcaaccatgtcatgtgcgtggattgtataaaaaatatgacgacgcccttgtcgtgcccagtgtgtcgtcgtgtgagtatcgccgtgcgcagcacttggtttggaaactataacaatgaagtctggtcgtacgatttgaagagtttgatcagtgaaattaaaaaattacctaaaaagccaatcaaacaaacgaaaaacagtctactgaccagtattaacaattcgtttacctatggtgttataaaaactacaaattcacaaccgtcgacgtcatcagcaactatttctaatgaaattattaacgtcgaaagcgaagatgaagattttgtcacgaatcaagtttctaatgtttgtacttcttcagatgttggaaattctgatcctgttgcaagcacttctactagttctgttactacgacttctaatgattctgttgctagtacttctagtaatcatattgcaaacaattcaacggaaccggttgcgagcacttctacagaaaatatcgcgaatggtcctgttgacttgaacgatgccagaatcgttgcgatctctgcaaaaattaatgatttgatgaaccaacagtataacgctgtgctgacgttgagcaatgtacaacatcaaatcgacacgttacacgtggaacaaaatacattgctcaacattaacgaagagctcactcgtaccaaagaagagctcacttgcgccaaagaagagcttatttgcactcaagaagcgattacgcaagatataaattctaaacgacaggagatgacatatctagaggctagattgcatatttataattgtaatgtcaagtatgccgaaatggaattaaaaaaaataaataaagatttgaaagcgaaaagattgcaactttccaatacgaagacagagtcgaaatctacgtcgacagaatcaaactcgtcagaatcgaaatctacatcgacagattgtttgaaagactttataacgtcgtttgattccaacgaagagtttaataaaagatttgcaaataaaaatacaaaaaaatacgtagaatcattgataaactacattatgataaagacggagaatcaaaagaaatgtaccaatgaacaaatacaaaaattgctacaatcccacgacagatactttatcataaacgagaatattttttcacccaaacgcaaacaatgcgacaacagcaacggtgataatgatgacaacgacgacgtcgacatcatcatcgagattaacgacgaaaatgttaaaaaaaaactcaaaataaattaaagtataaattgtcaaaaatgtttgtattcttgtggacattgtaataaatatttataattaacaattttgttgttgttttaattaatattgtaaatcttcaatgtaatagattctatcctcttctaggttgcatgtaaagtcgtcggcgattgcgtgatcgggacacgccatgatttttccattgttattgaatatccatttgttatactgtactccgtcgtcggactgagtttcgacgttcgaaaacgttttaccttccgacacttgaactctaatcatgtccccgtcaaatgtcgatttgcatcccaaaccgtttcctagatacgcgtaagcgttgatgatgttggccaatcctgccgcgctctcgcattctgcatccactttaatattttcacgcaagttacgaatactttctttaatatgatcatcgacgacgttacacgcggacagtttaaacgttggtatggcatagaaacatgtgtaattcaattctttgtcggtcaaagttaacgaaggcggctctggttctggctcaggctctggttcaggctccggctctaccggaggtcgtgtacctcccccgccgccgcttccccctccgacatcatcaggatgtacgtcaatgatgtcacgagtcgaccacggatcgaatagcggttcgatgacgtcgtacctttcgaacaaatcaaacatgggcaccatctcggcggaggtgtcagtttcatcggcttcaaatggtatatttgcccgatatcgttcaatatattttgggccttttacactttcgtaattatatttgagtggaatcgtagtatatttgatcggaatcgtagtatatttgtcatgaatcttggtcaagatttcaccgcacacgtcagattgtagtatattggccattatttgcgtcgtaaaataatcaaactctactaaattttcagttgacactagacttttgaaacaccaatgttgatagtcggcgtcgctgcgtatctccatcgtcaccggcgctatatactgatcgggagttaatttgatgttttctacgagctcattgtctttgcatacgttcaattcggtagctgcgagcggatcgaacaaataatcgccgaaacaatctatggtgtccgaagtgctagcgtcgaagccgagaacgttaaaattgcgagcatataacactttattggccaaagtgtcgctgccgatcaattctcgtacttttagcgagtccccgagaatagacgttataaagtttatgttgtaatcgttggctacattttcgatgacatatgacaaaactctcggcgagggacgggtcgcagtgctgggcacgcattgacgagtatcgcgattgaaaatttccgcagggaccataatgtctgtgtagaatttattattatacaatttatcgacgtctatcaaattttcattgttgcatgtaatatcctcgacaatgttgtaattatcgcaaatcaatagaccgtagtcgtacctaaaggtattgtcgtcgtagttgcgtatctgttgaccggtaccgttttcgaacatggaacattgagggtcgcccgaacattcgtaattgtcgttgacaaacacgcgattgatacacgtgacgagttctacttgattctccgatacgcatctgtaaaactgcgtcggtccaatgtcgtcggtgatgtatgtatagtcggcgccgtggacagcgcaaggctcggcatcgacgcaagtcaacagacgacgatcgaagattttaccaaacggacacgacaccaccttgctttccccgttttcgcagatcatgtactcgttaatgttcaagtcttcgggaaacacgtttagtatgaaaccgtcgcttcggttgacgcaatcgttacgcaattcgcattctacgccgttgaatagatgattattgggacactcttcgacgacgtgagacccgccttcgaaacatctcaaatacatagttggatgaaactcgtcttgggaaatgttagcacgggaaacgcgatgattgagaatcaacgagtctatcaatcgctcggtcaacggatacattcccgaagttttgccgtcacacggaggtatgggcacgcatctattggtggtttcgtcgaaacgttcgtcgctcggacattctacctccatccacccatcgtctccgcgcaactcatacttggttcgatcgtcgctgtttttgcgtacgcgtttctcgggaggcggtgtcggttcaaagttttgttcgatgaaattgaacgtttgtaaacgatcatcgtgcacttcgttgttgatttctctcaaatccgtagtatcgatggtgcggaccacataaaagttcgagtctacctcggacacgtattgaatataatcaggcaacggatgttcggcattggtacgtttggcgtattcggtcacaacgcgcaatttgttgtcgaaatcttgttcgttatattccgaatatattattaagtagaagattgaaaagagtaaaatcaaaataatggccaccagcaacagcggtaccatagacatttctgttcacgttactctcgacaaggaatcgtcaaaaaggaatgttctttcgtttattgttcgagaagaatatcacttaaaaaaactagccgttggcgcttacaacatcaacattcttgatactcagctgctcaacaacttgtcgcaatacaaatgcaacacggtagcgtgtggtgattacgtaattgtgtacaatttcaccgaaaacactagcaatcgaatcaatgtcattttgttcaatataaaaccgaccattttgaaaaagggcaattgtatatttaaaatagtttacgaagacgacgctacagcaatcgttactactgataagatgttagacgagccggttaagcagcatcatcaagaacatgccaatagtcaactgttcaaatccgctttcacacgagactcagacgacgccgcctcctcttcctcctccgcatcctctgacgaatcagacgaagacggaaacgtcgcctcaagaacggcagcaaacgacttgtctgaggctgtcccagctaaacgacaaaaacttgacaactttgtcgagagtcaagtatgacgctgatcttttactacactacgttttcgatggaatcggtcgcagcgaaaacaccaacgtcatcagagtgtgtaaagttagagttaaaaagacatgcggtactctgttggctcactactacgctcaagtcgaaatctcaaacgggttctcgttcgaattccatccgggcagccaaccgcgtacctttcagcacgtacacacagaaggaaacgtgatattagtgttttttctctgcgacgattgctgtaaaaaagaattgacgcatttcgtacaaggcgaaaacgaattcaatgtcgcttttaaaaattgcgaaagcattctgtgtaaacgtagaagcgtgcaaaccgcctacatcaccatggcggttatagttttatttatcaacattatacatttttcatggtattttgtactatttattttcgtaatgatattcttgctatatctaaacaataattatatgataagtaatccaaaaattgtttactgccctcataaaaaacacaatggccaattacacgaggccaaattcaataactaaatcgtcgacaatgtcatcgtcttcgttgtcgtcgtctgcgtccgcggccataaccgaaccgtggatggacaaatgtgtcgattacgtcaataaaatcgttcgatactacagaacaaacgacatgtctcaattgactccacaaatgctaaacctcatcaacaccatacggaacgtttgcatcgaaacgtatcccgtagacgtcaacgccaccaagcgtttcgacagcgacgtcaaccttatgaacaattacaaacgactgcaaaaagagctgggcaataaaccgatcacgagcgacattttcaaagcttcgttcgtgtacagcgttttgccgtcgtacgctcaaaaattttacaacaagggcggcgatcatctagccagcggcagcgtcgaagaagcggcccgtcatttgggctacgctttacaatatcaaatcgcgcaagctgtgaccacaaacacgccgattcccttgccgttcgatcaacagcttgccaacgattatctaacgctgcttctgcagcgagccaacattccgacaaacatacaggagatcatcaacagcggcaatcgaacgcacggcaactcgcgcgttcacatgatcaacgctctcatcaacaacgtgatcgacgatctgtttgccggcggcagtgactattatctgtacgtgctcaacgaaactaacaaatctcgcattctcagtttgaaagaaaatatcagttacatggcaccattgtccgccaccactaacatattcaactttatcgcaacgctcgccaccaattcgggtaaaaagccgagcgtgttccagagcgcttcgatgttgaccatgcctctaactaaacctgtcgtcagcgaatccaaaaacgtgtgccaacagcaactgaccgaactggcgtttgaaaatgaagcattaagaagatttatcttgcaacagttaagttataaaaacgacatttcgcaacagtgataacaactgacgctagaaaaaaaaagatgagtcttgacgttccgtacgaacgtttaggcacagcgaccaaagtcgattatattccgctaaaattagctttgactgatttaccttcagaaaacactttagacaacaatgacgacaatcaaaaaaacaacaatacccaaaatcccaaaattgatattaatcaatcaaacgccaacaattataatcaacatcaatcggttcgttcaaaacaacagttttacgacattttagttttaggtatgctgacagtgttttgtattttggtattgctgtatgctatatattactttgttatattaagagacagacaaaaatccaacactataagacctagttatatgttttagcatgactgataacattttcaataaaacaaacaatgtgagaaatgaatattcgtttaattgttggaaatccaaaatccaaagtcattttagattcgagaccgtgtttcaactggccaccgatcgacagcgatgcacgcccgacaaggttcgtaacggtcggtggtccaagtttatttttaacaaaccgtttgcgcccaccacactgaaaagttacaagtctagattcattaaaatcatctactgtctaatcgacgagtctcatctcgacgaactaaacacctacgatcttaatcaagaattcgacagcatcgaacagcaaagactagtcgtcgatcccgaagaactatgtatacgtatgcacgaacttcgatccgtaaccaaagaaactcttcagctgaccatcaatttctacgtaaattgtatgggtatcgaagagtatcgtataccaaaagaggtaatgttgcccagagacactgaaattaaaaacattcgaaacaaggagaaaaatatcgtattaaaacacatactagacacggtcatagattgcatcaaaactcgtataaaatatttaaacagcgattatgtacacgataggggtttgctgcgaggagctataattttttgtatcatgctcggcaccggcacgcgtatcaatgaagctagacaaattacgctcgaaaatctcgaggccatcatcaaagacggcaaagtgagaagcaagatcaacttgaaacgcaaacgcgatcgtttgaatcctctgcatcgactcgaattggctcctttgctgttggcgcgagagatttacgccaaacacccgaccatattgcagatatcaaagaacacgtcgacgccgtttaaagatttcaaaaggctgttcgaggaagctggagtggaaatggacagacctcgctccaacatgatcagacactatttgtgcagtaatcaatacaacaaaggcgttccgcttcaacgcgtcgccaaactcatgaatcactcttcgttcaaaagcacccaacattacatcaacaagtttgacgtcgacatcgacgataccgacgacgatgacgacgacatcgaactacccagtcctgaatctagtaactaggaataattaaataaaactctgttaaacatttttcattagtttttttattatcactcttctcgaaaacaatacaattttcgttgaactaactacattgagattacaatacatgtcgacgaaaacgatacgactttcatcgaaaatcaattttaaaaaattttttacaaaattgtaaaagttaactttgtcgacaatatcatcattttcggcgaaaatacattatgtttttcgctgacaacgttatcgttttcgatgaaacgaacaatttaaattacgacgcaataaataaagatgatgcaataattgataaaatgacgtcataatatcattttcgtcgacgttgatatgagtttcgctgaaatgagattattaaatgagcatttcgatgacaatgctatggttttcgtcaaagtgagtttttagatttattttaaaaatttttacaaaatccacttcaacgaaagcaatgtcattttcgtcgaaattatatgtattgtttcgacgaaaacaacattagtttcgtcgaagatgcattttagaattttgtttaaaaattttgcaaaagtcgtttcgatgaaaatgccatagttttcgtcgaaacaacacattaaacttcatcgaaaacgatagcattttcgtcaaagtctaattttaaaatttttcaacaaaatttttcaaaaactcacttcaacgaaaatgctatcgttttcgtcaaagtctaattttaaaatttttacaacaaaatttttcaaaaactcacttcaacgaaaacactacccgtttcgttgacgttcttacaaaattaacacattgcctgcgaactgcgttttcactctgagtttagatatcggttctccgttgtacacgacgttgatgtacacgttctttttgtcgaaagtcacgtacacgtcgatgcgaggtaggatttgaaacgtgtgaaaagtgagatgatcgttaggatcggtcacgtagacgaatccatctagacatacggttccttgagctcctccggcgtctccggcgatcgggacgatgcagtgattttcatcgttcacatagtaatccgtaaccacagaaatcaatgttccgttgtcgtcgaaaatgggtgcgcttacgtacaactgcgacatgaactcggccagattgtttttttgcataattgccggcacttgaccatacacataatattttgcgtaatgatgatgcgtgtagtacattcgatcttgaatacgcatcctgcgtagatatggcttgttgttggcgttgaacagcaatatgttcacgtacgagttgcgtttcagttttaaaaattttacatcgctcgccacgccaggataatgatacttgatgtcttcaaagtctgctgaagtgtcatgttggccgaaaacgtgtatgcgtacagcacaatttttttcactaagtacataaactctacgagtcatatgattgacataatacgttatggattttatgtactctatcgtcgaagcgttcgcggcgccagcggctgcggcgacattttctgtcgcgactgaattaattcgcagtgacattgaaaatgatactgtttcgaagatcgctcaaggttttatataaagctctattgtaaaaatacttctagtttattttcgatagttttgcgacaaatacaacagactttgcatcgtttggcacattgtgagcatgttgaaacgtgacggcacggcaaaaagcatgtgtctctttctcgctcaaaacatattttgcacatgacatcatctttttgtgcgtcaacgaccgtcgatgataataacaacgtttgatcatcgctatttatagacatgctgatttcattggaaagatccgggtaaatacgtgacacgacggtcgtttgattagcgagttcatcgatctcgtcgtaatggggcgccgaaggtgcgttgaatacacaatcgggcgaatagcgtccgtgtatgagagcgacgttatcgttcttgttcagtttgactatgacaaatttgcatgaagaacatataatttcaaatttctttccatagtaatagaatccatttttggccagcaactcatacatgtctttgtaataatgacgagctgttttgaatttacaaaaggactgcttacgtaacgtttcgttctcgaatagtcgttttgttgccatgggacacgtggagaatttgtgatatttgagactgtgctcgtcgagttttttcaagtacagcggacaaaaggcgcatttgtagttgcacattgcgtcgcggtaaataccactggcgacgagatcgttgatgtattcccgtgtaagataactgttttcgaatgtttcaagacgatcgacaatgttctgatagaatcccggcggagcgagatcatacgtgttccagttgggagaggtcattttcaagaataaatagcaattgcttgagagcccgtctttgtctttcggcaaaatatttcgtatgcgcatcgaattgtttgtttaagaccacgttatctttgttcaatttacccttgcaaactagatatttttcagcgtttgctgctcgagatgctaacggtttgtatagataatgttcatcaaaatgagatatgaattcttgaagaagacttattgttgacgattcaaaagtgtcgaatatcttcaagacacaattaccgcccgtgcgtaaagcgccaaggattatcatacattccttggctatcaacggataagataacgattcttgattattttcattgcccttgacgtcgaaagcaccatcggcgacgaccaaatcgcatttaccccgacaaaaatacattagctcgaagacgacgttggcgtcgaaaatgtcgcccgtgtcgaagcagccgtacatttttcgaaaaagtacatggtcgaacgtgtagtcgcaatgattgcgaagagtgacgccgtaacccgaacactcgtgattgttgacgtcgaaaacgtatttggcaaactgtcccggacctccgcatagatccaaaaatgtttcaacgttcttgcaaacgttgtacttttcgtcgatgtctttgatttttaaataacatctgtttcgtatgcgaacgccgccttcgataaactgccgagcgagacgaacatggtgttcgtcatgattgcctagtcggtttttgagcgcgtccaattgtttcttcagtctaaacattatcgtcgttatctttatcgtcggagttgaattgaaatgcgactgctaccgtcaataggatggcgcaaataaaagcaaggaatacttgaatcgatattatattcattgcagacagcgtaccagtaggacgattgagcgctctgttcaagttttcatttgttatcaacaattttctatctttgatttcgtatatcagcggtcggctgtaatcgaaaaactcaaagtttgctttgttgatcatagtacgaaaagtactatcctgaggcgagtcgagcaacgtgtctataatcaaatctttccacgcgtgttccctgctctcgggcgatacttcaaccctatccctgttcaacattcgccaacgaaccatgtcttcgtctacggaaatgcctaccaagcgcggactgaactgtgaagaacaacaagaagtcggcagcagcaatgtcaagcgcggtcgctatgccagccccaagaagcggatgtcgaacggttccacgacgagcgtagattctgcgtcaaaaaaaattccaaaaagcgtaattggccagctggtgacgaagaatatgtacagcgtcaacaatgaagcttattacttatttaaatttttggttgacaatgtgccaaaaaattattatggaaaccatcacatgtttcaaacgctgaaaatggacgctgtttacgagattgagttggtgtacgagaataaaagtttgtcgattgccaaagtgaccgagtgtaagaatgtggacaagaccgtgttggccaaacactatgtcgagttgagcgactttgacggcgaagacaccattagcgtgttggccaagttgaagtttggatttaagattttggacaacgatacgtacaaggctgtatttgtcgtgaatcatggcaatagtttggataatagttgtcctgttcagattgaatgtatggccaacttgaaacgttgggcggcgagcattaaggacgagagcataaccgatgagaattcattgttggaatatttcaactatttccaaaatcaaatattcagtctgaatcgcatcaagtgtcagcagagcaatggaaactttaaaaactttgctcttcaaaatattacgcaaatatcggtgtctaaagtgaccgactttgagattgacgaagatttggacaacatcaacaacattagtagatcgaacaaacgcatcgtgaccggctttgtggataaggtgaacgtcgagagacaaagcgatgagagattttcaatttcatatttattgaaggagagagacgatgatgaatggattcgcgcatcgttctacgtaaagaatttacaaaacaacaacaacaacagtgggggcaacgacaagaagaatgataaaatggaaaaattggaaaagttggagactgatttgaatcagttgaacgatttgattgaaaatgatattttaaaggttcaaatttatgtcgcctatgattacgtaacaaagaattgtaatgtactcggtttgactaagattgaaattgatactgataactttgaaggtttttaaaatgtatattgtaaaaatgtgtattgaataaatgtgtattgaataaatgtaccaacgtttattatttgggtctaatagcttctttcctagccattgtctcgattctagttttaaactcttcaatatcagcggcagattgttcgattctattttgtaaaggagcaacttgttctatgagtttgtctattttgttggaaatgttaactttagaatcctctagacgttcttgtaaaatgatactttctcgattgagttccatgttgcacgtttctttgactgcttgattttccgcccgcaacaacgacacttcgtcggctagattctgcgtactctttttatttaacgctttaaagtctaaaacggacaaggcggcttgattgctgatcaaagattcgacttgcaaacgagtcttttgtaattcgtttttgataatttcgatgacttttgtggtgctttcaatgttttcattgtatcgatttttaatctcttgcatttgttgatttttaaccttttgatcgttcaagagttgctgagtttcggctattagtttagagttgatggtttgcgcgtccggccgacgcatagctttaactttacgtataggtttaatcgcgcccgtctttttctttagcgctttgtattcttcgagaatgttattgtatttgtcgtttattgatttgacttgagactgaagttcggtgtttttagtttgagcggcacgcaactgggactgtgcgtcttcgaaagaactaagtttgagatcgacggacgcgatatagctatctttttcgttaatgttttgagtgagttggttgattgtgtctttgtatgtcgattcgacttgattgtactgctgtcgaagttcgttgtactgttgttgcatttggttgtacgattgagtttgttgagagagtaattcgttttgttgattaacgttggttatagcttgagcgtttgaagtttctaaattctcaaagtactgaatcagattgcctacaaactgttgatcgtccgcgggaggagcgctgcctacgtgcgatctgtaaaagatgcgcagtttctcgttaacgttattcaatatcatgcttttactttccgcggaatcgtattgctgttgaagcatagtctgtttggtttgcagacgagtgatttcaatctgttgttgttctatggtgtttttgagcaattcaatttgttgatttttaagcgcttccacttcaggcgtgtacgtctgtatgggttgctgcggtaccggtgccggtggtggagcgttgatgacgttgacgacagccacttcaatttctgacatgactgcacgtaatctttcctcggttttgatggcggacaagacgagttcttgtttagtaatctttgcaattatcctacaaaaagacgatatcaaaaacgatatcaatcgacacacgttcggtccgttgggtaaagcccaacccgtttcgcgttcgatgcatagcaacagttcgtcgatgtcgttgtttatctgaatagaatcgaacgtttccaacaactcaaggctaagcgttatctgttcgtaacgtacgtatttgcgcaccaaatacacaatctgacgcaataattgtttgtatgtcgtaacgctgggcgttctctgcgcgttcacatacaatacattcaacgatgccagatcgctttgatctatagacggcaacgaaactactgtcgtcgtcgtcgtcggcaccgctgctgtcgttggagcgggcggaggtggttgtaatgtggtggaagattccgcgacggtgggtgccgctgtcgtagtcggcggcggtggcgcagcattgatcgtgaaagattgtaaaggagtcggttgctgttgtgaagcgttcggcatttgcattggctgttgcggcggcggcaggaagggattataggcgttcggaacgtttgagttgtaatcgtatttgtagttgaagttgtgtgtgatttgattggcgttcatactgttgggcatcaacgcttccattgttagttcgggcaattgaagatctatgcgattagctaaatgcggcctgtacatcaatataatagatcgtactctttgaacaatatcttcagtgttggcctggcctctacatcgttggctcatattgtttattgtttgcaaaagattttgtacggtgccggcgctgacgtcggtgtttttgtattttggattacgataactcataatggtggcgttttcgttgctcgattggaaatgtctgcagagcaaagtgaaaaattttgggaatcgtcaaggcgaggtgattatcaacaccgacgatgtctttcgaattacccgtatgatatacaaggataattacttaattgtgttcttcacgggacatttgaacacgggagaattgtaccaattctacttggagactaaatgcgatttgtattcgtatcgcatgtgttttaacacgcacgccagcacgacatgtttcaacaaatgtaccagctacaagaccatggtgatgccgggactacgaggcgtgcacagtgaacgcatcaacatcatcaaatacaaacgcaatccttccgacgattacaacaatagaaagagtctcgattcgtttctcaacgacattaacagagtgcacactcaaaccaatctaaaagaaggtaaatacatcaaatttacatcacggcaaagatgcctgaacaatcgtctacaatgtgccttttccgatttcgattccgtacaaaacatgttcaccatcgtcgatcccgattctctcaaatgcgaaattgttcctgtaatggcatgctacgacattgaaacgtactcgaacggtcaacaattctcaaacgccaacaacgatcccatcatctcgatcggtctggcgctcaaacgcagtgcgaaacttttaaagatttgtttgtaccacatgcctccgggtgtcgtcgacgacatgcagccgtttttcaacgaaaacaaagacaatgccgacatttatgtgtgtagatttgacacagaattggaaatgattgatgggttttttaaattgttgccactgctgaacatggattgtttgttggattttaacggtgacaagtttgatctgccctatatcatcgacagagtgaagaaagtcaattggtcaaaggatattttgaaaagatgcggacacgctagagccgaacaagtgatcaaaatcaaacgctacgatcacgaaccgctaccgataaagaccaaggcgttgtttgataaatttcagaataccatgaatacgcattttctaatctattacacgcacgtcgatctgtaccagtttctcagtaccgattctgaacaaaacgatgttgagaattttcaactcaacacggtttcgcaacattacttgaatagttccaaggtggatttgcccatttcgacaatgatcagtctttacgatcgtaaagagatgcaaaagattctagagtacaacgttcaggattgtgtgctgcccatcgacatattcctcaaactcgaagtcatggatttcatgtatacgcaatgcgaacttctctatcttagcaccgacgatgtgcttagtaatatttctcacaaagtgaacgtggtctttttctacaatgcgattaataatacgcgctacgacaaagttaaaaagatcaatgtgcccgatccgtactttttcaacaagcacgatttgaacgtaacttcgggcaggaaacgtactttcgaccaaatgaacgacggcaacgtcgacaatcaaatggtggacttgacacagttgaaacgcaaacccattcctgtcgccagcatacccgccgacgcggttcgactttgtcaccagaaacagagatgcgtgtacaccggcggcaaagtgctctcgccgaatcccggctttaagaaatgggtggtgacgttggactttaactcgttgtacttgtccattatgatgcaagaaggtatttgtctgtcgaatgtgtttgtcggtcaagacggctacgtgtacttgatgaaagatcaagaagctatcaatcccaaattactaaagactttgttggatctgcgtacaatgtacaaaaagaaacgagaccgtttcgagcccaattctttcgtttacaatttgtacgacaagactcaaaatgccgtcaagcgtatcgcaaacagtatctacggctactttggaatatttttcaaaccgctcgccaactacattacgaagattggccgcgaaaagttgatggaggccatcgaaaacatcgaggccatgagcgacgatcccgaaatactgcgcaatttcaacatgtcttcgattcgtttcaaagtcatctacggcgacacagactcgtgtttcatccaagtgctgttcgacgaaaacgaagtcaccggcagtatagataaaatgattcgtgacgtcatcaatgattatgtattgaagaagctcaacgccggatgggtgggttacaagatggcgttagagaatgtgatgactagtttgattctgttgaaaaagaaaaagtattgctatttaaacacggagaacagattaaagtacaaaggttggttgatcaagaaggatatgccgattttcatgcgcaagacgttccgcaccgtggtggattcgtacctgatgggacacagtgtggcgtgcggacttaaactgttgcacgatttgatgatcgaacatcacaaaaactttggcgtcgacaacaactatagcaactattgtttcagcatgagctataacgaaaacccgaccggtaaaaacaagaagaagaataagccagtcgacgtcaatgctcctccacgtaaacgtcccatcacgatcgctaaacattgtcgtgagctgctgagcaattcaggcacggattttttgcccggcaacggcgatcgtataccgtacctgctcatcgacattcagggcaacattagtcaaaagtcgtatccgctaaaattgtttagcgaacagaaccgagtcagttggataaaacacgtgggcatcatgtgtacgtttttcaacgaactaatacagattttcggcgatcgtaaagagtttgagtattactttaggaacatttgcgatgtgtacatgagaaaccaaatgttcgacgtaaagtttcccgttctcaagacggtgaatcaaaaggcgaaaaaatctaaaaaaaattgtaaaaagaacaacaacgacaacgacgatgatgatgacgacgaagacgaaaacgatgacgacgtcgaagaggaagaagaagatcaagttgtcgtgaatcacactcaacaattttcattgtataaatcaaaaagtaaacttgataacttttctccgtacactaaaagtgtttgtatcaaatgcgaaagggattgttgaagaaaaatgtgtgtaatttttttgtaaataaatttattcaaccataaaagcattgtctaattctttgactaggaactgtgatagatcgacgatttcatcttctgttagatgtctgtagtcgacgaaattttgcatgatcatcagtatatgatggttggagtacacttgcttgcaaaaataatcgatttgactgtaagtcagtctgatgccgtcgaccgcgtcgagtatgtcgtacacgtctttcactgtcatgtctctatcgatgaacatttctatgattttgatgaattttgtgcaaaacttttcgtctcgcacaatatccttgagatagttatttaaatgatgatttaccatggcaacctttgccgcgataggcatattgttgaacatttgattgacaaaattcttgaagaaatccattgtgtctgtgttgttgcgcgcgcgttctaatcgacaatgctcttattcttatttaaaattgcgtcaaacgctttttccaggtcgcgtttcttttttacactcttagctttaccggtgggtatgtgtactgttgtcgactcgtccttaatgtagtacacttgtaacagcaacaagaaaattacaaagaccaagagtagaaacagtaaaagattggagaatccttcatttttgtcaaaaatgaatccaattacaatcaatacgagaaacgtaaagtacatgaacatttcaattgtttcaataaacctcttattgtgtaacgatggtgttgtaaagcgacgacccacatgggttctacttactattcgcgtgtataagaatgaaatcgtggaccaaagcagttatatggttgttgataatctacgctatagtcatacacgtttacattgcgcacaacaataacagtaggaatgaagtcaaatcgtttacgcccggacctacgtgcaacacatattattacggcagagatttgcgcgtgtgtccgcaaggccttgaatttaatgtctacctacaagattgtgtattaatagacgacgacggcgacggatgtcatgtcaatgtcgttaataaaaattataatctgtacgataaaattaaatcgttgaaaaatcaagtatataaatatcgaacgagcgcacaaatgttgcagtataacaaagacgacaatgtcgtggttatcaaagaagattgaacctagtaaaaaatgttgtaaaatattacgacgcaataagaattggaatcgcagctatttatcttcgaacaaggtgaccgccaaaggtctattagagtatgtgcaagaaacaaaggatttacaaggcgctttgcgttatttgtttcgtgtgaacatgtactgcaaaatcatcaatagattcgaatggaacttggtgtccaatgcggatttctgcaaaaacgaccgaggatacgaggcgtacaaacaatttagaaaaatgaagcacaacgacgccacgcactcggcgtattctttgtgtaagctggtcatgttgtacatgtacatatcggcgaaatcgcaagcaaagaccggattacacacgagcaactgtatagatttgatagtgtttaataaatttcgatgttttacgtgtgagaaggcggtggccgcagcgcagtatagtgtgtatggagatgaagatgaatattttttctaaaattaatttataataaaaccaagattgttgtgtatgaaactttgtgttttattcaatccaaaaacatgtctaacaaatgttcggggaataggaattcggcgggtatcgtacacaagctggcgagtttaacgctgtcgtttttgtagcatatgttgtcgtttttaaagtagacgttttggagtacggtggtgttgtggtatatatctccgccgccgactttgactcgattgtgtgacgaaatgtaattgtttaaacttttgatggcgcgatccgcgatcacatcaatgtcggagctttttgtttttttgtaatttttcgtacttttgattaaatctttgctgcctttggcgccgcacttgatgatcgaattgaattcgccctgtaattcgaacagttcctcgtcgccgcataccatttcctcgtcgttgatcatttgactcaatctttgaaatagcaaatagctagcattcgagctgagcacaagagcgcagtctcgtaacaatagatcgagtcgtgtggcaaaaacagcgtgacgatgaatcttccatattttatgaagaataggcatagtggacaagagtgtgtcgatttgtttcttgttcttgtatagatagtatatttgctgggaaacgaatgccaatcgattcttgtcgaaacatatgaaattgtaacgcggatcgccgtacagcaagcattcgagatcgatgagcgaattgggtttgggcaggaacgtgatgactttcttgtcgccgtcgcagtcggtgttggcgccggtgaaaatgcctaggcctactttgacgctccagtcggtgttgtttttcggctggagcacgtcggaaacttgggtgctcaactgcgaaatgttgggatgacgagtcgtccaagcgcgaacgttgtccacgtcgcggccgtagtatcgttgaatgctgcttttcggcggcactacgacgttgggcgcgttcagacattgcacgtttgcgtaaaacgatgccgtgttcaagaacgtagagtataaaaattgtccagcgtatccatttttactttgaaattgatctttgataatgccatgattgaacttgatcttttgaagttgaccgctaatgtcgatgaggccggcgtcgtgtttagaattgaagcacttgtttagaaatatgatcacgttatggtcccagagaataaagttgggtatgattagataattaatgttgtctgtaaatttattgcgttttaactttttgagataaacattagacggcaagtcacgtactactatcgtcgtcgccattagaatacggctcagcatttcagtgtgttcgttacgctcgtcacgttcttgatagacgctgattaattgttcgatgagactgttgaaaaagttcatctttattcttttgatgtcggcaatgatgtttcggaggaattgtttaaattcactaatgttacaaaaaaagacattgtcaatgttatcgggatcgtagagcaattcaaaacttgccgaattcttcggaacaaaagtgatagacgtcgccgacgacatgatgtcgatcatcaactaaaataaataagagggtatttaatttatattttcgagtttttcataaactaacatgacgactgccactgaattaattaatgtgcctattttgaaaagcctaatcaaaaccgaaatcgaccgtaacgtcgccaacaatataacaattctaaatggtaaattgaaaaaactcgaagatcaccatctaaacgattgggtggaagtttacggtattcacgacacccgattacataacaaaaagattcgcaataattatgtgaaaaaaatttgtgcgttgttgcaactcgattacaagtcggtcgtagagagcgactatgaaaagaaccacatcaaattgaaactgaacgatgcgactacggcgcgtgaatggcagaatcgttctagagaggttcgactcaaaaactacgacctcgaaatcgattttgatggtccgataaagatttttgtagccgcttcgccggaacacaagaagctattgaagaagacacgcgacgctctcttgccgcactacaagtacgtgtcgttgtgtaaaaagggggtgatggtgcgcgaaaacgaacgcagtaagattttcatagtcaaaaacgaaaacgacatttacgatctgctaggtaaaatgtccgtggcggcacatccaatctaaatggatgagtgcgcaacgcgataaacgtttagtacttcatgttgactttcggtgaagtgtttcgctgaaagcaaacatcaaagtactaaatttagcgataagtgttatcaagttgaatctcatcatagtataaaagaaacgtattcttgccacaagttaaagactgacgaagacgacgacaacgataacaatgttcgcggcaataaataattttttaagagaaaaaatcaagtacaatgcgattacgattgtacaagaagaatttaccgatccgtattatacagtgaaaaaagttgaaagatctacggatgtaccgtattgttcatattgggacaatccgtttgtcatcgaaaaccaattgcctatcgacgacgaaaacgaggacaacaaggacaaaaacaacgacgacaacgacgtggtcgacgaagataatgttgcgacatcgacggtggactttggtgacatcaccgtaaaaagtttgtacgttaacaaaaagaaaaagaatattgtggcgtatttgaagaaaaaatacttgaacaaaaagaacgaaaacgataaagtcgacagagaaaagatcaagctgatgcgtcatcgactgctgattagcgtaccgaagccgttcaagagaacgcttagcatcgaaaaggaatttgtcacggatcatcatttgttcaagtcgaatctgaagaaattttcattcttcaaggaaaagaaaaatatacctacttcacatttatatccggacgtggacgcctttacgaagactttgctggatttcttggacgatagaagcgacgagtttgactttaacaaagtgtacaagaataggtacaatgcgttttacgagaacgacaatatttttaaaggaatcatttataaagatctagaacaagttcccgtgatgacgcataaggagcaatgtcggttggacgacgtggaagatgctcacgtcatggttgtgaggtttaatagattttatttgaaaacgtgtctcaaacctaaaattaaaaagcattttatggacgttctcgaatttccgctctttatgctcgataaaaattgtaatattttgataaatgaaaagtatggtcagtacgttcgctacagggatatcaaggacaagatgaacgactattccagtctgaacattgtcttgtcgagtaaatgcgacgactataacgactataacgttaaagatttttttttcgattcaaaaaacgcgccggccaacatgggccttttgtattatgtactgtactgggtatcgtttcacggtttggggctaaacttggactttcaagaatacatgttgactcgtgtgaaacaaacgagatgcacgctcggattgtccacgtcgtctgacttttttccgcaaattatggttccgttgccgaacgctttgttctactcgtgttacgtgagcactgttttatttaaaaatcatcccatgtacttttaccaagagcgtcttaaagaattgaattcgtactcttgcgacatgcgtttcttgctacagaaaatttttggcgaagactttttagattattacaacaaaaaagttagaaaccgatctaatgagattaaaaattacgatacgctcgataacgatataaagaatttaatactggaatcgtacaagagcgatgatttcggttatcattatacaaaaattctgcccggtagccgtttgcttcaattaaataaaattatcaattttaatataaatttcaacgattacatacacacgtattatgaacatgtacaatttttttttgcaagatcagtatcgtacaatattaaattgagttttattcatgattcgacaatgaggcccagattacaaaaacgtcgctattacgatcctgacgatgcagtatgtaacatacataaatttatacagggtatgatgaatcctatagttttgagcaacggtttaattatgaaacgaaacatttcaaagattaaattgtccaagtttctcgatttgtataaagcgtttcaagagtgcgtaggctcgctaagaaagtttcccaatttgaatgaattccacatctttgttcataaaaagtttgtcaatcgaaacaataaaatttgtttatttccggaaaatttcatatcattgatcgatggagtccataagtcgtacacgtcatttttaaaaaataaaaaagttaccgtgcctcagtttttacatgtgattgaaaggacaaacgattataaagtacgtgtaaaagaagaaggttttagcaaatatgtaagtagtgaaattgtcaagtatctagaaaatatttttgtggatgattgattaattgttgtacactgttgttggtatgaaataaaacatatacacactgatattgttgtcttttattaagaataatgtacaataatatattagagttgcctgtcagtacacgcgttttacccgttgagggtaaacgtatttacatgaaattttttaatcgtgcttacgaaaaatatcattctgatatcactgctgcacaaattgcctggcaagctgttaaaagaaaatacgtcaaagtcaacggcgagtggatacctcgttgcgacgccaacgaatacgacacgaccactaccgaagacgaagacgacgacgacgacgacgacgacgattatactaccaccgataccgagtaatataagcgccgcaataatgtttcatttaaacgaagcgttctatcatgaagagatgcccgctcgtgccaaacgtttgtttgtaaagacttttaaaaaatatcacaagctagacggcggcgatgaagatgtggctcttcacatggccaaacaggcagtagatagagagtatgtaaagctaaataatcgttggattcctaaaacggctgccgaggaaattgtacggcacgatctcgaagatgatagtttgagcgaatctgagacgcctccttctcctccaccacccactcgttcaaatatattacaacggtcccctgcggttgcttttcgacacaacaacaacaaaaacaacaacgtcaaccagtataatcgattaaaccacgacgacgaagatgacgacatggattttttgtccgacgaagaatctgatgatacagacgagtacgattacgacgaagatgaccattacaacaacaccatgtatagaaaaaaatctacgttgacgaatcgcaatagaggcaacggcggcggcaatattaaacaacaacacaagtttacacgtacaatgaaaagtaacaataaacaaacattcaagtaaattctaaaagttttatttcgtattcccgatcgagcgattgtctcgccttgtccgagggtcgcacatagtttttattgtgtctattttcaaagtctacatatccactgacacgtttatgatatgtttcttcaaagtaaattgtcttggccacgacgattctcgctctgtcttcattgtacagattgataattaacgatttgttgtgatcttgctcgttgtacgcgacacattcgccgagaggtttgtctttcttcagccgtgcaccgttgacggtacatatgctttcgtagtacagcaattctgccatagattctaatgataggtcgcagtaaactttaaaatttgtgtaaatggctcgttgaacattgaggaaaattatgtattcattgtcacataatccgtcggcactgaacgatatgtgtctgagatcgatagagattccatcaaaatcaagatatctattgttgacggaaaccattttcaccaaagatacgagatgacgtactaacggagcgccgccgcaagtttcggtttatatatacgcgtctccgttgtcgtagtaacgctcgacgacggctccatttgaacataatttccatgagtgtcgtcggacagcgatcgggtgtcgcaaaacaccgctatatcgtaaaggatttagggttgtttttcaaatgacacacaacgacaaaatatttggtatcgcaatatttatttacaaagttttatcctcgtcgaaacaacatttaattatactagtactagaagaaaagactaggggttccttaagtgaatttggcgacttaaccagcttacaatcaccaaaaacaattttgttaaactcatcatgtaatctaatataattctcaaagctaatagacataggcgctataaatttacaatttttacaataaacaacttgattcgtttcatcgacgcgtcgatccgccgaatcgttgttgtgctgcgagtggtcgtcgtctcgatcaaaatttgtattttttttcatcgaaaacagtcttgtaattcctctgtcccacaacattgtatgtagtgtttttgccaacgtacgaaacgtcgtttttcttttgttcataaaatgaagcaagactgtcttggtttctgtttctcagcttactatcaacggtgttgttgataacctccttgagtttaaagtttacttggctcatgttgcttgttatcagataacaacaacgtcttgatattgtgacacactttgtactgtgacctaaatattgcgacacccggtttttatactcggcgataattaattcactcgagaacaatgccaaaatatctgcgaaaatcatcgatgaaattataccaatccacggcaatgttgtttctaaaacggtcgctattgttatcgtcgaatcgtcgattcggcacgttttgggagacaattaattcgtaatagcgtctgtaattgttcggcgtgcgtgcccattttgcgtaatggaagattttcttgaccatttcccgtggaggacacatgatgtgaccgggcgcgttggcgccgtattgtagatcgcaacacttgactcgctccttgttttccggacacctgcccagcatgcgaaatattaccgatatattctttctgttgtcgttgaaaggattcggttgtttgagtatcatggtcagtatggtgttgtacattagaaaagttcgatagatgatttgcgattcgtgcgaagcgtccgtgctcggtacactaaatatattaacgtcgtcgttttggggcacgctttcgtgcgtgtaaatggatatcatcctttgcggcagtacaatggtgcggaatatagaatagacgaggtcgacttgcaaattgtccaccattaggatgtaacggtcgacacacccggtaaatgcgtcgatgatggttttgatggtattacgtaatatgaccatgtttcgttcgccggcggcgtcgatgcccatgaactgttcgtcgtcgtcgagagggcgcagttgcgtgtacgatatcatttcgttggcgttgttgtagatgcctcgaatttcataattgtcgttcgaggacgacgctttgacgagacacatgtagaacggttcgcgattgtagtcttggtacacgttgttgagaacggtgagatgattgtcgaaagcggtgtcgttttcgggatcatagtgtttggtcactttgcagttcgctcgtaatggatgaattggacactgggtacttgtcagcttcaacgttttgaaaggagttaatttctccaatacacactggtttaatcgaattagattcgtattcgacgacatttaaaattatataagtgttgtcgatcaattctaggttatctttgaggatagcgttcaacacgtcatccgcgatggacactgaagacatttttgacaaatttaaaataaaaatgttgtcttatgactcttacagcaaactcggtcgctacgtgtttatgcgtcaagaaaataacgatttcttgtatttagacaagtacactgataaggtgcaaaaattctcgcatcgttttcaaacgttaactgatctcggcgattacttgattctagtctattaacaagcaagcgtagcgcaatgagcggtaaatcgggatctaaacgaaaaccaacgtcgagtcgcctaagcgagattgtgaaatttaaacgcaaacaggacaacaaacccgctgtgccgacaacgaacgatctcgaagacgatggtgtcggtttgaattttgacccaatctctcccatcaattcaccgccatactttcccaactacgatcctgacgtttcaaactccgccatggacgtggatattagaaaacaagtggtgtttgtccggccgccaaccatgaaccaaatcactaaacaatacgacaacatcaacgacatacaaaatgcgatcgacaacatcacgcagtacatggacgctcttcagaataatatcagtagtgttaaatggacgacaaagcagcattttacttttttcgaaaatcaagtaaacaaaatcggatacaaagacataatgatttcagtgttgaacaacaacacggtgaacagggacaacttttacgcattatccaacatgttttacaactattacgtccatctgttcaacaacataatccccttggctcacattattgtcaacgtgaattacacgcacaacaaaactcgtattacacattctatgacggcctttctaaacatgtgtgcccactacgtcgtaagtaatattagacaactcttcaactcggaaccgactataaacattccggaatcgtattacaaaatcatcactgacaaacagcaatgcttgactaacttgtacaactttaaattaaacgatcttcgaaacttgacatttatcaaacatacgccggataatgacgtaaacacatataccagcacgaccgcgtctaatcctaaagatcgcgtaattagttttcccatttacgtcgtgtacaatataccaaatttgaaatttgaataaaggattacttgtagcgaagtaaattatcaaaatccaattccatgtcgagaacgtgatgttcgttgactcgattaaactcgtcggcgtaaaatctatccacgtcttgttcgccgataaatcccgtcatgttgtccaacaaagacgagcattcatctagagttttcggcgggaaattaaagtagaatttaacgtttctgttgaaaggatccctgttgcgttctcgtttccatctctgcaaacatgaggcgtggaacattttttctaaatttaaagttttatattcggtgacggcaacgacgccatcgttgttgatgcgttcaaagcatatttggcattcgaccacggaccgtttccaaagttcattgaacattctaaacagatggctttttttatcctttaaattgatcgttattaacatgtttgcgtataaaaagcaaacgtgcgactttatcgtgggcacaatttaatgtatcatggaacttataataccatttctacactactcgaagctttatcggctggccacgagcgaaaatgcgcgtcgcctaatctacgaccaatggtcaaaggacacgacgaatattacaagagatttgagttcaacaaaggccgtttcattatcgacaaattgcgtgttttgtcatgaaaaatttaatcaagacaaggaccggtatttttgcgatttctgtttctttcctcgaaccggtgtggacgaagagtttgccacgtactgcttgttgagcgtgtgttactacgagacggccggtgagatcatcgacgacaataatggcaccgaatcatcatcacaacgcgtcgtgtacagacaacggctcaagatgacgtggtacaattacgaacgtataggaaaaatgtacgaagttttgtatccccaatgctatcaatgtaaaagtttgacacgtaacttgggcgcaaagttttattactttgatgatcgaatgttctgcgacaactgtatgtttcccttgtttatcattatacttcataataaatgaccgtggattatattttttgtttttcttttatatcaacttcaacaataacacgtagtgtttcgtcgaaagagcaagggtaacgaaacgttacccttgaatatttaataaacaatctttgaaatggacacgttatcaaacataacaatttaatgtcttgatgtttgctttcgtcgaagtgtttcactgaaagcaaacatcaatagaatgacgcaataaaatgatgaaatcattgctaaaaataacaacataatgtgtccatgtttgctttcgtccatgtgtctcgctgaaagcaaacatcgagacattaaaatgtcgtcatgatgatggtggtggtggatgatgcaatacttgttttttatgtcacaatataacaatgtttgctttcggtgaagtgtttcgatgaaagcaaacattgttgttgttggctgatgcaatacttggcaacagtaacgatgtttgctttcggtgaagtgtttcgatgaaatcaaagatcgctacgcatgtgtcatggctgaaaatagattggtaacaatgttggctttcagcgaaacactttgtcgaaagcaaagattattgtcggttaccaactgacgcaataaaattaaattattgtgcaataatcacaatctttgcttttgatgaagtgtttcgctgaaagcaaagattgtaactattgcacaatgatttcatcattttagaatattattcttttagactttgttaatcgtttcaatgacctgctacgataacgtttcatggtgcgggttttgcgttgttttttataatctattccttgaatatcactactaatcgtcacgtacacaataatcattgtgataataatgttgtatttatacgggcgtctttcaagtgtatagtgcagttcagtaagtagacttaaacatgcaatcgtttgaagatcgacttaaaactttttcaaattggccggccaacgatcgagtgccgtcgcacatgttggctttggccgggttctattatacgggccgcaatgacgaagttcgttgtgctttttgtaaagttgaaataatgaagtggaaatatggcgacaatcccatattggatcacaaaaaatgggctcctcaatgtaaatttgcaaaactattaatttccaacccattaacaccgatcacggaaacgggaatcgatgaatgcggtacgtccaacaacccggttcccagaatgatacccaagtatcctgcgtacgaggacgtagaaaaaaggcgtcaatcctaccaaaatctacctattcctctatatcaagatttggacgacatggcatgtgctggattttattacaaccgcgacgatagtacgtttgtttgtttccaaggaggttgtactattgttcattgggaaagacgtgacgatccttggcgcgaacacgctcgttggttccccaattgcgaatacgtaaactacatcaagggtcgtgattttgttcaagaatcaatatctttgagttgcgtaatacgatccaacgaacaagagcagcagcaacagcaagacgaacagcagcagcagtatgccgtgaacaattctgtcgctgtcgataaaaacaaagatgatagagacgacgaagatgatttgctaatttgtaaaatttgctttgataatcgtcgtgatgtatgttttttgccatgcggtcatgtggtttcgtgtcgtcaatgttcgtcgaatgttaaacattgtcctttgtgtcgctcaaactttaccagcgttcatcaattgtactatgcctgaattgtattaaataaaacacacacaaaaatttttaatattttattttattaatcctggtccatggtttcatcatctgaatcgtagtacgacatctcttcatcgttgtcgtacgactcttcaccgctgctttccattttatcgaatgccgaactaaaattcaaacgacgttcaatgtcttgaagaatgacatcatcgacttcaatgttttttataaaattgtaatcgtttgatatcttacgttcgagttcgttgatatcgttttcgatgacgttaatcttgtctatttgaggagaatagtttgtgtgatctatacccatttcggcttcggtgtaatcgcgaacaataaactttttacgtttgctttcgcgttctacaatatccttgatgttgcgttgtgtcaatagttctattactgcgcaaccgggacgattgctgcacattatcaaagctactatgccgtcgtctttaaagtaattttctagatattcttcgtcgatggtatcatcctcgctgacctgccattcgaccaaaagtctattcgtaatggtagttttataatgtttgtttttgattttgagttgaatagttttgtagacgttttctttcaacattaattgaatgtccatttcgtaaaagtctttggcatcgttttgtaaacgtaaagcgctagaatagatctcgttggctgtctttctcagaggcattattttttcgttaaagtaatggttgattttatctttttcaaccatgtatctatcgtaggctttacgattttgcggtagacccaacacttgtaaagcgttacctaaagtactatggattttttcgtttgtgttctcgctagttgcggccacgtctctgccgacgcgagattttttataggtgtgttgaagattgttaactttactggtgatatctgcttttgtggcattgcggccaacgtttaaaagatcgtagtagctgatcgaatctaaatttaaaaagtacaaagcatcctcttttcgggaaactttagccgctacgggtttttcgctgctgcccgtcgaactagattcgctcgtcgatcttagactgccgccgctggttcgtttgcttttgtcgctagtggcggtgctagtagcgcgttttaaaaaagaagacggcggtgaaggcgcaacggatatcgtgcgttttctaggcgaaactacagccgtcgtggacggaacgttaagtcttgtagagctacgcgtcaccctgctcattgtcgtcgacgctcgtcacaatgacggacgtgattgtcgattttaataaactgtacgataaactgcgcgacaaatacggattgcgttactatctcaattgtaacaataaaacggtcaatacgtgtacgatcaaatatttgcaggagcgacgttcttatttttgctgtgctgtcgattcgctggaacgatgcgtgcttcacaaatgcgtactgatcatattcggcacctggttagatttggagtttagacgcgacgacgagaacaaatcgtctcaattcaacggcacgtttatgatcgacggccggtacttgagctttcccaacatcatgatgaacaacaacattctcatacacaatttttacgacaagctctatagcaaagattgcaaacgcatgtttctctatggcaatctggacgaggagaaaaacatcaaccgcgccattcaactcgtgtacgacaatcagcgcgatgtgttgtatgctcgcgacgtgtacgccagcgactacgtcgtcaccgaggatctcaacgatatcctcgaaatgtatttgaaaaacagcggcaaatggactccgttaaatttcgtctttgattttaacaaggaacagagcgcaaagttggttaatttgattaaaaaaattatggccaccgacatcaactacaccattgacaatctttccaacaaaatcatttacaaacacgactatttgttgtggttaatctataaaccaatcttgaaaaactatagtacaatcatggaaaataaaactactcaacaacagcaacaacaacaacataataatcatcaacagcgaaagaaaaaaatccaatctattctgtttcctaaagagtgtaaaaaagtcatcgataccgtggtcaatggtaaactaatctattcagtgtccaagacttttagcaaacagaaaaagaatttcatcaactaccaagacaacagtagtaacaataacatcgaaatcagtctgccatcgttaaagtaccgtatcggcaatgaagtggtgcgcatcacaaacgatactatgcgacaggatatgctcaaacaaaaattcgactttatcaagtttgttgataattttttccacggagaaatgacggtggccggcaagaaattttttctgtgccgcgatgttaaactgcccagtgtcgattatgaatccgtagctgaaaagtttaaatctttgttggctgccaatctcatcaaaatcgcttcctacgacaacgaagacaacgttttaatagcgtttaataatcgtcctacgatctttgaatgcgatagagacaaaatgatctacatcatatatcagttgaaacgtaacaagtttcccattgaaattaaatttcacaacaacattttgtttctgaaccatcacgaaggtatgatctgtataaagaaaagattaaagattaacagcgatgtgggaatcgtaaaaataatgacattgttgacgccgtacgaatatcacaatgaattgtcgtacttgcgaaccgtgcccggcatctgcgtcgaagaagagaaacattgttcgcaattaatgtccaaactgttacactactattacaaagatatactatccatgtttgctacgacgccggtgcccaagtctatagtttcgttgaccaatttgaaaaacgccatgcctgtcgtcgcgtacaataatctgggcaacaacgatatatttttggacaatttaccggcaggcaacagcgttgtcgtcgctcccgagattatgcgcaacgacaaaatgtttcgtttgtggacactggtgcgcgatcataaactgatgaccgccgaagatccctatatccccgacaacaaactgcccattcgtctgttcaacaataaaatcaacaaactcaaaggaaaacttaacacgttaaaaaacgaagtgcccaagataaaatattacaagagtagagtcaacaattgcgtacaagtcaacggaggccatattcttcacatggccggtgtggtcgtgtccaacattaaaatcggttggatctacgacggaaaaaggtacaagattgaaacgtgtcgaaataaaacattctacgtgtacaaaatatacgtatattacagacaaatagacaagcaacaaattgaaaaactagattcttctttgaccgtgaccaacaatacggtgtacgtcaaattgatcatcatcacgtcgacggccaacttggaaggtgttaaaatttgcggcatccacggacaaaaaggagtgatgaacggcagcgaggatctgaccgaatggatggccgaagacggcacgagcgcgcaaatttgtctatcgcccatctcgtacttgtcgcgtcaatcaaactttgaaaatatcgaaatgaagtatgtggtgcgcggcggcgatcactcgaaccccgacgcaaaacgctatcctattttcaacattccatacatgttcttcagcaatacaccggacaatattttcaaagaattcatcaaaggcaactacactggccacgaaaaagtcgaaggcactcgactcgaccaatggaccatcaaccaatcttttgcgggcaaccgatgggcggaaagtttacaatgcgttcgaggcggcaacaatttgcccgacaacagtggcgaatttaatgtaatgtctagtttgttacactgtaataatacaattatgagataagcttcaacggcgatgaaaacgtacaacttttcgtcgaaagaggaagggtaacgaatcgttacccttggtgtttcgtcgaaacgttacgtgttgttgttgaagttgtttagaaagatatctttgtaacattcgatacacatgattttgttgtttttaattatggctctgaatctgttgaaaaatctgtcgctgcttgtgttcgtgtactttttgtgtaaataaaccatttcttcacaaaaatagcattttgaatatttattcattcttatacaaaatgaaacttgaactgattgccaagagtgttttcaaaggtgaacgctaaagacgttctggtgatgaggtctgatttcaaatctgcaatacgcattcccacttgacccaagatgtgttcgatttcatcaaagtacatgatatgcgtgtgtttggtggtctgatatgcgttgttgatgctgacttgtcgagttttattatttttgattaatacataggggaactcgaaaatgcactcaatttcattgtcgtttgtgaccaccttgaaattgcgttttaatttattctctttagatatttttagcgatgatgtaatatcatcggatgatgttgatgtcatcgaataacacacggtgccctcgccctcgtttacatctttgacgggcacacgttgatatagaaaaactatatcgttgtcattgtcgttgtcgtcgtcgtcgtagttgttattgttaatagatccgggttcaaagggaggtagctgtttgtagttttcaatgttatgatgttgaaccgtgtaaagctgagaatttttgtaaacgttattcttttctatgatggcaaatttatttttctgattgtggttcaactttaggacgacacaacgagcgtcgcggtttgtgctattgacagctttgatttggtcgtacaattgtaacatgcccgttttgtagaatagagtgtacgagtagaattcaagattaagtgcgtcgaatttactgaatgacatcaccgctgcgcaatcatcgaggttaacgatcgcggtgaaacccggattcggtaggaacgacattgtcgtgttgggcgatatcagaacgtcgttttcgggcagtttaacaacgtcgttttccaacaagataacacctgcgtcaatatatgtcgacatttcatcatccttctcgttatcgttccatatacgtttagtcatgctccacagcaacgcgtgcgttctaaagtcgtaattggcctgataatatgccacgttgttgacaggcgcgactaacgacgaaaagtagtactcgttcctgacggtaactactttactgaaatctgcggcatacacgccgggtttgtagtacataatctttgaaatgacaggcatgacgtggacgccgtttttgtataaaagtgcgggatgaattcgtccggaattgctgcccaccatgtgtaagctatgatcgacgttttgcatgacgacaaagttggggccgaacaaaaagttgtaatattggaaggtgtaatagttttcgattaggaaagagtaatttcgtacgttggcgtcgacgtagttgataaaatcgaatctgatgccgctaccttcgttgtgcacgtagttttgtacatcttcaagaacgttcatgaccaaaggttgagatttgatttcggacaattcgacacctttacacaattgagcgtaaatgtacggcagacacattcgtgtgctgaaaccaatgtttcgtcgccagcccaacgacatattcggttcggttatatagttgtcgatgatcttgacggccactttgctgacatcgacgtatggtctgagcgtgatggacaagagcattgcgcattctgttaaaccgatactgaaaagattccagtacatttcgtcaaagttccatggtagatgcttgtacggtgtaggtgttggtagtttattggctataatttttagcgctcgagccaaacgagtggccacgacattgttggtttcgtcgaaattaacggttgcggcggcggcgttcatgtaaatcgcgtgagcgttgagagaacgcaaaagttgcgcaaaatctttagcgttctcgaaaggttttaaacttttaaaaatgtcaatgtcgtaatcgtcttcgacaaactcgaaaagtatatcggtttcgttgtattcggttcgtaaatctttagttttccaaagagtggaccgatagaaatcctcaaaagtatataggttaacttcatgattgaaattatcaatttcgttatcgatgtagtcgtcgattggtcgcgaggcgttgttgttgttgctgccgctgttgctattgctgctgttgtaatagtacagaacaacgacaattactaatataatcacgacgacagaaacaataaaccatacggcgtttgacatatttcacggttgaaacttaaagtattataatgggttttgcgcgtttttacgagacgagatcgcagtcattgccgcaatgttgcaaatatttggctgatcccttagctttgtacatgttgtatgtgaagtacgacgactacgatccgacacagacacgccatgtcatcaaaaacactatcgaggtggacgacgaaggtttcattgtgatgcaacagctttgcgtgtgcttcgatctcgacaatttgtccacggcttcgcctaaagaattaaactgttacgtggacgttacgcgaaaatctatgagcgatcacgacaaacgcatctataaacttttactgcacgacagatggtgtaaaggagattttgttcgtctgcgcaaaatgctcgttcagccggatgttagcaaactcatcacgttcggttgcaacgttttatgggaacgcggctacgaaaaccactacactttgggccaacagctgagcattcgcataacgacaaaactcatccaaagcggtctggattttaaacatcaacaggactcggacaacaccaacgtcgtcgacgccgtgcccggtcgcggctggaacagcaaagctttcgaaaaactaatcggaaacattacgtcaatatctgacgtcatcaaaagacataaaagttcacacaagtacattgttctcgaaatagatccaataaattgcgacgcagtaaagaaatgtttaagcgatcaattcacgttgatttttaatgcgcacatgcaaaacgtgtgcgcgatccacgtcgatgaagacaaaaattccgttcaatatttagtgaagctatcaaagttgataaatgataaacttgtaaacgtgctctttgtcaccgacgtggaatattatctgaagcaaaacaactacatgttttatctgtacaattctctgaaaatgtattattactgtttgtgtaacaagtttgtctttgaatataaagattacgaaataatattcttgttgaatcttatcatttcgttggaatggcacaacaacggccacctaaactcgttcacactagaaaaatcccagatctacaatccgctcgaactgtcgacccgtcgtctaaattcgataaaacgcgccgctgcacaatctagggtcataaacaacgataacgaaataaaaatagatttcattaaaggcaaacgaatgaagatgggcacacactacggtcaaagacttgtaacattatattgataacggtataaattagtagtatataaacgacgatgaaaactgcatcgttgtcagtgatcttaacactttagcacaagcaactatgaacgtgggcacggtgtgtttgtgtgtggacagttgtctgctgttaacgaaaaaaatccattcaaacgtcgtacagacaatatgccagttggactttgaaaaaggaaccccgaatctggtgccgatacgcaaaagtttaataatgttacgcaataacttggactatttatatgtatatatcaagaataatttggatcaaaaggacaagaagcttaacgaattcataaattccgttgttgtcaaggagttgaacgatgccctgtacaattacatgaacgatatacaaattttggacgaagacatgaagaatattgaagtgtgcagtcacatgatgttcactttgaagtcttttaataaaaagaattttcaactccacttggagattaacgattataatcttacgtacacatttatcaatatgataaatgtgcacgtgttacctccgagcaagttgttcaactacgtagttgataaaattaattacatgcaacgtgtgtgcgataggattcatgctcgtctatacgaattggaaacgatttacataatggtgaacatggcaaccgcggctagtctcgatgataacgacacagtatataaagcgacgccgaaagatgcattgctttagttgaagtctaacatcggcgaggagctaacaacacgacaacggctactatggacgaaggaaaattgtgcttggtcatagacagcttcaggaggcaattgaggctgttgaataaacgagtgctgttaattcaagaaaatattgattttgaagaacctttaccggacttggaaaatttgaagaataatttgactaaaattagaagcgatttaaagtacgtgctgcagtgtgccgagaacaacattgatacaaatttggacgtttacatatcgcatcatacgattaatgaattggattttatgctgcgttcatgggaaaagtgtttgcttgacaatattccctttgatattgatgagtttaatgttactctaaagacgtgtgtgtacatgtttaataatgtgaaacgtataaactttaaaaactatatgaccacaaagcctttgtacgataacaataatattgaatcgtttgtccatcaagtgtgtatgtattgtaaaagaagtaacaaatacacgtacatgttgaatagtatacgtaatcttgtagatatatgtgataaaattaaagtaaggttgtcaatgtatgaacttggtaccgaataagaattgtacaatgcattcatatataataaataatatattatttcacaaacagtatattttatttaatcatgcgttgcgcaggtttgtttatgattctcgaagcggacaaggcggtgttgctgtgcgctcgccgttcgtacaatagtaacgtttcgtacagtgatcccaaacaattggaacgtgtaaactttttggaaaagatttcaattccccgaggcaaacgggacggtagagatatattcgattacgagacggcggtgagggagttcatcgaggagacgggcacgttctttgaaagtgcttgggtgtatcgtgtgccgtttgtgctgcaatggaacgatgccggtgtcacgtacaagtatgccatttacgtgggcatagttaaaggtttgctacagcacgtttctcgagaaccgaacacgtattgtgtaaagctcaattgtgataggccaaacgattacaagtttaatttagaacttcgcagatataataacgagattcctcgacatctatatattctgccgttgcaagattattttcagtatatgaacgaaaagcaattgatcacgtacgattccagcaactatctggaatttttcgagtttgtaaagagcgtcaaggcaaaatttaatcaacgtacgttgaataatttttttttattaactctgaaattggacacgtttaatttctatcaaacatggataaacaatcgtcgaggcagcagcagcaacaacaatcatcgcgacacttcaacattgctaacgtcgaacaaggatttgaaaaagataatcaatgttgtttgactcgcagcgaaacgtatgcgttgtgccgtgaagttatcaataaacgtaagcacgacaatgatctatccaacgtgtggacgcacatttttgagccaggctttcaagaacaactcgaatatataagggcaaatcttgacaaagcgttcattactgttggcggcgagcaaccacactgcaagcgactgttttatcacgcgcagaaaattgaaaaaatatttaatttaaccacgtcccttgaaaacgaatacaaaactgcctacagtaaatatgaacaccacaacgaacgctttaactgaaatgtacaacagtttcggcggaggcaacgttctttgcgacaaagcgcacatggaacacgttaccgccgtcatcaacacactagaaaagaaaaagatcaagtacaaaatcattcccatgcccgtgtacggcgatgacggcctcgaagttacctttgcaatcgttatcatggtggacaaaaagaatctgaagcgaaataaaaaatccattagcaacaacaagtatatcttgttcaatagctggtacacgaaaaatcgcaaatcgtcctggccgaacagccatacgatgtggaacttgatgaaaactcacactacggccaaaccgtttgtagacatttttgattttatggaaaagattggcaaatccattgaaactaaaaaatcatcatcaccaccatcgtcgtcgccgtccgaagataacgcagtcgtcaccgatgttaatactcaaaacaatgaaaacaatgcgcgtcgtatgaatctatacaatgagttttacaaaattaccacgtatacgtttgtcaacgacgcagccccttcgaacagtttcatatacgacattaaattggacggaaacgacggtttggaaaagttgaaccgtcaaactttggagaatggcgtgattgcgttcaagaacattttaaagtctgcagttccgtcctctgtggaatttttaactacaactacggcaacgacaacggtagctacatcaaagaagaatgataaaactttaaaatcttcgtcatcatcgtcttcgtcgtcttctcgaaaacgatcggcaacgatggcggcggtgtcgagcagcaagaacgtgaagagcgtaaaaaaatcaaaaaaaagtcatgtggtgcccgccttgaccatggaaaacgatcaaagcgacgacacacaaatgtcatatagttaatataaaatgacaagttggtcagttcgaaatgttagttttattgaaaaacagctacaagacagccatgtttgcctttaaatgtccctacactgaaaactatatcagtgtgtatttaaattacaaaacatattatttcgactattatgaactgatggtgttgctggaaattttttgcggcggtcaagacaatattttgaagaagggcggctgcgacaagatcatcaacagattgaaaaaggcgagatttctcaaaattcacgaggcaatagttttactggattgttttttaaacacggaaaagattcaaaaatacattttgaacgaaatatatcccaagctgttaaaatacaaaaagaatatgttgtagtaataattttttttactttaataaaaatacattttataaatgtgtgtgtatttatttatttcaataatagtaaatctatatgaggaccgttgtgtctgattatgatagcttctaaaatgagaccttcgtaaatcaaacgacgatgagatgtggtaggtggcacgatcaaggctgccgtgtagtttttaagttggccttccatgactatggcttcatgtattccgttgaggcgggttttggtcactttgaccattaccatttacaaataccgccacgtagtctcaaaactaaatgtatcgtagattctttttgaatgttgtaatcgctcatagtacgagaatcttcaagttgtttaccagcgtagatgagacgctgttggtccggaggaatgccttctttgtcggcaatcttttgttttagctgttcaacggtgtcggtggattcgacgtccacggtcaccgttttgccggtcagagtttttacaaaaatctgcatggcttaattattcctttcctctcttagtaagatattttaaaaatgagcagtcaagaattcatcaacgatctacaggctttgatcgatagagtttcggcgaaacacaagcccgatcccaattcaaagctcggagatgtaatccagcatatgggtcgtaatggtttgcttttgcaaaggaaaaaagaggatgactttagtatcgacgaaaagattgagatttcagacacggcacgcgattacttgaatctgctgcaaacggaaaaactgtccgaatgtcgtttgtgttaccacaacgatgacacgtcgcgctgtgactttcatcaaaaatacatcttcaccaaagatcctaaacaatactacgatgattatgtgaatttcctcaacagcgagatgggcatcattagtttcgtcgaactctactacacgtatctagcggtgtccacatacaaaattgtgtcgctgatgatgatgcgtgacctgaccaacttttcgtccgttcgtgaactattaacatactacaattacgaatgtgccaacgacatcgacgtcgtgccatacgaaactatggattgtgaatagtagtttgcgcaacaacttgtttttattgcatcataattttatctttgctttccgcgaaacactttaccgaaagcaaagattgataaaatgacgccatagaatgacgcaatattaaatcatcgctaaaaataaacgtacaatgtttgctttccgcaaaacacttcaccgaaagtcaacatcgttgtacgtttatttttagcgatgatttaatattgcgtcataattttattttcaatctttgctttccgcgaaacacttgaccgaaagcaaagattgaaaataaaattatgatgcaataactacaatctttgctttcagcgaaacaatttggcgaaagcaaagatcgaagtgttattgcatcataatttgtcatcgctaaaaatagatattgcgcagtagtggtttttattcttttacgattgtgatcgtgttttcctgtagcggcggcgccgacgatgacgacgacgacgacgtggtcggtgtaaccactgtaaatttaatgtcatcgtttgtaaaatctacagagatcgtgcctgcgattgatttgtcttctactttcatgacgtatatggtgtcgacgttgccgtctttgatgtacatgtcgtagtcgaatatgttggtaatgtaatcggtgagcgggtcgacgcgtaaattaagtttctccaattgtttcacagaatcgacgtgaacgttgttactgattacacctttgcaaaagtttatcagtgtcatataatgatcaaaacgtttgtaatgctttgacgaataggacgtgtttttcaacaaatcaccggtagtaaactccatttcaacacttactataaatattgtgcatattgataatactttatacacctgaaactggttgaactaacaggacgtattgcctgcctgaacaacatggcttctaaacgcaaaattactgaaattaaagacgaggcggtggtggccaacgttaaacaacagcgtagagacagtgtcgaaggagaagaagaagaagataaccaacaattagatatttacaaccacaacaatgctttggctaacattgaagacaatccgatggaggacgatgaagtgtcgcatcccgatcatgacaaaatgctatgcgtgtttaaggttcctacgataacgagagcgatgacatgggtcgacttgctgctgtacaatttgcaagcgaaaaacatcaccgtattaagatgcgaagcggcctttaacaaacttttcgattgtctcactttcctcgaacagggattgtgcatcgatactcatatggatgaattcgaacctgaaatctctaaagaaattataattttgaaaccaaaaccgcctcgtgtcgtgtacaacgtaggcaaagtggtcagaggcggatcgaaaccctttttctttttcgattatgccaaagtgaagagatgcgtaggtaactttggcgaatttctgagcatctcttggtccaaccaatatattcacaacaaggcgtacggaaaggtgatcatcaagtacaaagactttgattgtgatagcatgaaacttcaagacggcgcaatagtcaaccttcccggcgacgacactccatctaaaaagacgataattgttcgtaaatttttcgacgtgttccagcatcgcaacgaaagggtgtacatgacgggtcgtctagtgaaaagaatcaattgcgatccctttactattgaacggttcaataaagtgttcgagttcgagaccgataccaaatcttccgccgaagtggacatgttggtggggatacaaatagacggattcaagcagagcaaggaggaagtcgaatttgaaacagtaaacaataagaaggtgcaggagcgtacttactcgttggctattagacctatgatttttataaaaattgaagaaatttaaataaaattttattaaaaaaaatgtatgtattttatattaacggtactaatgtggaaaaacgttttggtcaagagtttatcaacttcatttgtggaggtaaaatcaaacacgaaattgaacatgaagagtgtacgaggaaaaagttggtggtgagatcgagctatgctgccaaaaaactattgtccgtcaacggtaaagcgttttggcccgacggcacaaggtttcgctgcaaacaaatcaacagtagaagattcgaccatcaccaccaccaccaccatcatcatagaagacaacgaagacactcttacgaaaacgattgttgtactaaaaaacttgcgtcttcgtctacgtatgaaaacaacagcaacaacggaggcggcggcagtaacagcagaccgtttagtcccaccttgagcaacaaagaatggtacgcgtcatatgataacattaactttgacgaaagtgataaactttatgataaaaaataaaacaatttatgtgcgaaaaacagtctttttatttaaaaattagtcgagccaaagttgagaaactccattctcatcttgtgaagtttgtcatcgatctcggcaatctccttttggtcctttgtgattcgtttgatacgttcccaatgctgcatctgaatgctgagttgtttcttggccatgactatttcgttgagtttctgtcgcaccgattcgtctttgctcttcttgtaagcctcggaggcgtagatattcttttttcctccaaacgaaggcatcttgttgattctgtgtgccggttcgattgtttttgtacacattgaacaaaaaatcaagattatatactaatcctaaatgtcataggttaaaaaaataataaatagtaagtgtataatgtataaaatgattagcgtattggaaatcttggtagtcgcgttcacgctctgtgtctctacgtcgccgttatctgccatggcaacgttcaatgtaaaatacactgtcgatcacgaaaccaaacgcatcaacgtaaactctgtgttggacgcgcccgtgtctatacacgtgatcccgcccaactctgatacgaacggtgacgaaaagttgacgttattgcatcagtttccgggcgtggccactaacattatatttccgtcgatcacaaaggacgatgacctgtacgtgcaacttaacaacggtgttttgtacaaaactcgagcgactcgtgtctacactaatttccatacgcacaagaatcgcatgatctacggccaactatacacaatcgccgtcgacgatttcgagatcgccaacaagatttacatcggcgcgccgatctatcgcaacaaacagctcgtgtccgtgatcacgtgccgctttgatgactacgaggccggcttggtcatgtatcccgtgactggaattcgaccgcaaggattgatttcgggccagatccagtacgatgaccgggtcggcgttgaaacgttacgaccgagcatgtctgtgtacggacgccagcagttgccatacaaaagcgcacacatgtcggtgaaacagtttgccatgaccgccgacatgaatcgccagctgtacagagatcttcctcgaagtgtgatgctgtttcacaataacaaagagatcaccattaccgtggtagagggtgaatttgaaatgtatcgcattcgattagacgggcctctggtcaccaatcaagaatcgtccaatgaagaaaaataagtttattcctcaaattataattatattaaacattatgagtcaaaatattttacttttaatccgttccgacatcaaagacgtcgacgccaaagttgacgctttgcaaagttccgtggatgatgtaaaggctaacctccccgacaccacagaactatccaacaaattggacgctcaagctactactttggacactatcgtatctcaagtcaacaatattctcgaagttttgaaccctgaaattccagatctaccagtgcccaacttgcgtaaaaagacaactaccactactaccactaaaaagtaaatttgattctttttttaaaacacataggttatttgaaataaaaaaattttggtaaaatttcattgtgtatttatttcatataaaaagagacaagtatttatcggtttcgatgcgtaattctgtatcgtggccgaacaatgcgatgcacattagttgattgcgtcgtatgcgagtcgaagacatctcatattcttgcacgtttccaataaaatcgaaattcatgcgtttaaaaaaggaacgcaaaaagttggaaaacatttggtgaggcgtcttgataaactggatcagtcttgaccatgcttgttcttcgaacaccacaaacgaacctcgatcaaagtttttgagcgaataaaacagttccattttgttaatgtctctttcgaggtagttgcgtattagatttttgatgggtgttgactttttatgcgtatagtcaacgtttaaacaatagtagcccaccacaaaagtaaaataacaattaccgtacaccgtatctccggtaagggtcaaattaacaagattgtacattttgtagtaactgttaaagatgtcagtgtagcagttttgcaagtactcgttcacattgttctccagatgaatcttgtccttgatactattcaacttgtgcttcaacgccaaacaacattgcgggtgaatcttgttcgtgttcacataaattgttgcgacttctttgctcaattcccactcgtctttcattgcggccaattcgtttagaaaaacaaaggtgttttcatgattgtccattgacaaatatttaaactcttccgccatataaaacgcctggccgttgatcatttctttgatgcgcgtattagtacaccattcggcaaagtaatgatacaacgccaatttaatttccagtgaaggtgaggccatcttcgttattgtgaatagaatcgtagacaatgttgattcgtaccagaacgtcgaataaatactcttcatagcgataagatataaagattattccgtataaagattatcgtaccaacgattattcaaatggccaatgtttcgtcgtagattaacatagtacgataaagcgtcgaccaatagcgtgtacagagctagaattaggaaaattataaatagggcagtgacgttagtgtcctgcggcataagtgccaaaacgatggcgccggctatgaacaataccggtacgaaaactgtggtctgagcattgaacagcaaatcgttatgtctttgcgtgtactgtagaaattctaaatgagtatacagagcgctggacgctaaagccgaacctaccaatgttacttcgtcaaagtcctctatcacttcgctattatcaaactctaacatttgaccattagagtttacggtcaacgaagctatatactctaaactgtccgctaaagcttctaatgttaaaatattttccacctcgctttcgaccaactcttcaaagtactctggcaaaaattcaataatgtctctgcggccgttttcaaaggacgaaaagtatgccgtcaaaaaggccatggacagatcgtcggggtagtctcgaggaaacatattgttatacccgaacgggtcccataatcctagaatcaaatccgctatgctcactatgatcaatatgatgcccactatcgatgcggctttgatagctattcgcgtgagcgctttggccaccattgaaattgttttaatggcaatcttgttgaacgcgtgcgtcacggccgctttgtaggtttctcccagcagtcgtgtcgtgacttgtcgcgacgtgttcaacaacatattttttagcataggaatcaacgtagtgttgatacgtttcaacatcgtctgtagagcttcgaacaataaatcgaaacctaccgcggttgctatgcccaatatcaattcgttatcttctagaaattgcgaaataatcgcttcgatgtcgtcatccgtggatgagtaattttgcgccaaactggtcgcgattctgtactgaagcggcctagcaacatgctcgcgggaaaatccaacttctgcttcatagattaatttgttcatgacgtcattcaatccgaggtcagacatagttttgtaatcggtaaagctgatctcgaaatctacgtccgcggcaggatcgcgcgtatccagccattcttgtaaaactttttgcgaatctatcgtcggcggcggaggtagcactgccgacggtcttttgtaattgaacccgcgcatatcgctaaacacattattcaacaatagtttcatggtaatgtatatactgtcgcccaatacgaatcctatcaaactctcccaccagctcaaactacacccgttattgacaagattcctaccgaagcgacgacagtacgcctcgttaaaggttcctctgaatctttcgggaaacaattcgtccgggtctgggtagacgttgaaagcgggcacgtcgtcgacgcccttgatgagatgctcgtccgtgcgcatgtacggactgttcaaatacattttagatacggtgtcgacgagaacgcactgtttggcgagggtgtaacgcaactcgggcgcttggacttcgttctcggctccctctctggtggcggcgactctgtctagattgtagcatgccggttgggcgtactgtatggcggtttcagatgtctgtgtgaaatttgtaccagtgtgcgcgttaatgggccccgtttcgtggaaaggataacaagtcatactttcgcaaccttttttgctaaatttcaactttacatggatcgccttgtgcgcaattgcctcgggaacgtagtagtcgttgttgttggcggcccgtatttcgtaatcgaccaaaatatgaggcaactttgttcgccattttggaatgtagcgtagtctatgcaaatgatcgctgtacacgttggcgttgtatatatcgacaaaggttaaagtggccatattgttgtggtggtgtcaacttatcattatgacaaagtatatatactacgatcatgtttgaaacgttatccattgataacaaagacagcgatatgaatctacgcgttagaaaattggtacagaaaatttttaataaaaacgtaccatacgtgtacgtcgtaaagttggactcgaacaatgactttcaagacatctacaaaatgatcgacaatgaagttgtcgaaataaaaaggccaccgtacaagtactacaaggtgcatttgaccataaatgcggccaaggaaaaattttacgtttttagagcagtaaataaaaaaccaatagaattgtgtgtaaaaatttataatgatgtgagcaatatagtaatacaataataaaatatattctaaaaaatacatttgtgtgtttaattctaacctaacctaattctaacagttgtcgttgtcctgtttggcggcagtctttttgtcgaaaatgatcttttcaaagttacatccaaagttgtcacaagctatcgagttcatctctttgaacgtgattaaacctccagtgtagtgcgatagcaatttgatcattccattgtgacgaatgttgatctctttgcggttacccgtgttgagattgtaaatgtaatgattgtacatgccgtactctttaacaatgtccggcacaacaatgtctttgatgacactggtgatccatagaaattccaagccaaacttgacaatggttatgcgttcgtcagcctttgtagtaatcaaaataaaactatggttttcaaaacgctctttgcaggaatgtagatagttttcaatcaactttgccgaaccgttctccttcttgatcttgaacaatttcttgacacccttgtggtttgagtcgtaaaattgacgagctatacatccgtacttgtacgtaaagttattggtagttttatccttgtaggcgttgttgttctttccctgttgttttcctaggtaccaaaatttaagattcctcgccacggtcgacagtaaatcgttgcgtgacattttatgttggttgggcaaattgtccatcgtcttgaactgaagacccgtagagaacttcatgatgtcatcaacgtacacggacatgtcgtactttttcaaatcttcctggttcggtgcctcctttctcgttatgcaaaacggcatagtgaacaagtgcttgttgtccgtcatctggttaacgtattgaagaatgtttttagacttttcctcgccgatagaggcaaacagcaaactagttttaccttgaacgtatacttgatcaagatgaaaagtgttgatgagcaaagacaaaaatccaaaatctttgacttcctcgaaatagcatagattcttgttggtgtccgagagtttcttttcgctaaactgatcttggatgggtatggaaattttcatgtcttgcagtaggtggtacgagatcatgaaacggtagcgttccaatgtaacgacgaggacaaagcggtcattgtgaagatggtgcgcagtatactcattgtaaacggaatgtacgcagttgatgtaacgaatagagtatatttcgtcagggttcgccgacttgctcacgataaacatgtagtaactcgtgtcgagtatgtggctcgcaaacatccggttgttgctcacgacactattattagcattagcagcgttctttttggaatattttttgatgatttcgttgaaaagtatctcagttgcaggatcgctgggcgtgggtttttccacgtgtaccggtttcatggcgctcgagatacacatctttttagcgtaacgacctcgaactttgggttttacgattacacgttgacaatcgctcgtcgatgtcgcaacattatttgcgattgcatcatcctcttcgacctcgcttttcttgttgttcttcttgacaacaacgttgttgtcgtcttcgtcttcgctactactgctactatcgctactgctgctattattattattgtcgctgacgcaatcttcgtaatgtaatcgtttcgacgccgcacccagtccgatgcccttggacgaaactcgtttcactcttttgctgccgcgagacgcgcgcgaagacgatcccgaagatgatatgcgtttcagcgaactgctgcttttattggtgggtttcattatagtgtcctcgacaatgggctgaagagtatcgaagagatcgagatcaatgtcttgagaaagattgccagcgtcaaagttttgattgatgatttcggcactgttgataagtagattgtagtttttgtcggtgtaagtgtcttcgaggccgttgttactattgcggtgatagtttaacatttccggagtgcgcggaatcgggtgttgaaatttgagaaattgcccaagattctcctggatgggagtgcggtttccgtgttggctcgatttgtatccgttgtacaggtactgctggacgttggcgttgtcgttgagagtgtgcatgtttacaagtaactgaaacaaataaaataatgaacgtcaatttataccgtgccagaactaatagtaatattataacattttctattctacacacgattatttcattaaatatattcgtatttcaattggaaatagaggaggagataacaaacaacgcaccttctacgttaacgtcaagaccgtcaataacaagattagttagcggatacgaaaaaggactgcgcaatatttcgatgaaaatgacgtgtatcttatcgtcgtcgttgtctgcgataaacagcgaccctaatgcgtatctagtgagctgcgttagaacaccgtgtgtgttgatgaaattgatagcttcgccaagattcacacgacccgtggcacccgtgcttgtacagtacaacgggacgaatcaagtgtggcacgtctttggcgttacgaaacgaaaggaattggcttcgatatccaagatacgcggcatcaccgccaccataaacggcgaggaaacgttcgtgaataaagagctcatcatgcttacgggaaacataccctcggctttcgtatcgtccatcatgaaaaacacacccaacgtcgaggacgtggacactatgaagttggtgtaccccgaacttgaaattaacacctacgacgtgatcgtagagaaaaataatggcagttaaagatgtaaaaaaaatttattaaacatacacataatattacaatagtaaacttttgtacaatcgtcctacacagcccgtttccagagaagcgggtacacaatcttgttcgagtacgtcaaactgttgtcccggcatgcagaaaaattgttggcgttccggacaaacgtagtaactgtcgcagtcgtaggggtccggtgcgagtccgtgatatccactgggacaaacttttcgtacatgaaaatcatggtgcaattcttgtagcttgtgaaacacgaccaattttacaataataaacagcgctaaaagtaaccacattgtttaattgttgccgctactgtagctgagtattcccagagcggttttgtttgaactcttttcgccgtcttcaactctaaacttaaatttactcaacagcatgttggaatagttactgttcaaggtgggcttacacttgttcgcaaacagtgtatgatctagcaaagattcggaataattgtaaatgccatagtttttcagtatagaatagtgaccgtacgcgtgttccattatgaacagtaaacacagatactgcgtagtttccacttcgctgatcttgatgtaagattcctgttcgtcttcgtcggacttgcgctttcgcggcggagcaaaagttttcatcagattcaccttttgcttttccttgacgttggtggtaacgttcatgtgcgtgtcaaagtttcgttctaacatttttacaatactgatacgatctatggtacacactatctcgtcacgatcgttcaaccgaaacatgatgggttcgccgggaatcacgaggtccggcctgtccgtgaccacaaattccattctattatcaaagttggtcacgagcggatgtacccgattgtgtatgaaacccagcgacgccaccactatggcggcaatgtcgcgtgtagcgcttcgatctcgaaagatggtcagcaaaggttgcgtcaactgtaacgtgttcaacattgccatgtactttactattactaacttgatctttaaagtgtcataactttcgagactgtttaggttctttgcatcaaattcggccaaatcatagtccttttgtagtttttcattgccgtttattatttcggttacggttctaatttttggggaattgttacacttgaactgtttcatagtggtgttgttacaatactactcttataacatgtttcgttgtggggtattaacaaaaggattagttctcatagttgcgtttaggggattcatgtacgccgccgtggctggcgaactgccgctgcctcctggttgactgctcgattggaagagcagtatgatgagtatgattatgacgagacctatgagtatggtcatcatggtgttggtgtttacgctgtttaagctaaagcctcctcccgacgtcgatggtgcccgattaatgtccattattattacgtaaagagcttgagcagtgtgtttttaacccaaggatttttcaaatgatcgagagttttgaattgagaactttcgtaatctcctcttataattaaatatgcaggcacagaggtaccgaaaatgtgcttagttaaatatattttgtgtttggcgtcgatgatgtacgtatccttgttcaaaacgtgaggtgtcgcaaattgcctgtacggtatgagccgatagtcaaagtacaaaagttcagtgctgccgaacaaactgttgctcgccaaaattataaacagttggttggagggcacgtagaaagcgttgaaagtgccttttatctgtataatatcgggccgaacgaaaatgtacgacgtttcgtttgcgcgtaacgtgggcagcacgctgttcgcatcagtttttatatttaattttcgatacttatttacggcgtaacgatctatcacaatctcgttgtaactactggccagtttaatattatgttcgccaaacttcattataaacttgtaattggatgttttcgtcatgtaatcattgagcaggtcaagcaggtcatctggaaattttgcgtcgtagatataatctcgttggataaattttatataatttgcgctgttctccaactcgaggttaatttcgttgaaaacctcatttggctttttagttgtaaaatgtttgctgtttatgatgcggaaattgttgcgcacaagcggcagacccttgtaaaagtttcttatgacgtaatcgctatttgcgggcatggttatgttttgtcgtaaaaaatgctgcgacattgcctctccgatcaagtacaatctatacggatagtcgggagtgtccactttcggcgaagcgcacatttgtacgcccgaccagtccacgtatgcattatcaaagatgaaaccctctcttccctcgagcaacacggactctgtattcgaggatacgaaaaatctatcgtcaaaaactttcataaattcactatacagcaaaagcatgaaaggttgaggatcgtttacgaaaaagtttgtcgcgtatattggagtgtttggctggatatagtgtcttttgtcgaactctactatgtctacgttgtcgcgcatagaaatgtacttgaactgcggttttatatacttgaacatattggggtctcggtccgccaccaaatgttttaagctcataccgctcaagtagtttacatacttcaaaagggtatcatcgtcgatggcgttgaaattactgcgcacatactcggtgatgaaaggccgcgcttctgacgcaatgtagtcaaaattgttgaggtcaaagtatgttgctagaaaaaggtattttagttgtttttgttctagcgaatgcttgaccgccgctaaactcattatgaatcttcttactaaagttgttctatgatttgttgtttagcggcagtgggagttttaaaactagtcttacatgcgggacacacgggatacaaacttgaatgtttccacagttgagcatagcaaatgtaacacacgttatagccgcagcactcgttgggtttcagaaaatgttcttcggcgctgctctcttggcaaatgttacactggtatattttcgggtcggtgaacacgtttatcaactgcatacgttcgttcattagtttgatgatgtgtagatacttttcgctgcggtcgtacaaatcgttgagtgttacactgtttgatttaacgatagcactacagcaaaaatcatttacgaacatgttgttgatcacacgtatctgtttaatgtacggtatgaaaatgtacatgttatattggaacttgggttgagtgttcaagttttgtataacatcaacgacgttctttatttcgttaatcaaatagtgtacgcatgtgtctcgcggaagagatatatcatcttcgttttcgcgaaagaaaagtaatttgtcaagattgcaattgtacgattgctgatatgtttctttaatgatcttgaaaacggccatcttaacattgtagtgagctcgaatgtttaaacttaaatctggtgtgtatagatggttaaagagaaaattgctaaaaacttgagcacacagctcggaatcatgattaatactcattttagtttaaaactatagtcattatcgtcatcaatagaaacaacaaacttatataacaaaaacttttattaaaaatatattaaaaattaacaacgctgtttcttttgtggaggagcactgtcgtagtcgtcgtcctcgttgtcctcgtcgttgttgtcctcgtcctcgtcgatcttgtgacatttaatgttaatcttgagtttgttttgccaagctttgagaatgttgcagtaaatgttgtcgtagttggtgtcgtgcaaaatcaattcttgcgtttggtcaatgtatggagaattttcaatcatgtaatcgagttgtttgtagtcagtctccgtgttaagtttaaacactttcttgatgtggtccggtatcaaatcattgttttgcacagacccattcttagtggccagtatagagttaataacggttggcgtgttgagattttcatgtgccaacgctctaacgagaacttcttcaatgtagttgattttttctatttcgtaaaggaaagaattgaagggtccgctggtagttttagtgcccagaatctcggaagtagttttgtacgtgttggaaacgaatgttacgtgtttgacgccatagcgttcgactttttcgtgtaaagaatgccagtcccacatcagagtctcgttgtcggtatacacgttgtagatttgttgggtgatcttctcgttcctgtaggatttctttttgccggactcgttgagttctttgctggcctcggccaactcgttactagcttctatggcgccgtagtagatggtttcggcgataagtttgttgagggcctttgcttcgaccgtatcgaaagtgtagccgagcaaaagtagagtttcggcgaaaccttgcataccaacggcaatgttgtgttttttgttttcaaatttatccaaaggatacatgttaatgttaatcatggcgtccaagtcgcgtaccacattcttggtaatgcgtctcaacagattgtagtcaaactgtttagtttcgaggtctacgcatttgtcgacagcaacggatgccgaaaacacaatgttatatttaacttgttcatcttcgatggaaatgttgttgttgttattaaaagcatccttgaacattacagacaagctgccgttaattctcatcgtatcaataatcagtttgagcaaatctttagctttaacaatcttggcattgatgtgttctttctcaaacttgacataggcttgctcaaacttgtcgccgtactttgaactcaaggaagtacaagactccggatcgagtaatgtccagtagccgttatttttcacacggttcataaaaatatcgggaatgtctatggtgaactgcaaaaagttaaccttgtcgctgttgttgcgaatagtttcgataaagtttttaacttcatgatgccatagttcaatgtgaatagtgggcagtttcacaattttattcttatgcaagatatgacgtaccatgttgttaaagacaggcagcatctttacggaaacgttttgcatgtacaagtcgacagctccgccgcaattcatgatcgtagcacattcttgaataatcttgtatgacaaaaagatattatcacaacagttgtctacgtagagtttatagtcgtttgccagttgagtagaattagtaccggcacgagcaaaagcaagatcggagtgggcgtacatacgcaggctcatcatgtgatagctctctataactttttgtatgtcgtcgccgtacatggtcagggcgacacgcatgaacatatgttgaaatctttccagcagaaaatttttattttcgtacaatcgaagatcgtaagtgtagtctcggtcatgacgtaacactttgttcaactcttcgccgtgaaccatggcgagacaatacaatctttgactgatctcgtttttatggtacagatttttcattacaacctggaacgattcgtcaatgtcttcgtgcatttctgttacaatttgaacaacttctttgtcgaaattactagtgtcttcaactgttacacgacaaacgttagtatagtcagttgataaataatactcgggtttgtccaatctcatacgatcttcacaaagattctttttaatcttcttctgcagctccatagtttcgtgtaactttttgtattgattatagaaatagtcattgtcttcatcatcactgtcttctacaatcttctcaggaacgtcttcgttctcgtcttcggtgtcttcgttattgtcttcggtggtaacattaggagtaaacaaagattcaggctcaccacttgtagaagcgatgggctcactaggttcacccacttgttgctgcgcctgttcttgactattctcaacgttatcttgactattgttataatattcttgattggagttgaacaactcttcaggcaaaatatcattcaagttattctcctgctgagaattctgggtagcgtcgttcgcaggggtagcgaaaagatccattgatagattttccattgcaaagtttgaagtgaggtgtttctcgttaacactgcacaattgttgtgatacttcaacgatcctgcacgacgcttttatatccaagacgcacatataaaaattacaaatatagataataactgtgatatgttatcaagtcacgtacgctgcacaagtcacgtagacgccatcataatcataatcctatgctgcattgtaatctaatcttttgccgcatttttatctaatcttttgccgcactgcgggaattgtaagtaattttttcctttcgtaaaacattgtgaaaaaataaatata

SEQ ID NO: 2 - DNA, 132801 bp, Spodoptera frugiperda nuclear polyhedrosis virus
  ref <308>: DATABASE ACCESSION NUMBER: ATCC PAT-121213
  ref <309>: DATABASE ENTRY DATE: 2014-05-09
  ref <313>: RELEVANT RESIDUES IN SEQ ID NO: (1)..(132801)
atgtatactcgttacagctataacccatctttgggtcgcacctacgtgtacgacaacaagttctacaaaaatctaggttcggtcatcaagaatgccaagcgcaaggagcacttagctctccatgaaatcgaggagaggactttggaccctctggaaaggtacgttgtcgccgaagatcccttcctgggacccggcaaaaaccaaaagctgactctgttcaaagagatccgtatcgtcaagcccgacaccatgaagctggtagtaaactggagcggcaaagagtttctcagggaaacttggacccgtttcatggaagacagcttccctatcgtgaacgaccaagaaattatggacgtgttcctagtgatcaacatgagacccactaggcccaacagatgtttcagattcctggcgcaacacgctctccgttgcgaccctgactacgttcctcacgaagtcattcgcattgtggagcccgtgtacgtaggaaacaacaacgaataccgcatcagcctggccaagaagggcggcggctgccctgtcatgaaccttcactctgagtacacgcactccttcgaagagttcatcaaccgtgtcatctgggagaacttctacaaacccatcgtctacgtaggaaccgactctggtgaggaagaggagatcctccttgaactgtcgctcgtgttcaagatcaaggagtttgctcctgacgcacccctgtacaacggacccgcgtactaataaaccataccacaaattcaaataaatcatacacacaattcttttaacaaaatttattaacattttacaaagtcataacatctaccattaatttatcaaattttagataataattgcgtaaggtatcagcgtcatcggcagtaatctgctttttagctagaatcatattaatagcggtcatcagcgtagtgatagattcgggcggctccattggaagctgctctaatttttttttcaacaaaacatattgagttttcaaagattcaatctccgacggagaggccaaccaatcgttatcttcttccggtatctcgctaccgctgctcgtcgacgaaggttgtatcacgttacgtctatgtagaatactcatcgcggactcggagcgagccatgagtggtgcttccgcgggcagcgaagatttaggtttggtggcagcgggcggattttgtaaagctttaagtttatcttcagatatgggagttttacgtttgagcctttctctgagttcgctttggaaatctacaggtttcggcgaaggtttatcatctttcgtcgattgtactactattttagaagaagttggtggtgggggtaaaggaatttctgacgtaggaggtgacatcatcggtggaggaggtggaggtggagggggaggcggtatattcaccgaaacgggggcattcggcgtggaaattaaattaggcattggcggcggcggaggagcaaagatatccatctgtggcggcgaagttgatactttgatcggcgaaggaggaggttgagatagtattgcatcatcattattttcgtctaatgttatcggtgctgatggcggcggtggcggtggaggtggcgcataagattgttcggcacccatttttggcgttgactgtggaatggatgtcgagacgggtgctttattcggtaacgcagatgaaactgtttcggcggagggttttgtttgatctagagtaactatttccgtgaacagtttgttgatatcgttttcctcgatctggtataatttgtaaaggtctaaaaatgtttttaataaagcgctaagattacttatgttgttttcattgacaatgctttctaaaatattttccaacttagatttaaagcgactactgtcgttgacgtggcgcaccatgtttttgagttgcgtaatttttgcgctcatagctacagggatcgatgtacttgctggcgcagcatctacacggaccattactttattttcgtagatagcccaggctaattgtaagacctccattagagtgcgagaattcagagtcacttcgtcgtcatacgagctcatgttctcgtcgcgataacacaccgcatcgaacagttccgtagacacgggtttggttaatttttgaaaaagatccacgacattattctgaggattttgtagaaattcatacacggtggtctgcatagtagccatggccaagaacggacttactaataataataatatggacactactctgcaagagatcaccgacttttactcggacctcgaactcgatcgtgaacataaaacactcaacggtagatacggtaaactatccgtgtggaaacacaagcccacacaaaaagtgtttgtaaaaaaacaaatcaagctgaagcactacaatgaagtcgagccgatgatacatacgctaatgaagaaaaatccatattacataaatttatattacagtatcacgacgttgaagagtcatgtactcatcatggactacataaagggaggtgatctttttgaactgctaaagatggaagagtatctcagcgtcgacgaaactaggctcattatcgggcaactgtgcgaagggctgcacgctctacacaaacatcattacgttcataacgacatcaagttggagaatgtgctgtacaatagatacaaacagatatacatcgcagactatggcttgtgcaaagttgcgggccagcaatcgtgtctcgatggtacgctagactattattctccggaaaagatactgggtcgccgatacgactatcactttgactggtgggctgtgggaatcatggcgtacgaactcatcaccggcgaacatccgttcaagggcgccgacgaagacgatagcttcgacattgaaaagttagaggaaagacaacaaagaaaactagtgtttaaaaatagaagcgttcctgcgaacgcacaatcatttattacaggaatgttaaagtacaatctaaattacagactaaataattacaacgacataataaaacatgtgtttcttaaaatctaataatattaatataataatatcacaataaagctatatatcacaaacattacaagactttaattttttggttttttcgaaggaggaggttgcatagagtcatcatcgtcatgatcctcgcttacaacatggctaatacgagtaatcgtaacgccaattttctttggtttaaataaatttacatcatcattgacaattattacatcatcctcatcgtcgttgtcgctatcatcaagaggttcgtacggagtacatttataaattactggctctttttctaattttataacagttctgttgtcgttagggtcacttttgttaacgtactcatattgataataggttacaggttcaatttgcgcattactgtttaatagttcgcgtttaattttaggtttttgttcccgcggcggagtagacgatgaagatgatgtaacaacattatttgatgtaacaacattattagcggtcgcgacatcagtagtattgtcattgacattatcgatattagtattttcatcattaatattcatagtgtcttcgtccgcgttatcaatttcgttatcgtcgtcactgtcgtcactgtcgctgttattattttcgacatcagtgtccgcgttagtttcgacgttttcatcatcttcgttatcactatgtaggctctcgacatcactatcattattgttatcgttattagtgttattagtgggagcgacaatattcgaagatgtactcattagtggaaaattagttagttcgttgtcggtgtcgttgtccatgttttctttgtcggacaactttttacacgcgccgcagcttttaattttgaatagactcttgaacgcgcatttagagcaaagtttgtggttacaatttttgtaatgataatagatgtgaccgcgtttacatttgtcgcactcggctataacgttgttaagtcggtacatcattagattatacgtatactttgccgtaagagttttgatatttttttgtaaagaaataaaagtactctgtaccgcgggaatgatcacgttacgaatagactcaatagattttctagtttgacattcaaaatctattgttttcatgtctggcgttaaagattgtacgcgttcgtgcaactgagtacaatcgatgagcacttcttccaaaggcgcggtcattaaacgcgaaacgaaattttttataatagaatagttttcatcaatagtcttgttactgtaatgatctagatacctctgcatacagataatgacatgctgtaacactatagatttagtgagcaaagttaaaaggttacgaaagccgttgctctccttgctaacctccatgtttgcgatgacattgtaaaattcagtgtatctatcggtgaacaggtaatactgcttgtacgtaatcttaacaaacttccaagttttggtgcggatgtctacctccccgacgtgatgaaagggggccgtctgaaccatctgcaggtacacactaacacttttgcgcacggggttgtgcgaaaattgtatgtgtttatgaggcttgagcgcctagaacaatcgaaaaaaaaaccacatgaataattttgaaaataaccccaaccacaaaaacgttaatgatatacacatacagatggacagaacaaagaacgataacatgaacaggacactcacctcgttattgttagaagtccaaaaattcattatggcggtgggtggcgcgtaacagggtgaatcacggtgctggcggtgctgatgaaaaaggcactcggcttgatgcacagcaaagatagcttcaaactgcaacgcttgaatgaaggcaaagatcgttgtatgggcatatataccctcttatcgcggatgtatgatgatatcatctgcgataacgtcggtcacgtacaccaccgccagtcacgtacgccatagtcggtcacgtacgcacggtcgttgcactcggtcggtcgaggtcaccgctcacgccaccggcgcacgagcatccggtcacgtaggccattggtgccgtgtgcacgcagtcgttgaccgcattaggttcaaggcctgtgcgtcatcaacttttttgcagtgcaaaaaagtaccttaaagtgtagtatatggagagcatattctacagtgtagactatgctagtataatagtctacgattcgaactattgttctgtatatagatatgccatatctaatcaacatttttgcactacaaaaaagtacactaccatctttatcttgaaaaggaggggatacgggcttatatataaagcgtcggcgacgatgagcatcagttcaagttggttgaacattctcaacgcactaactaacgtttgtgaataccttccacagtactacttcaccaccagcatcatgtacgcaaacgagcacatcaactcttcggtcgagcagcttcgtggcttcgtggacgacgagctgttggacgagctgggcttcaacttcgagcaacagcagcagcagcgccaggcgaaggaggagcaagacggcggcgacgacatgcagacaactaacgatgacgctgattctatttgtgagttaactaacgagctagataatttgctctgtgaagatgaaaacgaggaggttactgttaactctaataattttgactctaaccctccgctcatcgattactctgccgacagtgacattaagaaaactatgcgcagcattcatactacgtttggcatactggccgagctagataagttgcgcagtattattaaagttaaacttgcaaactttaaagacaacaatgtaagcaatgatactaccaacaacgacaacaccaacgtaagcaatactcttgacgaaaagattgcaaactatgagcctagcgaagaaacttatgaaccatcttcatcagaatttttggattcggaatccaagtttcgtcctgaagaaatgaaagatatcatcataaactacattcatctgtacttgttaaaacatcacaaaactaactctgtctctgattccgttaccaaaaccctcagagaagacttgagaatttggatgcttctcggcattcagctattgtctaacgtcgcagatgttgaactgtataacgttgctactaaacaatgggccgatatctttaaagaacttacagatgccgtcgagacactgaccattgactctaaagagttttacactacaaagagactgcgcaacgtcatcggagacatctttttcttcgataacctttacaataacggtgcgtgtaagtttcacgaaaatgtttctgaaatctttgaatatattgataaatgttttgcaccagtagatgacgatattgtgtctcgattggtgaatgatgatattctgtctcaattgcaaactatgaacaatctggcatccgaatgctgtgtatctgtcacgccgtttaaagctgaagacgtctctaacgacgacaacatgccttcgttgtttagcgagtcgcgtgttaagagcacacgcaaactctcgcacaagtttaagccgcgtcgtaactctaaaaaggcccgtcgtgctctcaagtttgagcaaatgtacatataattgtattttgtgtgtaaaaactatgtgttattgtgataactattgtattattattgtaaaactgtgttataataatgttgtaataatttttatgtgtctttaaaatgtgtctatatattgtacctttatatattattgtaagtttgttattaaaatttaataaatacttttaactcaacaaacatggtttttcgtcctctccgtaatgttaacaaaacctatagcaatatcacatcctttgtggcagacaacaatagtgtagtcaatgcgagtccatcaggattcagaaatgtactcaataaacccacatctgtacctgcgggaaataatcaaatagtacccggctataacttgccgaacaataactttatttctacatcagaactaaacagtattatgagaaacaatgacactaacggtatgcgcagagtctttggtaacacacccaccaacaatgactacaatagtttaactcaaattagacgagcagataacatacccgacgctaacatgcacagtaaacagttgaaacgagacgctgtgaaaaataacaatccttcgactcgcaccaacactcctgaaggtatacaaaacagtctgaaccaaaatccctctctcaacgattacctacagggtatgaaaactgccggcaaagtggctctgctaggagtaggtgtatacttaacgtttgaaggtatatctttgatacaagacattagggaagcattggccagaacgggcggcagctaccacaccacaggcatagacggaggtaacgaagtttccgtatgtttgcttaggtacaggacttgtcgtttaccagatgttatcgataatgaaaaagtcactatatgtcgtaacgacccactgattgcagacattaatcaattacaaaacatatgcgcaaactttaactatgaagaagaaggtagtgtatgtcgcgccagcgaccccaacgccaatcccgacagtcctcaatacctggacatatctaacctaggcgtagatcaaatgatcaattgtatagaaccatacgactttggtgatcttatcgctgaccttggccttgacgggttgctaggtgaaaatgggctgttcaccaagtcgtccaatagcagtaaaagcattagtgatagtttgttgcccgcaattctgatgattggtatcattatctttatcgtgttcataggctattttatctttaaaagactaggcaacacgcaaacgcaaactatacaactagaacctatacgtactagtgtgccgatgacctcggtacctattcaggtggtacgataagaatcaaccgaaaaggatataaaaggacgatcctgccgaagcactacacttcaacatcgcccaacaaactggcagcacgagcagcaaatactcggtaagttttttcttttaacaatctttaccattgattatgttgttatgattgttgtatagtgtgttgaactaaaggggttttcttctttttccatagaaaatgaagaagtttatgtacaacaagggcggcaagaacaccacggtgtctgccaaagtcaacacgaatctctcgccagacgagaagcccaaagatctgcgcggacgtttcctcagcaaccaaaattatcggctgatgaagtccgttctccgattctccagcgagtatgtgcaaggcgtgcggcgcgtcaatgatcttgtggagatgaagtgcgacgacaccaagtgtaaagagatcacgacaaacgccaagtgtcagggagaatgcggtaaaatttttaacaatggtaaatttttgttctgtgtctacgacacttcggctgctgctgcaacagcttcatctagcggcgaagccaagtttaaactagtttgtaatctctgttctaacgattattcgtacaacaacaggtacgaagttttacaactgtttcctaccgtttctcagcaagtggtagaacgtttatgtgagattggttttttgactaaatacatctttcccattaaactaaactataccgagacacaaactaaaaccgaagtcattaactatcatgatttttacaagatggttaaatctatcatcaaagaaaagaaaagcaacgaacaaatcacagagattaaactcagcacctatggtcgagacttgttcgtcgagactgatgatttttgcgttatgagaaaagacagtaacgagtttaacgaaaccgtctacgagttggacttctttcctaaacaaagttccatgttagagtttttgaaaatttacaaagacaccaaacctctcacatacttttatacagttaaaaaacgagtctacttgagcatctttgattatgcagtgtattttcctttcaagtgcgtactattttgtaaagtttgtacaaagaacaaaatgtatctaaagacacatcccgttttgtattgtagcaagtgcggttttaccgatcccatgttctttaacaattccgcttttctagagtctaccatatttctggctaaatgtgtaaagtacaaaactatgccagtagcccgcatcttttactacgacatgactttgtacaaaacttttataaagaaaccttttgttaaaaaataactccatgtgataatcgatagttttgtaagatctctatattaatctcttagcacgtaagcaagcaatgttaatcgatagttttaagcaagcaatgttaaccaaatattgatactctttagttttaagcaagcaatgttaaccactctttagttttaagcaagcaatgttaaccactctttagttttaagatatacattgatactctttatattataatctctatattattcttttttatactaataaatttttatatcaaacatataatttttcattaactaaccaccatcaaacactattcaactaaccaccatctttgctttcggcgaaacacttcactgaaagcaaacatcaataggatgatgacgtaatatacttccaactttgctttccgcgaaacactttaccgaaagcaaacattgttaaaattatgcgcaatagaacatgacatcattccaataaaaacatgatatcattctaactaccaagaagatgacatcatctatcaaactttgctttccgcgaaacactttaccgaaagcaaagatcgaggacatcgacctacagtagagttgttaatctttgctttccgcgaaacacttcaccgaaagcaaagttcatacaaaatgatgaaatcatcacatcgcatgacatcatcctaaaacatgatatcatcctatcatgacatcatcctactgccacaaactttgctttcaacaaaatacttcgccgaaagcaaacatcaatgttaaaatgatgtcatcatatgatgatgcaataaaaacaggatgttaaataaaactacacgatcgttgacgttatgtttgttttaaagattatagaacttttagaaaattattctttaaagttttaaaactaaatagtttgttacagttaatctttgcaatcgcatacactttatcgattgttatagttaaactttgattgttatagttaaacttgtttttaattggtaatctttgaaacgataccattttcaagattgtgaaaatttaacatttgttaaaatttttcaaaatttccactttgttgaactttatgtagtttccaagatcgttaatgaaaaacattatttagtgcatgatcttgaactaacaactatttgcaagatcgtaaattacaaacgcttactaaaaattctttgagatggatacaaagtcaatcccattacacaaaacttaaagacgatcttgaaaacaccatagatttcgagtaaacggatttttgtaaattttgttaaaaagtttgcaaatcgcgttacgacgaaaactctatcgttttcaagatcatattagttaaactcgataactaaaaaactattagttaaactcgattattagttaaactcgataactaatagttttcaatagacaatctcgaaaacaccataggtttcaagataataagatttgcaaacttttgcaataaaatttaacaaaaattcatttactcgaaatctatggtgttttcgagattgtttattgaaaactaaaaccaagtgctccatgtttgctttcgtcgaagaatgtcatcgaaagcaaagatcgactttatctctgctgacgtcatttgacctcgatcgctaactgcgcagtattaaatgcgcaataaatctatttttagcgatgatttaataatgcggacattcaatgatgtttgctttcggcaaagtgtttcgctgaaatccaagatggttacacatatctattgacgtcatacaattgtttcacaatatttaatcatccatacaagatccgcgtagagacatatcgtttcgccgaaaaagcaagggtaacgattcgttaccctttgtgtttcgaggtcgatgatatgattatcggcgaaacgtgaccggtctacgtgacttttgtaacgttccgcgcattaacattaattacacgaaatgggttatcggtacaatgattgtgttctataagcgataatgtcgttcgacttggtataaaagaacgatcctgcaagtgatgctcacagtcttgaccacgaacactcaacatgttgtgtctcaaacaagttttcttctacatcacagcggcagcgttcctgctggttccgatcgccgccaccgcaaccaccagcaccagcaacgctgaagatatcatcaaagtaacccagctgccttcaacttctggtctgtatttccaacacattaaccgtatgcagtttgtccagaacatttggcactttgttatcgaaatggaccatggttctgtgttctaccgcctgcagaccatctataaccaagctcaaaagatacacaatgaattaacaaaaatctattataaatcgagcgataataaacgttacgatgattgtaatagcatgagatttataaaattggacttggaaaatattatccataatattatacccaatcttgcacagcaacataacctgctggatcaaaaagtacccgtcactccgtccactgccaacttgaagaaggtcaatttagaggaaaaaacaaacacgcgttcgaaacgtggaattttgaactttgtcggccaagtggacaagtatttgtttggcgttatggacagcgatgacgcccatgaacttcacatgctcgccaacaccaccaacagtttgaattcccaagttaaacaattgacggacgagcttattgttttgagcaaatatgtggatcagttatcgagttatagcgacaaagaaaatgtacaatgtagaataattgctgaaaattttaacattttgtgtagacagttagatgaagtagctacgttgtacaacaaactggatttggctgtggacaatgccaagttgaatcgtctcaactcgtttgtcatctctcccgaacgactcctcaacgagatgaggaacgtgagcggtcatcttgccggtctctcttggccggtgccgcttaccaacaaggcggtgcacaccctggtcgacaatgtgatcaacgtgcacgtgttcgttacggccgaacgtaagttgttgtttatcgtcgaagtgcctttagtgagcagtgaagcttttgacgtgttccatagtattcctttgccgtactgtgacagtgaccacaaatgtgccgttctattgcccgatagtaaatatttaggcgtatctctcgaccgtagaagttatgtgcgtctggacgatactaatacttgccgcatcaccgataaaattatgttgtgtttcaagccacaaataatctacgatgtgaatcaggccaagttgtgcgacattaaaattttcataaagaacgacaaagacattgattataatagagactgtgacattagagtgggcaagtttgagagtgaattgttttacgcgacctctgactacaataattggttgtacgttttacagaacgacatcgatctcaatattcaatgtataccctccgatactcgtcctgatggtatgtcaattgaacctgttgttttgcacgccggtgtgggtttgatccatgccacgggcaaagataattgtaaacttactacgaaaaagtcccgattgactgtccacgatctgtacaataatttgaatagtgtcatcgaaatacctatcggtttaacgttcaacttttctttggctctacaggacattgataaaattactgtcgataatatgaaaattaataacgatctggaacacaccaaccttcacgagctgactaaccgtttgtacgacttgcggcgccagatcgccaacaataccatcttttccggtgatcaaatttacgatgataacaatgacatttttgccggtgtgtcacaatggtttgccggaatcggtattaattttcattacataaaaatcattgtcatgtggatcatactggctctgttaactttagccgtaattaaaatttatcgtacttgttgttctggtgcgtgtagcactttgtgtggtggctgctgtagaaaattaaagtttcgtcgtacggctagcgatcacactgtagtgcgacgcgacgaccgagaaatgtattatttgaacactttgcctagaaaacaaaagaacaacaagtttcagactaagttagatgctcctagaaagtatcatatggaagatgaggatattgatattgatcttttacaaagaaacataacttttgatgataaaatataaatgtgtatgttataatgtaaatttatgttcatgtaaatgttaatggatctgtgtatgaaataaaaccaaaaaattttttatattaatatatttttataaaaaagtgtatacagttgttttatttattgtccagcaaactatcaatcttgccgttaataacatcaatctttcgactgttgattactatcaagtctatagaatgtttggtcatgttgtgtaacttttccaagttttgcactatagtttcatgatagtcagacatgagttttgtgacatcgtcaaactttgtcgtagtagtttcgtagattaacaatagaaattttttaacgaggttaatttcattggccatgctgtttgcataccacaagtatgctgcgagcaaaactagtgccgctccggaaaaattcattgtcgtagactccctcttactttttacgattcatcatgtctttaatggacttgaaaccgtccaacattgtgtcgttgctgaaggcaatcttattggtcaagtccgcttgcatatttttaatattttccaaagcgttattaatgtttgcgaatagagaagcgtgttctgctcgaatcatgtcacgcaatagttcatcattattaccactgttgttgttattttttaaattatcgtagtttgattcgtacaacgtagaagattgattattggttatacattcttgtagtgtacaaagttgagttttaatatcgagtagaggatctacgatttgactctgtgcgcccaccagcaaatcgcctatcaactgtttcaaagttaataattcggggcgatgaacaccttgcggtggcggtttgtacattgacagatatttgccgagagaaaagacgtgaacgtaatttttattgttgcgtgttagtttctgagagttgtgagtgtttgcccacatgaccgcaggactgaatccccgaatgctggctatgggagctataagctgtgcggctgcgttgagttcgacgtaaccgtcacgatcttcgtcgctgttttcaataatcaccacttctagcagtttatcattgtattgaaattgtaacgacgaattatcggaggcggaggcggcggtaccggcgctgttggtggcaaagttgttacttgcgtagctatcgaacatttttcaagcatcttattgtacacagatggataagataatttcattttatgaagtcaatctacccatcgtatcgcaatatgttttattcgtcttcgccctatatgcgcacttaccgcagttgcggtaacgacactatgatgaattacaacaacaaccacgaaacactacgtcacgatctgcaaaccttaaaatatcaagtttacgaaatgtgccaacagtctaccacggatcgtaacctatgcgaccgtatcaaatcttccatagacacgtcgagcagtagttacgttgttcgccctgtagtgccgaccaccaaaactattctcagcaacaacaagaaaaatggcggcggaacagttactgtcgtggacaccgtcaaatattgaaactattgacaaaaactcagactatagcgttctgctaagcgacgtgccatcgttacaagtgaccgcgttgacgcctttcctcgacaatggactacgtgtcaagatcaacggtctacggttgttctacgtgattaaaaattccatgaacaacaacaacgataaccacaacatcaacatgggcaatgtaaaagttttcaaaggaaccaaaagtaaaaaaaacgtgtgtttcgccgaagcgtgtaccaagtccgaaatagtacaagtgctacagagaaaactgaacatgcccgattgtatgcaaaagtttttgatggactttcaagtttgtcctaggggcaatcgttatcgtaaacgctttatattcaattcgtacatagtgaacgttttaacttgcaccaaatgtaacaagctatgtcttgtcaaagccatggcacaattatacaaccacgacgataaatgtgtacaagagtttgatagattgttgttcaaaaacgcaactttgtacaagcctccgaattgtgaaaacttgaaaaacaaagacaaactatgtttcaaaatgggcaactgtaaaggaactaatcctatttgtaactattgaaataataaacatgtgtgttataaaaatacaatttttttattaattacaaattttcatttataaaagatttcacagtgtccacagaagtgttcacctttagcaaacgtcttgaacgttttgtgtaatcgatctgtttcatgtccaactcttcgttgatacaatgcatggccaggagcgggttaggatgtacgctatcgtagatcatttccatattgttaaattttcgtttaagagcaccgtgtcttcgctgctgagccgctagaaacgccacctctgtgccctcgccgtcctctgacggttttacaaatacagagagacgaggatgtttggtcgaatctcgcggaaaacgaaccatttcatagcgaggaccgttgccgttgttgttacacgtgttggcgttgttgttgttgttgaaaaatgatgcgctgtcgttgatgtctaggattcctgacgaatttgaatcagaaaatagttgttttttatttctatggtaatcctgcaaacgtttatacaggtcatcgacgttgttcaacatgttaactttattgtgtaactctgaaatttgtacgttgaaccattcgaaacgttcgatgacttgagttttgaactcatcgtttgtacgtacaacttcgctgttgtgctctttaatggtgtcgacagcggccaaaactttactcaatttctcatctatcggcgaaggtataaacttgttttcgagttcgagatacacatcttcgatgatccacgttataaactctgctttgttcttgaaatccaagttatccaacaattgtactacgccatgtttgttgatgcaaattaaactattgttgtcgtcatgatgattgttaaacaccaactcgttaatggtgcgtttgtacttgtcgtcgaccaatttatcgatggctctttgaggatctgggtatcccatggcttcggcaaactttgctcccgctatccacacttcgtcgttgaacaaatagttgatcgtaaacgaaaattgatcatcaaactttaaacgtttacttttaaaaaggtacgagaatcctgcaagtaatcctcctcccgttctttttttcggtaataatgtttgttcttcttcttcttgttccttctcgttgttgtggtggtcattttcatcatcatcatcgttgtcgtagtatccaaataattttgcgagaaactgaagcaacattttttaagatttggtagtagttagtaaatacgaaaacttttttcctttatgattataactgtacggtacacgaatttgactgttattacaaaacacgtgcttatctacattgggccaccaatacaatagtggtttgtcgtcgatgatcttctcgtctttgtacttggcaattgcttttaaaaatgattcagcaaaacacccttcgggcaccgccgatgtaacgtccaacgcgtcgggcggcacaaacaccttatagtattgttcgcgcgtctctttcgaagcgtgcatgggaaatttatctattcgaagccaaacgtgaatacctctattgccgctgtgcatgatcctcgaaatgttgttcttgaaaaagtttctaaacgtttcggctgccacgttaatcttggtttcgagatctttggaattttccgcttccacgtcaacgtcgatgacccattctctgccgccgttgtcctccaaaggtttcacgtgtacgtcactgatgcgatagcgtttcaagtattgcgcaaattcatcgcaagtggcgaaaaatacgtttgggtggtaccatctgtgaccatcgaaaaaaccaaagcgacgacaatcgttgaaggatacgttgtgccacatttgttctatttgatcaggagaatacacaaacttttttctaaacatgattctcgcccagaggtccgagtgctgaatgcaggataagaatgatgccatttgcgttattaactagcgcgcttatcacatcatatattgaaaataatataactttattacgcagcaacagcgacgatgatagcagcaacggcggcgaagataaactaaaagatatcgaaaccgtgtttgcaattatcatggaggagatatcgaaaattgaaaaaacggaaaatagcgatgttagctataccaaaataattatcggcatactagtgctgatggcgttgttcactttgaaaacgaaaatataccgcatgtcaagttgcttaaggaaaaaaagactctacaaagctcgtgaagaaatacctctcgaaaatatcactattcaggaactcaactacaatgttatagaatcgccggcgacgtcttcgcctccaccacctttaccgcctaaattacttgagcgctagacgctagttcgttaatcataccgcacgaatttactccgcggcgaacgcggacgtacccatcttcgccgtaatcgctaccccaagaatttttgatgatccaatagggtacattgttttctacgccgtaacctactaggagcacggcatgattgagaccattgtttttgcagaaactgacaattcctccgtaataatcggtaagatctaccgcgtcgacggcaatggctatgggtccaacgtaacgtaacaaatcctcgagacgctcctcgttcatcagaacgtaacgatagcaatttcgtacaccggccgcaaacttgtgaggttttaaagcgcacggctgacgctctgctttgtatggatagtcaaactcttgttcgactccgcccatcctcataatttgttcgtatgcggtgtgaatcaacccaccatcgcatcccatgtctacaaaatcgcaatctaccaactgttgttcggccaagtctattaacctatcgtattttatggcgtactggctttccagagcccctagtcctgcaaaggcccaacacgcaccgcacataccctgatctttgaccgaagtgactttgttcagagttcgccaatcaaaattggccggtcgttgtcgttgagccggtccgtcgaccactaccgtttcgcaaaaatttgctcccaactctcccgaagccagacccgtatggcggatgactatttcattcttggtcatgtccgcaaaacgattgatcttgtaaactgcagaatcgttacgactattcttttggtttatcgattcaatgttgtgtctgaatatgttgtagcgatactttttttcatcttcgctcttgtattgtttattgtattgcgcaataaatttttcaaagtaaagcggtgcgctgttaatattgtaaagcatgggtttgttgttgttgttgtgatttgcatgattatcttggcgcgtcaacgccgcgtttagtaacagcaaaagtatgagtaatttgttcattataaattcaataaacaataggtacttaaatacaccgatgtttgctttcaactaaaattttagttgaaagcaaagatgaaataaaacaattaaaaaatttattatagttatttatttaattaataatcatcaaaacttcccatatcgtctaatatatccatcaaaaaatcatcttcatccaaagaaccgtcaaaatcaacaaattcaaatggtaaatttaacaaacagtatgcacatttagaacaaaacaaaaccatatagaaatctttccacttgttttgagttacaaatttttttatgcttaaaaggcaaatcgagcgttcgtaacattgtgcacaatatttactcaaaatataagacgaatgttgaagacaatttgaatattccatcaaaacttttgctaaatttatcggcattaaacattcgggaaatgcgattttgtgaagtcgaaagaatgcgtaaaacggtacagttcttaatttgcgcatcaataataatttagaagttgaagaattgctagcctcgaccaatctcaaaattttatcaaaagcaattaaacgattataacactccacaatatttttcatatttattcccgttcccaaatatcgcattaaaagcgtatcttttgtgacatcgtatgaatcttcctgttttaatgtatactcgcaaaattttccatcgatcaacagaggcaaattcgtaatgtcttcgttgttgaaaaatttttttagcaacgtatcctcggtcataacataattttcgggttgagtctttacgtatgtctggaaaagacaatccgccagcagcggcaatcgtgcgacgcagtcttcttttaaatattttgccaatcgaaagtcacgattcgggcaatagtcgaaaggacgagtatcaaaatactttttaaatagtccttgggacagtagaatattatctgcgcgttcataatctccggttactgcgataaatgtatcgtaatccgcgtattctagaattttccactttatctctgtgggcagcaaacccaacaaagattgttcctcgtcgttgttggccacaaaatacgaatttgtagtagtcatcgttccacacttgatctttttatagtgtcgcgtcaagcaaactactggctctgtcaagttcgctaaccgtttatatactacacgaacacacatactttatctttataaggtttaattatataatgccctatattattacgttgttcttggcttgcgtggtgttatctgccgcaaaagcagctattcccggcgtgccgagtattgattggtcggatcgaaactatgcactcgtaaagattgatcacgaagccacatcgtatgaaaaattgatcaaaagcacgtctaatgtcgaagtgccggtgtcgtggaacgtatggtcaggcgactctggtgacgtagcttacgtactcttcgacggagtgcaaatgtacaaaggtgatgccggcgtgaaaaaagcaactgtgaacgcgagcaaaggaggcaaatacaacatgaccgtgaaactttgcaatgttgacggctgttcaacaagcagcgcggttcaggtcactatcgccgacaccgatggatctcatttaaaacctttgatttatgattacattgaaaataataaacgtttcgagaaacgtaccgataaaatcattgctgcatattttgtagaatggggcgtttatcctagacaatttcccgtcgacaaggtacccgttcccaatttgtctcatctgctatatggctttgtgccaatttgcggcggagatggtatcaacgacgcgttaaagttgtcccagccggatagttatttagctttgcaacgttcgtgcgcgggccgagacaattttaaagttgccatacacgatccttgggccgctctacagaaaccacaaaaaggagtatcctcttggaacgaaccgtacaaaggaaactttggtcagttgatggcaacgaaactggcgtatcctcatcttaaagtgctaccttcgatcggcgggtggactttatccgatccgttttatcatatgcacgatccgatcgtacgccaagttttcatcgattctgtggaagagtttttgttgacgtggaaattctttgacggcgttgacatagactgggagtttccgggaggcaaaggtgcgaatccgaacgtgggcgatcccgaacgcgatcgagaaacgtatacgcttttacttcaagagcttcgtttgcgattggacgcactcggcgtcaaaactaacagatactacgccctaacttcagctgtaagtgcgggtaacgacaagatcgccgtagtaaattacaccgaagcgcaaaagtacttggatgtaatatttttaatgacgtacgactttaaaggctcatggagtaacacggaactcggacatcacaccgctttgtttgcgccaacttggcgtcccgacgaatcctattgtgccgaccgcgccgtagaactaatgttgaatcaaaacgtgcccgccggcaagatagcgctaggtgtggccatgtacggccgcggttggacaggtgtggtcagcgacaatgacgacaatccatttttgggcgtcgctaccggccccgtgcctggcacgtgggaagcgggtgttgtagattatcgtcaaatattgcacaatatttcacaatatgattacgaatacgacgacgtggccaaaggcgcgtacgtgtacaatcgcgaaaccggtaatctaatcacgtacgacgatccaaagtccgttgccgataaaacaaagtacgtgctcgatcacgagttggccggcgtgtttgcgtgggagatcgacgccgataatggggatttactgaacgccatcaacaaaggactgggcggaaagcaggcagattatctatacaccaaggatgagctgtagcaacaacaaataaaagtcttgttcatgttattatgttttattgtttttcacattttagatttaacatgaggcgcgaagatgtcgtcgtcgttgttgtcgtcgcattctaaactattatgcattttttttgatgattgtgcgacgctgaaacgtacgagctgttatctcctgatgataatttcttgattacgacaagtataaaacgatatactgtatcgatgttaacaccaaaacaatagtcgcgttgccggagaaaatggatgtggagtttgccaagaagaagctggccaggaaaattaaaaaatgtataaatatttactatgatgaagacgatgacgaagatccagaagagtattgttgtgtacgttgtaatgtcaaagaaattcgaattgccacgaacaaatactatatgaaaaaattgttgagaacatgcgaacggtggtttaagaaactgcatcgtgaaattaaatgtcaaaaacaatttgtcgaaacattcgtcgcatcgttacctaccgacgtgtttgtatacctggacgatatagttgtggaaaaggctgattattgctttaattgtgaaacgaaatgcaagcgttaccccggtgtaaacggagacgacctttacattgactatggattcatctcgttggccagctgcttttgtcacgtttgcggcaatgaagtgttgttctcggaatatcacgacacgttctctatagttcattgtaaatgcgaaaccgattttgtcgtatttgaaggacttcatgaattctactgtctcggttgcggtaacgaaaaaaaagatgacgacgacgacagtgatgatgaggaagatgaagaggaagagaaaaaggaagagaaaaaggaagaaaatgtttttgaaaaattaatcatagctttcttttgatgtaatgtttaatgaaaaataaagaatattaataaaatgatagttttagtactttatctttgcttttaatgaaattctttaatgaaatccaacattgattttagtgtttcatttattaagtatgttggtgtttttgatgttaacttttgccacggtggtctatggccacggctatctatcgtatcccgcggctcgccagtacaaatgttttagagataatcatttttggtggccggacaacggcgacgagattccggatcccgcttgcaggatcgcatacaaaagcgtttacgcaaagtacagaagcatgggcgagtcgcacggcgtcgctgccaacgcggcacagtacatgttccaacagtattacgagtatgcggcactagccggccccaactacaacgacttggactacgtaaaaaaacatgtcgtttcgcataaattatgcgccggcggtgccgatgaccgcttacatccgtttggcgacaagtcgggtgtcgacgaaccgacacctttatggcacgccacctcattatttcacaattccaaagaaaaataccaaagcggccatcaaatggtcattcacttttgtcccaccgccgtgcacgaacctagctatttcgaagtgtacgtgtctcggcccgaatacaattacaccaccgaactgacttggaacgatctagaacttatcggaggagacggttcgcagttgataaagaacgacggcagcgacgattcatgtactgccgacgaactgtatactatacccgttcgggttccgtttaggtcacaaaaattcgtaatctttgttcgatggcagagaaacgacgttgccggagaaggtttctacaactgcgccgacgtagagtttgacaagtacagcttacgtcaccatcatagtcattagaattccatttgcgattcggccatgtgcagctgtagcagaaacgattggttgggtcgaatttcgcgtttgtccaagacgagctggtacgcttcggacaaactgatttcgcaatatttcataatgaaatacaccactatggtcgcgctgcgagatatacccgcgtgacaatgaacatacacgtttttgccttcgtccattttgcgctttataaaattgtacaataaatcaaagtattgcattatgtttgccgattgattgtcgtgaatgtacacgtatagataatcttctggcaaaaggccaaggtttgtaacgttaatcatcgaatcgtcccagatgctgacgatggcattgatatttttatcttgaacaaaaaccttgaaatcgtcccagttgtaaattattccgcccagatataaattatttaaaatttgtgtcacgttcaccaagtttccgttcgagagacgcaaaaaattattgttgctatccgaagagttgtccatggtgacgtaagtttcttatatactggtttccgtttggccaagtataaaataaatttttggggtcacctacttactgtgtttggtcatctgtcaccatgaacggttgctttggtgtcctcattttattttttgcgatatgtgggtcgtcggttgtgaaatcggccaggattttggccgtcttccctacaccctcttttagtcatcatagcgtgtacaaagtgtacattcaagcactggtcgaaaaaggacacgaagtcgtagtcattaaatccaccaacaacgtaaactacaaagatattaacgacaattacgccgacgactatgaagcgacacgctacaacgtcaccgaaattgatgcgacgttatcgcaagattatttcaaaaaactcatgaagagggcccaagtgtttcgaaagagaggtctcgtcgccgacagctactctgtcaccgcagataactacatgggaatcgtgcgcatgatgagcgatcaatttaaactgcctgctgtgcaaaaatttttaaacaacaaaaaacaaaaattcgatttactcatcaccgaagcgttcattgattacacgttggtgtactcgcatctattcaacgatatacccgtgattcaaatctcgtcgggctacgctgtggcggaaaactttgaaactatgggcgcggtcggcagacatcccgtctactaccccaacttgtggagagataaattttacaatctcaacgtgtgggatctgatcaacgaactgtacgtcgaactgaggttatacaatgaattttataaattggccgatcaacagaatcgtttgttgaaagaacagtttggtcaagacacgccgaccatacaagatctacgcaatcgagtcgaactacttttcgtcaatacgcatccggttttcgataacaatcgacctgtgccaccgagcgttcaatatctaggttctctacatttgacccacaaacatccgaaaccgatatacggcacaatcggtgaactcctcgataatgcaacaaatggagcgatttacgtcagcttcggctcgggcatcgacaccgaagagatggagtcagagtttatcgaaatgctacttaagacgtttgaagcgttgccgtatctggttttatggaaatacgatggctatttgaatagaatgcctgaaaatgtttatattcaatcatggttcgagcagtacgacctgttgcaccataaaaacattcgagcattcgtcacccagggaggagttcagagcacagacgaagccgtcgaagcgctcgtgccggtggtcggcatgccaatgatgggagaccaagctttcaacactaacaaatatatagaattgggaataggtagagtggtaaatacggttagtgtaaattccaaagaattgattgacgcaatcacggatgttgtcgaaaatccaaattatcgcaaaaaaataaacgaattacgccacgttattcgtcatcaggcaatgacgcctttacacaaggcaatttggtacacggaacacgttattaacagtaaaaatcggcgaggtgaaacgatgttaaagaccaaggcctcgaatgtcaattatagcgactacgtcatgtcatacatatttgtgccattggcttcgtacacaattatgaaccatttgcgtcaattgctgagacttaattttgtgtaacaggatatgtaattgtatgttatcaatgtgtaataaatattcattagaattatgagtttgtatttttgacgtgtgtgtctctgttacgccgccgcgcaaacacccactgcgcaaggtttatatataaaaggtcgttgccgaatcggtgttgttcagtattcaaatcgacatcgcgcacttgctaccatggctacaataaggaataaaagtctgttgcgaagcatagaacaggagaataaatttcgtcaagttcccgtgtctgacttgaaaaaggtatctcgcgcgatatcaatacttcaacagtcgaacgcaaatttagctaaacatgtaaataatttacaaagttattacgagagaaaatataaaaataaagtgttggaactccaggcggatctgcagagaaaaactcaggaaattgcgcaaatacaaaaagactttgttgtcgaatacctgtttatcgtacgtatggataattgtgtgtacatgtttgatgatttttcaaacgtaaacgcgatgctcggagaagataagcaatgtcgagtaatattgtgtcgaatatctaaatcggccgctgccgaacgagccctggtcgccgcgttggccgtgtccaagtgccaagaacgtgttctaatcaacaacaatgaattgatttttcaactgtcttcagatttggatgaatttgaaaacgaggtgcgaataatattttcgtaatttacaatgactactactactacgacgacaactggccactgttgttgcgcaattaaagtgtttgccaatgacgctgaattagttcctgtcgcaaaattttacaatgcagccgaaaagttgataactttagactataatatattttttagttgtagtgaactaaaagtgatactagaatcatcgcaacgagacaaatttttacaagccttgtttcaatgcggcaataaatacatgtgtattgtgaatcgttctcgagacaagccgatattatttgacggcttcgtgaatgagttcgaggacgagtgtaaaactaaaagtttcaacgttggaaatctcaaagagttgaacgaattgcacggactttgtgtacgagaaatggctcgtgcgatggaatctcctacggttttacatgtttatgtaaacgaggctataatctcagaccatcttgccaaacacgaccacggcgccgactatgagcaattgttgaataaaatacgggtaaacatagatggcggcggtattgccaatgtgatcgcacctcctcgatgttcaaaaaaattcaacaaaaccaagtgggcgccggtgtcgtgtaaaacgggtaaacatttacttacagcaatcttaacttttaggaaaattatacacaatcaatgatttatttatattttatttattagtcaaataacggcaatgtacattcatcgttaacataaaacttttgtttgttaaattgtagcgtagaagatattaaattattagttttattattgtaatgtttaataattgtaattgtaattactgtgttacaagatactcctatgaaaattaaaactacaatgactgtaattgatattaaagagacttttacaaattttacgtgtacgtttgatttaccatccttttcaatcatagtaaaatttccatgacggagttctggctcctcaatctttggtgtcgtcgtcgtcgtcgtagtagttgatgatggcactgtagttgttgttgttgtcgtcgtcgcaggtgtaaaaatgtattgttgataggtggtagtgtttaaagaagaacgcggcatcactttggttaaacaatacctgtagggttcgtggcgaaattgacggatatacttttgagtattgtcgaatagccaaatgggtgtttcgttgaacgcgttgagtaggcctcgagtatgattattatacaatctgtagaagcgcagaaacaaatttttattgaacatggcagccagatgaataatggtattgttgagagcgtcaacgggtttggtcaagtgtatggcggctatgtgttgttcttgtaaattgggtaaatgttcgacgtaactgcgaagactgcccgttacgttgaccgcggctggagggataaacgtttggcatgctagcgcaattttttcatacttttgcaccgtttgtttgagttcgtaatttgtcgcggccaaagccaagattgtttctttacgaacgggcatgttcatcaacatttggctcatgacttcttcgtttcgacacgaaacgttcgacttgtacgaatagttattgtcgatgttaaagtatttgtcgacgatgcgcatgggatccgaacgatcgatcaacattagagaaacgataaactttatcaatcctttttgtaaaaagtccagatccagactcgtgacttcgtatcggttcagtttgattattctttgcggatacacgattgaatcgtttttaatgttgttttcaatgtacaatctttgcggtgtcaggacgaacatacaatcgtcaaatctaaccgtgattacgttgtgacacttttcaaaggccacattcgatatcattttcaagtagttattttgaaccatatccttgacgcgcaattgattctttctgtcgagatagatacattcttcgtatttgtttagtttgaaaattttaaactcatgctcgagcgcatcgtttgtcggaatcgaatagttgtcggtcaagattgcgttcctcaataattgtggatgtttttcgaaaagaaacgatacgagagcactgcccatgggatagagcatgtcgtcgttgtagtttgatcggacaatgtcgtcgatggttttgtgttgatagttacgcatattgaaatagtttcgccagtagcattgttcgtaaccgaagcgattagccgtaccttcgatgaaccatcgagattcttttatatttttcatgttgaccatacgattcgttgtgtacatcaaacaatgaaacagttcatgattgaaagcatgaggaacaatgtcttctttgacaaaataaacggcggccgtgggtttgttatttctgaacagggtgatgcctccgtttttaatgctgggcgtgatcaatagtcccattcgattgtattcgctacgattcgcgtacacgtacatgtccacgttaaacatttcatgtataaacgacacgtttaaaaaattgtagaatcgtttaaagttatcgtacacgatcttgctctgttcttgcatcttgttgagcacgtttaaatcttgaatattgtggtgtagcgtaatttttacaacgccactgtaaatggtggtactttgcgggaactcggttatttcgaccgtttcgtacaatgtatcgaatttttttcgttcataatcctgcagcttggacacgtaatgaacgtaagcagcttgtttcacgtgctgtttagacattttattcaacggatactgcaatgcgacattgatgacacgtatggtcgcgtcgtcgatctccgtcttgtagtattttgaaaattttctatattttctcatagcagcgtacatcttcaatattgcgtcgagtttcgtcggcgaccatataacaaaattgttgtagaaatgttgaaacgtcgcccagctatctttgaaacgtagaatagtttcgacgtaataatatccaatttcggtgccgatctcgtaaagtctatcgcctagtatttgcatgtcgtacgatatattttcgttgacaatcacaaaatttttcaaatgtttcaccactgtaaagatcatgggttccgaaggaattttatgacgaagataatcttgaatcagcgtaatccaatcaatgaaacggcgcaatccggtcactctggtaaagttaaacttttccgttttgaatttggccaatatttcgtttccattgcaagggtggcggttgatgacgatagaaatagaggtagtagtggttcgattatttttaccactacaactttcaaagtaatcttgaacattgcactttattgaatttattaaaatcacagcgatgaaaattgatacgacggcgaaacgcatggttggtgcgttatgaatacataaaaatcatcgacgatgcagtattttatagcaaaattttatataagcaccattgaatatttattgattatttgatgtgctaaattataatgtcggtgaataaattcgctttaacaacaattaatatgtacgacaagatcgagcacctgacgcgtgcgctgaacgacttgcaagactttgtcgatagtcacgtaaccgaacgtgattatctcggtctagggtatacgtcacgagaaaagtttgtgaacgatgtcgtcgacgctatactatacattcaggttcgtgagagttttatcaaattatgtacggacgacaatgaaaactttaacactttgacgtcgcgtcgactagtcaacattgaacagtacaaatgtaaaaattgcggtacgttaaacaacttttgttccacgatcaacgataacaataaatacaacaacgacgaagacagcgacaacgacgaaaaacacaacaacgctattgttagcgacacgaacaacaatgcgtatcgtcgaaacgctatgcttttcatatcaaaatggacaattttcaaacttttgaccgaattatttttatgggcacgatgttggctttggggctattgtgaaaaaccatggtacattttatggagcaaaaagaataaataattattgcttgcacgcattttcataagtacaccaccagtcttgtatcgtctttgatgatatacgttcgtgtcaacaatcatgcagctccaccaacagcgacagcaacaaaacgaaaggataacaatactggcggaaaaagaaaacaatctacgcaatcaatacgacaataaagtactggccgttttgaaaaaaggacaatttacggacgccgtaaagaacgaattgaaaataatggcagccgaacagtttggcatcgaagaacagctttacaatcttcgacacaacaacaccgacaatcgtcgaagagattttattaataatttcaacgaactggtcgattttaacaacgagcacattgaacacatgtataacaacgatgaaaacgatgatttgctcattaaaaagtacaacattaccgaaaaaccaaaggctattcaggatgtgttcaagaaaaacaacaggaaatttatcaaaattttaaaacaatttatcgataaacgcaacgtttacagatcgaacgaaaacgttgccttgttggaagaattggttttgctcaagtgtaatctaattaaacatttgtgtgtcatggaaaagttgtgtaaataaaacaacatcattatattgctttgtattttatttaaaaccaattggtatgaatgccactatctacagaatccgagtcgctaaagttgtcatatccgtcggtatcgtcgatataatcttcaatgttcttcaacttgttgttatccataaaaaagcgactgattttgtcttttgtatgttcctttattatctttggataatcgacgactattctagtatcaaaattgggcaacgcggtcaatacaaaaattttattaaatttgtcattgtacatacattcaaaagtcttttctattaaaagcgttttatcggcatccacttgcaaaatttcgccttcttcaaatatttcaattttattaaaaacccttgtcacattttcaaaatgttcacacaccatcattgcgccttttctattgactatgcccaaatataaaaaaaattgaacttttcttaattttctaataatattaacattatcgtttatgtcaaaaagataattggcttttaaaaacactctacaatacatccgctttgttgctggtgttgtcgtcatcgtggttaaaaatccaacttttaggcggcgacggtagcagtaaactttgatgatccaaacaaaaatttacagtagcagatgttgttggtattgcagtcgatgttactataggttgattatttttcggcgtatcgtaatatagttcttttttatcttcaaggtcgtaagataattttgatactgacgtggcgacagatttcgatttcgcattgtaataaacattgtgcaatttccacaaattgtaacaattcaacaccatcaacaacaagatcataaactgcaatcgaaatctatattcttttatgaacgtcaactcgtcatagtagtattcaatacggcaagaagcacaataaatgtacttgtcgacagtgaagcagtttttgccatagattgtgttgtagtccaatcgctctatgccgttccagcactttgaatctttatcgtagctgcgaacttcgacgtctagtatgggtacgtggccgtatctcacgatccagttgtacgtgcaaaagaacaccattgtggtcaacatggcgtgagaacacagcaacgtcagaatcatcagtagataatccgatttgcgtagttttttcgttagtaaaacgcccacaccgacgagtatgagattgataccgtagacgagcacaaagccgctacagttaaagacgggcgtgtcgttttcataatcgatgagcaatgcgtactgccgagtttgaattccggccaatccgagagatgtgaacacgataccgacggtgatggtcacgatcgagcgcgcaatacaactaagtgccaccgtcatgtttttactgttactgatcgtttgtgtgatactctttctctttttattatgcaaacccatgtacgacgcccacgctcaaataaaaaaatctcaaaaggcgtacaatgaaacggtcgatgaacgcatatattacatgcaaaacgttttacaacgtagaagattcgtgccgttgagcgcgctgccaaatattatttttaacaccaatctcggcacaattaacgagggcgaacaaaaatgtctttcggtgccgatatacgtaggtttcggcaacacgccaaatttcgactgcgccgctttgtgcgataatccgacagcttcctacttttacgttaatcaatatgataaattcgtgataaacggtcaaatgttaacacaaggcggatactgtacgacgagcagcgttcctagaaactgtaatcgagaaactagcgtagtgttgcacagtctaaaccaatggacgtgtatcgccgaagatcctcgctacttcagcggacctcaaaacataaaccaagtggcgggacgccagcacattgatcgtatcgcaccgggccaagcgaatcgcaacgtacttttcgataggctgctcggcgtcgaagtggacgtgactcgaaacacgttccgcagccattgggacgaagagatgcccgacggcagcggtcgtagattcgaaatgcgttgtaacgccctcgacgatcataacaatagaatgttcgtgaatccctttaatcccatcgaatgtttacccaacatttgtaccaacgtcaactttgtgtcgccggacgtcaggcccaactttgaaacgggcgaatgcgagtgcggcgactttgccgtgacacgcgtccgccacgtcgatcccaacgatcgtacatcgctgtgcgccgccgtcacggacacttacgacgagagaacgcgatctttgcagtatcgcgtagaatgtatgaacatggacatgcctgtggaaaaatatagacgaaacatgctctggtgtccgtccaacattttcaccaccaacacggacaacgcattccttttcactgtacccggatcgtttcccacgtcgggtaacggtttagacgaacccacatggcgtttcatgatggaggttcagagtagaatagatttcggccaaattcgagcttcgccgcccgtctaactctaagagtctataaaatgtataatatattgttgattgtcttgttgatctttatcattctaatagtctacaacaacattactcttttacaatacgttcaacaagactatattccggttctgacgagattcgacaatacacatgtgccgttgatcgaaccacctaccgaaatcgtcatcgaaggaaacacgcatgaatgccacaaaacgctcacgccgtgctcgactcacatggactgcgatgtgtgtcgagaaggtttagccaattgccagtactttgaaaacaaaaccatcatcaccatcaccgacgaagacaacgtggaacgccagttcacaatcgaaccgggcgaatcgtattgtatggctcttgaccgcgaacgcgctagatcctgcaatcccaacacgggcgtttggattctcgcccaaagtcctgtaggtttctctttactctgcagctgcctgacgcccggtctagtcacacagctcagtctatatcacgattgcgatataccgataggatgtcaaccgcacggcaatatcatcagcatcaacgaaagacctatgcgttgctcgtgcgaagtcggctatgtcgctgactttaacaccgaaacacaaactccctactgtagaacaagacgcatacgcgatgtcatacaaaatcctgactttttcccattggcgccgtgctcttggccatacatacccatcgaacatcccggtctcgaccctgcctacttacaatcgacaaacgcaagaaacgcgtgcgtcatcgatccatgcaccgtagaccccatcaccggacaacaggtcgtgggttggctggtgacgcggtatctcaacgatgaagacaaagatactcagtttttctgtaattgttcagcgggacacaatctgttcggcgtgtacaatgaccaaccgaacatgattaggccgagcgccgagaaactcgttaacgcttgcattcaacctttcaacgtacacgttgctcaattgcccgccatcgagtacaaatggttttggggacagagaaatctatacacgtccgacgatgacgtggtcgccactgtgagacccgaccaaatcagttcgccgagataccgtcgcatgcttttcacatatctcacacctcacccgtttttccccgaaagtgtcaactttatggtgatgaaattctctaccgcctacactcccatattcaacgaagcagataggaattatcataatttgtttacgcattactatgaactaaaccatcggagcgctacgtgcttctatccgggtttcggtagatgtgtcgttcacgatcacacaaattgtattcgacggttcggcagtgttcaagtgggcacggcggaaaatttaaaaggcacccaatgttatttaagtcgagaccgatggtggattcgaatctggtacaagccgcaagtgtacaccaatagacgatatgctgtggcgttgtacgtgaacggtcttttcttcgttatcaacacaagagactttagaacggttcgttttgtgagcgccaccgatcttttgatcaatcaaaacgacctcaacaattctctaatcactctactcaacacgtaccaccacatatcggtggtttgaagaaaacatggacaatgtcaccgaagcattattactggccaaacaatatgaaaaacgtcaattgtacacgaaatccatggcttgttacaatttggccatacattttttagtgttgcttaaacgcaaaagattgcaagaaatagttttacgaatgattgacgataaaattctacattgtgtcagagaacggaagaaaatcaatgaattaaacaaacaaatcattttaaaaaagtatatattaatcgatgataatgatgtatattaaacgtacgacatacaatatcttagaatagtttgatggaaaaacataaaaggatgaatgtttatagtgatattcttcgcagccaccagctttgaaacgacagtttcgacatgttccgtgcgatcgtcgtccagagtgcgaatactgaattcttcgtcgtggtaataaaaattctttggtatggctccttcctttgttttattcaccttcgacgaatccaagtagacaacgtcaggaacttcgtttgtaatttcgtgtttcatacgatcgactattgtggcgttgacgacaacgctattattgagattcttcgttgacgacaattctgatgaattggtcagaatgattacatcagaattattcattatttcatcatacatttcgtctatgatcatcgacatgtctggtattttcttaaccgtagacgacgacgaaccttcttcaagtttttgtagttttttaacacgaggaggattcttgcacgtctttggaggcatgtacgtcaactttttactactgtacgatttacatcttttaaagtgcctcgtcgagtttttgagaaacatgtgcgccgactcaaaatcgtccaacagcttttccttcttcaatagcaagacgcggtgcgttttaccggcagcgtctatggccagatatgcggtgcggttgttttcaaattgtttaatcaaaaaacgattacgaaaactgtcgtagtattcgttccacaaacattcaaaagtgggcgttttcgaggaaaatacatatccgcactcgtttatacataaaaaactacagtgggcagatgaccgtaataaaatcttgcgattgggttgagcgatgcgatgaaacacggtgtcgttggagttttcaagtttcgttccgtcaatgtcgcccacgagattcatacgcagcagataatgattaaaatataatacaatattattctgcgttccggtaagagtgtcgttgatcgacaacaatgttggcgatgatgtcttcgacgatgttgatgtagagttcttgttggaatcggtagtatttttcttcaccaccacagaatcattactctttgccgtcgtatcgatggctagtccacttgcgatggttgcgaccagaagcaaaattgaaattactttaacgaacatgataatgccgtgatgttcggttacgacgaactaacgatgaaactacaaatgcttgcctttttatactaaccatcaaggttagtcggtgcgcaattaaccgcaaacacttgacgtcagtgttgtacttacttaataaaaaactttaatttaaaatcaataataggtcgtataaaggtcgattcaattgctatttgcatatcattgaacgacaagtatcggttctccaaggtggtggataatggagcgttttgaattaatttcggaccaactgagctggaacaaatgtttcgctgataaattcgccgcctgctactacattgatcctcgaagagcgtccatgaaaacgatggccttgttcaggttggcgtcgtttttcatcgccgccgcactcatcgtcgccgccatttacgaagcgtcgaaccgaaacgatttcatgttatactacacccattggtcattactggcgctgatgataatgtgtttttccggatcgttaacctcaatattcatcatgcaacaaacgtacggcaacattatttatattccaaagcatgctgtagtgtttcgcacgttgtacaacattgcttgtacggcaaacattgtatcgactataggatactttgtgataacgttcacctatccgaacgcgaccaaaaaagtaatcaatcacgttatacattcggcaaacacgttgttggttttgatggaagtgatcgtaaacgccgtacccatgagattgtttcacgtgtaccagccgttgttgtataccttggcatatgcgacgtttgcatacctgtaccaccattacaccggtcgagctatatacaaatgtttggaatgggaagaccaacgcgaactgacaaaactaacaataggtttcatcatattaatgtttgtagtgtacatgtttttgtatgtaattagttttattaaaaacaaaatgttaaaattgtaatctcaacaacaaacacaaaatttcaaattgtataatctcatttattttaacattaaataatatttacaataatctttgtgttttattggtctggtgtcgtcgttgatttgttcgtcgcagcaaaaggttctcaaagcaccgtgatcggtacagtagacgccgtgatcggccagctttacgtcgattccgttgtttatcaacgattgttgtctcttattgtggctgatgtaatttttgtttgagattgtcgacattttttcaatactactatcgccacattgcttgtttctatcgataatttccgatatcggcgcttcaatgtcaaacggtagatcgcaatagatacatttcaattcgccaaactcgtacaccaacccgctttcggccaattgttgtgcgtcagacaaactaaagtgatttgtcaaagttttcaatctattctgttcggatttgtagagtaatcgatgtctattcttttgtacgtatctttcgaacaatcgtttttcaatttgatattctttgatgtaaaacgtttcgtttctgagcacgggcaggataacgtatgaattgtaaaatttgaccacgtacacacacaacggcgcgttcatgacgtacatctgtctttgcatttgacggtaatgaggatcggtttgttcgacggcgaataaaaagtcaccatgcttgttcaccgacaacgccgtgtgtttgactcgataacgatcgttacgattgccgaaagttttacgcatttcctcgaaattggtctctttgtacgtgtgcggacatttaatttccaacggcacaaatacttctttggcgtcatcgcccgtcgtcgttttaaccacaaagtacgcatcgggcgaagcggaaaacaatccaaactcggacatgaacattccgcagttgagaacttggtcgacgaccttgccgcctatggtggtttcgatgacgttgatgacttgttgtatgagaaacttgtcgttttttaatagtttctcttgacgcaagccgtaagacatggccgccgattgaggcaccgacgtcgcatagctacaatttccagaagctgtttgtcgatccaaacgcagcatgttccatagcggattgagagattgttctctagtagcgtgttcgacaaacaggatgtcgtttcgagacagcctaaacttgggcgattgaagcgtcgacacataattattatacgaatatttatctataatttccatttgacttttcgtcaatttacgtgtttccatcatgatgtttaattatcaagtgtgattttattcgcacccttacaccatacgatctttgctttcaacaaaacacttcaccgaaagcaaagattgataggatgacgcaatattaaatcattgctaaaaatagcactattgcatcattcaatacgatctttgctttcaacgaaacactttgccgaaagcaaagatcacattgttaactgcgcaataaagtcttcaatctttgctttcggtgaagtgtttcgcggaaagcaaacatcatcgaaccatgatatcatcgactgacgcaatagttatttttagcaatgacatcagcacacaatgtttgctttcagcgaaacacttcaccgaaagcaaagattgtatacaaatgatgaaattgtggcgtaataaagtctttaatctttgcttttggcaaagtgtttcgcgtaaagcaaagattgcgattattgcgctataatttcatcattctatggcgtcatcctattgaagtttgctttcggcaaagtgttttgcggaaagcaaacattgtgtgtttaatcaatatattttgtcatcaaataatctaaacattgagtattgatcatgcattgtagactgtcgccgtcgctgttgttggctagaaaacctaaaacaaaatttagcactcgttgtttgttgaaaatcatcctttccagttgtattatcctgtcgaacgcatacactatggttttaaacgctgcgttcttctgtacaacacttgtcctaacataagaaaaacacagggcatagaacatttgttgtgcattagacttggacgtatctatcaagttgttgatatttaagatcacgtcaagcaattgatttggatttttcaacttgataaaattcatcatggaacatttacagaatcgtttgttaaaacacagagcaatcccttacatttctaaaaagtttctcaacgatcggttgaccgatctagtgctgcgtaatgccgataaaacgtttcatcgaagaatcttgccggcaacggttcgagcgctcgacacgttgagtgtgatcaaaggaggtgctgccatagccgttcatttgaacaacaacacgcctacccatctatccgatctcgatctggaagtgtacgtcgacgacgagaatgttaccattgaaaatttatcatcgtacgtgtcgttggaacgagtcgaacaagaactcaagacgatctgtgaaaactattacgacaacatcgaccaagcgctggccacgatcaacattaaccgattgatgaacaacacgtatgcgcgtgataaaatgttcatctttaaatcgtacatcaacgaagccatcgaatttgttcccgatcacgttcgtttctgcctcaaccacgcgcagccgttcaagagcaccgtgtctatcgtaaacgatgattatttcctcgttcgatactcgttcaacgtgcacatgaaaagctttacatcgatcagattgcacaaggaaaacaaccacatcaccaatctacaattctttcctttggacgtgttctttttagacgtgagcgtcaaacgttctcccgcttcttttgttaacatgtactcgttgggtaatatctttggcgttgacgtgtatatagaaaacattaaatatctcatcgctgatcaactcgaatgtatattgtttaacattttcaacttttactggaacaaagttgaatcgaggatcgatagagttggcaatcttttaaacgcacaaaccgattactttatacccaccaatcatgaaatcgaacgttacgaaaagattgtcaaacaaacacatcaacgattcacagctcgtgatgttaaaccattgttgtacgctttaggaccgctcggtcctcaagccatcattgaattgtattttttaaatcgtttcgacaacaacatcaaatacattactcatcaggtcaattttccctatcatgtttgggaacaggattactattctaaatgttggaaacaatttttgtacattttaaacaatctttataatcttaaatacaatgttgaaatttaacaattgttttattattcacaataagcttcctttatactatcgggaacggtccatggtacagagtcgttttcaaagtacagcatgttgatgtgaacggctacgcttctaatgtctccggttggcgacaatacgtgcagaaccacatgtcccgtcggataatagtccaccgattctggcacgtagtatccgtcgttgatgcgaactttaaattgctcaggtataagcaggttcatcaaacgatcattttcgctgtcgaatatgtatttttcgtcgacgtcgaacaattcaatttttacatcgtgcattccattgtcgagtagagtttgagagaattgtatgtttgaagtgtctttgtcgaaagatgccaccgcgggataagccattgtggtcgcgactgcttattttaacagccgaataattaataaaaattttttgaaaaaaactttattcaatatattcaacaataaccttaggattatgtatgttatgctcgaatattagttttgaatttaacaacatgattatttcctcgtaataaaaagcaattggtctgttggttacttccaaaaacttcctaatcgtcataccataactcaaacagctattataattgtcgttccaaacgtcatacactttcttgaacgcaatgggatttaaaaattgtttcatttcttccagtcttttctttaacgcagtcaaagcttcgttcttgagcatcgattgaatgtttctcttcatttcattggaaactttaatgttattggtaacgttaaagtaatactgttgcagtaattttgtatctggacaaacttcaataaacgaattcttcctcttgagacacgtttgatactctttcaacgccgctacaaaattcaaatgatccacatacattatcggagaaattgtaaacatcatcctgttcaagcgtctctcttcgtcatcgtcttcgtttaaaaaaactctattaaactttttatccgttacttttacatcgaaaccatacggatacacattgttctgcaaataaaactctttgtaaaacacatacaaaactttggaattttcctcccattcaatgtcttcaacgtatcgcagcagatactcgacaatattttttttgatacggtccaaaaatcccgaataataaaatatattccaaatcttgtggtagtttaacatctccaagtttaacgtctttatacgccatactccttcgttccaaacttcgggcagagttctatgatctacgtcacagatttttattccgtaatcaataacgcagttaatcttactctcgatgctagagtctccttcgagaattgcagccaccgttttgtttgcgttatgtaatctcaacatccaatcggtaaaaaaattattttgtatatttttatatcttctatgtcgttgaacagcatcgaacacaggatccagtttgcgtaaacgatccaaattgaaagaaaaattgtaatatttttcaacaaattcttcaaacttgaacagcgccaacagatcaaccacttcgtcggccatgtacccgatcgaaccgacccaatactcgctggtccaccatgcgtacttgtaacttggcgaaacgttcattgtagtctcttcttcggcgacacaatacattacaatcttgacttcagtactttatatacattatgcactggcgtacgtgaccatttatcaatagattaattgattaaattgtgttatcaaggcgtacgtgacttgtttatctataaatgaagtgcatacgtgactgataaactttcaggaaagtatatataacgtcgccgatgctaaaacattaatcattcatcgtcgctgtgcatcatggaaccctctacttccactgcaaccaaacgccgcaacgaagacaccgacaacaacagcagcaaatcaaagattctcatggtcgagaaccaacattgggtgaatattacatctaaaccgtttgaagctatcaacttgatttggtacaatatgaatatcaaagataaaataaaaatgttggccgtcatcaacaaggaatcagagtttgacgttttgctgcgactgactaaaccgactgcgtttgccgtgaccagcttgggtaacgacaccaactttgccgtttttaacgacgcaatagccatgactattacgccgttcaaagacacgagttatgatcaaatatatcgaacttattgcggtatcgtcgacattagccatgatgaaagatattacttgtatccatttgaaaaattttacgacatcgacactactgactatgtcgaaggcaaagcggacacttcgaaagccatcgaagttaacaattcctactcggtctacgaatatacacgcatggtgatcgatggaaaaatgtacgacacttatgaagaaatcaagatgccgcccgtcgaaattacacactgtaactgtttggtaaaaaccaagatcgtttttgagagttcaaagagaagaaacatgaagccaaagataaatcatacgtttcccaacaacgatcgaccaataatcaatccgtcgcccagcgaatatttactattgagcaattattacgctgatgatattatatatgttaaatacttggataatcctttgattgaaaatgtaataaagttttaaaaatgtgtatgtgtttattttttatttaaaggaggttgcagaatacccaatattgtttcgagtaaatctagaatgttgttgacggtttgttgtagttcggcaataatgtttcctaaattcaaaccgttaatagcttgcagtaaattgtttagtgtggcgttaatgtttgtaacgctagatgaaagattgttcaatacggcattgattcccgtcaattcgttgcgcagttgttcggacaattggtttatggcattgagtaaattgttggacaaagcagtgaggcgcgtatctatttccgccagcaatctgttaaaatcgccggtgatgtcacccaaaccttcaacggcctctagtatttgtgctacttgagcaccgatggtgctttgttgtaaacgtatagcgttcaaaagattggacaattcgagatgttggttagcgttgttgacggtgagttggttgaggttggtggcgatgagatcgttttgacgcgctatgcgttccaatagctcagtatgatggttatgatgatggtggtgatgatgatgaggacagcatctggaagatcgacgtctagaacgcgatcgagaacgcgatctacgacgtcgagggctcggcgatcttcgactcggcgacctgcgacggggactgggcgagcgacgtcgcggtgacacggatttgcgtcgaggcgagcacgagcggtggcgacgacgttcttcgtacacttcggcgacgaacaacgtcatcaaataatcggaaacttgagaattgacgcgattgcacagattgcccagtccgtataaatctacaaacaccttgttaccgtcatgagaacaatggttaggacaacgaaaatcgacccaacattttttgtgtcgaggcgcaatactttgcaaagtggacgcgggcaatcttaaaaggtgaagtacgtcatcggcgctgacccacaaaacgcagaattggtcaaagtacaccgagacgtttatatcttgacatttttttgtaatcaacgacattatattgtaacaaaaagtaaatttttgttaaaagacttactaacagccaaaataaataaaatcacatatataaagttttaaaatcgtttattgttgacgaaagttatacatttcaacaagattgtaaattctatccaacaacaattccacgtcctcgtactggtccacgattttcatgtcgtgttcgtcgttgtcgtcgttgtgattgataaaatttaaaaaatccacaggttcattttcatgcgtcactgcgctcttgcaaaccgccagacaatcgccgttgatacacatgacggcgtcgacgacgtcaaagtgtcgcaaaattttataatacacgtccttctgatccaggtttaggtggttgtgaattgaaatgacaaagtttaccaatttcattcttgtaaaacagaaaacaataaacaggctcgggcttttttctatatatgatcagttcaaagtagtacaatacagaatcgttaacgtgtttcaagtcggttttaaactttaaccatttagctatcctgttggccgctattgaaacttgaacttttttcctaatgttaaatccaccggttaattcgcgtcgtaacaattgcctacacaacaaactatcgattcgttcgcgcgacaacattgttgaatgaccgcgaaacagtactgcttttcgtttaaatacacgcaataaattagattattaagaactgtagccaataataccgcaggcaatgcgagctcccgaattacctgtcgttttactgagaacgtgaccgcccacacccaaatcgtcacgatccgcatgaatcactagactgcgacctataatactgttttcgccgaaaagggtcatgacgttgtcgatcttgtctatttcagttaaactgttcgactttttcgcttcaatgttacctaaatcgccgacgtgacgctctatcgaattgggtgcgccatgattgcgaccgaaaggattgaaatgttcgccggcggacgtacatccgttactggtgtcgccgaattcgtgcacgtgaaatccatgaagacctttggttaaatttacgacatagccttttatgtacaaaagatgattgggcgattgttgttcgaatattacttgaccatgaacgacgtcgtcgcttgatagtatacaaatggccttcattattatggtttgccttaacaatgatgcgcgctgtcattattcgcacatactaagcaagatgaataataatgccttcatcgtcgatatacggaatcgtcgtggcactagtcgtattgttcatcgtatgtttttatataatgggaacgattcaaactttaaacgacagagaaacccatgaaatcgtcaaagcaaacgtcaatcctcaaaccgccatggacattgtgttcgatcgtaacggagtcgtggattgtaacagcactcgactcccttgcatctccgatcaacagtgccaagataattgtttgattcaaaccgcaatcggcgggatagcgtgtaacaacgggttttgcggcaccagagacgcttctttcaccggcagacctgacgatttcgaatgtgatcctaaactgggtctgatcaaagcgtatgtggcgagcgagtttgtcgtcgatcaaatgtgcatcagcacgtaccgagacattgtggacgacttgggcgaaccgagaccgtatctctgcgacaatggcgttctggacattgatttaattagtaaacaattttccgccgccgattgtacttgttccagcggatacactaaaatgttatttaaccaaaccgcgttggctagatccattccagtgtgtattccgaaccgatcgagaaacgtatttagcaaaatttacgaagtgttgcaataaaatgttgtcgcacgatataatatgttatgcttgcgaaaagcacaccaacaaaaaatacaacaagatcctcgtcgagtttgttaataatcatgcaaacaccaacatttgtacactgatggatcgaatactgctggcgttcgaagtggacgtcgtgtacgagattgttgaaaatgtaaaatccatcatttgggccgtcgtaaagattgtgtatgcgcgcaatcgaaatttaccatcgtttcaacatcacctgccgttcataagttttgctgccaccctcatcccgcagattgcaaactcaaaggatctatacaatttttccaaatcgttatacaaacgtttctgcgtgtacgagatgaaagaaaactacggcactttcgacgacgtaatagcaattttaaaaaaattagattctttatgtacagtaatagaataaagtattattattatagctgtatacttgtcttttatttgttgtgtgttatattgaattcaataagtaagctatacaatatgaatttgcaacaagataacgagtcgcttatatcacaatacacatctgttcaaaagcgaataatcaacactttaaaattgatcgttgattataaaaataacaatgacgacattgaaacgttgcacgattacaaagaatatattaaagaaaacaacaactatcttcaaactttacgcattgtaaagatgaacttggaaaacatcaacgatggcaactcgaaaagaataattgaaaatttagaagacgacgagaaaacattgataggcgtcaacgatctgttgcatagaaaaattcgaaatgcgtcatctgcgacgttgccttcttcgtttgtacccaacgtaatccaacagtataacgtcatcgacaaattgaacgatttggtcgacaaggtgggttttttcgagtttagaaatacaccttacgcggccgaattggaacgactgatgaacgttaacgtgtttcaaattcgactacacaacagggtaaatttgaatcgtttagattgcgcagcaatcatactacaagcgctcatcaaagagaataatagatcaattgatttttcaaagtttgtccacggcgacagtccgttgatgttggaaaagttaaaatgtctattgtattacgtgtttgaagtgtgcaagtttatggtggagaacgatgaagtggcgctcacgacgccaatctctatacatcattacatcatagaccctaaaactatatctttgggcgacaactatacaaacatcaacattgacaacgtatttgtcgacaagtttgcgccgtacaataaatacgaagcggcagaggtggaggaatctactacgtacacgattctctatatcaacggcaacataaacgatcgcatgttcgacgaatacgctacccatcaagacgtttggtacatgaaatgtcccgaactgtatgttttacctcattttatcaaagataaacttggcgaaaacgaatcgtacatcatacacaacgtgaaacaatataacattctcactaaccaatcgtacaacacgaaacgcgtccacgacgctgaaatgtacaacaaaccgctgccgatgcaaaactttttaatttacgaatcgtgcgattacaaaacgtacacggacattgatcaatccgacatcaagcatctggatagggaaattgctaaactgatgtcgggcatccactacgaacaggccacttctagtaatacgtcattaatattccgttcgggcccgcacaactgtcacgacaataggacgtttcaatttttaatcgaagttctagtgtgttccaacgagggcagtaaattgtattattgcgcatcaaactttgaacaacaaaacgaactcaacgacacgttagaatgtatccaaaactacacagtatcacaactatacaacaagctggccaattataatttcaacacgacaggaccgatgaatttttatcgtagtagatagtagtaagtgatcggtcgttgaaatggaatcgatcaatattgatgattttgcccggcagctgatagccgacaaatgtagctcgctcatcgaatcttcagacatgttacccttcaacaatctggcaatgttaaaaacggtccgcgacgagtactttaaaaatccaacggcaaaaaactatgaaaacctaaaaaagatattcacgcacaccaaatactttgacgactccatcgactacaaagactttagtagacgtattctattaatagccatcaagtttggtttaaacaaaagtaaagaacactttcaatcgtataaaggtatactcgaagtcgccatcaaacgtctcgacacgatcaatcccgatttacgcagttctaaaagggctctgttgcaacactaccacgaatgtttggaaaactttgacagcccacgcaaagatgaacaccatctcatcacgttcgccaaggaaatagccacgaaaatcttcatcgataccatcgacttgtacaactacaacaacaagagtcccatcgatttcaacgaaaccggcaccaaatctatagaggctccttcaaaaataactccgctactcgaaactgccattgcagagaatagaaaacgaaagctgccaaaaacgcccgactataaaatagctacgcctttgtttcaattgtaataaaagacattctggtatattatacacattgttttattattcattatttacaaagtaatcttcatcattgtaaaagttaatgttacaattattagcaaaataactatcgctaggtaaagtaccatttacgtcatcgccgcagaagaaagaattttttcgatccagttcgatgccgtatttagccaacaaataatcaatgctgccagtcatcggctttcgatacttgtcgttcttcgaagctatcatcacgatcatcggcaaacccagtatttcacaaatgcttctaattttatgctccacgtcttgtacggtgattttattgaaacttatgcccagttgattggtgacaataacaatagtgtacttgttattgatcaaattcaaaaaagtttcgtaaatgttgccgtacttccattgccagtcgaaacgatctcgcggaaaaacattgccacttttagttacgatcaaagtgttgtccagatcaaaaccggcaattttaatcttgttattcaccttgggaaatttaggtaaacgacactcgaaaacatacatcgaatcgtcgacaacgtaccaattgtaaattattttcatcaggcattgctcaatttccgcttgctgctgtgcgagatcttttgaaaagtcaataacgtactcgtacgcgttgtagtaacgagtcatttgttcgaatcgttcattctgctttttcacatagtcttcggtgatgagatcgatgccgttgtttcgtttgcgcatcattttcactacatgagactcggtgccgggttttacgcgtaaaaacaccagtttccagtcatcgaagaaattcattttagcgctctgttcgaaaattttacacgaatcctcaagattgatgtctcttctgatcgcctcgtacaccaaagattccatcggatgacggtcgtagacgttcaacgttttattgtcgacatctcgatcgtccgtcatcaatctatgcgctatccaaaagagactgcccacacggtgatccattttgtactcatcgttaaactctttgtaatcgctcagatgtacagaaatgttgttggattttttgtcgagctttttcaacaaagtggttttagtggtgcaagccactccacccaatgctaaacggtatgacatgtttgcaaattgtgtgtcgcatcaagcgacaatattgattttatactctgttacaatttcattatctcgaaacaccaagggtaacgattcgttacccttgctctttcgtcgaaaactacaccagtttcgtcgacatcaacacagctaacaatctttgctttcggtgaagtgtttcgctgaaagcaaactttgataaaatgacgcaataaaatgataaaattattgtgcaataaagtcttcaatgtttgctttcggcaaagtgtttcgctgaaagcaaagattgcgattattgcacaatgatttaattttattgcgtcatacaaaattatttttaaaaatgatgtcaacggtgctcgatgttagctttcgtcaaagtgtttcgctgaaagcaaagattagtggaatgatgaaatcattgtgcaattgttcgatgatgtttgctttccgcgaaacactttgccgaaagcaaacttcaataataaagataacaaaatggtatttaacggtctcttcgtttgcgatcaacacacatcgggatcgaaatgacaatcaaaatgaacacattaaaattcacaaagtcgcccaatggctacacgcctcgtatggcgaccgcgggcgctgctggatacgatctttacacgcctgtcgatttcatcataaaacccggccgtcaaattatcatcgatacagaagtaagcatacaattgccggaaggaacgtatgcgcagattgcaagccgatccggcaacgccgtcaaatacgaagttgtagttttggcgggcgtcatcgacaacgactatcgcggctccatcaaagttttgctgcgcaatttgggaaagaagaatcgacagtttcaacgaggcgacagaattgcgcagttgatcataaacaattattacaagttttcgtgggaacaggtggacgaattgtgcgacacggatcgcggcgaacaaggattcggatcaaccggtcaatgatgtgtatataaatatatgtattgtttgtgttttatctttagtttaaatcagaatacacgataaaaatgacgttgtctaaagttttatttgaaccctgcaatcttcaaacgttggccttttccaaagtcgtatacaatgaaaaaattaaacataaatttgaacagtatcatttgcccgaatattataaaaagtttttgtggttcaactacatcaactcgtacacgttaacgtgtacaggtcttttagaatatgtggataaaacgcaagatttgaaaggggctttggattgtctactgaagcacaacaattattgtcatgaaaaggaaaagttcaactggaacatcaaagccgactgcgatttcttcaatattaacattttagaagagtttgacaatttgattagttgtaatgtaatgtacctgtcgaaaactaacatgttaaagtttgtcatgttgtatatgtatattttgttgaattttgaacattttctacctcaatctttttttgtgttctattacaatttaacggatttgtgtttcgagtgtgatgttgataaaatgttatttccaaatttgatgactttattcgtcgatcacgacgacatgatcttgtgtatcaacaatttaataatgaatcccatacatagatgtaaaaaatgtagcaatttcttgtttaacattgaatgaatatagataaattattagtataaatatacgttgttttattaaaagtgtccataaggtgaaaagaaaatgtttgcgtacgttacgttggtcatgctcggagacgagtacgttaaaggcgcaaaggttttagctaaaagtcttttagcgagcaacacaactcacgctttggtgtgtatggtaacgtgcgacgtcagcgctcaggctagaaacgaactcgcacaaatttacgatcgtgtcgtcgatgtagattacatcgtttacgagtgtccgtcgatgttgacgaaaagacagaatcaaatgtacggcaaatggatcgacaaatcgtttacgaaatggcaatgtctcaaattgaccgactatgaaaaaatcatatacctagacgctgatcatttggtagtgaaaaatatcgatcatttgtttaatctaaaagcgccggcgatatgttttaccgatgacaattacggatactacgatcgtttgcagtttggagacaccattccgccgaatagcgtagccacgttcatgcgatacaataaaattttatgtaaagccggcacagtgttgttcgagcccaacttgaccttgtatcacaccatcctcaactatctaaacgcgttcaacaaatacttgacaaagtctcattttcacaacggtttcgatgaacaagttttactacaggcgttgatacatctcgacatcccaatcacgcagttgtcgattctgtacgcgtggaatgccggatcctattatagattatgcaagaaacaagaaccttacgtcatcaactattacggcgatgtaaaaccgtggcatttcaacgatcgttgttgcatcgactacatggacgtgtacatttggaaatactttgaaaacatgaacattgtaaaaaagattcaataaaaatgcttgtgtaactattaaagttttttattcactcacacatataagatatcaatacaatgtggtggttgtacgtgtttatgattgtactttttattatattcgtagtcatcataatgtggcaaacgaacattgttattgtagatttagaaacacaaaattacttgaaggatcttgaatattacaattattacaattataacaatgacaattattataacaatgacaatgaagaagaagaagaacaaaactatgacgacgatcgaaatgatttagacgactttgaaaaacactacatcgacacgttgcctttgaaatttttacaaaaggcagagaaaatatcgaaacccagtcggcaatttagctacgacgacaacatctttatcggcctcgatccttggacacgtgtcgccgatttcggtacacttcttcatactctcatcggttacggtgttcgtttccgagacgtcgacgatccgctgtatctcaatttcgatcttgccgaacaattgtaccaagccatgtacaccatttacgaccatttacccattccggcgccgacgcaacaagcgccatggggcaatcctgccgattggtatcacttcagcataactatgcccgagtgtttccaacacgtttgtatagtgttgcgtggattctttgatttgacacagctcacagaatcgttgttgtactactatttgccttcgccgactcattccatgggctggcaacgtacggccggcaacgccatacgcatgtgtttaccgtacacgtacggacaactattgcgaggttattacttgaacgaaatagcaaaagaacaacaagtcaagtacgtgatagatttaatacatttttcactcgtcaaatccggcaacggcgtacactacgactacgtttatttcgatcacattgacgtcagagcatatggttatttgatcaacagctatttcacattcagctattataactatcttttcggcgaaaacacggtcaatttagacaacgtcaatagatcgctcatgctcatcagtaactatcgcggttttgctaatcccgctctcatatctcgcaccggttcgactcattccgaagtgttggctcactttatcgaatatcccaacgacgtcgtcgccgcagactttaataaaattttgaccgtcagaaacaaactctatttcggatcgttggtaggacaaacttttaacgttgcctattacgaagcggatacgacgaatagtttacacgctccattgtgggcgatgacgcgcaaaatctggtcgaaccaaagccgcgctcttcgctatagaaacttgggtttggaatcgggcattctgctcacgaccaacttgaacggtgtcgtcgacgtgccgtccactagcaccagcaccagtagtttctatcctaccattgcgtataccgctatcgctaccacgaacacggccggagtcatgatcatgcatgttcgtttcgaagagttgaatatcgagtttcatagttacacactgtatcatcgaaacggcatgttccatctttacgatagaataaaaagtcttcaaactatcgccaataacgcaagatgcgtagttattacgcgagacaccacttttgaacctaaatggattacatcatcaaacattctgcatgcaaacggcgtcgtcgccaagcaccataatatcatcaacaatagtagtttgtccaatttcgatgtgaggacttttgacaatttagatttacaaacggccgaacaaataatcagcgctgacgcaatcaacaacggcgttggcgtggcgtgtttcagtttattggctcaagaaacgttcaatgcggacaacacgataatagaacgcgtcttcgacaccgacatatttatcatcaccaccgataataataacattagctgcgtaattgattttccgattgtggtgcttaaagatgatgaaactcgtcaattgactatcaacgacgccacaaacgcttcgaaaactacgcatcatttaagtttcgataaaatcattcgacccctgtctttgttgtcgttgtcggtggacaatttaaaaatatcttcttcggatatggttcgcgatcatgatcgattcactctttttgacgttcatggtaaccaatttaaatttacgtttacgtaggtgctggaggagcgagaaccgacgtcggtatctgcgccgttttttgaggtcggttaagatcgatcacggcgggtgcggttcgcgcgcgcagatccaaattggccaagaatgttcgttgtcgttccaatttttgcgcagtagaaatttccatactcttgttgatgctaaccacgagaattatgaacaacaatagtattacaaagaccacaatacaacatatgaccagtaaaaagtctctgagaaacgattgattgcgcgacaccaattgttctagctgatcctgattcagcaccgaggacgcggtgttggtttcgaaaaaacgagcgttgcgcctcatgtcgtcgtcctgtgctggctaggtgctcttaaaaaaagtaattgtatctggctgccgctacaattagattgtctatacgtgtcgcttcgtatagtttgtcgcgcatcgtgttcaaatgaaacggcgtattgatgtcggagtagtcgaaagctacgggattgtcttcgaccatgttgttgggcaccggcccgaaagaagtgattgcgtagctgtcgtttttgcaatagagacgaacttttcgttcgcggtccaaactgtttagtttcacgattatatcgggtacgatcaagtagggcaataccaaaacttgaaacatgttgataaagtttacaatagtctgtgacgaatccggcaccaatagtagttgttgagcgcgtttgataaactgcaaatttggaatgttaacgtacggacagtgtacgcttctgtcgcccttgatcaatctcctgaccaattcgtttttgagttcgacgtaaatgtcttgcacgtcggcgtcggacatctcaaacgcgaatcgagcaggcgttttacgatactcgaaaatagtcagtatacgcttcacgttaagttgtcgcacctttttaggagtttcgcgcaaaaagcatatgagaaagttgttttgtgtgtcatcggtcatgacgttgaactttgccaacggagtttcaccagcaatgggagtgtaatcgcgtatcgtttttgatagtatctgtataggacacatgggcgtgttcgagtcgactaccattgttttacagtagaacagactgtcggtgagcagtacgctgaaaacgcacgtttctccgacgcccacttcggtgatgggaaatatgggattccagtagacgatggtggcggaggcgttttttgtaggtgcgcgactctccaccttttccgaccattcgtgcctttgtaccattaccgagctaaactctgaggaaaaagttttgtctacaaattgaatatcttcctcggtgggcacgtacacgatcagattgcgtacataggcaccgccgaggtctttttgcggatgaacgcgattgtaaggaaaagcaaagtaacgatcgctgatgtagacacaaatattaaacgggcacgtcatctttcaatcggtgcactttaactacgctcaataacgtttctggtatataattgtattggtagtacaacagctctttcaactgaaatttatttattcttagtgttaacaaaacaattatagccacgacgaatatcacgacgagcaacagcacatacacgatcatgctagaatatctgacgtggaaacgaccatcaacttaatgacattgtccacgggtccgacgtatacgcgattttgattgaatcgatactgaattctgttgtattctttttgcacgaccgtgtcgtacacgtgcaggacattgataattttttggttttgatacggcgtcgtcggcgacgacagcagttgatctagaaattgtcgatagaaccacaaaaagttgtatcttatcatgaacaagcctacacgatcgacgtcgttggtcaacgaagtcaaactgctgcgtagatagttgaaattgcgcgcactattgatgaaatagtttgcggatcgcgcaatgaaccacaatactttggtgatgagagcatcgctcgatccgtaatacgctagcggttcgagcaacttgtaaaagtacacattttcattgatcacggtcaaattcattttgttaatgttcgttatggtggagttgtaattttgagcgctcactttgacatcgctcaacccgttcgtaggacacgtgagcaacgaagaaggcacgtcgatcggcaccacggttacaatttgcataatcaactttggcacaatggtgttggattgatatctttgtttcacgttgtcgataaaagcctcatcggaaaccatattgcttgttgtcgacgaagacgaagacattgtacccggagtcgtttgatcgggcactcttttagatttagaaacatttggaagaggagcacgcatttttcgtttacgacgcatagtttgcgtcatttgctggtaatcttcttcgtcggtgctactgacagcgacggcagcgacattaccggacgaaggagcagtaaatttaatgggtctatgttcagagtaacgatttaataaattttttacaacattattggtgatttgattgaactcttgaggattgatgttgatttctaacgtgtaagcttgttttttggcgacaaggtccagtatgctcactacggccgctctcacagtaggcggcagagattccaacagttcaggattgttgcgtacacgttcggcgtaatagttgacatcggtaactacgcagttgaccggtccgtattttttgtaaatttttataaatctttgcaaatcatcgttgaggtcacaatctacagagtcgagttcgatgccgtctttgaaataatcttcgtatacgtcgttttgaatgagcttttcgaaatcaaacaacgattgtttcgtcaacgattgtagactgttggagtcgagcaattgttttgtcaaacttttaattttaaccatgttcatttctagatcatcgttgccgttggtgtcgttttcactgtcgtcttctgaattcgattcgctatcaacgtcgtcatcgtcgttttcgtcgtcgttacgttctatcaaaactgtcgtgtccgatgaacctatcgatctcgcagaaggaggaggaaaagacgaagtggacggttgaggcaacgtgtcttcgttggtcgattcgagaatctgacgcaagttgtcgatgacgttgtctaatgcgtacttggcgatgccgatcgatgaagattccgcgtttttaacgcgcaacatgaaatttttaatcgtttcgcgctgattaggatgaatcttgagcaaatatttactcaacaatctagcatattgatattttatttcgaaagtgtttctttctatagtgttttcaatgttgccgcctcggttcatattgtcgataatataagtagcaaagtaaaacgtgtaacacaatgaatcctgttgaatacacgttgaggttcaacaaatttgacgtgtttataaatgttaaatttaaagtaaacttgagtgtggatgaaattgattccttagcgttcttatactccaaatactttaatcaaagtgacaatgtgcgcgttaagggtttaacttttttcaatgaattcaacaaatgtgttgaatttgtaaaagcaaactttgaaagcaagcaagaaaacaacgacgtcaaaaagatcttttccatattctttaaagatgaattcatgagtcaggtgcccaaattcagagacattttaaattatctacaaaaatactttaaaccgatacaaaaacctaacgtgcccgccatttcggcgctatgcaacaattgttccgtcaacaccatcgcctgcttacaatgtaaaatcaactatttgtcggagagcatcaccaccttcgatacgggcattcaagacggctgggatatattcttacgtcccatgttcggtctaccgctgctactcaccgtactactaaagaccgagtttaacgaagagtttcaagtcgacgatctaatcacaaattcgttttcgcaattcctatacaatctgctgtgcgataaagcgaccgccaattttgtcgaccacaaggcgtgcgcaccaatcatcaaacaatgccagcgcgtcacggtctcgttgcgtgatcatgatctagaacgtttgctgtgtatgttacgcaacaataattcgtgcgactcgaaactctttatgccgttcaaagtgtttattgtcgaactggcgagaaagacaaaaatcaagccggtcaagatcaacaaaatggcttcggtagtgtttaccggcttttatctacggcaatatttagaagcggctccgaacaaaactcgcaccgccgccgaactcgaactgcgcaacgtttgcaggttcattttgaacaagtacaccgatgatcagttggaatattttattagaaagctagaaaacataaaaatcgatctgtttaaggaatcgatggaacagtacattgtgtccgaaaactttataagacatttagtttacaaatacaaactagacgaagaactcaaagtgctgctagaagaaaatgtcgacacgtcgccgttacaatagatccgacgacacggacgacgacgaagatatcaacgctaatcaactgttgcaaaacctcaacgaaacacacacggtcgccgatttgatcctcaacgacacggaccaccagaaacgcgtcgccatcggcgccatgagcagacactcggccatcgccaagaccatcctcgacaacatcgacgaagacgaatcattgcgtctcggcaccgtgaacactattaacgtgttaaagttaatgtctgatatttacgataataaaatacccattgttaacaatcaataagtgagtatcttaaccagtcaaaatgagttctgttatgctttttcttgagatcgaaaggatcaaaaataaaatcgatagtgaaatgaacatggccatatggccaaaattttttcctttgctcgccgatcccgacagccacattaacctatcgctcagcgaatttcaacagtttctagtgacggtcgcgcaaatggcgcgtaccgctcagatcgagaataacgctgcgttagcaagcgccgctcgagacaacacattgagcggtgtcgccgaacaacaaccgccgccgcctccttctacttcacagtcgagaaacattctcaacttgttcggcggcggcggaggcgtgtctcgacgaaaagacattaatgcgtccgacgccgtcaatcttgccacgttccgtaaaaactgtcaaaaactcatcaaacactatacgttgagcaacacttcgtcgtcagagtttagagtggcggacattgtttcgtgtatggtttatcttgcaaaatcgcccaaattcagacccctgtactctcttttggaattgtccatgaccgacgagtacgattgtatgcccagctacaccgccgacgaaatgcatcatattgtcgacttgattaaaagtcttctagatatgcccacgtatttgatcgatttcggcagcgtaaaactgctaaagagcacgttcaacaaggccatgaactatcccatcactagattcgctcgagtcatactcttaccgaccgcttcgttggccagcgacaaacgctgtacgatcgaggaattgttagtagaaagaggacaagaaatcaataaactcgaacctcaacagtatttgaattcgagcgaaggcaccaaaattccctattgcgacgacgaaaactttattaacgatctgctgaaattgattgacgatttcaatctgcaccgtatgttttacaacgccgcaaactcaatcttttacacgaccatggaaaactacgcggtggccaattgtaaattcagcgtagacgactacaacaacatattcaaagttatggataatttaagagagtatcaaactcaatgcggcagcagcacactgatcaatcccaaagataagatagattctctcaacatatttttaggaggcggcgaatactcgtcgtccggcacttcatcaaaacgtaaaaaatattaattattataaggtagaaatataaaactcgagtgtgttgtattgcatagtcaaagtttaatatacaaaaagaatgtatcgtcgcgcatccaccggtcggcgtcgtagtagtagacgtagatcaagtcatcgtcgcagcagcagcggcggcggttatcgtagaagaagcagcagcggtcacagacggcgcagcagcaggcgcagatcgagccaccgcagacgctccagcggctatcaccgaagacccggcaggccgcgtcgtcgttccggaggatacaggcgtcgtcattattagttaaccgtgacgatgcggttacacttttgacaatatttaacaaacgaaacggcttcgtccccggcccgcatctgtttctcgacaacgacaaatttatgttcgcactcttttagcgaaaggccgttcaacgacggcaaaccgattgtgtattttgtatgttgtaaataaattatatcgtcagtgagaatgtttctttttgtgaacatacgtttcttcctcatacacttgctgttgtttgcttcagcaatctttttttttagcaaaatggtcttccaattgatcgagtacttgaataggatattgtcgatcggttctttttttagctttggtggtttactgtcaaattttttacttttaatgacgttgacgtagtttttgatttgatcgaggcgacgaaccaacaattcgcacggacacttttccggcaagctgtcgcgttgaatgtactcgaaaatctccgtgtataatttgaaatcgttgatagtgttgatgaacagtttatgaatacaatcctcggaaagacgtatttgtttgcgttcgcgttccacattgctgacggccggtatataagcatacaacgaatgaaacaaatgtcccgtgtggacaaaattaaacgttttattcttcacattggccgagtagttggttactagaaagtcgatcagttccttgtacgcttttcgacgacggaattctttaaagattaaaaataaactgtaacaatcatgctgacttggaccgttctgcagcgtcgagacgtcttcttgcgccgacacattcttgtgttggccgaaagcgccgacttggcatgcgttagctttagacatttgaaaatgtttgaattcgtaatctttgcttttgacgcgaaacaacgtatcgacacacgagactatatggtgcagattataaaatgtgccgacaacatgaccgacatacgatcaaacttgaaactggcgtataaaacgtccgcgttaggacacgtgtatgttattaatgaaaagattccaatgtacgggtttcttaaagaatggtacttgcaaaactatctcgaagtttatcaaattaaatacgacaagtacgtgtgggaaataccgcacgttatcgtctttgatctcgacaacacgctcatcagcgacgaagaacacgtgcagatccgcgacgaattcgtttatgaaagtttgacggagctaaagaacaagggctgtgttttggtgctatggtcttacggaaatagggagcacgtcacgcattccatgaacgaaaccgatctcaacggttatttcgacattgtaatatgcggcggtcaaagattaaccgatctgcgaagtcgcgtcatcgtcgacaacagttccgacatggtgtttgtagagaaacctttttacttggacgtcgacctcaccaacgatcgcctgccgaaatctcctagaatcgttttatactacctccgcaagattggagtcaactacaacaaaactttgacgctcgtcgacgatctcaaagataacaattattcgtacgactattttttgaatgtcagccgatgtattgaacctcgtcgtgattgggaaaagtatcatcatcagttgattgcaaacattgacgattacgaaagtaatcatttattgtaagcaaatttacaatgattaaaacaatatttgttatattaatagtgattattgtgataacaatattaatggttttttatttacaaaatttaaaaaatgaaataaacgaagcacgatacttgccaaattacaattacaacgatatattttatgatcgtgaaactggcaaaacgtacaattgtcctcgatcgtgtcccgtttttagctttaagaaacaaaaatgtgttagaggcaatcttcaagcttacaacgtttgtccatctaatatattcggtaacatccgacacaagtatagatgtgatgtgtttgtattttgtgcacaaaattcatcgaatgtggtatatttatgcacagaaacaacgtgttttgataatgagcaaggtatgtgtgtgccatgtccttcaaaaaactgtagctcaaacgatagcattgaatgtacagattgttgtgaaataaaagacgaaaattaaacacatttttttattgtcaacattttttgtatagcagtcttgaatctttccgcatcttcagaatcggtaaagctcagcgatcgtgattttttcttcaatgattcttccgaaaattcatttttcaaactttgcgtaacgtttgtccaatccaacatcggattaggccgtttcgattctacgataatttcataattgtaaaatttacgtttcaacttttgcgctctaacgtacttttgttggcctacgatcacttcaaaatcattgttgtttttagctatacaaatcataggcatctttgtatcgtcggcaggatattgaaccactctttcggaaacatcgtccaatttgtcgagtagtttattgatttgcttgtctttttgtaacattatttcgttagttttggatacggtgtcctgcaacttggtgtacatgttgttgacgttgttcaataacttttcgatcatggcatttttatgctcggtcactttgctcaattgtacaatcaaattttgaacttgttcatccttgtttttcatttgtacttgaaaatgtttgacgatttctttttcttcggtcagcgccgcctgcggtgaatatttgccggtacacaaaacttgcggtatcacttcttcgaacatccattcttgcaactcgaccgcgtacgacaatttgcttttcataatcaactgaatgacgccgctcttgttgatcattatcgtttgcgggtgtacgtagagaacgttgggacatggtagcaaattgttaatttttagtaatctcgcgttgactatgaccgatggcatcaatgttttaatgtcgccaaacgcaatcttgtacttgtcgtccacgtgatcgataatggcttttttagtgttttcatatttcaacattgtagcgatatctttgccgacgaaaaacacctcgtcgttgtcgccaatcacatatcgcaactcgatttcttcgtcgccaaatttaaacaagtttattttaacagaggccatgtctgctgtacgaatactttcaaaatgaaatgaaacacaattttcacattgtaaaataattatatttttctgtatatctttaaatatcttacactttcataggatttacaattatcatagtcaaaatcgttgttaatgtcggcaaagtgttcgagaaaccccgctaaaaattgaaacatttcatacttgtaaaagggtacattttcgcttctgtcgattatcggattgatgcgtagattcttgtccaagtcgtcttcgttaaatatcacgtccctctgcacaaacttgtacggtatcgcgtccaacaaccgatagtttcttggattgagccgaatcgttttgtcgaaagcatcgcatacgacgaataaattatacccgttgtcgttgaagcaccgatagaacactcgatccggcaaactgcacgcaataatgttcttgtcttcccaacaatagtaattgtgtagtctcttgtacaatagcgcttccattgtaacgtttctcttagtcgtcgatgattgtacaacgtttaataatgaacaagtttatatactttattttatctgtacatatccgaaatatatcagataattgatgatatcgagtatactgtacgcaatctcgtcgaccgtgagcactatacgtgcgtcgctaatattgttttttaacgttaaaaagtgttcgagaatcactaaactgttaatgttcgtacagaccgtgttgtatatgcccgagtcgttgatggattcaatgtcgtttttcgtgaatcgaaccagagaaaactcgcgcggacaaaacacactcgacgattccaaataatagaagagcgttccgtcgagattgtacaacaatacattttcgggtttaatgacaaacaaacgttctattcgatcgggcggcaccgacaagtcgtatacttctatatacgctccaaactgtaaagtgttcttgtggtcatttattaattttaatacttgagttcgataaggatttaaatagagaagcgcaatcaaaaatattagtagacacataatggcgactgcggagataagtgttgacattatattcgatgaaatttacaatgatatcgatcaacaaaaattgtaccaagtcgacacgctcaacaacgccgataacgtgattctagtcaatagcgtgtctcaaaagaccaagaccatcgatacgtacgataattttgtaaagcttctcaccttaatgaccaacacgcaaacggacacaaacgaccgagtcgtgtgttccaaagatcacgaaggcgtcgccataaaaccgcacgattggtgcgcgcgtcagaacatctttcgcatgtttgtgcgcccgtttatcgacgccgaaaactatagtcgtgtcaaggatcagatatgtttcaaaaaattcatatcgagcaactttcccggatacgccaacaagtgcgtcaagtcgggagactattattattggcccaacatggccgtcatatactgcggctggaacatttacctaaagaaacagttcaacatcaacatggactgttccataccgttgatcaacaatcgtcgtttgggcaacgtgaacctgttcatcaacaatcccgaagatttccttaacgtcgaactgagcctcgtgactagcgacaaccgaacgctgtttgtcaacggccgctcaatttttaccgaagacgacgacgatttgttcattatcaccatggacgacgataccgaatcgaaatgtaaaatcaagccggaactcgtgtatagcaacaaaaatttttttgactacataaaagatgacatcagcctcaagaattgcgtcaccgccgacgattacaaagatatcttgtacattgacctcaaaagtttacgcgtgtttgaagataaaatcatgcctagtaacgctatcgttaccaaggaaaaattaaaagtatttcacaacatcacgccatccagcgaaaacataaagatgatgcaacaatgtatatcggattgcgtcaccctgatcaaggaaaaaatgatcgaggtgatgtcacacacggatcaggcggacgccagcgttctcagaaattattttatcaaaagcgactttgtcaactttgactacattattatcgttttgtggagaacgatctcgaagaatgaagacatgaacttgaccaagaccgatttgaaattatactttgagttgctatgcgaaacgatctttggcgaaaagggccccgcttacgacatggccaaagagagatgcgaaccgtattgtttgctaacgccgaaaatatttttgagattgtgcaatcattggaccatgttcaccaacgaaaatccttgcgtaactttggccttttattacgccatacattttttgatttattgcaagcatggcaactgggaatacacgtacgagaacgccaaaacgtgcggcacgatccccgaagtgctatgtgctggttttttcaaaaagattgtgtcgtcgggcaacatgacgtttgtcttcaacggcaaacattacgtttcggtgcgcaaagatgacgacctgtacaaacttacggaaaattgcacgagcatcacgatgcccagtatcaagtttaacaattggaaatacatgtacttcactgacgaaggcgtctacaatcttttcattaacgattttcatagcggttgtccgtttttgatgggcaatactctggtcaaatctttaacgcacaaaaacgagaaaatctatctgcctcaaacggtcatccaatacatgctggacacgggcaaggttgagattgaaatttacaagatttaccacatggccaaagtgtgtcgtgaaatcaaaaatgtcaaaaacaacatgacttcaatattggcgtttaacaattgcgcgtcgtgcaagtttgaagaacaaaaaactctcaacgatttgtacagagaaatttgggcgtacaatcatgacgaactcataatcaccggcgtctatctcaacgacattaaaatgtccgacttgattatcaatcttaaatgctacgagtgtaaagaacatatgcacagtccgtacaaaagagactgttcctgtttgaacaaactcgagattgacgttaaagcgttcaaagttgttctcatcatggaactgtttacgaattgtaaagagatactggaattggtatggtcgttgctgtacacgtcatcgctgtacactaccattttgttgtacaaatattcgtgcgacaacaacatgaagctgttgcatgattacggagagtttttcaccgccaatcgtaccaaaatcatcaacttgctctacaattggttggataacattgattttgtagattcgtttgttgcgtcgttgtcttgtcccgaggtgtttttgaatcgactgaaaaattccgtactaaacaactcgaacgacgttaatgaattcgaaactatcgacgccattgaaaagtttatcaatagcgatagcctgagcgttgtgtcttacagcggcaacaatgacaacgacgacgacaatgacaacaacgacattcataattatgaaaaatcttttttaaaagacttttactacaagtattacacaacgttgagcaatctgaaaacatggaacgtttggtgggacaaactgatcgtgatgcgtcacaacgacgatctcaacacgtggctaacgagattctttatgaggatattcatgacgaaaatggaacttgaaggttacccgtacgtttttatcaaacaagtggtcatcggctatttgtactttagacaattcaccaactttaattacaccaatagtttggtggtcatgcatttcgccgccggtaccggcataccgaccgactacgagaaatgttgtttgtacctcaacggcaagcctggctcgggcaaatctagtttttttgccgttttagaaccttcggtggtcgtgcataaacacgattcggcaaagtacacgctcaccaaaaaagacaccaacgaaatggaagcagacaagatgatttcacaactttacgtcatcaacgaaatgaaggtgtgcgatgactcattctttaaaagtacagcagattcgacgaaaagcaacagcgtttgcagaaagtacgaaggtagccaaaagtacgaagccaactttaagctgttgatcgtcaacaataaacctttgcacatttccgattatgacaaaggagtgagaaataggtttgccgtcatttacacggaccacttgttcgaagaaaacgtaaagtttaacggatccatttactggcacatcaagaacaaaatgtatcccatggagaagagctacatcgacgaaatggccaaacccgtgcgcatgtttctatcgcacattctaatgtacaaacgcaactctagcgacggatacgtgtcgtacaagtgtttcctagaaaaggatcccatacacaatcacaatctaatgtgtctcgacgtcaacaacagcccgttgaacgcgctcatctatgtgctaaaggtgaacgtaaagccgggcgcgagaatggtggacgagtctaaaattgaaaaaatgatcgaactggccgtgccgtacgtcgaaacgatgctccacgagctgctggtcaccaaacgtaccaacgtggcccaacgtaccgccatgctgtttgacgcattcaaaagaaagtttaaaaagtactacttggaagagagaaaaatattctacaacattgacatggcgtggaacaagggagatttcaacattaatcaacctgaatttatatgttagtcaaataaaaaaaatatctatgtacaaaatggtttattaataataaaaattcttaaaacaataatacaattgttttattattgtagcctaaaggatttttcgtttttgatgaaaagcgttccggcgtcgttgatctgtgtgtagtcccattctctgagtttgagttgaacgttggcgtttgtagtgtacacggtgatggggtatgaagcgtcgcgaagatcgggaaccgacgtggtcgtagaatcgatcaggcaatacaccattccgtcggcttcgaatcgggccatgttcaccgattcctttatcttgttcgagtccaagtttttgaatacgataaacatgttcttgaactgtttaatgtcaaagttggcagctacgcggttcgaagccgagctggattgagccgtctcttgcagcgtgcctaggaacacgcagttggtgcccactgatttattgccctcttccacgatatcggtgtaagtcaacggtctttcggcgacgtaaatcttgctaatcttgttttcgccgtgcaggacgcgaagtgattttattttggcgttgttgaatttcaacgtggtcgtgccgttttcgttgacggtaatcgaaccgctgctgtcgttgagactgggcgacgattcgttgatagagttcatgtttagtttattgttggtccacaaatagtacaataacgccagtacgattagtatcactactattgttccgatcattttgaagcgatttcaacaatcagattgtccgataaatgttttaatagcttacttaaattattttcttccaaattaatattattcggcaacacgattattatacgactttgtaatctgaacaatttaaatatactcgtcaagtaatctagaataatgtttaaaatgttcttgccgaacgcagtatcgtcgaccaattcaacatacgtcttatacattagaagcgcgtcgatcaacacgtgatttttgcagtagtttccgctgctgtgtactactatatcattcaaagttttcctcattagtaaggcctcttcttttttcacctcgtacaacacactgagtttatcgcaagactcgtcgtcgaggcattcgataacgccgccaaagtaaacgacgtcgtcatcgtttggtttatcgttgttgatcccttcaggcatatcgactagatacaaatagattattcgttcgttactattcattatgatacctcaatcgccgttgttcacacaatacaaagacagttttctgttgtacacttttcgtcacttggaccgaatacgcgcatcaaagtccaaacagttgagtaaaattttagccagcgaattaacttatctatacgagattgcgtgcctgataatttataaagatgttcaaaacgaagagattgaaaagctcaaggaatggtctctgcagctgtctccagactttgatttggaacaaatgaaactgttgtttcgagaaaaggtccaagagttaaacttgcgcagccttcaaccaaaaaattactcgtacacgtttacgacaatatggaacactattcatttcatcgcgctgctcatcgacgacatgatagcgaatcgggaaaagatgacatacgattttatcattaatcacttgcgacaaatgaaaattttatattataatttatttttcaaattggattgtgccatgtgtcgtgatcattacatgacggtcaaaggctttctaatacaatcgatcgaacggatcgaactgtgtctctatcgagaacgctacggcgaaaagatcaccatggtcgacgagataaccgtgcaaaacgtcaacgacaacgttttaatgaaacacggcatgttatacacgacaatggtgtttcataatcacatcaacgactatcgatggattcaaagaaacattaaacctcccgtcaactatgaaaagatgaagtggtccacctacaaagccatgttagaactgaaataaaacacaattgtttcaaattaaaataatttaatacactacaatcttaattaggcaccaaacgatctggacgctgtttaataatctttatatcgtcgtccgtgataattgcttcgtatattgcgccgttttcaagtttatctatcgtcggcgaaatcaatgtgttctgctgcaacagcggaccctctaaatttttaaacagtttcatttcggcgtcgtattccacttcgacggttttaacgcgtttgtacttaacgtagttcatttccgtgtttaacactacaaacccatcggtgggcatggtcgagtatccgttttgcacaaacggcggatcgaagaattgttgaaacttcaccgaaacacattgattcgtcatcgtacgcaaacaaatcgaggcgttgttaaatttttgcgctaaaaaatttatcgaaaccacggcgtcgtcgggcgctatctgatacggatcgagagatatttcgtattgagtgcgattgttataattgtacttgaaaacgtgcaacagatcggtgacgtataatactttctgatcgatgagttcgcattgaaacgccaccacgttgttcaactcgaaaaggccgggctctatctcgccggagaacatttgcatgtcgtccatgaacacgatcataaaatttttcgtgcaaaatcctttaccgcgtatgccgtccaatttcaaagcccacttgtaaatgctatcgttttcaagatcatagtctgcaatgtcatcgagaagcttttcgtcttcgaatttgcggtagattatgttattctgcagggtcgtgtacggtaacaacggactgatattttgatgcgcagttattttatcaatttgtctaattacgtcactcatatactctagaacgttgtaatcaatagcgttttcctcgtactcgtactcgagccgtatagctgccaaaatttcatcggaccccaagtggctttctttaatcaatttttcgtttttgttttgcagcaagttccagagcattatctgcttcgacgccattaaagaatcgaatttatcaattagacttcgattaaagtaaatgtgttcaaatttaatttcaaactccggacactcttcgttcttgtacacgatacattttacgatacgattcagtatcggagacttgtctttgctcggctcttctgtactctctcggtccacgaacggaatcaacacgttttcgttgacgtagacaaactttttcaagtttacacattttttgattacactcgaaaaggagccatcggaaaacagacgtgtcctcacgttgttctcgtcgacgaaatcatagtagtgttcaaaggctttaaactttttagaaatataagaatctaaaataatatacagcaagtcttggctgaaatttattgaataagaaatttcgttttcgacaaccatggcactgacgcctatttcatcgactgccacgaaacgtaataacttttgtattttcggagcggttcaatcgtttgacccgtgttacacgtacacgaaaacttgttctcccgatgccgctttcgacgacggatggtacatttgcgagtatcacgcttcgatacgatttaaaattgaaaaaatggttatgcctattcctgacggagagggcagaacctattacagaacggtcggcaagagtctggtcagcgacaaagccgagggcagcgagcgcattctcatacccacagcggacaattacgaaacggtgttgaacatcaacgcaatgtcgttaccggagcagctaattttccatatgatatacaaaaatcgagagaaacaggagcgcgtctgtcaaatgctacagttcaacgagcatttccaaacggaaatttacaagataatcaattccgtgtacaacaacacgagaaacgtgctcgcaatgacggatcccactcaatattgctccagggtcagccagaacagcactcgggtctacggcgttaacgatgaaaacgatatcgccgaacagaccatcaatcgaatgcccggcttcctgcagaatctcatcaacaagtgcgtcgctcctcaaactatgaacattgaaaaccaaacgctagaatttcgcaattgcgctacctgccgcatcgacaccaccggcttggttgccgacgttaaactatataatcccatcgaacccaagtacaaatcgaaacgggacgaaaattatctaatcgtagataacgtatttaaatttcgaggaaacactaaagcgctccagacttctctcaaccgatacgaacaataccccgtcgtggttcctctcattctcggcagcgaaattgtggtgaccgccaacagtttgaagcaggttcctttgccgcgcatcctgcccctgcccgaagaagtcaatcctcctattccaccagtaaacgtggtgcccgtataagtggtgtgaccttcaaaatgacttttatttaacctctacatggacattgacctcattaaataataataataacgataatctattgataaagttatttttttggaaaattatcattgcatacgtgactgataataataatagcgtacgtgactgatatagcaaggatttcagtatataaaccgtcgacgaaaatgtaattgcattcagtcgccaccatggatttagtgacaatacagtgcatagtgtgtctcgtcgagaccacaatagatcacaccaaagtcgaagaagtttttgtggtgccttttttaaagttgaacaactgcaaccatgtcatgtgcgtggattgtataaaaaatatgacgacgcccttgtcgtgcccagtgtgtcgtcgtgtgagtatcgccgtgcgcagcacttggtttggaaactataacaatgaagtctggtcgtacgatttgaaaagtttgatcagtgaaattaaaaaattacctaaaaagccaatcaaacaaacgaaaaacagtctactgaccagtattaacaattcgtttacctatggtgttataaaaactacaaattcacaaccgtcgacgtcatcagcaactatttctaatgaaattattaacgtcgaaagcgaagatgaagattttgtcacgaatcaagtttctaatgtttgtacttcttcagatgttggaaattctgatcctgttgcaagcacttctactggttctgttactacgacttctaatgatgctgttgctagtacttctagtaatcatattgcaaacaattcaacggaaccggttgcgagcacttctacagaaaatatcgcgaatggtgttgttgacttgaacgatgccagaatcgttgcgatttctgcaaaaattaatgatttgatgaaccaacagtataacgctgtgctgacgttgagcaatgtacaacatcaaatcgacacgttacacgtggaacaaaatacattgctcaacattaacgaagagctcactcgtaccaaagaagagctcactcgcgccaaagaagagcttatttgcactcaagaagcgattacgcaagatataaattctaaacgacaggagatgacatatctagaggctagattgcatatttataattgtaatgtcaagtatgccgaaatggaattaaaaaaaataaataaagaattgaaagcgaaaagattgcaactttccaatacgaagacagagtcgaaatctacgtcgacagaatcaaactcgtcagaatcgaaatctacatcgacagattgtttgaaagactttataacgtcgtttgattccaacgaagagtttaataaaagatttgcaaataaaaatacaaaaaaatacgtagaatcattgataaactacattatgataaagacggagaatcaaaagaaatgtaccaatgaacaaatacaaaaattgctacaatcccacgacagatactttatcataaacgagaatattttttcacccaaacgcaaacaatgcgacaacagcaacgctgataatgacgacaacgacgacgacatcatcatcgagattaacgacgaaaatgttaaaaaaaaactcaaaataaattaaagtataaattgtcaaaaatgtttgtatttttgtggacattgtaataaatatatttataattaacaattttgttgttgttttaattaatattgtaaatcttcaatgtaatagattctatcatcttctaggttgcatgtaaagtcgtcggcgattgcgtgatcgggacacgccatgatttttccattgttattgaatatccatttgttgtattgtactccgtcgtcggactgagtttcgacgttcaaaaacgttttacctcccgacatttgaactctaatcgtgtctccgtcaaatgtcgatttgcatcccaaaccgtttcctagatacgcgtaagcgttgatgatgttggccaatcctgccgcgctctcgcattctgcatccactttaatattttcacgcaagttacgaatactttctttaatatgatcatcgacgacgttacacgcggacagtttaaacgttggtatggcatagaaacatgtgtaattcaactctttgtcggtcaaagttaacgaaggcggctctggttccggttcaggctctggttcaggctccggctctaccggaggtcgtgtacctcccccgccaccgcttccccctccgacatcatcaggatgtacgtcaatgatgtcacgagtcgaccacggatcgaatagcggttcgatgacgtcgtacctttcgaacaaatcaaacatgggcaccatctcggcggaggtgtcagtttcatcggcttcaaatggtatatttgcccgatatcgttcaatatattttgggccttttacactttcgtaattatatttgagtggaatcgtagtatatttgatcggaatcgtagtatatttgtcatgaatcttggtcaagatttcaccgcacacgtcagattgtagtatattggccattatttgcgtcgtaaaataatcaaactctactaaattttcagtcgacactagacttttgaaacaccaatgttgatagtcggcgtcgctgcgtatctccatcgtcaccggcgctatatactgatcgggagttaatttgatattttctacgagctcattgtctttgcatacgttcaattcggtagctgcgagcggatcgaacaaataatcgccgaaacaatctatggtgtccgaagtgctagcgtcgaagccgagaacgttaaaattgcgagcatataacactttattggccaaagtgtcgctgccgatcaattctcgtacttttagcgagtctccgagaatagacgttataaagtttatgttgtaatcgttggctatattttcgatgacatatgacaaaactctcggcgagggacgggtcgcagtgctgggcacgcattgacgagtatcgcgattgaaaatttccgcagggaccataatgtctgtgtagaatttattattatacaatttatcgacgtctatcaaattttcattgttgcatgtaatatcctcgacaatgttgtaattgtcgcaaatcaatataccgtagtcgtacctaaaggtattgtcgtcgtagttgcgtatctgttgaccggtaccattttcgaacatggaacattgggggtcacccgaacattcgtaattgtcgttgacaaacacgcgattgatacacgtgacgagttctacttgattctccgatacacatctgtaaaactgcgtcggtccaatgtcgtcggtgatgtatgtatagtcggcgccgtggacagcgcaaggctcggcgtcgacgcaagtcaacagacgacgatcgaagattttaccaaacggacacgacaccaccttgctttccccgttttcgcagatcatgtactcgttaatgttcaagtcttcgggaaacacgtttagtatgaaaccgtcgcttcggttgacgcaatcgttacgcaattcgcattctacgccgttgaatagatgattattgggacactcttcgacgacgtgagacccgccttcgaaacatctcaaatacatagttggatgaaactcgtcttgggaaatgttagcacgggaaacgcgatgattgagaatcaacgagtctatcaatcgctcggtcaacggatacattcccgaagttttgccgtcacacggaggtatgggcacgcatctattggtggtttcgtcgaaacgttcgtcgctcggacattctacctccatccacccgtcgtctccgcgcaactcatacttggttcgatcgtcgctgtttttgcgtacgcgtttctcgggaggcggtgtcggttcaaagttttgttcgatgaaattgaacgtttgtaaacgatcgtcgtgcacttcgttgttgatttctctcaaatccgtagtatcgatggtgcggaccacgtaaaagttcgagtctacctcggacacgtattgaatataatcaggcaacggatgttcggcattggtacgtttggcgtattcggtcacaacgcgcaatttgttgtcgaaatcttgttcgttatattccgaatatattattaagtagaagattgaaaagagtaaaatcaaaataatggccaccagcaacagcggtaccatagacatttctgttcacgttactctcgacaaggaatcgtcaaaaaggaatgttctttcgtttattgttcgagaagaatatcacttaaaaaaactagccgttggcgcttacaacatcaacattcttgatactcagctgctcaacaacttgtcgcaatacaaatgcaacacggtagcgtgtggtgattacgtaatcgtgtacaatttcaccgaaaacactagcaatcgaatcaatgtcattttgttcaatataaaaccgaccattttgaaaaagggcaattgtatattcaaaatagtttacgaagacgacgctacagcaatcgttactactgataagatgttagacgagccggttaagcagcatcatcaagaacatgccaatagtcaactgttcaaatccgctttcacacgagactcagacgacgccgcctcctcttcctcctccgcatcctctgacgaatcagacgaagacggaaacgtcgcctcaagaacggcagcaaacgacttgtctgaggctgtcccagctaaacgacaaaaacttgacaactttgtcgagagtcaagtatgacgctgatcttttactacactacgttttcgatggaatcggtcgcagcgaaaacaccaacgtcatcagagtgtgtaaagttagagttaaaaagacatgcggtactctgttggctcactactacgctcaagtcgaaatctcaaacgggttctcgtttgaattccatccgggcagccaaccgcgtacctttcagcacgtacacacagaaggaaacgtgatactagtgttttttctctgcgacgattgctgtaaaaaagaattgacgcatttcgtacaaggcgaaaacgaattcaatgtcgcttttaaaaattgcgaaagcattctgtgtaaacgtagaagcgtgcaaaccgcctatatcaccatggcggttatagttttatttatcaacattatacatttttcatggtattttgtactatttattttcgtaatgatattcttgctatatctaaacaataattatatgataagtaatccaaaaattgtttactgccctcataaaaaacacaatggccaattacacgaggccaaattcaataactaaatcgtcgacaatgtcatcgtcttcgttgtcgtcgtcctcgtccgcggccataaccgaaccgtggatggacaaatgtgtcgattacgtcaataaaatcgttcgatactatagaacaaacgacatgtctcaattgaccccacaaatgctaaacctcatcaacaccatacggaatgtttgcatcgaaacgtatcccgtagacgtcaacgccaccaagcgtttcgacagcgacgtcaaccttatgaacaattacaaacgactgcaaaaagagctgggcaataaaccgatcacgagcgacattttcaaagcttcgttcgtgtacagcgttttgccgtcgtacgctcaaaaattttacaacaagggcggcgatcatctagccagcggcagcgtcgaagaagcggcccgtcatttgggctacgctttacaatatcaaatcgcgcaagctgtgaccacaaacacacccatccccctgccgttcgatcaacagcttgccaacgattatctaacgttgcttctgcagcgagccaacattccgacaaacatacaggagatcatcaacagcggcaatcggacgcacggcaactcgcgcgttcacatgatcaacgctctcatcaacaacgtgatcgacgatctgtttgccggcggcagtgactattatctgtacgtgctcaacgaaactaacaaatctcgcattctaagtttgaaagaaaatatcagttacatggcaccattgtccgccaccactaacatattcaactttatcgcaacgctcgccaccaattcgggtaaaaagccgagcgtgttccagagcgcttcgatgttgaccatgcctctaactaaacctgtcgtcagcgaatccaaaaacgtgtgccaacagcaactgactgaactggcgtttgaaaatgaagcattaagaagatttatcttgcaacagttaagttataaaaacgacatttcgcaacagtgataacaactgaggctagaaaaaaaaagatgagtcttgacgttccgtacgaacgtttaggcacagcgaccaaagtcgattatattccgctaaaattagctttgactgatttaccttcagaaaacacttcagacaacaatgacgacaatcaaaaaaacaacaatacccaaaatcccaaaattgatattaatcaatcaaacgccaacaattataatcaacatcaatcggttcgttcaaaacaacagttttacgacattttagttttaggtatgctgacagtgttttgtattttggtattgctgtatgctatatattactttgttatattaagagacagacaaaaatccaacactataagacctagttatatgttttagcatgactgataacattttcaataaaacaaacaatgtgagaaatgaatattcgtttaattgttggaaatccaaaatccaaagtcattttagattcgagaccgtgtttcaactggccaccgatcgacagcgatgcacgcccgacaaggttcgtaacggtcggtggtccaagtttatttttaacaaaccgtttgcgcccaccacattgaaaagttacaagtctagattcatcaaaatcatctactgtctaatcgacgagtctcatctcgacgaactaaacacctacgatcttaatcaagaattcgacagcatcgaacagcaaagactagtcgtcgatcccgaagaactatgtatacgtatgcacgaacttcgatccgtaaccaaagaaactcttcagctgaccatcaatttctacgtaaattgtatgggtatcgaagagtatcgtataccaaaagaggtaatgttgcccagagacactgaaattaaaaacattcgaaacaaggagaaaaatatcgtattaaaacacatactagacacggtcatagattgcatcaaaactcgtataaaatatttaaacagcgattatgtacacgataggggtttgctgcgaggagctataattttttgtatcatgctcggcaccggcacgcgtatcaatgaagctagacaaatcacgctcgaaaatctcgaggccatcatcaaagacggcaaagtgagaagcaagatcaacttgaaacgcaaacgcgatcgtttgaatcctctgcatcgactcgaattggctcctttgctgttggcgcgagagatttacgccaaacacccgaccatattgcagatatcaaagaacacgtcgacgccgtttaaagatttcaaaaggctgttcgaggaagctggagtggaaatggacagacctcgatccaacatgatcagacactatttgtgcagcaatcaatacaacaaaggcgttccgcttcaacgcgtcgcaaaactcatgaatcactcttcgttcaaaagcacccaacattacatcaacaagtttgacgtcgacatcgacgacaccgacgacgatgacgacgacatcgaactacccagtcctgaatctagtaactaggaataattaaataaaactctgttaaacatttttcattagtttttttattatcactcttctcgaaaacaatacaattttcgttgaactaactacattgagattacaatacatgtcgacgaaaacgatacgactttcatcgaaaatcaattttaaaaaattttttacaaaattgtaaaagttaactttgtcgacaataccatcattttcggcgaaaatacattatgtttttcgctgacaacgttatcgttttcgatgaaacgcacaatttaaattacgacgcaataaataaagatgatgcaataattgataaaatgacgtcataatatcattttcgtcgacgttgatatgagtttcgctgaaatgagattattaaatgagcatttcgacgacaatgctatggttttcgtcaaagtgagtttttagatttattttaaaaatttttacaaaatccacttcagcgaaagcaatgtcattttcgtcaaaattatatgtattgtttcgacgaaaacaacattagtttcgtcgaagatgcattttagaattttgtttaaaaattttgcaaaagtcgtttcgatgaaaatgccatcgttttcgtcgaaacaacacattaaacttcaacgaaaatgctatcgttttcgtcaaagtctaattttaaaatttttcaacaaaatttttcaaaaactcacttcaacgaaaatgctatcgttttcgtcaaagtctaattttaaaatttttacaacaaaatttttcaaaaactcacttcaacgaaaacactacctgtttcgttgatgttcttacaaaattaacacattgcctgcgaactgcgttttcactctgagtttagatatcggttctccgttgtacacgacgttaatgtacacgttctttttgtcgaaagtcacgtacacgtcgatgcgaggtaggatttgaaacgtgtgaaaagtgagatgatcgttaggatcggtcacgtagacgaatccatctagacatacggttccttgagctcctccggcgtctccggcgatcgggacgatgcagtgattttcatcgttcacatagtaatccgtaactacagaaatcaatgttccgttgtcgtcgaaaatgggtgcgcttacgtacaactgcgacatgaactcggccagattgtttttttgcataattgccggcacttgaccatacacataatattttgcgtaatgatgatgcgtgtaatacattcgatcttgaatacgcattctgcgtagatatggcttgttgttggcgttgaacagcaatatgtttacgtacgaattgcgtttcaattttaaaaattttacatcgctcgccacgccaggataatgatacttgatgtcttcaaagtctgctgaagtgtcatgttggccgaaaacgtgtatgcgtacagcacaatttttttcactaagtacataaactctacgagtcatatgattgacacaatacgttatggattttatgtactctatcgtcgaaacgttcgcggcgccagcggctgcggcgacattttctgtcgcgactgaattaattcgcagtgacattgaaaatgatactgtttcgaagatcgctcaaggttttatataaagctctattgtaaaaatacttctagtttattttcgatagttttgcgacaaatacaacagactttgcatcgtttggcacattgcgagcatgttgaaacgtgacggcacggcaaaaagcatgtgtctctttctcgctcaaaacatattttgcacatgacatcatctttttgagtgtcaacggccgtcgacgatgatgacaacgtttgatcatcgctatttatagacatgctgatttcattggaaagatccgggtaaatacgtgacacgatggtcgtttgattagcgagttcatcgatctcgtcgtaatggggcgccgaaggtgcgttgaatacacaatcgggcgaatagcgtccgtgtatgagagcgacgttatcgttcttgttcagtttgactatgacaaatttgcatgaagaacatataatttcaaatttctttccatagtaatagaatccatttttggccagcaactcatacatgtctttgtaataatgacgagctgttttgaatttacaaaaggattgcttacgtaacgtttcgttctcgaatagtcgttttgttgccatgggacacgtggagaatttgtgatatttgagactgtgctcgtcgagttttttcaagtacagcggacaaaaggcgcatttgtagttgcacattgcgtcgcggtaaataccactggcgacgagatcgttgatgtattcccgtgtaagataactgttttcgaatgtttcaagacgatcaacaatgttctgatagaatcccggcggagcgagatcatacgtgttccagttgggagaggtcattttcaagaataaatagcaattgcttgagagcccgtctttgtctttcggcaaaatatttcgtatgcgcatcgaattgtttgtttaagaccacgttatctttgttcaatttacccttgcaaactagatatttttcagcgtttgctgctcgagatgctaacggtttgtatagataatgttcatcaaaatgagatatgaattcttgaagaagacttattgttgacgattcaaaagtgtcgaatatcttcaagacacaattaccgcccgtgcgtaaagcgccaaggattatcatacattccttggctatcaacggataagataacgattcttgattattttcattgcccttgacgtcgaaagcaccatcggcgacgaccaaatcgcatttaccccgacaaaaatacattagctcgaagactacgttggcgtcgaaaatgtcgcccgtgtcgaagcagccgtacatttttcgaaaaagtacatggtcgaacgtgtagtcgcaatgattgcgaagagtgacgccgtaacccgaacactcgtgattgttgacgtcgaaaacgtatttggcaaactgtcccggacctccgcatagatccaaaaatgtttcaacgttcttgcaaacgttgtacttttcgtcgatgtctttgatttttaaataacatctgtttcgtatgcgaacgccgccttcgataaactgccgagcgagacgaacatggtgttcgtcatgattgcctagtcggtttttgagcgcgtccaattgtttcttcaatctaaacattatcgtcgttatctttatcgtcggagttgaattgaaatgcgactgctaccgtcaataggatggcgcaaataaaagcaaggaatacttgaatcgatattatattcattgcagacagcgtaccagtaggacgattgagcgctctgttcaagttttcatttgttatcaacaattttctatctttgatttcgtatatcagcggtcggctgtaatcgaaaaactcaaagtttgctttgttgatcatagtacgaaaagtactatcctgaggcgaatcgagcaacgtgtctataatcaaatctttccacgcgtgttccctgctctccggcgatacttcaaccctatccctgttcaacattcgccaacgaaccatgtcttcgtctacggaaatgcctaccaagcgcggactgaactgtgaagaacaacaagaagtcggcagcagcaatgtcaagcgcggtcgctatgccagccccaagaagcggatgtcgaacggttccacgacgagcgtagattctgcgtcaaaaaaaattccaaaaagcgtaattggccagctggtgacgaagaatatgtacagcgtcaacaatgaagcttattacttatttaaatttttggttgacaatgtgccaaaaaattattatggaaaccatcacatgtttcaaacgctgaaaatggacgctgtttacgagattgagttggtgtacgagaataaaagtttgtcgattgccaaagtgaccgagtgtaagaatgtggacaagaccgtgttggccaaacactatgtcgagttgagcgactttgacggcgaagacaccatcagcgtgttggccaagttgaagtttggatttaaaattttggacaacgatacgtacaaggctgtatttgtcgtgaaccatggcaatagtttggacaatagttgtcctgttcagattgaatgtatggccaacttgaaacgttgggcggcgagcatcaaggacgagagcataaccgatgagaattcattgttggaatatttcaactatttccaaaatcaaatattcagtctgaatcgcatcaagtgtcagcagagcaatggaaactttaaaaactttgctcttcaaaatattacgcaaatatcggtgtctaaagtaaccgactttgaaattgacgaagatttggacaacatcaacaacattagtagatcgaacaaacgcatcgtgaccggctttgtggataaggtgaacgtcgagagacaaagcgatgagagattttcaatttcatatttattgaaggagagagacgatgatgaatggattcgcgcatcgttctacgtaaagaatttacaaaacaacaacaacaacaacagtgggggcaacgacaagaagaatgataaaatggaaaaattggaaaagttggagactgatttgaatcagttgaacgatttgatcgaaaatgatattttaaaggttcaaatttatgtcgcctatgattacgtaacaaagaattgtaatgtactcggtttgactaagattgaaattgatactgataactttgaaggtttttaaaatgtatattgtaaaaatgtgtattgaataaatgtgtattgaataaatgtattaacgtttattatttgggtctaatagcttctttcctagccattgtctcgattctagttttaaactcttcaatatcagcggcagattgttcgattctattttgtaaaggagcaacttgttctatgagtttgtctattttgttggaaatgttaactttagaatcctctagacgttcttgtaaaatgatactttctcgattgagttccatgttgcacgtttctttgactgcttgattttccgcccgcaacaacgacacttcgtcggctagattctgcgtactctttttatttaacgctttaaagtctaaaacggacaaggcggcttgattgctgatcaaagattcgacttgcaaacgagtcttttgtaattcgtttttgataatttcgatgacttttgtggtgctttcaatgttttcattgtatcgatttttaatctcttgcatttgttgatttttaaccttttgatcgttcaagagttgctgagtttcggctattagtttagagttgatggtttgcgcgtccggccgacgcatagctttaactttacgtataggtttaattgcgcccgtctttttctttagcgctttgtattcttcgagaatgttattgtatttgtcgtttattgatttgacttgagactgaagttcggtgtttttagtttgagcggcacgcaactgggactgtgcgtcttcgaaagaactaagtttgagatcgacggacgcgatatagctatctttttcgttaatgttttgagtgagttggttgattgtgtctttgtatgtcgattcgacttgattgtactgctgtcgaagttcgttgtactgttgttgcatttggttgtacgattgagtttgttgagagagtaattcgttttgttgattaacgttggttatagcttgagcgtttgaagtttctaaattctcaaagtactgaatcagattgcctacaaactgttgatcgtccgcgggaggagcgctgcctacgtgcgatctgtaaaagatgcgcagtttctcgttaacgttattcaatatcatgcttttactttccgcggaatcgtattgctgttgaagcatagtctgtttggtttgcagacgagtgatttcaatctgttgttgttctatggtgtttttgagcaattcaatttgttgatttttaagcgcttccacttcaggcgtgtacgtctgtatgggttgctgcggtaccggtgccggtggtggagcgttgatgacgttgacgacagccacttcaatttctgacatgactgcacgtaatctttcctcggttttgatggcggacaagacgagttcttgtttagtaatctttgcaattatcctacaaaaagacgatatcaaaaacgatatcaatcgacacacgttcggtccgttgggtaaagcccaacccgtttcgcgttcgatgcatagcaacagttcgtcgatgtcgttgtttatctgaatagaatcgaacgtttccaacaactcaagactaagcgttatctgttcgtaacgtacgtatttgcgcaccaaatacacaatctgacgcaataattgtttgtatgtcgtaacgctgggtgttctctgcgcgttcacatacaatacattcaacgatgccagatcgctttgatctatagacggcaacgaaactactgtagtcgtcgtcgtcggcaccgctgctgtcgttggagcgggcggaggtggttgtaatgtggtggaagattccgcgacggtgggtgccgctgtcgtagtcggcggcggcggcgcagcattgatcgtgaaagattgtaaaggagtcggttgctgttgcgaagcgttcggcatttgcattggctgttgcggcggcggcaggaagggattataggcgtttggaacgtttgaattgtaatcgtatttgtagttgaagttgtgtgtgatttgattggcgttcatactgttgggcatcaacgcttccattgttagttcgggcaattgaagatctatgcgattagctaaatgcggcctgtacatcaatataatagatcgtactctttgaacaatatcttcagtgttggcctggcctctacatcgttggctcatattgtttattgtttgcaaaagattttgtacggtgccggcgctgacgtcggtgtttttgtattttggattacgataactcataatggtggcgttttcgttgctcgattggaaatgtctgcagagcaaagtgaaaaattttgggaatcgtcaaggcgaggtgattatcaacaccgacgatgtctttcgaattacccgtatgatatacaaggataattacttaattgtgttctttacgggacatttgaacacgggagaattgtaccaattctacttggaaactaaatgcgatttgtattcgtatcgcatgtgttttaacacgcacgccagcacgacatgtttcaacaaatgtaccagctacaagaccatggtgatgccgggactacgaggcgtgcacagtgaacgcatcaacatcatcaaatacaaacgcaatccttctgatgattacaataatagaaagagtctcgattcgtttctcaacgacatcaatagagtgcacactcaaaccaatctgaaagaaggtaaatacatcaaatttacatcgcggcaaagatgcctgaacaatcgtctacaatgtgccttttccgatttcgattccgtacaaaacatgttcaccatcgtcgatcctgattctctcaaatgcgaaattgttcctgtaatggcatgctacgacattgaaacgtactcgaacggtcaacaattctcaaacgccaacaacgatcccatcatctcgatcggtctggcgctcaaacgcagtgcgaaacttttaaagatttgtttgtaccacatgcctccgggtgtcgtcgacgacatgcagccgtttttcaacgaaaacaaagacaatgccgacatttatgtgtgtagatttgacacagaattggaaatgattgatgggttttttaaattgttgccactgctgaacatggattgtttgttggattttaacggcgacaagtttgatctgccctatatcatcgacagagtgaagaaagtcaattggtcaaaggatattttgaaaagatgcggacacgctagagccgaacaagtgatcaaaattaaacgctacgatcacgaaccgctaccgataaagaccaaggcgttgtttgataaatttcagaataccatgaatacgcattttctgatctattacacgcacgtcgatctgtaccagtttctcagtaccgattctgaacaaaacgatgttgagaattttcaactcaacacggtttcgcaacattacttgaatagttccaaggtggatttgcccatttcgacaatgatcagtctttacgatcgtaaagagatgcaaaagattctagagtacaacgttcaggattgtgtgctgcccatcgacatattccttaaactcgaagtcatggatttcatgtatacgcaatgcgaacttctctatcttagcaccgacgatgtgcttagcaatatttctcacaaagtgaacgtggtctttttctacaatgcgatcaataatacgcgctacgacaaagttaaaaaaatcaatgtgcccgatccgtactttttcaacaagcacgatttgaacgtaacttcgggcaggaaacgtactttcgaccaaatgaacgacggcaacgtcgacaatcaaatggtggacttgacgcagttgaaacgcaaacccattcctgtcgccagcatacccgccgacgcggttcgactttgtcaccagaaacagagatgcgtgtacaccggcggcaaagtgctctcgccgaatcccggctttaagaaatgggtggtgacgttggactttaactcgttgtacttgtccattatgatgcaagaaggtatttgtctgtcgaatgtgtttgtcggtcaagacggctacgtgtacttgatgaaagatcaagaagctatcaatcccaaattactaaagactttgttggatctgcgtacaatgtacaaaaagaaacgagaccgtttcgagcccaattctttcgtttacaatttgtacgacaagactcaaaatgccgtcaagcgtatcgcaaacagtatctacggctactttggaatatttttcaaaccgctcgccaactacattacgaagattggccgcgaaaagttgatggaggccatcgaaaacatcgaggccatgagcgacgaccccgaaatactgcgcaatttcaacatgtcttcgattcgtttcaaagtcatctacggcgacacagactcgtgtttcatccaagtgctgttcgacgaaaacgaagtcaccggcagtatagataaaatgattcgtgacgtcatcaatgattatgtattgaagaagctcaacgccggatgggtgggttacaagatggcgttagagaatgtgatgactagtttgattctgttgaaaaagaaaaagtattgctatttaaatacggagaacagattaaagtacaaagggtggttgatcaagaaggatatgccgattttcatgcgcaagacgttccgcaccgtggtggattcgtacctgatgggacacagtgtggcgtgcggacttaaactgttgcacgatttgatgatcgaacatcacaaaaactttggcgtcgacaacaactatagcaactattgtttcagcatgagctataacgaaaacccgaccggtaaaaacaagaagaagaataagccagtcgacgtcaatgctcctccacgtaaacgtcccatcacgatcgctaaacattgtcgtgagctgctgagcaattcaggcacggattttttgcccggcaacggcgatcgtataccgtacctgctcatcgacattcagggcaacatcagtcaaaagtcgtatccgctaaaattgtttagcgaacagaaccgagtcagttggataaaacacgtgggcatcatgtgtacgtttttcaacgaactaatacagattttcggcgatcgtaaagagtttgagtattactttaggaacatttgcgatgtgtacatgagaaaccaaatgtttgacgtaaagtttcccgttctcaagacggtgaatcaaaaggcgaaaaaatctaaaaaaaattgtaaaaagaacgacaacgacgatgatgatgacgacgaagacgaaaacgatgacgacgtcgaagaggaagaagaagatcaagttgtcgtgaatcacactcaacaattttcattgtacaaatcaaaaagtaaacttgataacttttctccgtacactaaaagtgtttgtatcaaatgcgaaagggattgttgaagaaaaatgtgtgtgtaatttttttgtaaataaatttattcaaccataaaagcattgtctaattctttgactaggaactgtgatagatcgacgatttcatcttctgttagatgtctgtaatcgacgaaattttgcatgatcatcagtatatgatggttggagtacacttgcttgcaaaaataatcgatttgactgtaagtcagtctgatgccgtcgaccgcgtcgagtatgtcgtacacgtctttcactgtcatgtctctatcgatgaacatttctatgattttgatgaattttgtgcaaaacttttcgtctcgcacaatatccttgagatagttatttaaatgatgatttaccatggcaacctttgccgcgataggcatattgttgaacatttgattgacaaaattcttgaagaaatccattgtgtctgtgttgttgcgcgcgcgttctaatcgacaatgctcttattcttatttaaaattgcgtcaaacgctttttccaggtcgcgtttcttttttacactcttagctttaccggtgggtatgtgtactgttgtcgactcgtccttaatgtagtacacttgtaacagcaacaagaaaattacaaagaccaagagtaaaaacagtaaaagattggagaatccttcatttttgtcaaaaatgaatccaattacaatcaatacgagaaacgtaaagtacatgaacatttcaattgtttcaataaacctcttattgtgtaacgatggtgttgtaaagcgacgacccacatgggttctacttactattcgcgtgtataagaatgaaatcgtggaccaaagcagttatatggttgttgataatctacgctatagtcatacacgtttacattgcgcacaacaataacagtaggaatgaagtcaaatcgtttacgcccggacctacgtgcaacacatattattacggcagagatttgcgcgtgtgtccgcaaggccttgaatttaatgtctacctacaagattgtgtattaatagacggcgacggcgacggatgtcatgtcaatgtcgttaataaaaattataatctgtacgataaaattaaatcgttgaaaaatcaagtatataaatatcgaacgagcgcacaaatgttgcagtataacaaagacgacaatgtcgtggttatcaaagaagattgaacctagtaaaaaatgttgtaaaatattacgacgcaataagaattggaatcgcagctatttatcttcgaacaaggtgaccgccaaaggtctattagagtatgtgcaagaaacaaaggatttacaaggcgctttgcgttatttgtttcgtgtgaacatgtactgcaaaatcatcaatagattcgaatggaacttggtgtccaatgcggatttctgcaaaaacgaccgaggatacgaggcgtataaacaatttagaaaaatgaaacacaacgacgccacgcactcggcgtattctttgtgtaaactggtcatgttgtacatgtacatatcggcgaaatcgcaagcaaagaccggattgcacacgagcaactgtatagatttgatagtgtttaataaatttcgatgttttacgtgcgagaaggcggtggccgcagcgcagtatagtgtgtatggagatgaagatgaatattttttctaaaattaatttataataaaaccaagattgttgtatgaaactttgtgttttattcaatccaaaaacatgtctaacaaatgttcggggaataggaattcggcgggtatcgtacacaagctggcgagtttaacgctgtcgtttttgtagcatatgttgtcgtttttaaagtagacgttttggagtacggtggtgttgtggtatatatctccgccgccgactttgactcgattgtgcgacgaaatgtaattgtttaaacttttgatggcgcgatccgcgatcacatcaatgtcggagctttttgtttttttgtaatttttcgtacttttgattaaatctttgctgcctttggcgccgcacttgatgatcgaattgaattcgccctgtaattcgaacagttcctcgtcgccgcataccatttccttgtcgttgatcatttgactcaatctttgaaatagcaaatagctggcattcgagctgagcacaagagcgcagtctcgtaacaatagatcgagtcgtgtggcaaaaacagcgtgacgatgaatcttccatattttatgaagaataggcatagtggacaagagtgtgtcgatttgtttcttgttcttgtatagatagtatatttgctgggaaacgaatgccaatcgattcttgtcgaaacatatgaaattgtaacgcggatcgccgtacagcaagcattcgagatcgatgagcgaattgggtttgggcaggaacgtgatgactttcttgtcgccgtcgcagtcggtgttggcgccggtgaaaatgcctaggcctactttgacgctccagtcggtgttgtttttcggctggagcacgtcggaaacttgggtgctcaactgcgaaatgttgggatgacgagtcgtccaagcgcgaacgttgtccacgtcgcggccgtagtatcgttgaatgctgcttttcggcggcactacgacgttgggcgcgttcagacattgcacgtttgcgtaaaacgatgccgtgttcaagaacgtagagtataaaaattgtccagcgtatccatttttactttgaaattgatctttgataatgccatgattgaacttgatcttttgaagttgaccgctaatgtcgatgaggccggcgtcgtgtttagaattgaagcacttgtttagaaatatgatcacgttatggtcccagagaataaagttgggtatgattagataattaatgttgtctgtaaatttattgcgttttaactttttgagataaacattagacggcaagtcacgtactactatcgtcgtcgccattagaatacggctcagcatttcagtgtgttcgttacgctcgtcacgttcttgatagacgctgattaattgttcgatgagactgttgaaaaagttcatctttattcttttgatgtcggcaatgatgtttcgaaggaattgtttaaattcactaatgttacaaaaaaagacattgtcaatgttatcgggatcgtagagcaattcaaaacttgccgaattcttcggaacaaaagtgatagacgtcgccgacgacatgatgtcgatcatcaactaaaataaataagagggtatttaatttatattttcgagtttttcataaactaacatgacgactgccactgaattaattaatgtgcctattttgaaaagcctaatcaaaaccgaaatcgaccgtaacgtcgccaacaatataacaattctaaatggtaaattgaaaaaactcgaagatcaccatctaaacgattgggtggaagtttacggtattcacgacacccgattacataacaaaaagattcgcaataattatgtgaaaaaaatttgtgcgttgttgcaactcgattacaagtcggtcgtagagagcgactatgaaaagaaccacatcaaattgaaactgaacgatgcgactacggcgcgtgaatggcagaatcgttctagagaggttcgactcaaaaactacgacctcgaaatcgattttgatggtccgataaagatttttgtagccgcttcgccggaacacaagaagctattgaagaagacacgcgacgctctcttgccgcactacaagtacgtgtcgttgtgtaaaaagggggtgatggtgcgcgaaaacgaacgcagtaagattttcatagtcaaaaacgaaaacgacatttacgatctgctaggtaaaatgtccgtggcggcacatccaatctagatggatgagtgcgcaacgcgataaacgtttagtacttcatgttgactttcggtgaagtgtttcgctgaaagcaaacatcaaagtactaaatttagcgataagtgttatcaagttgaatctcatcatagtataaaagaaacgtattcttgccacaagttaaagactgacgaagacgacgacaacgataacaatgttcgcggcaataaataattttttaagagaaaaaatcaagtacaatgcgattacgattgtacaagaagaatttaccgatccgtattatacagtgaaaaaagttgaaagatctacggatgtaccgtattgttcatattgggacaatccgtttgtcatcgaaaaccaattgcctatcgacgacgaaaacgaggacaacaaggacaacaaggacaacaaggacaacaaggacaaaaacaacgacgacaacgacgtggtcgacgaagataatgttgcgacatcgacggtggactttggtaacatcaccgtaaaaagtttgtacgttaacaaaaagaaaaagaatattgtggcgtatttgaagaaaaaatacttgaacaaaaagaacgaaaacgataaagtcgacagagaaaagatcaagctgatgcgtcatcgactgctgattagcgtaccgaagccgttcaagagaacgcttagcatcgaaaaggaatttgtcacggatcatcatttgttcaagtcgaatctgaagaaattttcattcttcaaggaaaagaaaaatatacctacttcacatttatatccggacgtggacgcctttacggagactttgctggatttcttggacgatagaagcgacgagtttgactttaacaaagtgtacaagaataggtacaatgcgttttacgagaacgacaatatttttaaaggaatcgtttacaaagatcttgaacaagttcccgtgatgacgcataaggagcaatgtcggttggacgacgtggaagatgctcacgtcatggttgtgaggtttaatagattttatttgaaaacgtgtctcaaacctaaaattaaaaagcattttatggacgttctcgaatttccgctctttatgctcgataaaaattgtaatattttgataaatgaaaagtatggtcagtacgttcgctacagggatatcaaggacaagatgaacgactattccagtttgaacattgtcttgtcgagtaaatgcgacgactataacgactataacgttaaagatttttttttcgattcaaaaaacgcgccggccaacatgggccttttgtattatgtactgtactgggtatcgtttcacggtttggggctaaacttggactttcaagaatacatgttgactcgtgtgaaacaaacgagatgcacgctcggattgtccacgtcgtctgacttttttccgcaaattatggttccgttgccgaacgctttgttctactcgtgttacgtgagcactgttttatttaaaaatcatcccatgtacttttaccaagagcgtcttaaagaattaaattcgtactcttgcgacatgcgtttcttgctacagaaaatttttggcgaagactttttggattattacaacaagaaagttagaaaccgatctaatgagattaaaaattacgatacgctcgataacgatataaagaatttaatactggaatcgtacaagagcgatgatttcggttatcattatacaaaaattctgcccggtagtcgtttgcttcaattaaataaaattatcaattttaatataaatttcaacgattacatacacacgtattatgaacatgtacaatttttttttgcaagatcagtatcgtacaatattaaattgagttttattcatgattcgacaatgaggcccagattacaaaaacgtcgctattacgatcctgacgatgcagtatgtaacatacataaatttatacaaggtatgatgaatcccatagttttgagcaacggtttaattatgaagcgaaacatttcaaagattaaattgtccaagtttctcgatttgtataaagcgtttcaagagtgcgtaggctcgctaagaaagtttcccaatttgaatgaattccacatctttgttcataaaaagtttgtcaatcgaaacaataaaatttgtttatttccggaaaatttcatatcattgatcgatggagtccacaagtcgtacacgtcatttttaaaaaataaaaaagttaccgtgcctcagtttttacatgtgattgaaaggacaaacgattataaagtacgtgtaaaagaagaaggttttagcaaatatgtaagtagtgaaattgtcaagtatctagaaaatatttttgtggatgattgattaattgttgtacactgttgttggtatgaaataaaacatatacacactgatattgttgtcttttattaagaataatgtacaataatatattagagttgcctgtcagtacacgcgttttacccgttgagggtaaacgtatttacatgaaattttttaatcgtgcttacgaaaaatatcattctgatatcactgctgcacaaattgcctggcaagctgttaaaagaaaatacgtcaaagtcaacggcgagtggatacctcgttgcgacgccaacgaatacgacacgaccactaccgaagacgaagacgacgacgacgacgacgattataccaccaccgataccgagtaatataagcgccgcaataatgtttcatttaaacgaagcgttctatcatgaagagatgcccgctcgtgccaaacgtttgtttgtaaagacttttaaaaaatatcacaagctagacggcggcgatgaagatgtggctcttcacatggccaaacaggcagtagatagagagtatgtaaagctaaataatcgttggattcctaaaacggctgccgaggaaattgtacggcacgatctcgaagatgatagtttgagcgaatctgagacgcctccttctcctccgccacccactcgttcaaatatattacaacggtcccctgcggttgcttttcgacacaacaacaacaaaaacaacaacgtcaaccagtataatcgattaaaccacgacgacgaagatgacgacatggattttttgtccgacgaagaatctgatgatacagacgagtacgattacgacgaagatgaccattacaacaacaccatgtatagaaaaaaatctacgttgacgaatcgcaatagaggcggcggcggcggcaatattaaacaacaacacaagtttgcacgtacaatgaaaagtaacaataaacaaacattcaagtaaattctaaaagttttatttcgtattcccgatcgagcgattgtctcgccttgtccgagggtcgcacatagtttttattgtgtctattttcaaagtctacatatccactgacacgtttatgatatgtttcttcaaagtaaattgttttggccacgacgattctcgctctgtcttcattgtacagattgataattaacgatttgttgtgatcttgctcgttgtacgcgacacattcgccgagaggtttgtctttcttcagccgtgcaccgttgacggtacatatgctttcgtagtacagcaattctgccatagattctaatgataggtcgcagtaaactttaaaatttgtgtaaatggctcgttgaacattgaggaaaattatgtattcattgtcacataatccgtcggcactgaacgatatgtgtctgagatcgatagagattccatcaaaatcaagatatctattgttgacggaaaccattttcaccaaagatacgagatgacgtactaacggagcgccgccgcaagtttcggtttatatatacgcgtctccgttgtcgtagtaacgctcgacgacggctccatttgaacataatttccatgagtgtcgtcggacagcgatcgggtgtcgcaaaacaccgctatatcgtaaaggatttagggttgtttttcaaatgacacacaacgacaaaatatttggtatcgcaatatttatttacaaagttttatcctcgtcgaaacaacatttaattatactagtactagaagaaaagactaggggttccttaagtgaatttggcgacttaaccagcttacaatctccaaaaacaattttgttaaactcatcatgtaatctaatataattctcaaagctaatagacataggcgctataaatttacaatttttacaataaacaacttgattcgtttcatcgacgcgtcgatccgccgaatcgttgttgtgctgtgagtggtcgtctcgatcaaaatttgtattttttttcatcgaaaacagtcttgtaattcctctgtcccacaacattgtatgtagtgtttttgccaacgtacgaaacgtcgtttttcttttgttcataaaatgaagcaagactgtcttggtttctgtttctcagcttactatcaacggtgttgttgataacctccttgagtttaaagtttacttggctcatgttgcttgttatcagataacaacaacgtcttgatattgtgacacactttgtactgtgacctaaatattgcgacacccggtttttatactcggcgataattaattcactcgagaacaatgccaaaatatctgcgaaaatcatcgatgaaattgtaccaatctacggcaatgttgtttctaaaacggtcgctattgttatcgtcgaatcgtcgattcggcacgttttgggagacaattaattcgtaatagcgtctgtaattgttcggcgtgcgtgcccattttgcgtaatggaagattttcttgaccatttcccgtggaggacacatgatgtgaccgggcgcgttggcgccgtattgtagatcgcaacacttgactcgctccttgttttccggacacctgcccagcatgcgaaatattaccgatatattctttctgttgtcgttgaaaggattcggttgtttgagtatcatggtcagtatggtgttgtacattagaaaagttcgatagatgatttgcgattcgtgcgaagcgtccgtgctcggtacactaaatatattaacgtcgtcgttttggggcacgctttcgtgcgtgtaaatggatatcatcctttgcggcagtacaatggtgcggaatatagaatagacgaggtcgacttgcaaattgtccaccattaggatgtaacggtcgacacacccggtaaatgcgtcgatgatggttttgatggtattacgtaatatgaccatgtttcgttcgccggcggcgtcgatgcccatgaactgttcgtcgtcgtcgagagggcgcagttgcgtgtacgatatcatttcgttggcgttgttgtagatgcctcgaatttcataattgtcgttcgaggacgacgctttgacgagacacatgtagaacggttcgcgattgtagtcttggtacacgttgttgagaacggtgagatgattgtcgaaagcggtgtcgttttcaggatcatagtgtttggtcactttgcagttcgctcgtaatggatgaattggacactgggtacttgtcagcttcaacgttttgaaaggagttaatttctccaatacacactggtttaatcgaattagattcgtattcgacgacatttaaaattatataagtgttgtcgatcaattctaggttatctttgaggatagcgttcaacacgtcatccgcgatggacactgaagacatttttgacaaatttaaaataaaaatgttgtcttatgactcttacagcaaactcggtcgctacgtgtttatgcgtcaagaaaataacgatttcttgtatttagacaagtacactgataaggtgcaaaaattctcgcatcgttttcaaacgttaactgatctcggcgattacttgattctagtctattaacaagcaagcgtagcgcaatgagcggtaaatcgggatctaaacgaaaaccaacgtcgagtcgcctaagcgagattgtgaaatttaaacgcaaacaggacaacaaacccgctgtgccgacaacgaacgatctcgaagacgatggtgtcggtttgaattttgacccaatctctcccatcaattcaccgccatactttcccaactacgatcctgacgtttcaaactccgccatggacgtggatattagaaaacaagtggtgtttgtccggccgccaaccatgaaccaaatcactaaacaatacgacaacatcaacgacatacaaaatgcgatcgacaacatcacgcagtacatggacgctcttcagaataatattagtagtgttaaatggacgacaaagcagcattttacttttttcgaaaatcaagtaaacaaaatcggatacaaagacataatgatttcagtgttgaacaacaacacggtgaacagggacaacttttacgcattatccaacatgttttacaactattacgtccatctgttcaacaacataatccccttggctcacattattgtcaacgtgaattacacgcacaacaaaactcgtattacacattctatgacggcctttctaaacatgtgtgcccactacgtcgtaagtaatattagacaacttttcaactcggaaccgactataaacattccggaatcgtattacaaaatcatcactgacaaacagcaatgcttgactaacttgtacaactttaaattaaacgatcttcgaaacttgacatttatcaaacatacgccggataatgacgtaaacacatataccagcacgaccgcgtctaatcctaaagatcgcgtaattagttttcccatttacgtcgtgtacaatataccaaatttgaaatttgaataaaggattacttgtagcgaagtaaattatcaaaatccaattccatgtcgagaacgtgatgttcgttgactcgattaaactcgtcggcgtaaaatctatccacgtcttgttcgccgataaatcccgtcatgttgtccaacaaagacgagcattcatctagagttttcggcgggaaattaaagtagaatttaacgtttctgttgaaaggatccctgttgcgttctcgtttccatctctgcaaacatgaggcgtgaaacattttttctaaatttaaagttttatattcggtgacggcaacgacgccatcgttgttgatgcgttcaaagcatatttggcattcgaccacggaccgtttccaaagttcattgaacattctaaacagatggctttttttatcctttaaattgatcgttattaacatgtttgcgtataaaaagcaaacgtgcgactttatcgtgggcacaatttaatgtatcatggaacttataataccatttctacactactcgaagctttatcggctggccacgagcgaaaatgcgcgtcgcctaatctacgaccaatggtcaaaggacacgacgaatattacaagagatttgagttcaacaaaggccgtttcatcatcgacaaattgcgtgttttgtcatgaaaaattcaatcaagacaaggaccggtatttttgcgatttctgtttctttcctcgaaccggtgtggacgaagagtttgccacgtactgcttgttgagcgtgtgttactacgagacggccggtgagatcatcgacgacaataatggcaccgaatcatcatcacaacgcgtcgtgtacagacaacggctcaagatgacgtggtacaattacgaacgtataggaaaaatgtacgaagttttgtatccccaatgctatcaatgtaaaagtttgacacgtaacttgggcgcaaagttttattactttgatgatcgaatgttctgcgacaactgtatgtttcccttgtttatcattatacttcataataaatgaccgtggattatatttttttgtttttcttttatatcaacttcaacaataacacgtagtgtttcgtcgaaagagcaagggtaacgaaacgttacccttgaatatttaataaacaatctttgaaatggacacgttatcaaacataacaatttaatgtcttgatgtttgctttcgtcgaagtgtttcactgaaagcaaacatcaatagaatgacgcaataaaatgatgaaatcattgctaaaaataacaacataatgtgtttatgtttgctttcgtccatgtgtctcgctgaaagcaaacatcgagacattaaaatgtcgtcatgatgatggtagtggatgatgcaatacttgttttttatgtcgcaatagttacaatctttgctttcggtgaagtgttttgttgaaagcaaagattgttgttgctggctgatgtaatacttggcaacagtaacgatgtttgctttcggtgaagtgtttcgatgaaagcaaagatcgctacgcatgtgtcatggttaaaaatagattggtaacaatgttggctttcagcaaaacactttgccgaaagcaaagattattgtcggttaccaactgacgcaataaaattaaattattgtgcaataatcacaatctttgctttcggtgaagtgtttcactgaaagcaaagattgtaactattgcacaatgatttcatcattttagaatattattcttttagactttgttaatcgttttaatgacctgctacgataacgtttcatggtgcgggttttgcgttgttttttataatctattccttgaacatcactactaatcgtcacgtacacaataatcattgtgataataatgttgtatttatacgggcgtctttcaagtgtatagtgcagttctgtaagtagacttaaacatgcaatcgtttgaagatcgacttaaaactttttcaaattggccggccaacgatcgagtgccgtcgcacatgttggctttggctgggttctattatacgggccgcaatgacgaagttcgttgtgctttttgtaaagttgaaataatgaagtggaaatatggcgacaatcccatattggatcacaaaaaatgggctcctcaatgtaaatttgcaaaactattaatttccaacccattaacaccgatcacggaaacgggaatcgatgaatgcggtacgtccaacaacccggttcctagaatgatacccaagtaccctgcgtacgaggacgtggaaaaaaggcgtcaatcctaccaaaatctacctattcctctgtatcaagatttggacgacatggcatgtgctggattttattacaaccgcgacgatagtacgtttgtttgtttccaaggaggttgtactattgttcattgggaaagacgtgacgatccttggcgcgaacacgctcgttggttccccaattgcgaatacgtaaactacatcaagggtcgtgattttgttcaagaatcaatatctttgagttgcgtaatacaatccaacgaacaagagcagcagcagcaacagcaagacgaacagcagcagcagtatgccgtgaacaattctgtcgctgtcgataaaaacaaagatgataaagacgacgaagatgatttgctaatttgtaaaatttgctttgataatcgtcgtgatgtatgttttttgccatgcggtcatgtggtttcgtgtcgtcaatgttcgtcgaatgttaaacattgtcctttgtgtcgctcaaactttaccagcgttcatcaattgtactatgcctgaattgtattaaataaaacacacacaaaaatttttaatattttattttattaatcctggtccatggtttcatcatctgaatcgtagtacgacatctcttcatcgttgtcgtacgactcttcaccgctgctttccattttatcgaatgccgaactaaaattcaaacgacgttcaatgtcttgaagaatgacatcatcgacttcaatgttttttataaaattgtaatcgtttgatatcttacgttcgagttcgttgatatcgttttcgatgacgttaatcttgtctatttgaggagaatagtttgtgtgatctatgcccatctcggcttcggtgtaatcgcgaacaataaactttttacgtttgctttcgcgttctacaatatccttgatgttgcgttgtgtcaatagttctattactgcgcaaccgggacgattgctgcacattatcaaagctactatgccgtcgtctttaaagtagttttctagatactcttcgtcgatggtatcatcctcgctgacctgccattcgaccaaaagtctattcgtaatggtagttttataatgtttgtttttgattttgagttgaatagttttgtagacgttttctttcaacattaattgaatgtccatttcgtaaaagtctttggcatcgttttgtaaacgtaaagcgctcgaatagatctcgttggctgtctttctcagaggcattattttttcgttaaagtaatggttgattttatctttttcgaccatgtatctatcgtaggctttacgattttgcggtaaacccaacacttgtaaagcgttacctaaagtactatggattttttcgtttgtgttctcgctagttgcggccacgtctctgccgacgcgagattttttataggtgtgttgaagattgttaactttactggtgatatctgcttttgtggcattgcggccaacgtttaagagatcgtagtagctgatcgaatctaaatttaaaaagtacaaagcatcctcttttcgggaaactttagccgctacgggtttttcgctgctgttcgtcgaactagattcgcttgtcgatcttagactgccgccgctggttcgtttgcttttgtcgctagtggcggtgctagtagcgcgttttaaaaaagaagacggcggtgaaggcgcaacggatatcgtgcgttttctaggcgaaactacagccgtcgtggacggaacgttaagtcttgtagagctacgcgtcaccctgctcattgtcgtcgacgctcgtcacaatgacggacgtgattgtcgattttaataaactgtacgataaactgcgcgacaaatacggattgcgttactatctcaattgtaacaataaaacggtcaatacgtgtacgatcaaatatttgcaggagcgacgttcttatttttgctgtgctgtcgattcgctggaacgatgcgtgcttcacaaatgcgtactgatcatattcggcacctggttagatttggagtttagacgcgacgacgagaacaaatcgtctcaattcaacggcacgtttatgatcgacggccggtacttgagctttcccaacatcatgatgaacaacaacattctcatacacaatttttacgacaagctctatagcaaagattgcaaacgcatgtttctctatggcaatctggacgaggagaaaaacatcaaccgcgccattcaactcgtgtacgacaatcagcgcgatgtgttgtatgctcgcgacgtgtacgccagcgactacgtcgtcaccgaggatctcaacgatatcctcgaaatgtatttgaaaaacagcggcaaatggactccgttaaatttcgtctttgattttaacaaggaacagagcgcaaagttggttaatttgattaaaaaaattatggccaccgacattaactacaccattgacaatctttccaacaaaatcatttacaaacacgactatttgttgtggttaatctataaaccaatcttgaaaaactatagtacaatcatggaaaataaaactactcaacaacagcaacaacaacaacataataatcatcaacagcgaaagaaaaaaatccaatctattttgtttcctaaagagtgtaaaaaagtcattgataccgtggtcaatggtaaactaatctattcagtgtccaagacttttagcaaacagaaaaagaatttcatcaactaccaagacaacagtagtaacaataacatcgaaatcagtctgccatcgttaaagtaccgtatcggcaatgaagtggtgcgcatcacaaacgatactatgcgacaggatatgctcaaacaaaaattcgactttatcaagtttgttgataattttttccacggagaaatgacggtggccggcaagaaattttttctgtgccgcgatgttaaactgcccagtgtcgattatgaatctgtagctgaaaagtttaaatctttgttggctgccaatctcatcaaaatcgcttcctacgacaacgaagacaacgttttaatagcgtttaataatcgtcctacgatctttgaatgcgatagagacaaaatgatctacatcatatatcagttgaaacgtaacaagtttcccattgaaattaaatttcacaacaacattttgtttctgaaccatcacgaaggtatgatctgtataaagaaaagattaaagattaacagcgatgtgggaatcgtaaaaataatgacattgttgacgccgtacgaataccacaatgaattgtcgtacttgcgaaccgtgcccggcatctgcgtcgaagaagagaaacattgttcgcaattaatgtccaaactgttacactactattacaaagatatactatccatgtttgctacgacgccggtgcccaagtctatagtttcgttgaccaatttgaaaaacgccatgcctgtcgtcgcgtacaataatctgggcaacaacgatatatttttggacaatttaccggcaggcaacagcgttgtcgtcgctcccgagattatgcgcaacgacaaaatgtttcgtttgtggacactggtgcgcgatcataaactgatgaccgccgaagatccctatatccccgacaacaaactgcccattcgtctgttcaacaataaaatcaacaaactcaaaggaaaacttaacacgttaaaaaacgaagtgcccaagataaaatattacaagagtagagtcaacaattgcgtacaagtcaacggaggccatattcttcacatggccggtgtggtcgtgtccaacattaaaatcggttggatctacgacggaaaaaggtacaagattgaaacgtgtcgaaataaaacattctacgtgtacaaaatatacgtatattacagacaaatagacaagcaacaaattgaaaaactagattcttctttgaccgtgaccaacaatacggtgtacgtcaaattgatcatcatcacgtcgacggccaacttggaaggtgttaaaatttgcggcatccacggacaaaaaggagtgatgaacggcagcgaggatctgaccgaatggatggccgaagacggcacgagcgcgcaaatttgtctatcgcccatctcgtacttgtcgcgtcaatcaaactttgaaaatatcgaaatgaagtatgtggtgcgcggcggcgatcactcgaaccccgacgcaaaacgctatcctattttcaacattccatacatgttcttcagcaatacaccggacaatatttttaaagaattcatcaaaggcaactacactggccacgaaaaagtcgaaggcactcgactcgaccaatggaccatcaaccaatcttttgcgggcaatcgatgggcggaaagtttacaatgcgttcgaggcggcaacaatttgcccgacaacagtggcgaatttaatgtaatgtctagtttgttacactgtaataatacaattatgagataagcttcaacagcgatgaaaacgtacaacttttcgtcgaaaaaggaagggtaacgaatcgttacccttggtgtttcgtcgaaacgttacgtgttgttgttgaagttgtttagaaagatatctttgtaacattcgatacacatgattttgttgtttttaattatggctctgaatctgttgaaaaatctgtcgctgcttgtgttcgtgtactttttgtgtaaataaaccatttcttcacaaaaatagcattttgaatatttattcattcttatacaaaatgaaacttgaactgattaccaagagtgttttcaaaggtgaacgctaaagacgttctggtgatgaggtctgatttcaaatctacaatacgcattcccacttgacccaagatgtgttcgatttcatcaaagtacatgatatgcgtgtgtttggtggtctgatatgcgttgttgatgctgacttgtcgagttttattatttttgattaatacataggggaactcgaaaatgcactcaatttcattgtcgtttgtgaccaccttgaaattgcgttttaatttattctctttagatatttttagcgatgatgtaatatcatcgggtgatgttgatgtcatcgaataacacacggtgccctcgccctcgtttacatctttgacgggcacacgttgatatagaaaaacgatatcgttgtcattgtcgtcgtcgtcgtcgtagttgttgttgttattgttaatagatccgggttcaaagggaggtagctgtttgtagttttcaatattatgatgttgaaccgtgtaaagctgagaatttttgtaaacgttattcttttctatgatggcaaatttatttttctgattgtggttcaactttaggacgacacaacgagcgtcgcggtttgtgctattgacagctttgatttggtcgtacaattgtaacatgcccgttttgtaaaatagagtgtacgagtagaattcaagattaagtgcgtcgaatttactgaatgacatcaccgctgcgcaatcatcgaggttaacgatcgcggtgaaacccggattcggtaggaacgacattgtcgtgttgggcgatatcagaacgtcgttttcgggcagtttaactacgtcgttttccaacaagataacacctgcgtcaatatatgtcggcatttcatcatccttctcgttatcgttccatatacgtttagtcatgctccacagcagcgcgtgtgttctaaagtcgtaattggcctgataatatgccacgttgttgacaggcgcgactaacgacgaaaagtagtactcgttcctgacggtaactactttactgaaatctgcggcgtacacgccgggtttgtagtacataatctttgaaatgacaggcatgacgtggacgccgtttttgtataaaagtgcgggatgaattcgtccggaattgctgcccaccatgtgtaagctatgatcgacgttttgcatgacgacaaagttggggccgaacaaaaagttgtagtattggaaggtgtaatagttttcgattaggaaagagtaatttcgtacgttggcgtcgacgtagttgataaaatcgaatctgatgccgctaccttcgttgtgcacgtagttttgtacatcttcaagaacgttcatgaccaaaggttgagatttgatttcggacaattcgacacctttacacaattgagcgtaaatgtacggcagacacattcgtgtgctgaaaccaatgtttcgtcgccagcccaacgacatattcggttcggttatatagttgtcgatgattttgacggccactttgctgacatcgacgtatggtctgagcgtgatggacaagagcattgcgcattctgttaaactgatactgaaaagattccagtacatttcgtcaaagttccatggtagatgcttgtacggtgtaggtgttggtagtttattggctataatttttagcgctcgagccaaacgagtggccacgacattgttggtttcgtcgaaattaacggttgcggcggcggcgttcatgtaaatcgcgtgagcgttgagagaacgcaaaagttgcgcaaaatctttagcgttctcgaaaggttttaaacttttaaaaatgtcaatgtcgtaatcgtcttcgacaaactcgaaaagtatatcggtttcgttgtaatcggttcgtaaatctttagttttccaaagagtggaccgatagaaatcctcaaaagtatataggttaacttcatgattgaaattatcaatttcgttatcgatgtagtcgtcgattggtcgcgaggcgttgttgttgttgctgccgctgttgctattgctgctgttgtaatagtacagaacaacgacaattactaatataatcacgacgacagaaacaataaaccatacggcgtttgacatatttcacggttgaaacttaaagtattataatgggttttgcgcgtttttacgagacgagatcgcagtcattgccgcaatgttgcaaatatttggctgatcccttagctttgtacatgttgtatgtgaagtacgacgactacgatccgacacagacacgccatgtcatcaaaaacactatcgaggtggacgacgaaagtttcattgtgatgcaacagctttgcgtgtgcttcgatctcgacaatttgtccacggcttcgcctaaagaattaaactgttacgtggacgttacgcgaaaatctatgagcgatcacgacaaacgcatctataaacttttactgcacgacagatggtgtaaaggagattttgttcgtctgcgcaaaatgctcgttcagccaaatgttagcaaactcatcacgttcggttgcaacgttttatgggaacgcggctacgaaaaccactacactttaggccaacagctgagcattcgcataacgacaaaactcatccaaagcggtctggattttaaacatcaacaggactcggacaacaccaacgtcgtcgacgccgtgcccggtcgcggctggaacagcaaagctttcgaaaaactaatcggaaacattacgtcaatatctgacgtcatcaaaagacataaaagttcacacaagtacattgttctcgaaatagatccaataaattgcgacgcagtaaagaaatgtttaagcgatcaattcacgttgatttttaatgcgcacatgcaaaacgtgtgcgcgatccacgtcgatgaagacaaaaattccgttcaatatttagtgaagctatcaaagttgataaatgataaacttgtaaacgtgctctttgtcaccgacgtggaatattatctgaagcaaaacaactacatgttttatctgtacaattctctgaaaatgtattattactgtttgtgtaacaagtttgtctttgaatataaagattacgaaataatattcttgttgaatcttatcatttcgttggaatggcacaacaacggccacctaaactcgttcacattagaaaaatcccagatctacaatccgctcgaactgtcgacccgtcgtctaaattcgataaaacgcgccgctgcacaatctagggtcataaacaacgataacgaaataaaaatagatttcattaaaggcaaacgaatgaagatgggcacacactacggtcaaagacttgtaacattatattgataacggtataaattagtagtatataaacgacgatgaaaactgcatcgttgtcagtgatcttaacactttagcacaagcaactatgaacgtgggcacggtgtgtttgtgtgtggacagttgtctgctgttaacgaaaaaaatccattcaaacgtcgtacagacaatatgccagttggactttgaaaaaggaaccccgaatctggtgccgatacgcaaaagtttaataatgttacgcaataacttggactatttatatgtatatatcaagaataatttggatcaaaaagacaagaagcttaacgaattcgtaaattccgttgttgtcaaggagttaaacgatgccctgtacaattacatgaacgatatacaaattttggacgaagacatgaagaatattgaagtgtgcagtcacatgatgtttactttgaagtcttttaataaaaagaattttcaactccacttggagattaacgattacaatcttacgtacacatttatcaatatgatcaatgtgcacgtgctacctccgagcaagttgttcaactacgtagttgataaaattaattacatgcaacgtgtgtgcgataggattcatgctcgtctatacgaattggaaacgatttacataatggtgaacatggcaaccgcggctagtctcgatgataacgacacagtatataaagcgacgccgaaagatgcattgctttagttgaagtctaacatcggcgaggagctaacaacacgacaacggctactatggacgaaggaaaattgtgcttggtcatagacagcttcaggaggcaattgaggctgttgaataaacgagtgctgttaattcaagaaaatattgattttgaagaacctttaccggacttggaaaatttgaagaataatttgactaaaattagaagcgatttaaagtacgtgctgcagtgtgccgagaacaacattgatacaaatttggacgtttatatatcgcatcatacgattaatgaattggattttatgctgcgttcatgggaaaagtgtttgcttgacaatattccctttgatattgatgagtttaatgttactctaaagacgtgtgtgtacatgtttaataatgtgaaacgtataaactttaaaaactatatgaccacaaagcctttgtacgataacaataatattgaatcgtttgtccatcaagtgtgtatgtattgtaaaagaagtaacaaatacacgtacatgttgaatagtatacgtaatcttgtagatatatgtgataaaattaaagtaaggttgtcaatgtatgaacttggtaccgaataagaattgtacaatgcattcatatataataaataatatattatttcacaaacagtatattttatttaatcatgcgttgcgcaggtttgtttatgattctcgaagcggacaaggcggtgttgctgtgcgctcgccgttcgtacaatagtaacgtttcgtacagtgatcccaaacaattggaacgtgtaaactttttggaaaagatttcaattccccgaggcaaacgggacggtagagatatattcgattacgagacggcggtgagggagttcatcgaggagacgggcacgttctttgaaagtgcttgggtgtatcgtgtgccgtttgtgctgcaatggaacgatgccggtgtcacgtacaagtatgccatttacgtgggcatagttaaaggtttgctacagcacgtttctcgagaaccaaacacgtattgtgtaaagctcaattgtgataggccaaacgattacaagtttaatttagaacttcgcagatataataacgagattcctcgacatctatatattctgccgttgcaagattattttcagtatatgaacgaaaagcaattgatcacgtacgattctagcaactatctggaatttttcgagtttgtaaagagcgtcaaggcaaaatttaatcaacgtacgttgaataatttttttttattaactctgaaattggacacgtttaatttctatcaaacatggataaacaatcgtcgaggcagcagcagcaacaacaatcatcgcgacacttcaacattgctaacgtcgaacaaggatttgaaaaagataatcaatgttgtttgactcgcagcgaaacgtatgcgttgtgccgtgaagttatcaataaacgtaagcacgacaatgatctatccaacgtgtggacgcacatttttgagccaggctttcaagaacaactcgaatatataagggcaaatcttgacaaagcgttcattactgttggcggcgagcaaccacactgcaagcgactgttttatcacgcgcagaaaattgaaaaaatatttaatttaaccacgtcccttgaaaacgaatacaaaactgcctacagtaaatatgaacaccacaacgaacgctttaactgaaatgtacaacagtttcggcggaggcaacgttctttgcgacaaagcgcacatggaacacgttaccgccgtcatcaacacactagaaaagaaaaagatcaagtacaaaatcattcccatgcccgtgtacggcgatgacggcttcgaagttacctttgcaatcgttatcatggtggacaaaaagaatctgaagcgaaataaaaaatccattagcaacaacaagtatatcttgttcaatagctggtacacgaaaaatcgcaaatcgtcctggccgaacagccatacgatgtggaacttgatgaaaactcacactacggccaaaccgtttgtagacatttttgattttatggaaaagattggcaaatccatcgaaactaaaaaatcatcaccaccaccatcgtcgtcgccgtccgaagataacgcagtcatcaccgatgttaacaatgaaaacaatgcgcgtcgtatgaatctatacaatgagttttacaaaattaccacgtatacgtttgtcaacgacgcagccccttcgaacagtttcatatacgacattaaattggacggaaacgacggtttggaaaagttgaaccgtcaaactttggagaatggcgtgattgcgttcaagaacattttaaagtctgcagttccgtcctctgtggaatttttaactacaactacggcaacgacaacggtagctacatcaaagaagaatgataaaactttaaaatcttcgtcatcatcgtcttcgtcgttgtcttcgtcttctcgaaaacgatcggcaacgacggcggcggtgtcgagcagcaagaatgtgaagagcgcgaaaaaaatcaaaaaaaagtcatgtggtgcccgccttgaccatggaaaacgatcaaagcgacgacacacaaatgtcatatagttaatataaaatgacaagttggtcagttcgagatgttagttttattgaaaaacagctacaagacagccatgtttgcctttaaatgtccttacactgaaaactatatcagtgtgtatttaaattacaaaacatattatttcgactattatgaactgatggtgttgctggaaattttttgcggtggtcaagacaatattttgaagaagggcggctgcgataagatcatcaacagattgaaaaaggcgagattcatcaaaattcacgaggcgatagttttactggattgttttttaaacacggaaaagattcaaaaatatgaacgaaatatatcccaagctgttaaaatacaaaaagaatatgttgtagtaataatttttttttactttaataaaaatacattttataaatgtgtgtgtgtatttatttatttcaataatagtaaatctatatgaggaccgttgtgtctgattatgatagcttctaaaatgagaccttcgtaaatcaaacgacgatgagatgtggtaggtggcacgatcaaggctgccgtgtagtttttaagttggccttccatgactatggcttcatgtattccgttgaggcgagttttggtcactttgaccattaccatttacaagtaccgccacgtagtctcaaaactaaatgtatcgtagattctttttgaatgttgtaatcgctcatagtacgagaatcttcaagttgtttaccagcgtagatgagacgctgttggtccggaggaatgccttctttgtcggcaatcttttgttttagctgttcaacggtgtcggtggattcgacgtccacggtcaccgttttgccggtcagagtttttacaaaaatctgcatggcttaattattcctttcctctcttagtaagatattttaaaaatgagcagtcaagaattcatcaaggatctacaggctttgatcgatagagtttcggcgaaacacaagcccgatcccaattcaaagctcggagatgtaatccagcacatgggtcgtaatggtttgcttttgcaaaggaaaaaagaggatgactttagtatcgacgaaaagattgagatttcagacacggcacgcgattacttgaatctgctgcaaacggaaaaactgtccgaatgtcgtttgtgttaccacaacgatgacacgtcgcgctgtgactttcatcgaaaatacatcttcaccaaagatcctaaacaatactacgatgattatgtgaatttcctcaacagcgagatgggcatcattagtttcgtcgaactctactacacgtatctagcggtgtccacatacaaaattgtgtcgctgatgatgatgcgtgacctgaccaacttttcgtccgttcgtgaactattaacatactacaattacgaatgtgccaacgacatcgacgtcgtgccatacgaaactatggattgtgaatagtagtttgcgcaacaacttgtttttattgcatcataattttacaatctttgctttccgcgaaacactttgccgaaagcaaagatcgaggactttattgcgccatagaatgacgcaatattaaatcatcgctaaaaataaacgtacaatgtttgctttccgcgaaacacttcaccgaaagttaacatcgttgtacgtttatttttagcgatgatttaatattgcgtcattctatggcgtcattttatcaatctttgctttccgcaaaacacttgaccgaaagcaaagattgaaaataaaattatgatgcaataactacaatgtttgctttcagcgaaacactttggcgaaagcaaagatcaaagtgttattgcatcataatttgtcatcgctaaaaatagatattgcgcagtagtgatttttattcttttacgattgtgatcgtgttttcctgtagcggcagcgccgacgatgatgacgacgacgacgtggtcggtgtaaccactgtaaatttaatgtcgtcgtttgtaaaatctacagagatcgtgcctgcgattgatttgtcttctactttcatgacgtatatggtgtcgacgttgccgtctttgatgtacatgtcgtagtcgaatatgttggtaatgtaatcggtgagcgggtcgacgcgtaaattaagtttctccaattgtttcacagaatcgacgtgaacgttgttactgattacacctttgcaaaagtttatcagtgtcatataatgatcaaaacgtttgtaatgctttgacgaataggacgtgtttttcaacaaatcaccggtagtaaactccatttcaacacttactataaatattgtgcatattgataatactttatacacctgaaactggttgaactaacaggacgtattgcctgcctgaacaacatggcttctaaacgcaaaattactgagattaaagacgaggcggtggtggccaacgttaaacaacagcgtagagacagtgtcgaaggagaagaagaagaagataaccaacaattagatatttacaaccacaacaatgctttggctaacattgaagacaatccgatggaggacgatgaagtgtcgcatcccgatcatgacaaaatgctatgcgtgtttaaggttcctacgataacgagagcgatgacatgggtcgacttgctgctgtacaatttgcaagcgaaaaacatcaccgtattaagatgcgaagcggcctttaacaaacttttcgattgtctcactttcctcgaacagggattgtgcatcgacactcatatggatgaattcgaacctgaaatctctaaagaaattataattttgaaaccaaaaccgcctcgtgtcgtgtacaacgtaggcaaagtggtcagaggcggatcgaaaccctttttctttttcgattatgccaaagtgaagagatgcgtaggtaactttggcgaatttttgagcatctcttggtccaaccaatatattcacaacaaggcgtacggaaaggtgatcatcaagtacaaagactttgattgtgatagcatgaaacttcaagacggcgcgatagtcaaccttcccggcgacgacactccatctaaaaagacgataattgttcgtaaatttttcgacgtgttccagcatcgcaacgaaagggtgtacatgacgggtcgtctagtgaaaagaatcaattgcgatccatttaccattgaacggttcaataaagtgttcgagttcgagaccgacaccaaatcttccgccgaagtggacatgttggtggggatacaaatagacggattcaagcaaagcaaggaggaagtcgaatttgaaacagtaaacaataagaaggtgcaggagcgtacttactcgttggctattagacctatgatttttataaaaattgaagaaatttaataaaattttattaaaaatgtatgtgttttatattaacggtactaatgtggaaaaacgttttggtcaagagtttatcaacttcatttgcggaggtaaaatcaaacacgaaattgaacatgaagagtgtacgaggaaaaagttggtggtgagatcgagctatgctgccaaaaaactattgtctgtcaacggtaaagcgttttggcccgacggtacaaggtttcgctgcaaacaaatcaacagtagaagattcgaccatcatcaccaccaccaccatcatcatagaagacaacgaagacactcttacgaaaacgattgttgtactaaaaaacttgcgtctttgtctacgtatgaaaacaacagcaacaacggaggcggcggcagtaacagcagaccgtttagtcccaccttgagcaacaaagaatggtacgcgtcatatgataacattaactttgacgaaggtgataaactttatgataaaaaataaaactatgcgcatgtgaaaaacagtctttttatttaaaaattggtcgagccaaagttgagaaactccattctcatcttgtgaagtttgtcatcgatctcggcaatctccttttggtcctttgtgattcgtttgatgcgttcccaatgctgcatctgaatgctgagttgtttcttggccatgactatttcgttgagtttctgtcgcaccgattcgtctttgctcttcttgtaagcctcggaggcgtagatattcttttttcctccaaacgaaggcatcttgttgattctgtgtgccggttcgattgtttttgtacacattgaacaaaaaatcaagattatatactaatcctaaatgtcataggttaaaaaaataataaatagtaagtgtataatgtataaaatgattagcgtattggaaatcttggtagtcgcgttcacgctctgtgtctctacgtcgccgttatctaccatggcaacgttcaatgtaaaatacactgtcgatcacgaaaccaaacgcatcaacgtaaactctgtgttggacgcgcccgtgtctatacacgtgatcccgcccaactctgatacgaacggcgacgaaaagttgacgttattgcatcagtttccgggcgtggccactaacgttatatttccgtcgatcacaaaggacgatgacctgtacgtgcaacttaacaacggtgttttgtacaaaactcgagcgactcgtgtctacactaatttccatacgcacaagaatcgcatggtctacggccaactatacacaatcgccgtcgacgatttcgagatcgccaacaagatttacatcggcgcgccgatctatcgcaacaaacagctcgtgtccgtgatcacgtgccgctttgatgactacgaagccggcttggtcatgtatcccgtgaccggaattcgaccgcaaggattgatttcgggccagatccagtacgatgaccgggtcggcgtcgaaacgttacgaccgagcatgtctgtgtacggacgccagcagttgccatacaaaagcgcacacatgtcggtgaaacagtttgccatgaccgccgacatgaatcgccagctgtacagagatcttcctcgaagtgtgatgctgtttcacaataacaaagagatcaccattaccgtggtagagggtgaatttgaaatgtatcgcattcgattagacggacctctggtcaccaatcaagaatcgtccaatgaagaaaaataagtttattcctcaaattataattatattaaacattatgagtcaaaatattttacttttaatccgttccgacatcaaagacgtcgacgccaaagttgacgctttgcaaagttccgtggatgatgtaaaggctaatctccccgacaccacagaattatccaacaaattggacgctcaagctactactttggacactatcgtatctcaagtcaacaacattctcgaagttttgaaccctgaaattccagatctaccagtgcccaacttgcgtaaaaagacaactaccactactaccactaaaaagtaaatttgattctttttttaaaacacataggttatttgaaataaaaaaattttggtaaaatttcattgtgtatttatttcatataaaaagagacaagtatttatcagtttcgatgcgtaattctgtatcgtggccgaacaatgcgatacacattagttgattgcgtcgtatgcgagtcgaagacatctcatattcttgcacgtttccaataaaatcgaaattcatgcgtttaaaaaaggaacgcaaaaagttggaaaacatttggtgaggcgtcttgataaactggatcagtcttgaccatgcttgttcttcgaacaccacaaacgaacctcgatcaaagtttttgagcgaataaaacagttccattttgttaatgtctctttcgaggtagttgcgtattagatttttgatgggtgttgactttttatgcgtatagtcaacgtttaaacaatagtagcccaccacaaaagtaaaataacaattaccgtacaccgtatctccggtaagggtcaaattaacaagattgtacattttgtagtaactgttaaagatgtcagtgtagcagttttgcaagtactcgttcacattgttctccagatgaatcttgtccttgatactgttcaacttgtgcttcaacgccaaacaacattgcgggtgaatcttgttcgtgttcacataaattgttgcgacttctttgctcaattcccactcgtctttcattgcggccaattcgtttagaaaaacaaaggtgttttcatggttatccattgacaaatatttaaactcttccgccatataaaacgcctggccgttgatcatttctttgatgcgcgtattagtacaccattcggcaaagtagtgatacaacgccaatttaatttccagtgaaggtgaggccatcttcgttattgtgaatagaatcgtagacaatgttgattcgtaccagaacgtcgaataaatacttttcatagcgataagatataaagattattccgtataaagattatcgtaccaacgattattcaaatggccaatgtttcgtcgtagattaacatagtacgataaagcgtcgaccaatagcgtgtacagagctagaattaggaaaattataaatagggcagtgacgttagtgtcctgcggcataagtgccaaaacgatggcgccggctatgaacaataccggtacgaaaactgtggtttgagtattgaacagtaaatcgttatgtctttgcgtgtactgtagaaattctaaatgagtatacagagcgctggacgctaaagccgaacctaccaatgttacttcgtcaaagtcctctattacttcgctattatcgaactctaacatttgaccattagagtttacggtcaacgaagctatatactctaaactgtccgctaaagcttctaatgttaaaatattttccacctcgctttcgaccaactcttcaaagtactctggcaaaaattcaataatgtctctgcggccgttttcaaaggacgaaaagtatgccgtcaaaaaggccatggacagatcgtcggggtagtctcgaggaaacatattgttatacccgaacgggtcccataatcctagaatcaaatccgctatgctcactatgatcaatatgatgcccactatcgatgcggctttgatagctattcgcgtgagcgctttggccaccattgaaattgttttaatggcaatcttgttgaacgcgtgcgtcacggccgctttgtaggtttctcccagcagtcgtgtcgtgacttgtcgcgacgtgttcaacaacatattttttagcataggaatcaacgtagtgttgatacgtttcaacatcgtctgtagagcttcgaacaataaatcgaaacctaccgcggttgctatgcccaatatcaattcgttatcttctagaaattgcgaaataatcgcttcgatgtcgtcatccgtggatgagtaattttgtgccaaactggtcgcgattctgtactgaagcggcctagcaacatgctcgcgggaaaatccaacttctgcctcatagattaatttgttcatgacgtcattcaatccgaggtcagacatagttttgtaatcggtaaagctgatctcgaaatgtacgttcgcggcaggatcgcgcgtatccagccattcttgtaaaactttttgcgaatctatcgtcggcggcggaggtagcactgccgatggtcttttgtaattgaatccgcgcatatcgctaaacacattattcaacaatagtttcatggtaatgtatatactgtcgcccaatacgaatcctatcaaactctcccaccagctcaaactacacccgttattgacaagattcctaccgaagcgacgacagtacgcctcgttaaaggttcctctgaatctttcgggaaacaattcgtccgggtctgggtagacgttgaaagcgggcacgtcgtcgacgcccttgatgagatgctcgtccgtgcgcatgtacggactgttcaaatacattttagatacggtgtcgacgagaacgcactgtttggcgagggtgtaacgcaactcgggcgcttggacttcgttctcggctccctctctggtggcggcgactctgtctagattgtagcatgccggttgggcgtactgtatggcggtttcagatgtctgtgtgaaatttgtaccagtgtgcgcgttaatgggccccgtttcgtggaaaggataacaagtcatactttcgcaaccttttttgctaaatttcaactttacatggatcgccttgtgcgcaattgcctcgggaacgtagtagtcgttgttgttggcggcccgtatttcgtaatcgaccaaaatatgaggcaactttgttcgccattttggaatgtagcgtagtctatgcaaatgatcgctgtacacgttggcgttgtatatatcgacaaaggttaaagtggccatattgttgtggtggtgtcaacttatcattatgacaaagtatatatactacgatcatgtttgaaacgttatccattgataacaaagacagcgatatgaatctacgcgttagaaaattggtacagaaaatttttaataaaaacgtaccatacgtgtacgtcgtaaagttggactcgaacaatgagtttcaagacatctacaaaatgatcgacaatgaagttgtcgaaataaaaaggccaccgtacaagtactacaaggtgcatttgaccataaatgcggccaaggaaaaattttacgtttttagagcagtaaataaaaaaccaatagaattgtgtgtaaaaatttataatgatgtgagcaatatagtaatacaataataaaatatattctaaaaaatacatttgtgtgtttaattctaacctaacctaattctaacagttgtcgttgtcctgtttggcggcagtctttttgtcgaagatgatcttttcaaagttacatccaaagttgtcacaagctatcgagttcatctctttgaacgtaattaaacctccagtgtagtgcgatagcaatttgatcattccattgtgacgaatgttgatctctttgcggttacccgtgttgagattgtaaatgtaatgattgtacatgccgtactctttaacaatgtccggcacgacaatgtctttgatgacactggtgatccataaaaattccaagccaaacttgacaatggttatgcgttcgtcagcctttgtagtaatcaaaataaaactatggttttcaaaacgctctttgcaggaatgtagatagttttcaatcaactttgccgaaccgttctccttcttgatcttgaacaatttcttgacacccttgtggtttgagtcgtaaaattgacgagctatacatccgtacttgtacgtaaagttattggtagttttatccttgtaggcgttgttgttctttccctgttgctttcctaggtaccaaaatttaagattcctcgccacggtcgacagtaaatcgttgcgtgacattttatgttggttgggcaaattgtccatcgtcttgaactgaagacccgtagaaaacttcatgatgtcatcaacgtacacggacatgtcgtactttttcaaatcttcctggttcggtgcctcctttctcgttatggaaaacggcatagtgaacaagtgcttgttgtccgtcatctggttaacgtattgaagaatgtttttagacttttcctcgccgatagaggcaaacagcaaactagttttaccttgaacgtatacttgatcaagatgaaaagtgttgatgagcaaagacaaaaatccaaaatctttgacttcctcgaaatagcatagattcttgttggtgtctgagagtttcttttcgctaaactgatcttggatgggtatggaaattttcatgtcttgcagtaggtggtacgagatcatgaaacggtagcgttccaatgtaacgacgaggacaaagcggtcattgtgaagatggtgcgcagtatactcattgtaaacggaatgtacgcagttgatgtaacgaatagagtatatttcgtcagggttcgccgacttgctcacgataaacatgtagtaactcgtgtcgagtatgtggctcgcaaacatccggttgttgctcacgacactattattagcattagcagcgttctttttggaatattttttgatgatttcgttgaaaagtatctcagttgcaggatcgctgggcgtgggtttttccacgtgtaccggtttcatggcgctcgagatacacatctttttagcgtaacgacctcgaactttgggttttacgattacacgttgacaatcgctcgtcgatgtcgcaacattatttgcgattgcatcatcctcttcgacctcgcttttcttgttgttcttcttgacaacaacgttgttgtcgtcttcgtcttcgctactactgctactatcgctactgctgctattattattgtcgctgacgcaatcttcgtaatgtaatcgtttcgacgccgcacccagtccgatgcccttggacgaaactcgtttcactcttttgctgccgcgagacgcgcgcgaagacgatcccgaagatgatatgcgtttcagcgaactgctgctgcttttattggtgggtttcattatagtgtcctcgacaatgggctgaagagtatcgaagagatcgagatcaatgtcttgagaaagattgccagcgtcaaagttttgattgatgatttcggcactgttgataagtagattgtagtttttgtcggtgtaagtgtcttcgaggccgttgttactattgcggtgatagtttaacatttccggagtgcgcggaatcgggtgttgaaatttgagaaattgcccaagattctcctggatgggagtgcggtttccgtgttggctcgatttgtatccgttgtataggtactgctggacgttggcgttgtcgttgagagtgtgcatgtttacaagtaactgaaacaaataaaataatgaacgtcaatttataccgtgccagaactaatagtaatattataacattttctattctacacacgattatttcattaaatatattcgtatttcaattggaaatagaggaggaggagataacaaacaacgcaccttctacgttaacgtcaagaccgtcaataacaagattagttagcggatacgaaaaaggactgcgcaatatttcgatgaaaatgacgtgtatcttatcgtcgtcgttgtctgcgataaacagcgaccctaatgcgtatctagtgagctgcgttagaacaccgtgtgtgttgatgaaattgatagcttcgccaagattcacacgacccgtggcacccgtgcttgtacagtacaacgggacgactcaagtgtggcacgtctttggcgttacgaaacgaaaggaattggcttcgatatccaagatacgcggcatcaccgccaccataaacggcgaggaaacgttcgtgaataaagagctcatcatgcttacgggaaacataccctcggctttcgtatcttccatcatgaaaaacacacccaacgtcgaggacgtggacactatgaagttggtgtaccccgcactcgaaattaacacctacgacgtgatcgtagagaaaaataatggcagttaaagatgtaaaaaaaatttattaaacatacacataacattacaatagtaaacttttgtacaatcgtcctacacagcccgtttccagagaagcgggtacacaatcttgttcgagtacgtcaaactgttgtcccggcatgcagaaaaattgttggcgttccggacaaacgtagtaactgtcgcagtcgtaggggtccggtgcgagtccgtgatatccactgggacaaacttttcgtacatgaaaatcatggtgcaattcttgtagcttgtgaaacacgaccaattttacaataataaacagcgctaaaagtaaccacattgtttaattgttgccgctactgtagctgagtattcccagagcggttttgtttgaactcttttcgccgtcttcaactctaaacttaaatttactcaacagcatgttggaatagttactgttcaaggtgggcttacacttgttcgcaaacagtgtatgatctagcaaagattcggaataattgtaaatgccatagtttttcagtatagaatagtgaccgtacgcgtgctccattatgaacagtaaacacagatactgcgtagtttccacttcgctgatcttgatgtaagattcctgttcgtcttcgtcggacttgcgctttcgcggcggagcaaaagttttcatcagattcaccttttgcttttccttgacgttggtggtaacgttcatgtgcgtgtcaaagtttcgttctaacatttttacaatactgatacgatctatggtacacactatctcgtcacgatcgttcaaccgaaacatgatgggttcgccgggaatcacgaggtccggcctgtccgtgaccacaaattccattctattatcaaagttggtcacgagcggatgtacccgattgtgtatgaaacccagcgacgccaccactatggcggcaatgtcgcgtgtagcgcttcgatctcgaaagatggtcagcaaaggttgcgtcaactgtagcgtgttcaacatggccatgtactttactattactaacttgatctttaaagtgtcataactttcgagactgtttaggttctttgcatcaaattcggccaaatcatagtccttttgtagtttttcattgccgtttattatttcggttacggttctaatttttggggaattgttacacttgaactgtttcatagtggtgttgttacaatactactcttataacatgtttcgttgtggggtattaacaaaaggattagttctcatagttgcgtttaggggattcatgtacgccgccgtggctggcgaactgccgctgcctcctggttgactgctcgattggaagagcagtatgatgagtatgattatgacgagacctatgagtatggtcatcatggtgttggtgtttacgctgtttaagctaaagcctcctcccgacgtcgatggtgcccgattaatgtccattattattacgtaaagagcttgagcagtgtgtttttaacccaaggatttttcaaatgatcgagagttttgaattgagaactttcgtaatctcctcttataattaaatatgcaggcacagaggtaccgaaaatgtgcttagttaaatatattttgtgtttggcgtcgatgatgtacgtatccttgttcaaaacgtgaggtgtcgcaaattgcctgtacggtatgagccgatagtcaaagtacaaaagttcagtgctgccgaacaaactgttgctcgccaaaattataaacagttggttggagggcacgtagaaagcgttgaaagtgccttttatctgtataatatcgggccgaacgaaaatgtacgacgtttcgtttgcgcgtaacgtgggcagcacgctgttcgcatcagtttttatatttaattttcgatacttatttacggcgtaacgatctatcacaatctcgttgtaactactggccagtttaatattatgttcgccaaacttcattataaacttgtaattggatgttttcgtcatgtaatcattgagcaggtcaagcaggtcatctggaaattttgcgtcgtagatataatctcgttggataaattttatataatttgcgctgttctccaactcgaggttaatttcgttgaaaacctcatttggctttttagttgtaaaatgtttgctgtttatgatgcggaaattgttgcgcacaagcggcagacccttgtaaaagtttcttatgacgtaatcgctatttgcgggcatggttatgttttgtcgtaaaaaatgctgcgacattgcctctccgatcaagtacaatctatacggatagtcgggagtgtccactttcggcgaagcgcacatttgtacgcccgaccagtccacgtatgcattatcaaagatgaaaccctctcttccctcgagcaacacggactctgtattcgaggatacgaaaaatctatcgtcaaaaactttcataaattcactatacagcaaaagcatgaaaggttgaggatcgtttacgaaaaagtttgtcgcgtatattggagtgtttggctggatatagtgtcttttgtcgaactctactatgtctacgttgtcgcgcatagaaatgtacttgaactgcggttttatatacttgaacatattggggtctcggtccgccaccaaatgttttaggctcataccgctcaagtagtttacatacttcaaaagggtatcatcgtcgatggcgttgaaattactgcgcacatactcggtgatgaaaggccgcgcttctgacgcaatgtaatcaaaattgttgaggtcaaagtatgttgctagaaaaaggtattttagttgtttttgttctagcgaatgcttgaccgccgctaaactcattatgaatcttcttactaaagttgttctatgatttgctgtttagcggcagtgggagttttaaaactagtcttacatgcgggacacacgggatacaaacttgaatgtttccacagttgagcatagcaaatgtaacacacgttatagccgcagcactcgttgggtttcagaaaatgttcttcggcgctgctctcttggcaaatgttacactggtatattttcgggtcggtgaacacgtttatcaactgcatacgttcgttcattagtttgatgatgtgtagatacttttcgctgcggtcgtacaaatcgttgagtgttacactgtttgatttaacgatagcactacagcaaaaatcatttacgaacatgttgttgatcacacgtatctgtttaatgtacggtatgaaaatgtacatgttatattggaacttgggttgagtgttcaagttttgtataacatcaacgacgttctttatttcgttaattaaatagtgtacgcatgtgtctcgcggaagagatatatcatcttcgttttcgcgaaagaaaagtaatttgtcaagattgcaattgtacgattgctgatatgtttctttaatgatcttgaaaacggccatcttaacattgtagtgagctcgaatgtttaaacttaaatctggtgtgtatagatggttaaagagaaaattgctaaaaacttgagcacacagctcggaatcatgattaatactcattttagtttaacactatagtcattatcgtcatcaatagaaacaacaaacttatataacaaaaacttttattaaaaatatattaaaaattaacaacgctgtttcttttgtggaggagcactgtcgtagtcgtcgtcctcgttgtcctcgtcgttgttgtccttgtcctcgttctcgtcgtcgtcgatcttgtgacatttaatgttaatcttgagtttgttttgccaagctttgagaatgttgcagtaaatgttgtcgtagttggtgtcgtgcaaaattaattcttgcgtttggtcaatgtatggagaattttcaatcatgtaatcaagttgtttgcagtcagtctccgtgttaagtttaaacactttcttgatgtggtccggtatcaaatcattgttttgcacagacccattcttagtggccagtatagaattaataacggttggcgtgttgagattttcatgtgccaacgctctaacgagaacttcttcgatgtagttgattttttctatttggtaaaggaaagaattgaagggtccgctggtagttttagtgcccagaatctcaaaagcagttttgtacgtgttggaaacgaacgttacgtgtttgacgccataacgttcgactttttcgtgtaaagattgccagtcccacatcagagtctcgttgtcggtatacacgttgtagatttgttgggtgatcttctcgttcctgtaggatttctttttgccggactcgttgagttctttgctggcctcggccaactcgttactagcttctatggcgccgtagtagatggtttcggcgataagtttgttgagggcctttgcttcgaccatatcgaaagtgtagccgagcaaaagtagagtttcggcgaaaccttgcataccaacggcaatgttgtgttttttgttttcaaatttatccaaaggatacatgttaatgttaatcatggcgtccaagtcacgtaccacattcttggtaatgcgtctcaacagattgtagtcaaactgtttagtttcgaggtttacgcatttgtcgacagcaacggatgccgaaaacacaatgttatatttaacttgttcatcttcgatggaaaagttgttattaaaagtatccttgaacattacagacaagttgccgttaattctcatcgtatcaataattagtttgagcaaatctttagctttaacaatcttggcattgatgtgttctttctcaaacttgacataggcttgctcaaacttgtcgccgtactttgaactcaaggaagtacaagactccggatcgagcagtgtccagtagccgttatttttcacacggttcataaaaatatcggggatgtctacggtgaactgcaaaaagttaaccttgtcgctgttgttgcgaatagtttcgataaagtttttaacttcatgatgccatagttcaatgtgaatagtgggcagtttcacaattttattcttgtgcaagatatgacgtaccatgttgttaaagacaggcagcatctttacggaaacgttttgcatatacaagtcaacagctccgccgcaattcatgattgtagcacattcttgaataatcttgtatgataaaaagatattatcagaacagttgtctacgtagagtttatagtcgtttgccagttgagtagaattagtaccggcacaagcaaaagcaagatcagagtgagcgtacatacgcaggctcatcatgtgatagctctctataactttttgtatgtcgtcgccgtacatggtcagggcgacacgcatgaacatatgttgaactctttccagcagattatttttattttcgtacaatcgaagatcgtaagtgtagtctcggtcatgacgtaacactttgttcaactcttcgccgtaaaccatggcgagacaatacaatctttgaccgatctcgtttttatggtacagatttttcattacaacctggaacgattcgtgaatgtcttcgtgcatttctgttacaatttgaacaacttctttgtcgaaattactagtgtcttcaactgttacacgacaaacgttagtatagtcagttgataaataatactcgggtttgtccaatctcatacgatcttcacaaagattctttttaatcttcttctgcagctccatagtttcgtgtaactttttgtattgattatagaaatagtcattgtcttcatcatcactgtcttctacaatcttctcaggaacgtcttcgttctcgtcttcggtgtcttcgttattgtcttcggtaataacattaggagtaaacaaagattcaggctcaccacttgtagaagcgatgggctcactaggctcacccacttgctgctgctcctgttcttgactattctcaacataatcttgactattgttataatattcttgattggagttgaacaactcttcaggcaaaatatcattcaagttattctcctgctgagaattctgggtagcgtcgttcgcaggggtagcgaaaagatccattgatagattttccattgcaaagtttgaagtgaggtgtttctcgttaacactgcacaattgttgtgatacttcaacgatcctgcacgacgcttttatatccaagacgcacatataaaaattacaaatatagataataactgtgatatgttatcaagtcacgtacgctgcacaagtcacgtagacgccatcataatcataatcctatgctgcattgtaatctaatcttttgccgcatttttatctaatcttttgccgcactgcgggaattgtaagtaattttttcctttcgtaaaacattgtgaaaaaataaatata

(19) United States
(12) Patent Application Publication
(10) Pub. No.: US 2016/0010062 A1
(43) Pub. Date: Jan. 14, 2016

(54) Isolated Spodoptera Frugiperda Multiple Nucleopolyhedroviruses and Methods for Killing Insects

(71) Applicant: The United States of America, as represented by the Secretary of Agriculture, Washington, DC (US)

(72) Inventors: Robert L. Harrison, Laurel, MD (US); Holly J. Popham, Columbia, MO (US)

(21) Appl. No.: 14/327,033

(22) Filed: Jul. 9, 2014

(51) Int. Cl. C12N 7/00; A01N 63/00

(52) U.S. Cl. CPC C12N 7/00; A01N 63/00; C12N 2710/14031; C12N 2710/14021

(57) ABSTRACT

Novel viruses capable of infecting fall armyworms (Spodoptera frugiperda) were isolated and sequences of the viruses were determined. The viruses were shown to be capable of being used as a biopesticide to control populations of fall armyworm.

### BACKGROUND OF THE INVENTION

Novel viruses capable of infecting fall armyworms (Spodoptera frugiperda) were isolated and the complete genetic sequences of the viruses were determined. The viruses were shown to be capable of being used as a biopesticide to control populations of insects (e.g., fall armyworm).

The moth genus Spodoptera (Lepidoptera: Noctuidae) contains generalist crop pests found throughout the Americas, Southeast Asia, and countries around the Mediterranean (Ellis, S. E., New Pest Response Guidelines: Spodoptera, 2004; Meagher, R. L., et al., Florida Entomologist, 91: 517-522 (2008)). One of these species, Spodoptera frugiperda, known as the fall armyworm, is a significant pest of maize, sorghum, rice, wheat, vegetable crops, and pastures in the Americas (Sparks, A. N., Florida Entomologist, 62: 82-88 (1979)). Fall armyworm infestations of corn and sorghum result in yield losses which are generally avoided by chemical insecticide application directed against fall armyworm larvae (Chamberlin, J. R., and J. N. All, Journal of Economic Entomology, 84: 619-624 (1991); Marenco, R. J., et al., Journal of Economic Entomology, 85: 1285-1292 (1992)). Chemical insecticides are not an option for organic growers, nor are transgenic crops. Also, transgenic corn expressing Bt toxins exhibit only a moderate level of insecticidal efficacy against third-instar fall armyworm larvae, and the prospect for the development of Bt-resistant fall armyworm populations is real and significant (Hardke, J. T., et al., Crop Protection, 30: 168-172 (2011)).

Naturally occurring fall armyworm pathogens can be used in organic systems. Isolates of a virus, Spodoptera frugiperda multiple nucleopolyhedrovirus (abbreviated as SfMNPV), have been identified and characterized from fall armyworm populations in North, Central and South America (Berretta, M. F., et al., J. Invertebr. Pathol., 71: 280-282 (1998); Escribano, A., et al., J. Econ. Entomol., 92: 1079-85 (1999); Loh, L. C., et al., J. Virol., 44: 747-751 (1982); Loh, L. C., et al., J. Virol., 44: 747-751(1991)). This virus is a member of the genus Alphabaculovirus in the family Baculoviridae. Members of this virus family, referred to as baculoviruses, are large, double-stranded DNA viruses that establish lethal infections of their arthropod hosts (Harrison, R. L., and K. Hoover, Baculoviruses and Other Occluded Insect Viruses, In: F. E. Vega, H. K. Kaya, Eds., Insect Pathology, Second Edition, Academic Press, Boston, 2012, pp. 73-131). Baculoviruses have a narrow host range and generally have no effect on non-host species. As a consequence, they are regarded as exceedingly safe biological control agents that can be used to target specific insect pests.

SfMNPV isolates have been evaluated in field trials as a potential biopesticide to control S. frugiperda on maize (Armenta, R., et al., J. Econ. Entomol., 96: 649-61 (2003); Cisneros, J., et al., Biological Control, 23: 87-95 (2002); Moscardi, F., Annual Review of Entomology, 44: 257-289 (1999); Williams, T., et al., Biological Control, 14: 67-75 (1999)) and cabbage (Behle, R. W., and H. J. Popham, J. Invertebr. Pathol., 109: 194-200 (2012)). Applications of SfMNPV caused significant levels of mortality among fall armyworm larvae in maize plots without affecting populations of natural enemies (Armenta et al., 2003; Williams et al., 1999). A significant number of studies on the population and molecular genetics of SfMNPV have been published (Simon, O., et al., J. Virol., 82: 7897-904 (2008); Simón, O., et al., Biological Control, 44: 321-330 (2008)), and the complete genome sequences have been determined for three SfMNPV isolates (Harrison, R. L., et al., J. Gen Virol., 89: 775-90 (2008); Simon, O., et al., J. Invertebr. Pathol., 107: 33-42 (2011); Harrison, R. L., et al., J. Gen. Virol., 89: 775-90 (2008)). In addition, SfMNPV has served as a model for studies into the ecology of alphabaculoviruses (Fuxa, J. R., Agriculture, Ecosystems and Environment, 103: 27-43 (2004)).

We have carried out extensive bioassays to characterize the pathogenicity and virulence of many of these SfMNPV isolates from a USDA insect virus collection, as well as isolates recently collected from Missouri, USA, against different strains of fall armyworm, either individually or in combination with a fast-killing clonal isolate of SfMNPV (Harrison et al., 2008).

We have now determined the complete genome sequences for two isolates, SfMNPV-459 and SfMNPV-1197, and compared them to previously determined genome sequences of SfMNPV isolates from Missouri, Brazil, and Nicaragua. In addition, the viruses were tested and shown to be capable of being used as a biopesticide to control populations of fall armyworm.

### SUMMARY OF THE INVENTION

Novel viruses capable of infecting fall armyworms (Spodoptera frugiperda) were isolated and sequences of the viruses were determined. The viruses were shown to be capable of being used as a biopesticide to control populations of fall armyworm.

An isolated Spodoptera frugiperda multiple nucleopolyhedrovirus strain having SEQ ID NO: 1 or SEQ ID NO: 2, and optionally a carrier.

A biocontrol composition containing an effective amount of Spodoptera frugiperda multiple nucleopolyhedrovirus strain having SEQ ID NO: 1 or SEQ ID NO: 2 or a combination of said strains and optionally a carrier to at least reduce the number of Spodoptera frugiperda in an area.

A biocontrol method for killing insects (e.g., Spodoptera frugiperda), involving spreading the above composition at or near an insect infestation.

A method for killing insects (e.g., Spodoptera frugiperda), involving treating an object or area with an insect killing effective amount of a composition comprising the above isolated Spodoptera frugiperda multiple nucleopolyhedrovirus strain and optionally a carrier.

A method of reducing the population of Spodoptera frugiperda in an infestation or eradicating an infestation of Spodoptera frugiperda involving spreading the above isolated Spodoptera frugiperda multiple nucleopolyhedrovirus strain and optionally a carrier in areas around a Spodoptera frugiperda infestation or in areas where the Spodoptera frugiperda population feeds.

A method of protecting plants from Spodoptera frugiperda involving spreading the above isolated Spodoptera frugiperda multiple nucleopolyhedrovirus strain and optionally a carrier in areas around said plants.

A method to reduce or eradicate a population of Spodoptera frugiperda involving applying the above isolated Spodoptera frugiperda multiple nucleopolyhedrovirus strain and optionally a carrier to a field in a prophylactic manner before Spodoptera frugiperda larvae appear.

This summary is provided to introduce a selection of concepts in a simplified form that are further described below in the detailed description. This summary is not intended to identify key features or essential features of the claimed subject matter, nor is it intended as an aid in determining the scope of the claimed subject matter.

### Deposit Of The Microorganisms

Spodoptera frugiperda multiple nucleopolyhedrovirus virus strain 459 has been deposited under the provisions of the Budapest Treaty on 9 May 2014 (ATCC PTA-121214) with American Type Culture Collection (ATCC), 10801 University Blvd., Manassas, Va., 20110-2209, U.S.A.

Spodoptera frugiperda multiple nucleopolyhedrovirus virus strain 1197 has been deposited under the provisions of the Budapest Treaty on 9 May 2014 (ATCC PTA-121213) with American Type Culture Collection (ATCC), 10801 University Blvd., Manassas, Va., 20110-2209, U.S.A.

Thus a deposit of Spodoptera frugiperda multiple nucleopolyhedrovirus virus strain 459 (ATCC PTA-121214) and strain 1197 (ATCC PTA-121213) has been made in a depository affording permanence of the deposit and ready accessibility thereto by the public if a patent is granted. The depository is American Type Culture Collection (ATCC), 10801 University Blvd., Manassas, Va., 20110-2209, U.S.A. All restrictions on the availability to the public of the materials so deposited will be irrevocably removed upon the granting of a patent. The materials have been deposited under conditions that access to the materials will be available during the pendency of the patent application to one determined by the Commissioner to be entitled thereto under 37 CFR Section 1.14 and 35 U.S.C Section 122. The deposited materials will be maintained with all the care necessary to keep it viable and uncontaminated for a period of at least five years after the most recent request for the furnishing of a sample of the deposited microorganisms, and in any case, for a period of at least thirty (30) years after the date of deposit for the enforceable life of the patent, whichever period is longer.

### BRIEF DESCRIPTION OF THE DRAWINGS

FIG. 1 shows gene parity plots comparing ORF content and order of SfMNPV-3AP2, the reference strain of Spodoptera frugiperda multiple nucleopolyhedrovirus, with (a) SfMNPV-459 and (b) SfMNPV-1197 as described below; ORFs present in only one of the compared genomes appear on the axis corresponding to the virus in which they are present.

FIG. 2 shows phylogenetic analysis of (a) amino acid and (b) nucleotide sequence alignments for five genes from SfMNPV isolates with completely sequenced genomes as described below; minimum evolution phylograms inferred from the concatenated alignments of the genes orf1629, vp80, cg30, pp31, and rr1 are shown with bootstrap values ≧50% at each node where available.

### SEQUENCE LISTING

Any nucleic acid and amino acid sequences listed herein or in the accompanying sequence listing are shown using standard letter abbreviations for nucleotide bases, and three letter code for amino acids, as defined in 37 C.F.R. Section 1.822. In at least some cases, only one strand of each nucleic acid sequence is shown, but the complementary strand is understood as included by any reference to the displayed strand.

SEQ ID NO: 1 shows the nucleic acid sequence of Spodoptera frugiperda multiple nucleopolyhedrovirus virus strain 459 (ATCC PTA-121214).

SEQ ID NO: 2 shows the nucleic acid sequence of Spodoptera frugiperda multiple nucleopolyhedrovirus virus strain 1197 (ATCC PTA-121213).

### DETAILED DESCRIPTION OF THE INVENTION

Novel viruses capable of infecting fall armyworms (Spodoptera frugiperda) were isolated and sequences of the viruses were determined and set forth in SEQ ID NOs: 1-2. The viruses were shown to be capable of being used as a biopesticide to control populations of fall armyworm.

An isolated Spodoptera frugiperda multiple nucleopolyhedrovirus strain having SEQ ID NO: 1 or SEQ ID NO: 2, and optionally a carrier.

A biocontrol composition containing an effective amount of Spodoptera frugiperda multiple nucleopolyhedrovirus strain having SEQ ID NO: 1 or SEQ ID NO: 2 or a combination of said strains and optionally a carrier to at least reduce the number of Spodoptera frugiperda in an area.

A biocontrol method for killing insects (e.g., Spodoptera frugiperda), involving spreading the above composition at or near an insect infestation.

A method for killing insects (e.g., Spodoptera frugiperda), involving treating an object or area with an insect killing effective amount of a composition comprising the above isolated Spodoptera frugiperda multiple nucleopolyhedrovirus strain and optionally a carrier.

A method of reducing the population of Spodoptera frugiperda in an infestation or eradicating an infestation of Spodoptera frugiperda involving spreading the above isolated Spodoptera frugiperda multiple nucleopolyhedrovirus strain and optionally a carrier in areas around a Spodoptera frugiperda infestation or in areas where the Spodoptera frugiperda population feeds.

A method of protecting plants from Spodoptera frugiperda involving spreading the above isolated Spodoptera frugiperda multiple nucleopolyhedrovirus strain and optionally a carrier in areas around said plants.

A method to reduce or eradicate a population of Spodoptera frugiperda involving applying the above isolated Spodoptera frugiperda multiple nucleopolyhedrovirus strain and optionally a carrier to a field in a prophylactic manner before Spodoptera frugiperda larvae appear.

“Biocontrol agent” and “biopesticide” are interchangeable terms and are broadly defined as a composition containing a protein, glycoprotein, polysaccharide, lipid, or other substance produced by animals, plants, bacteria, viruses, phages, fungi, protozoa, etc., that, when a pest ingests, touches, or otherwise comes in contact with the composition, exerts a deleterious effect on the pest. Such deleterious effect can include, but is not limited to, inhibiting reproduction and/or killing the pest. Viruses, bacteria, phages, protozoa, fungi, etc., can be biocontrol agents or biopesticides in that these organisms can infect the pest, injure, and/or kill the pest. Further, some animals are biocontrol agents or biopesticides, such as endoparasitic wasps. The virus strains disclosed herein, including 459 and 1197, can be a biocontrol agent or biopesticide for fall armyworms. These strains can be mass-produced by infection of its host insect in a rearing facility, followed by isolating progeny virus from cadavers of the infected host, and formulating the isolated virus for application in the field.

A biopesticide can optionally include a carrier which can be a liquid or a solid material. The carrier usually is an inert agent that does not repel the pest. The carrier may assist in the delivery of the biocontrol agent that targets the pest or may prolong the persistence and activity of the biocontrol agent in the field. The carrier may be a food source for fall armyworms. A carrier can be a liquid or gel, such as, but not limited to, water, sugar water, saline solution, oil, or any other liquid or gel that does not adversely affect the viability and/or activity of the biocontrol organism or compound. A solid carrier can be, for example, the pest's food or a substance that assists with the application or distribution of the biocontrol agent. The carrier as used herein is defined as not including the body of an insect (e.g., Spodoptera frugiperda).

Optionally, a chemical pesticide, insecticide, or synergists can be included in the biopesticide. Non-limiting examples of pesticides, insecticides, or synergists for this invention include, abamectin, dinotefuran, avermectins, chlorfenapyr, indoxacarb, metaflumizone, imidacloprid, fipronil, hydramethylon, sulfluramid, hexaflumuron, pyriproxyfen, methoprene, lufenuron, dimilin, chlorpyrifos, neem, azadiractin, boric acid, their active derivatives, and the like. These pesticides/insecticides act as stressor which may be required to initiate replication of the biocontrol organism which, in turn, results in death of the pests.

An “effective amount” or “amount effective for” is the minimum amount of a biocontrol agent to affect the desired effect on the organism targeted by the biocontrol agent. For this invention, an “effective amount” or “amount effective for” is the minimum amount of the virus(es) or composition containing the virus(es) needed to cause the death of fall armyworms. An effective amount of the virus(es) will infect and kill a sufficient number of fall armyworms such that the colony is reduced in size as compared to a similar colony that is not treated, or such that the colony collapses completely thereby eradicating the fall armyworms. The precise amount needed may vary in accordance with the particular virus used, the other components of the biopesticide, the colony being treated, the environment in which the colony is located, and the environment before, during, and after application of the biocontrol agent. The exact amount of virus needed per dose of biopesticide and/or the amount of biopesticide needed can be easily determined by one of ordinary skill in the art using the teachings presented herein. An “effective amount” of active agent is a dose sufficient to either prevent or treat armyworm infestation in plants to which the active agent is administered.

The term “treatment” or “treating” as used herein covers any treatment of a plant, and includes (i) preventing the armyworm infestation from occurring in a plant or (ii) fully or partially eradicating the armyworm infestation of a plant. References in this specification to treatment or treating include prophylactic treatment as well as the full or partial eradication of the armyworms on a plant.

The method of using the virus(es) described herein to reduce or eradicate a population of fall armyworms may involve spreading, distributing, or administering the virus(es) described herein or the biopesticide described herein to fall armyworms, their colonies, areas around their colonies, and/or areas where the fall armyworms obtain food. The amount of biopesticide used is an effective amount for killing fall armyworms, reducing the size of the colony compared to an untreated colony, or fully or partially eradicating the fall armyworms and their colony. The method of using the virus(es) described herein to reduce or eradicate a population of fall armyworms includes applying the virus(es) to a field in a prophylactic manner before fall armyworm larvae appear. For example, the virus(es) can be added to an early season spray with another insecticide/herbicide long before the target pest appears.

The terms “isolated”, “purified”, or “biologically pure” as used herein, refer to material that is substantially or essentially free from components that normally accompany the material in its native state. An isolated or purified virus is a virus that is separated from other viruses with which it is found in nature or from the virus' host.

Unless defined otherwise, all technical and scientific terms used herein have the same meaning as commonly understood by one of ordinary skill in the art to which the invention belongs. As used herein, the term “about” refers to a quantity, level, value or amount that varies by as much as 30%, preferably by as much as 20%, and more preferably by as much as 10% to a reference quantity, level, value or amount. Although any methods and materials similar or equivalent to those described herein can be used in the practice or testing of the present invention, the preferred methods and materials are now described.

The following examples are intended only to further illustrate the invention and are not intended to limit the scope of the invention as defined by the claims.

### EXAMPLES

Virus isolates and insects: S. frugiperda eggs were purchased from Benzon Research (Carlisle, Pa.) or provided by Rob Meagher, USDA-ARS, Gainesville, Fla. The Benzon Research strain was derived from the corn strain of S. frugiperda, while the colony insects provided by USDA-ARS were established from the rice strain of S. frugiperda. Larvae were reared on an artificial diet either from Bio-Serv (NJ) or from Southland Products (AR) as previously described (Behle and Popham, 2012). SfMNPV isolates examined in this study consist of samples from an insect virus collection maintained in Beltsville, Md. (Rowley, D. L., et al., Virus Genes, 40: 458-68 (2010)) and also a series of more recently collected isolates from Missouri (Harrison et al., 2008).

Bioassays: To produce occlusion bodies (OBs) for bioassays, third- and fourth-instar larvae were inoculated with stocks of the individual isolates, and OBs were isolated from virus-killed cadavers as previously described (Harrison, R. L., Virus Genes, 38: 155-70 (2009)).

OB stocks were counted on a hemacytometer before the start of each bioassay and diluted in water. Neonate S. frugiperda larvae were infected per os by the droplet feeding method developed by Hughes (Hughes, P. R., et al., Journal of Invertebrate Pathology, 48: 187-192 (1986)) with six doses of occlusion bodies (OBs) ranging from 1×10⁵ to 1×10⁸ OBs/ml. Larvae were placed on fresh food, maintained at 28±1° C. at a photoperiod of 14:10 h (L:D), and monitored two or three times daily for 7 days. The LC₅₀ (concentration of OBs required to kill 50% of the test larvae) for each virus was calculated by Proc Logisitic using SAS vers. 9.1, and hypotheses concerning the parallelism and equality of probit dose-response lines were tested using the same program. Median mortality times (LT₅₀) were calculated with survivors excluded using the Kaplan-Meier Estimator and mortality data of the 1×10⁷ OBs/mL dose for all the viruses. Comparison of LT₅₀s was computed using the log-rank test by SigmaPlot version 11 (Systat Software, Inc., San Jose, Calif.).

SfMNPV isolates 459 (Colombia) and 3AP2 (Missouri) were combined in different ratios and tested in bioassay on Spodoptera frugiperda. Results from three bioassays of the SfMNPV isolate mixtures tested over five doses per mixture (30 insects/dose) were combined for LC₅₀ calculation, but LT₅₀s were calculated separately for two iterations of the survival time bioassay.

Genomic DNA preparation and sequencing: Genomic DNA of SfMNPV-459 and SfMNPV-1197 was isolated from OBs extracted from virus-killed S. frugiperda cadavers as previously described (Harrison, R. L., et al., J. Invertebr. Pathol., 116: 27-35 (2014)). Genomic DNA samples were sheared and size-fractionated. A multiplexed Roche GS FLX Titanium library was prepared for each isolate and sequenced at the Georgia Genomics Facility (https://gsle.ovpr.uga.edu). Reads were sorted and assembled using the Lasergene SeqMan NGEN V3.0 (DNAStar) assembler program with default parameters. Gaps were closed and sequencing ambiguities were resolved by PCR amplification and dideoxy sequencing of the corresponding genomic regions from viral DNA. The SeqManPro V9 sequence editor was used to prepare the final contigs.

Genome annotation and phylogenetic analysis: Open reading frames (ORFs)≧50 codons in size that did not overlap adjacent ORFs by more than 75 by and did not occur in a homologous repeat region (hr) were selected for annotation and further analysis. If two candidate ORFs overlapped by more than 75 bp, the larger ORF was selected for annotation. ORFs with annotated homologs in other baculovirus genomes were also listed. Gene parity plot analysis was carried out as previously described (Hu, Z. H., et al., J. Gen. Virol., 79(Pt 11): 2841-51 (1998)). BLAST queries and alignment of amino acid and nucleotide sequences were carried out using the software of the DNASTAR Lasergene suite (version 11). Phylogenetic inference from amino acid and nucleotide alignments were carried out using MEGA6 (Tamura, K., et al., Mol. Biol. Evol., 30: 2725-9 (2013) using a minimum evolution algorithm with the Jones-Taylor-Thornton (amino acid) and Tajima-Nei (nucleotide) substitution models and gamma parameters of 2.12 and 0.05, respectively.

Results: Biological activity of SfMNPV isolates: Bioassays were carried out on a selection of SfMNPV isolates that were evaluated in two different groups on S. frugiperda from the corn strain. The first group (Group 1; Tables 1 and 2) included isolates 651, 652, 653, 654, and 1197, all from Georgia, USA; along with isolate 2705 from an unknown source and isolate 459 from Medellin, Colombia. The second group (Group 2; Tables 3 and 4) included isolates 459 and two additional Colombian isolates, 635 and 636; 3AP2 (MO), Sf3 (MO), 281 (GA), and 3146, an isolate from an unknown source. LC₅₀ values exhibited a 4.1-fold range among the Group 1 isolates (Table 1), but a wider 14.3-fold range among Group 2 isolates (Table 3). The results from the lethal time bioassays (Tables 2 and 4) suggest the classification of the isolates into two categories: those with a rapid speed of kill, and those that kill larvae more slowly. Larvae infected with the isolates 459, 651, 653, 1197, 3146, and 3AP2 surprisingly die with LT₅₀s that are consistently lower than isolates 635, 636, 637, 638, 652, 654, 2705, and Sf3. LT₅₀ values for isolate 281 seem to occur between the two categories. Six of the SfMNPV isolates in the previous bioassays were selected for testing in S. frugiperda larvae from the rice strain (Tables 5 and 6). Surprisingly, the rice strain larvae were found to be more susceptible (2-5× lower LC₅₀s) to all of the SfMNPV isolates than larvae from the corn strain (Table 5). In lethal time testing, no differences in LT₅₀s were observed between the tested isolates though several (#459, 636, 652, and 1197) surprisingly had slightly lower LC₅₀s than #281 and #3AP2 (Table 6). Interestingly, the LT₅₀s of the SfMNPV-infected rice strain larvae were roughly 10 hours longer than for the corn strain larvae.

To identify potential additive or synergistic effects of infections with an inoculum containing two different SfMNPV strains, lethal concentration and lethal time bioassays were carried out with mixtures of the fast-killing 3AP2 and 459 isolates (Tables 7 and 8). Mixtures consisting of ratios of 100%, 75%:25%, and 50%:50% were tested. The LC₅₀s were found to be tightly grouped for the two individual strains and for the mixtures (Table 7). The 25% 459/75% 3AP2 blend had a slightly lower LC₅₀ though the difference was marginal. LT₅₀s also grouped very closely (Table 8). Without being bound by theory, any statistical differences in LT₅₀ were more likely to be a result of a difference in check times versus setup times than true differences between the test groups.

Genome sequences of SfMNPV isolates 459 and 1197: Based on bioassay results, we selected SfMNPV isolates 459 (COL) and 1197 (GA, USA) from the group of fast-killing isolates from the corn strain S. frugiperda bioassays for determination of complete genome nucleotide sequences. The total genome size and nucleotide distribution (% GC) of isolate 1197 were very similar to those of isolates SfMNPV-19 (Brazil) and SfMNPV-B (Nicaragua) (Table 9). The 459 isolate, however, surprisingly had the largest genome recorded among SfMNPV genomes sequenced to date, with a higher G+C percentage. Pairwise alignments of all SfMNPV genome sequences determined to date revealed that the genome sequences all share high nucleotide sequence identity (>99%; Table 10). While the 1197 isolate shared sequence identities of 99.5-99.9% to other SfMNPV isolates, the sequence identities of isolate 459 ranged from 99.1-99.2%. Also, both the number of gaps required to optimize alignments and the total length of the gaps in base pairs (bp) were surprisingly larger in alignments between 459 and other SfMNPV isolates. These trends surprisingly indicated that SfMNPV-459 possesses a more divergent genome sequence relative to other SfMNPV genomes sequenced to date.

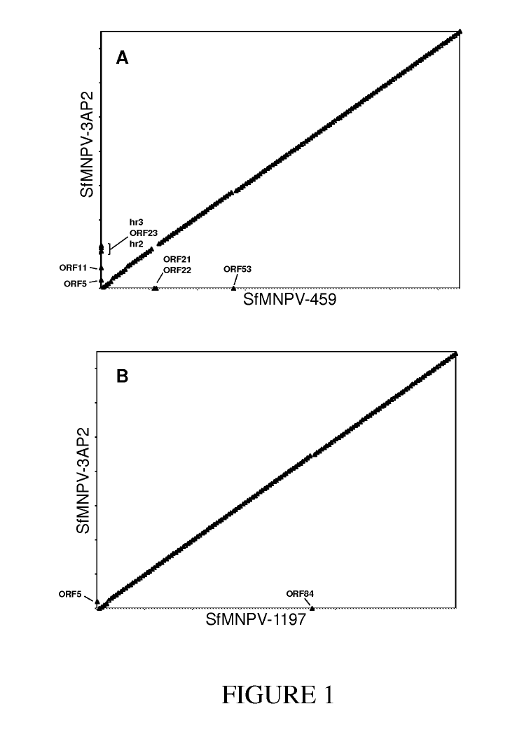

To detect differences in gene content among SfMNPV isolates, gene parity plot analysis was carried out to compare open reading frame (ORF) distribution and position between isolates 459 and 1197 and isolate 3AP2 (whose genome sequence is the reference sequence of species Spodoptera frugiperda multiple nucleopolyhedrovirus). The results (FIG. 1) indicated that the genome sequences of isolates 459 and 1197 were largely co-linear with that of isolate 3AP2. Surprisingly, while both 459 and 1197 did not contain SfMNPV-3AP2 ORF5, 459 was also missing 3AP2 ORFs 11 and 23. While 3AP2 ORFs 5 and 23 were also found in the genomes of SfMNPV-19 and SfMNPV-B, ORF11 had only been identified in isolate 3AP2 to date. Isolate 459 surprisingly was also missing two homologous repeat regions (hrs) present in the other SfMNPV isolates. Surprisingly isolate 1197 contained one ORF (ORF84) and isolate 459 contained three ORFs (ORFs 21, 22, and 53) with no homologues in other SfMNPV isolates. ORF84 of isolate 1197 and ORF53 of isolate 459 surprisingly did not match any baculovirus ORFs. Isolate 459 ORF21 was a homologue of Spodoptera exigua multiple nucleopolyhedrovirus (SeMNPV) ORF21, while ORF22 was a homologue of SeMNPV ORFs 22, 23, and 24 combined. These two ORFs were surprisingly present in a novel, 2.4 kbp sequence present in isolate 459 but not in any other SfMNPV genome. Surprisingly, this sequence shared 94% nucleotide sequence identity with an isolate of Spodoptera litura nucleopolyhedrovirus (SpltNPV), suggesting (without being bound by theory) that isolate 459 may have acquired this sequence by co-infection of a Spodoptera host followed by recombination between the genomes of isolate 459 and SpltNPV.

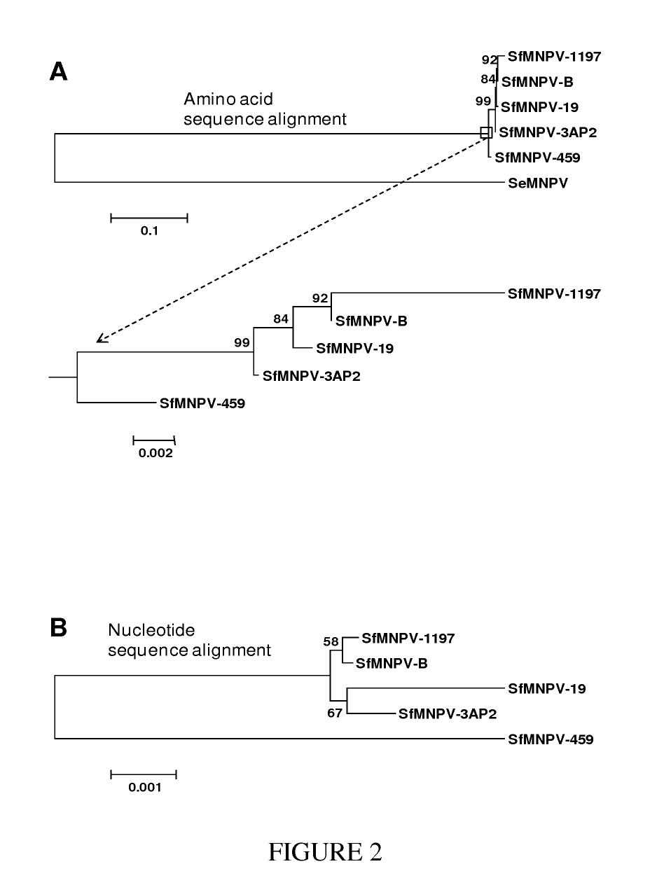

Relationships of fully-sequenced SfMNPV isolates: To assess the relationships among the SfMNPV isolates with fully-sequenced genomes, phylogenetic inference was carried out using concatenated alignments of the genes orf1629, vp80, cg30, pp31, and rr1. These genes were selected because they appeared to be relatively divergent in sequence among the five SfMNPV genomes. Surprisingly, phylogenetic inference with alignments of both amino acid (FIG. 2A) and nucleotide (FIG. 2B) sequences indicated that isolates 3AP2, B, 19, and 1197 form a distinct group, while isolate 459 was placed by itself on a separate branch. Furthermore, B and 1197 grouped together in a clade with moderate to strong bootstrap support, suggesting (without being bound by theory) they are closely related. However, the branch lengths separating isolates B and 1197 surprisingly were close to half the total length of the SfMNPV clade inferred from amino acid sequence alignments (FIG. 2A), indicating a relatively high degree of divergence between these two isolates.

Discussion: Lethal time bioassay data in this study for S. frugiperda originating from the corn strain indicated that individual SfMNPV isolates can be grouped into a category of highly virulent, fast-killing isolates and one of less virulent, slower-killing isolates. Pathogenicity (LC₅₀) in two out of three groups of isolates tested varied by <5-fold, while one group exhibited a >10-fold variance. Bioassay data for the rice strain of S. frugiperda demonstrated little variation among SfMNPV isolates. Overall differences were noted between the corn and rice strain LC₅₀s and LT₅₀s. Rice strain larvae were found to be susceptible to lower doses of SfMNPV isolates but had somewhat longer survival times relative to corn strain larvae.

Faster-killing isolates (i.e., those with lower LT₅₀ values) are of greater value for control of fall armyworm than slower-killing isolates because of the reduced quantity of fall armyworm larval feeding damage and subsequent reduced yield loss that would be expected with a faster-killing isolate. We determined the complete genome sequences of two isolates from the faster killing category to obtain a profile of the genetics behind rapid speed of kill. A comparison of the genomic sequence of these two isolates, 459 and 1197, with other previously sequenced isolates revealed that 459 surprisingly had three ORFs (ORFs 21, 22, and 53) and 1197 had one ORF (ORF84) that were not found in the other sequenced SfMNPV isolates. Isolate 1197 grouped with the other SfMNPV isolates in a phylogeny based on five genes, while isolate 459 was surprisingly more divergent and occurred on a branch by itself. Surprisingly, there seemed to be little congruence between the geographic locations where SfMNPV isolates originated from and their relationships inferred from amino acid and nucleotide sequence alignments (FIG. 2).

SfMNPV offers considerable promise as a safe, ecologically friendly means of controlling infestations of S. frugiperda where they occur. This study identifies isolates with superior properties for control of fall armyworm and provides extensive information on the genetics of two of these viruses.

All of the references cited herein, including U.S. Patents, are incorporated by reference in their entirety.

Thus, in view of the above, there is described (in part) the following:

An isolated Spodoptera frugiperda multiple nucleopolyhedrovirus strain having SEQ ID NO: 1 or SEQ ID NO: 2, and optionally a carrier. The above isolated Spodoptera frugiperda multiple nucleopolyhedrovirus strain, having SEQ ID NO: 1, and optionally a carrier. The above isolated Spodoptera frugiperda multiple nucleopolyhedrovirus strain, wherein said strain is ATCC PTA-121214, and optionally a carrier. The above isolated Spodoptera frugiperda multiple nucleopolyhedrovirus strain, having SEQ ID NO: 2, and optionally a carrier. The above isolated Spodoptera frugiperda multiple nucleopolyhedrovirus strain, wherein said strain is ATCC PTA-121213, and optionally a carrier.

A biocontrol composition comprising (or consisting essentially of or consisting of) an effective amount of Spodoptera frugiperda multiple nucleopolyhedrovirus strain having SEQ ID NO: 1 or SEQ ID NO: 2 or a combination of said strains and optionally a carrier to at least reduce the number of Spodoptera frugiperda in an area.

A biocontrol method for killing insects (e.g., Spodoptera frugiperda), comprising (or consisting essentially of or consisting of) spreading the above composition at or near an insect infestation.

A method for killing insects(e.g., Spodoptera frugiperda), comprising (or consisting essentially of or consisting of) treating an object or area with an insect killing effective amount of a composition comprising the above isolated Spodoptera frugiperda multiple nucleopolyhedrovirus strain and optionally a carrier. The above method, wherein said insects are Spodoptera frugiperda.

A method of reducing the population of Spodoptera frugiperda in an infestation or eradicating an infestation of Spodoptera frugiperda comprising (or consisting essentially of or consisting of) spreading the above isolated Spodoptera frugiperda multiple nucleopolyhedrovirus strain and optionally a carrier in areas around a Spodoptera frugiperda infestation or in areas where the Spodoptera frugiperda population feeds.

A method of protecting plants from Spodoptera frugiperda comprising (or consisting essentially of or consisting of) spreading the above isolated Spodoptera frugiperda multiple nucleopolyhedrovirus strain and optionally a carrier in areas around said plants.

A method to reduce or eradicate a population of Spodoptera frugiperda comprising (or consisting essentially of or consisting of) applying the above isolated Spodoptera frugiperda multiple nucleopolyhedrovirus strain and optionally a carrier to a field in a prophylactic manner before Spodoptera frugiperda larvae appear.

Other embodiments of the invention will be apparent to those skilled in the art from a consideration of this specification or practice of the invention disclosed herein. It is intended that the specification and examples be considered as exemplary only, with the true scope and spirit of the invention being indicated by the following claims.

TABLE 1
Lethal concentration bioassays of SfMNPV isolates, Test Group 1 (459 and
1197 characteristics are highlighted in grey)

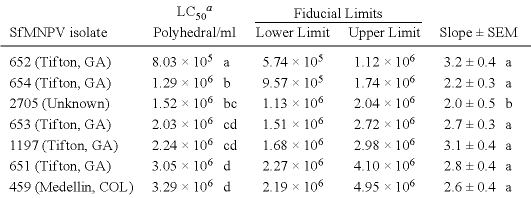

^{a}Isolates listed in order of increasing LC₅₀ value. Values with different letters are significantly different at P < 0.05.

TABLE 2
Lethal time bioassays of SfMNPV isolates, Test Group 1 (459
and 1197 characteristics are highlighted in grey)

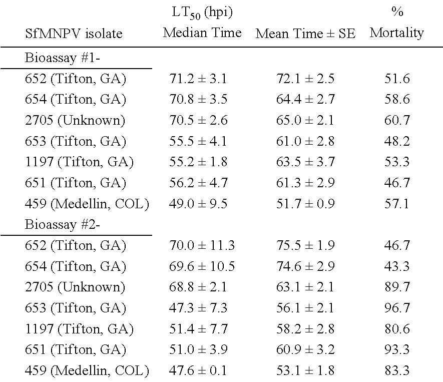

TABLE 3
Lethal concentration bioassays of SfMNPV isolates, Test Group 2
(459 characteristics are highlighted in grey)

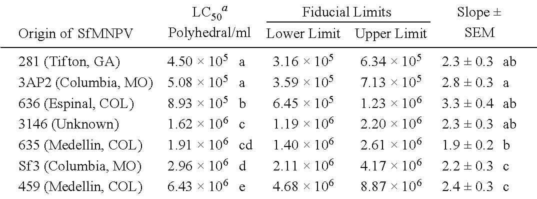

^{a}Isolates listed in order of increasing LC₅₀ value. Values with different letters are significantly different at P < 0.05.

TABLE 4
Lethal time bioassays of SfMNPV isolates, Test Group 2 (459
characteristics are highlighted in grey)

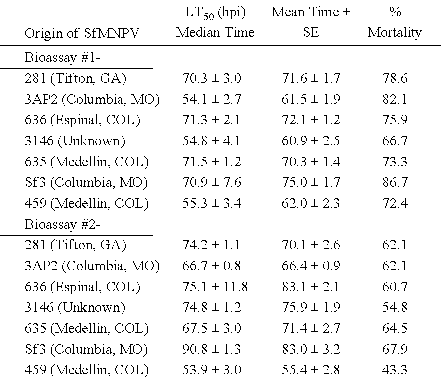

TABLE 5
Lethal concentration bioassays of SfMNPV isolates on rice strain S. frugiperda
(459 and 1197 characteristics are highlighted in grey)

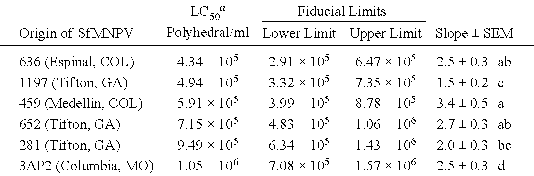

^{a}Isolates listed in order of increasing LC₅₀ value. Values with different letters are significantly different at P < 0.05.

TABLE 6
Lethal time bioassays of SfMNPV isolates on rice strain S.
frugiperda (459 and 1197 characteristics are highlighted in grey)

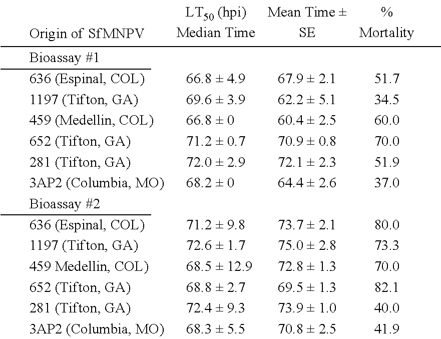

TABLE 7
Lethal concentration bioassays of mixtures of SfMNPV
isolates 459 and 3AP2

| | LC₅₀ ^{a} | Fiducial Limits | | |
|---|---|---|---|---|
| | Polyhedral/ | Lower | Upper | Slope ± |
| Isolate mixture | ml | Limit | Limit | SEM |
| 100% 459 | 2.51 × 10⁶ a | 1.92 × 10⁶ | 3.27 × 10⁶ | 2.4 ± 0.2 ab |
| 75% 459/25% 3AP2 | 2.83 × 10⁶ c | 2.19 × 10⁶ | 3.67 × 10⁶ | 1.9 ± 0.2 a |
| 50% 459/50% 3AP2 | 1.82 × 10⁶ ab | 1.40 × 10⁶ | 2.36 × 10⁶ | 2.6 ± 0.3 b |
| 25% 459/75% 3AP2 | 1.51 × 10⁶ b | 1.14 × 10⁶ | 1.99 × 10⁶ | 2.6 ± 0.3 ab |
| 100% 3AP2 | 1.87 × 10⁶ ab | 1.41 × 10⁶ | 2.47 × 10⁶ | 2.1 ± 0.2 a |

^{a} Values with different letters are significantly different at P < 0.05.

TABLE 8
Lethal time bioassays of mixtures of SfMNPV isolates 459 and 3AP2

| | LT₅₀ (hpi) | Mean | % |
|---|---|---|---|
| | Median Time | Time ± SE | Mortality |
| Origin of SfMNPV | | | |
| Bioassay #1 | | | |
| 2.5 × 10⁶ OBs/ml | | | |
| 100% 459 | 50.6 ± 2.1 | 52.5 ± 1.3 | 55.2 |
| 75% 459/25% 3AP2 | 51.0 ± 1.4 | 56.3 ± 2.6 | 48.3 |
| 50% 459/50% 3AP2 | 57.1 ± 2.1 | 59.1 ± 3.1 | 69.0 |
| 25% 459/75% 3AP2 | 50.3 ± 0.7 | 51.1 ± 1.3 | 63.3 |
| 100% 3AP2 | 50.2 ± 0.7 | 49.2 ± 1.8 | 31.0 |
| 1 × 10⁷ OBs/ml | | | |
| 100% 459 | 44.3 ± 6.8 | 50.2 ± 1.3 | 90.0 |
| 75% 459/25% 3AP2 | 51.0 ± 1.6 | 51.3 ± 1.6 | 66.7 |
| 50% 459/50% 3AP2 | 50.3 ± 1.4 | 54.6 ± 1.8 | 90.0 |
| 25% 459/75% 3AP2 | 50.7 ± 0.9 | 50.3 ± 1.1 | 86.7 |
| 100% 3AP2 | 50.2 ± 2.5 | 49.2 ± 0.8 | 93.3 |
| Bioassay #2 | | | |
| 2.5 × 10⁶ OBs/ml | | | |
| 100% 459 | 55.0 ± 1.6 | 55.2 ± 1.9 | 66.7 |
| 75% 459/25% 3AP2 | 54.8 ± 2.4 | 57.3 ± 2.3 | 66.3 |
| 50% 459/50% 3AP2 | 54.7 ± 1.8 | 55.4 ± 2.3 | 50.0 |
| 25% 459/75% 3AP2 | 47.3 ± 7.1 | 53.1 ± 1.7 | 69.0 |
| 100% 3AP2 | 54.1 ± 1.7 | 55.7 ± 2.2 | 69.6 |
| 1 × 10⁷ OBs/ml | | | |
| 100% 459 | 55.0 ± 5.5 | 54.7 ± 1.9 | 78.6 |
| 75% 459/25% 3AP2 | 54.8 ± 2.2 | 56.7 ± 2.0 | 88.5 |
| 50% 459/50% 3AP2 | 54.6 ± 1.3 | 54.8 ± 1.6 | 92.9 |
| 25% 459/75% 3AP2 | 47.3 ± 8.9 | 50.9 ± 1.3 | 94.7 |
| 100% 3AP2 | 54.0 ± 1.0 | 56.6 ± 1.7 | 55.6 |

TABLE 9
Characteristics of SfMNPV genome sequences (459 and 1197
characteristics are highlighted in grey)

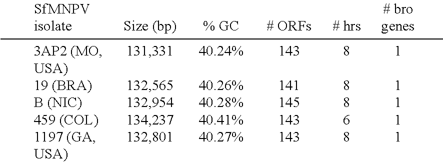

TABLE 10
Pairwise alignment and comparison of sequenced SfMNPV genomes

| | % sequence | Number | Total gap |
|---|---|---|---|
| Alignment | identity | of gaps | length, bp |
| B × 1197 | 99.9% | 8 | 585 |
| 3AP2 × 19 | 99.6% | 59 | 2106 |
| 3AP2 × B | 99.6% | 64 | 2365 |
| 3AP2 × 1197 | 99.6% | 66 | 1806 |
| 19 × B | 99.5% | 62 | 1149 |
| 19 × 1197 | 99.5% | 63 | 596 |
| B × 459 | 99.2% | 110 | 4239 |
| 459 × 1197 | 99.2% | 113 | 3702 |
| 19 × 459 | 99.2% | 127 | 4090 |
| 3AP2 × 459 | 99.1% | 117 | 5256 |

We claim:

1. An isolated Spodoptera frugiperda multiple nucleopolyhedrovirus strain having SEQ ID NO: 1 or SEQ ID NO: 2.

2. The isolated Spodoptera frugiperda multiple nucleopolyhedrovirus strain according to claim 1, having SEQ ID NO: 1.

3. The isolated Spodoptera frugiperda multiple nucleopolyhedrovirus strain according to claim 1, wherein said strain is ATCC PTA-121214.

4. The isolated Spodoptera frugiperda multiple nucleopolyhedrovirus strain according to claim 1, having SEQ ID NO: 2.

5. The isolated Spodoptera frugiperda multiple nucleopolyhedrovirus strain according to claim 1, wherein said strain is ATCC PTA-121213.

6. A biocontrol composition comprising an effective amount of Spodoptera frugiperda multiple nucleopolyhedrovirus strain having SEQ ID NO: 1 or SEQ ID NO: 2 or a combination of said strains and optionally a carrier to at least reduce the number of Spodoptera frugiperda in an area.

7. A biocontrol method for killing insects, comprising spreading the isolated Spodoptera frugiperda multiple nucleopolyhedrovirus strain of claim 1 and optionally a carrier at or near an insect infestation.

8. A method for killing insects, comprising treating an object or area with an insect killing effective amount of a composition comprising the isolated Spodoptera frugiperda multiple nucleopolyhedrovirus strain of claim 1 and optionally a carrier.

9. The method according to claim 8, wherein said insects are Spodoptera frugiperda.

10. A method of reducing the population of Spodoptera frugiperda in an infestation or eradicating an infestation of Spodoptera frugiperda comprising spreading the isolated Spodoptera frugiperda multiple nucleopolyhedrovirus strain of claim 1 and optionally a carrier in areas around a Spodoptera frugiperda infestation or in areas where the farm armyworm population feeds.

11. A method of protecting plants from Spodoptera frugiperda comprising spreading the isolated Spodoptera frugiperda multiple nucleopolyhedrovirus strain of claim 1 and optionally a carrier in areas around said plants.

12. A method to reduce or eradicate a population of Spodoptera frugiperda comprising applying the isolated Spodoptera frugiperda multiple nucleopolyhedrovirus strain of claim 1 and optionally a carrier to a field in a prophylactic manner before Spodoptera frugiperda larvae appear.
